(19) United States
(12) Patent Application Publication
(10) Pub. No.: US 2022/0296729 A1
(43) Pub. Date: Sep. 22, 2022

(54) METHODS OF DOSING CIRCULAR POLYRIBONUCLEOTIDES

(71) Applicant: Flagship Pioneering Innovations VI, LLC, Cambridge, MA (US)

(72) Inventors: Avak KAHVEJIAN, Lexington, MA (US); Alexandra Sophie DE BOER, Somerville, MA (US); Nicholas McCartney PLUGIS, Duxbury, MA (US); Erica Gabrielle WEINSTEIN, Newton, MA (US); Catherine CIFUENTES-ROJAS, Brookline, MA (US)

(21) Appl. No.: 17/619,638

(22) PCT Filed: Jun. 19, 2020
(86) PCT No.: PCT/US2020/038835

§ 371 Date: Dec. 16, 2021

Related U.S. Application Data
(60) Provisional application No. 62/863,725, filed on Jun. 19, 2019.

(51) Int. Cl. A61K 48/00; C12N 15/67

(52) U.S. Cl. CPC A61K 48/0083; C12N 15/67

(57) ABSTRACT

This invention relates generally to methods of dosing pharmaceutical compositions and preparations of circular polyribonucleotides thereof.

### CROSS-REFERENCE TO RELATED APPLICATIONS

The present application claims priority to and benefit from U.S. Provisional Application No. 62/863,725, filed Jun. 19, 2019, the entire contents of which is herein incorporated by reference.

### BACKGROUND

Certain circular polyribonucleotides are ubiquitously present in human tissues and cells, including tissues and cells of healthy individuals.

### SUMMARY

This present disclosure generally relates to methods of dosing circular polyribonucleotides. The methods as disclosed herein generally relate to a method of expressing a protein in a cell or subject comprising providing a composition of circular polyribonucleotide encoding a protein to the cell or subject, a method of binding a protein in a cell or subject comprising providing a composition of a circular polyribonucleotide comprising a binding site to the cell or subject, or both. A method of dosing comprise providing multiple doses to a cell or subject. For example, a multiple dosing is a redosing or a staggered dosing. A method of redosing of a composition of circular polyribonucleotides comprises providing two or more compositions, generally over an extended period of time, to a cell or subject (e.g. a mammal). A method of a staggered dosing of a composition of circular polyribonucleotide comprises providing two or more compositions generally over a short time interval.

In one aspect, the invention features a method of maintaining expression of a protein in a mammal, comprising: (a) providing a first composition comprising a circular polyribonucleotide that encodes the protein to the mammal; and (b) from 6 hours to 90 days following step (a), providing a second composition comprising a circular polyribonucleotide that encodes the protein, to the mammal, thereby maintaining expression of the protein in the mammal.

In some embodiments of these aspects, the circular polyribonucleotide is an exogenous, synthetic circular polyribonucleotide. In some embodiments, the circular polyribonucleotide lacks a poly-A sequence, a replication element, or both.

In some embodiments of these aspects, the first composition comprises a first circular polyribonucleotide and the second compositions comprises a second circular polyribonucleotide, wherein the first circular polyribonucleotide and the second circular polyribonucleotide are the same. In some embodiments, the first composition comprises a first circular polyribonucleotide and the second compositions comprises a second circular polyribonucleotide, wherein the first circular polyribonucleotide and the second circular polyribonucleotide are different.

In some embodiments of these aspects, providing the second composition occurs after providing the first composition and before a first level of protein expressed by the first composition is substantially undetectable in the mammal. In some embodiments, providing the second composition occurs after providing the first composition and before a first level of protein expressed by the first composition decreases by more than 50% in the mammal.

In some embodiments, the method further comprise providing a third composition of the circular polyribonucleotide to the mammal after the second composition, thereby maintaining expression of the protein in the mammal. In some embodiments, the method further comprise providing a third composition of the circular polyribonucleotide to the mammal after the second composition, thereby restoring expression of the protein in the mammal. In some embodiments, providing the third composition occurs after providing the second composition and before a second level of the protein expressed by the first and second composition is substantially undetectable in the mammal. In some embodiments, providing the third composition occurs after providing the second composition and before a second level of the protein expressed by the first and second composition in the mammal decreases by more than 50%. In some embodiments, the method further comprises providing a fourth, fifth, sixth, seventh, eighth, ninth, or tenth composition of a circular polyribonucleotide encoding the protein.

In some embodiments of these aspects, the first composition further comprises a pharmaceutically acceptable carrier or excipient. In some embodiments, the first composition further comprises a pharmaceutically acceptable excipient and is free of any carrier. In some embodiments, the second composition further comprises a pharmaceutically acceptable carrier or excipient. In some embodiments, the second composition further comprises a pharmaceutically acceptable excipient and is free of any carrier. In some embodiments, the third composition further comprises a pharmaceutically acceptable carrier or excipient. In some embodiments, the third composition further comprises a pharmaceutically acceptable excipient and is free of any carrier.

In some embodiments of these aspects, a first level of the protein expressed by the first composition is a highest level of the protein 1-2 days after providing the first composition. In some embodiments, a first level of the protein expressed by the first composition is 40%, 50%, 60%, 70%, 80%, or 90% of a highest level of the protein one day after providing the first composition. In some embodiments, a second level of the protein is at least 30%, 40%, 50%, 60%, 70%, 80%, 90%, 100%, 110%, 120%, 130%, 140%, 150%, 160%, 170%, 180%, 190%, or 200% of the highest level of the protein one day after providing the first composition for at least 1, 2, 3, 4, 5, 6, 7, 8, 9, 10, 12, 15, 20, 25, 30, 35, or 40 days after providing the second composition. In some embodiments, a third level of the protein is at least 30%, 40%, 50%, 60%, 70%, 80%, 90%, 100%, 110%, 120%, 130%, 140%, 150%, 160%, 170%, 180%, 190%, or 200% of the highest level of the protein one day after providing the first composition for at least 1, 2, 3, 4, 5, 6, 7, 8, 9, 10, 12, 15, 20, 25, 30, 35, or 40 days after providing the third composition. In some embodiments, for each subsequent composition provided after the first composition, a subsequent level of the protein expressed after each subsequent composition is at least 30%, 40%, 50%, 60%, 70%, 80%, 90%, 100%, 110%, 120%, 130%, 140%, 150%, 160%, 170%, 180%, 190%, or 200% of a highest level of the protein one day after providing the first composition for at least 1, 2, 3, 4, 5, 6, 7, 8, 9, 10, 12, 15, 20, 25, 30, 35, or 40 days after providing each subsequent composition.

In some embodiments of these aspects, an average level of the protein after providing the second composition is at least 40%, 50%, 60%, 70%, 80%, 90%, 100% or 110% of a first level of protein from the first composition, wherein the average level of the protein is measured from one day after providing the second composition to the day when the protein is substantially undetectable. In some embodiments, an average level of the protein after providing each subsequent composition after the first composition is at least 40%, 50%, 60%, 70%, 80%, 90%, 100% or 110% of the first level of protein from the first composition, wherein the average level of the protein is measured from one day after providing each subsequent composition to the day when the protein is substantially undetectable. In some embodiments, a first level of the protein is maintained after providing the first composition and the second composition for at least 6 hours, 1 day, 2 days, 3 days, 5 days, 7 days, 14 days, 21 days, 28 days, or 35 days after providing the first composition. In some embodiments, a first level of the protein is maintained after providing the first composition and the second composition for from 6 hours to 90 days after providing the first composition. In some embodiments, a first level of the protein is maintained after providing the first composition, the second composition, and the third composition of circular polyribonucleotide for from 6 hours to 270 days after providing the first composition. In some embodiments, a first level of the protein is substantially undetectable after providing the first composition and the second composition for 6 hours to 35 days after providing the first composition. In some embodiments, a first level of the protein is maintained after providing the first composition, the second composition, and the third composition of the circular polyribonucleotide for at least 6 hours, 1 day, 2 days, 3 days, 5 days, 7 days, 14 days, 21 days, 28 days, or 35 days after providing the first composition. In some embodiments, a second level of protein in the mammal after providing the second composition is at least 1%, 5%, 10%, 20%, 30%, 40%, 50%, or 60% higher than the first level of protein in the mammal after providing the first composition. In some embodiments, a third level of protein produced in the mammal after providing the third composition is at least 5%, 10%, 20%, 30%, 40%, 50%, or 60% higher than the first level of protein in the plurality after providing the first composition. In some embodiments, the second level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the protein after providing the first composition. In some embodiments, a third level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the protein after providing the first composition. In some embodiments, the protein is a therapeutic protein, e.g., erythropoietin. In some embodiments, expression of the protein (e.g., erthryropoietin) induces a response (e.g., reticulocyte production) in the mammal. In some embodiments of the aspects described herein, the therapeutic protein is an enzyme replacement protein, a protein for supplementation, a hormone, a cytokine, an antibody, a protein for immunotherapy (e.g. cancer), a cellular reprogramming/transdifferentiation factor, a transcription factor, a chimeric antigen receptor, a transposase or nuclease, an immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, an epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, or a CRISPR system or component thereof. In some embodiments, the protein is an antigen (e.g., tumor antigen, viral antigen, bacterial antigen). In some embodiments, the protein is a protein for vaccination.

In a second aspect, the invention features a method of maintaining expression of a protein in a cell or subject, comprising providing a first composition comprising a circular polyribonucleotide that encodes the protein to the cell or subject; thereby maintaining expression of the protein in the cell or subject.

In a third aspect, the invention features a method of maintaining expression of a protein in a cell or subject, comprising from 6 hours to 90 days following step (a), providing a second composition comprising a circular polyribonucleotide that encodes the protein, to the cell or subject; thereby maintaining expression of the protein in the cell or subject.

In a fourth aspect, the invention features a method of expressing protein in a cell or a subject comprising providing a first composition comprising a circular polyribonucleotide that encodes a protein to the cell or the subject, wherein the cell or the subject expresses a first level of an encoded protein; and (i) the second level is at least as much as the first level, or (ii) the second level varies by no more than 20% of the first level; thereby maintaining expression of encoded protein in the cell or the subject at least at the first level of the protein.

In a fifth aspect, the invention features a method of expressing protein in a cell or a subject comprising: providing a second composition comprising a circular polyribonucleotide that encodes a protein to the cell or the subject, wherein the cell or the subject expresses a second level of an encoded protein and (i) the second level is at least as much as the first level, or (ii) the second level varies by no more than 20% of the first level; thereby maintaining expression of encoded protein in the cell or the subject at least at the first level of the protein.

In a sixth aspect, the invention features a method of expressing a level of a protein in a cell or subject after providing a first composition and a second composition of a circular polyribonucleotide to the cell or subject compared to a level of the protein in the cell or subject after providing a first composition and second composition of a linear counterpart of the circular polyribonucleotide, comprising: providing a first composition of circular polyribonucleotide encoding a protein to a cell or subject, wherein the cell or subject comprises a level of the protein after providing the first composition of the circular polyribonucleotide; and (i) at least the level of the protein after providing the second composition of the circular polyribonucleotide, or (ii) a level of the protein that varies by no more than 20% of the level after providing the second composition of the circular polyribonucleotide; thereby maintaining expression of the level of the protein in the cell or subject after providing the first composition and the second composition of the circular polyribonucleotide compared to the level of the protein in the cell or subject after providing the first composition and the second composition of a linear counterpart of the circular polyribonucleotide.

In a seventh aspect, the invention features a method of expressing a level of a protein in a cell or subject after providing a first composition and a second composition of a circular polyribonucleotide to the cell or subject compared to a level of the protein in the cell or subject after providing a first composition and second composition of a linear counterpart of the circular polyribonucleotide, comprising: providing a second composition of circular polyribonucleotide after the first composition to the cell or subject, wherein the cell or subject comprises (i) at least the level of the protein after providing the second composition of the circular polyribonucleotide, or (ii) a level of the protein that varies by no more than 20% of the level after providing the second composition of the circular polyribonucleotide; thereby maintaining expression of the level of the protein in the cell or subject after providing the first composition and the second composition of the circular polyribonucleotide compared to the level of the protein in the cell or subject after providing the first composition and the second composition of a linear counterpart of the circular polyribonucleotide.

In some embodiments of these aspects, providing the first composition is to a first cell in the subject and providing the second composition is to a second cell in the subject and wherein the first cell and second cell are the same cell or different cells. In some embodiments, providing the second composition occurs after providing the first composition and before the first level of protein expressed by the first composition is substantially undetectable in the cell or subject. In some embodiments, providing the second composition occurs after providing the first composition and before the first level of protein expressed by the first composition decreases by more than 50% in the cell or subject. In some embodiments, providing the second composition occurs after providing the first composition and before the first level of protein expressed by the first composition decreases by 25%-75% in the cell or subject.

In some embodiments of these aspects, the method further comprises providing a third composition of the circular polyribonucleotide to the cell or subject after the second composition, thereby maintaining expression of the protein in the cell or subject at least at the first level of protein. In some embodiments, providing the third composition occurs after providing the second composition and (i) before the second level of the protein expressed by the first and second composition is substantially undetectable in the cell or subject, or (ii) before the second level of the protein expressed by the first and second composition in the cell or subject decreases by more than 50%. In some embodiments, the method further comprises providing a fourth, fifth, sixth, seventh, eighth, ninth, or tenth composition of the circular polyribonucleotide.

In some embodiments of these aspects, the second composition is provided to the cell or subject at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of protein in the cell or subject expressed by the first composition is substantially undetectable. In some embodiments of these aspects, the second composition is provided to the cell or subject at least 14 days after the first composition and no more than 90 days after the first composition.

In some embodiments, a first level of the protein is a highest level of the protein one day after providing the first composition. In some embodiments, a first level of the protein is 40%, 50%, 60%, 70%, 80%, or 90% of a highest level of the protein one day after providing the first composition.

In some embodiments, a second level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, or 30 days after providing the second composition. In some embodiments, a third level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, or 30 days after providing the third composition. In some embodiments, each subsequent composition provided after the first composition, a subsequent level of the protein expressed after each subsequent composition is at least 30%, 40%, 50%, 60%, 70%, 80%, 90%, 100%, 110%, 120%, or 130% of a highest level of the protein one day after providing the first composition for at least 1, 2, 3, 4, 5, 6, 7, 8, 9, 10, 12, 15, 20, 25, or 30 days after providing each subsequent composition. In some embodiments, an average level of the protein after providing the second composition is at least 40%, 50%, 60%, 70%, 80%, 90%, 100%, or 110% of the first level, wherein the average level of the protein is measured from one day after providing the second composition to the day when the protein is substantially undetectable. In some embodiments, an average level of the protein after providing each subsequent composition after the first composition is at least 40%, 50%, 60%, 70%, 80%, 90%, 100%, or 110% of the first level, wherein the average level of the protein is measured from one day after providing each subsequent composition to the day when the protein is substantially undetectable. In some embodiments, the first level of the protein is maintained after providing the first composition and the second composition of the circular polyribonucleotide for at least 6 hours, 1 day, 2 days, 3 days, 5 days, 7 days, 14 days, 21 days, 28 days, or 30 days after providing the first composition. In some embodiments, the first level of the protein is maintained after providing the first composition, the second composition, and the third composition of the circular polyribonucleotide for at least 6 hours, 1 day, 2 days, 3 days, 5 days, 7 days, 14 days, 21 days, 28 days, or 30 days after providing the first composition. In some embodiments, the second level of protein in the cell or subject after providing the second composition is at least 1%, 5%, 10%, 20%, 30%, 40%, 50%, or 60% higher than the first level of protein in the cell or subject after providing the first composition. In some embodiments, a third level of protein produced in the cell or subject after providing the third composition is at least 5%, 10%, 20%, 30%, 40%, 50%, or 60% higher than the first level of protein in the plurality after providing the first composition. In some embodiments, the second level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the protein after providing the first composition. In some embodiments, the third level of protein 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of the protein after providing the first composition.

In some embodiments, the level of the protein in the cell or subject after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days. In some embodiments, the level of the protein in the cell or subject after providing the first composition and the second composition of the circular polyribonucleotide is at least 5%, 10%, 20%, 30%, 40%, 50%, or 60% higher than the level of the protein in the cell or subject after providing the first composition and the second composition of the linear counterpart of the circular polyribonucleotide. In some embodiments, the level of the protein in the cell or subject after providing the first composition and the second composition of the circular polyribonucleotide is at least 5%, 10%, 20%, 30%, 40%, 50%, or 60% higher than the level of the protein in the cell or subject after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, or 30 days after providing the second composition of the circular polyribonucleotide.

In some embodiments, the protein is a therapeutic protein, e.g., erythropoietin, and/or wherein expression of the protein (e.g., erthyropoietin) induces a response (e.g., reticulocyte production) in the cell or subject. In some embodiments, the protein is an antigen (e.g., tumor antigen, viral antigen, bacterial antigen). In some embodiments, the protein is a protein for vaccination.

In an eighth aspect, the invention features a method of producing a circular polyribonucleotide in a cell or subject comprising: providing a first composition comprising the circular polyribonucleotide to the cell or subject, wherein the cell or subject comprises a first level of circular polyribonucleotide after providing the first composition; and providing a second composition of a circular polyribonucleotide to the cell or subject, wherein the cell or subject comprises a second level of circular polyribonucleotide and (i) the second level of circular polyribonucleotide is at least as much as the first level, or (ii) the second level of circular polyribonucleotide varies by no more than 20% of the first level after providing the second composition; thereby maintaining circular polyribonucleotide in the cell or subject at least at the first level.

In some embodiments of this aspect, the first composition comprises a first circular polyribonucleotide and the second compositions comprises a second circular polyribonucleotide, wherein: (i) the first circular polyribonucleotide and the second circular polyribonucleotide are the same; or (ii) the first circular polyribonucleotide and the second circular polyribonucleotide are different. In some embodiments of this aspect, the first circular polyribonucleotide comprises a first binding site and/or encodes a first protein and the second circular polyribonucleotide comprise a second binding site and/or encodes a second protein, wherein the first binding site and the second binding site are the same or are different binding sites and/or the first protein and the second protein encode the same protein or different proteins.

In a ninth aspect, the invention features a method of producing a level of a circular polyribonucleotide in a cell or subject after providing a first composition and a second composition of the circular polyribonucleotide to the cell or subject compared to a level of a linear counterpart of the circular polyribonucleotide in the cell or subject after providing a first composition and second composition of the linear counterpart of the circular polyribonucleotide, comprising: providing a first composition of the circular polyribonucleotide to the cell or subject, wherein the cell or subject comprises the level of the circular polyribonucleotide after providing the first composition; and providing the second composition of the circular polyribonucleotide to the cell or subject, wherein the cell or subject comprises (i) at least the level of the circular polyribonucleotide after providing the second composition, or (ii) a level of the protein after providing the second composition that varies by no more than 20% of the level of the circular polyribonucleotide; thereby maintaining the level of the circular polyribonucleotide in the cell or subject after providing the first composition and the second composition of the circular polyribonucleotide compared to the level of the linear counterpart in the cell or subject after providing the first composition and the second composition of the linear counterpart of the circular polyribonucleotide.

In some embodiments, providing the second composition occurs after providing the first composition and before the level of circular polyribonucleotide produced by providing the first composition is substantially undetectable in the cell or subject.

In some embodiments, providing the second composition of the circular polyribonucleotide occurs after the first composition and after the level of circular polyribonucleotide in the cell or subject produced by the first composition is substantially undetectable. In some embodiments, the second composition is provided to the cell or subject at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of circular polyribonucleotide produced by the first composition is substantially undetectable. In some embodiments, the second composition is provided to the cell or subject at least 14 days after the first composition and no more than 90 days after the first composition.

In some embodiments, the method further comprises providing a third composition of circular polyribonucleotide to the cell or subject after the second composition, thereby maintaining the level of circular polyribonucleotide after providing the third composition at least at the first level, and, optionally, wherein providing the third composition occurs after providing the second composition and (i) before the level of circular polyribonucleotide produced by the first and second composition in the cell or subject is substantially undetectable in the cell or subject, or (ii) before the level of circular polyribonucleotide produced by the first and second composition in the cell or subject decreases by more than 50%; or (iii) before the level of circular polyribonucleotide produced by the first and second composition in the cell or subject decreases by 25%-75% in the cell or subject.

In some embodiments, the method further comprises providing a fourth, fifth, sixth, seventh, eighth, ninth, or tenth composition of the circular polyribonucleotide to the cell or subject.

In some embodiments, the first level of the circular polyribonucleotide is a highest level of circular polyribonucleotide one day after providing the first composition. In some embodiments, for each subsequent composition provided after the first composition, a subsequent level of circular polyribonucleotide expressed after each subsequent composition is at least 30%, 40%, 50%, 60%, 70%, 80%, 90%, 100%, 110%, 120%, or 130% of a highest level of circular polyribonucleotide one day after providing the first composition for at least 1, 2, 3, 4, 5, 6, 7, 8, 9, 10, 12, 15, 20, 25, or 30 days after providing each subsequent composition. In some embodiments, an average level of the circular polyribonucleotide after providing the second composition is at least 40%, 50%, 60%, 70%, 80%, or 90% of the first level, wherein the average level of the circular polyribonucleotide is measured from one day after providing the second composition to the day when the circular polyribonucleotide is substantially undetectable. In some embodiments, an average level of the circular polyribonucleotide after providing each subsequent composition after the first composition is at least 40%, 50%, 60%, 70%, 80%, or 90% of the first level, wherein the average level of the circular polyribonucleotide is measured from one day after providing each subsequent composition to the day when the circular polyribonucleotide is substantially undetectable. In some embodiments, the first level of the circular polyribonucleotide is maintained after providing the second composition of the circular polyribonucleotide for at least 6 hours, 1 day, 2 days, 3 days, 5 days, 7 days, 14 days, 21 days, 28 days, or 30 days. In some embodiments, the second level of circular polyribonucleotide in the cell or subject after providing the second composition is at least 1%, 5%, 10%, 20%, 30%, 40%, 50%, or 60% higher than the first level of circular polyribonucleotide in the cell or subject after providing the first composition. In some embodiments, the second level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the second composition of the circular polyribonucleotide is at least 1%, 5%, 10%, 20%, 30%, 40%, 50%, or 60% higher than the first level of circular polyribonucleotide after providing the first composition.

In some embodiments, a third level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of circular polyribonucleotide after providing the first composition.

In some embodiments, the level of circular polyribonucleotide in the cell or subject after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days. In some embodiments, the level of circular polyribonucleotide produced by the first composition is 40%, 50%, 60%, 70%, 80%, or 90% of a highest level of the circular polyribonucleotide one day after providing the first composition. In some embodiments, the level of circular polyribonucleotide produced by the second composition is at least 30%, 40%, 50%, 60%, 70%, 80%, 90%, 100%, 110%, 120%, or 130% of a highest level of circular polyribonucleotide one day after providing the first composition, for at least 1, 2, 3, 4, 5, 6, 7, 8, 9, 10, 12, 15, 20, 25, or 30 days after providing the second composition. In some embodiments, the level of circular polyribonucleotide in the cell or subject after providing the first composition and the second composition of the circular polyribonucleotide is at least 5%, 10%, 20%, 30%, 40%, 50%, or 60% higher than the level of linear counterpart of the circular polyribonucleotide in the cell or subject after providing the first composition and the second composition of the linear counterpart of circular polyribonucleotide. In some embodiments, the level of circular polyribonucleotide after providing the first composition and the second composition of circular polyribonucleotide is at least 5%, 10%, 20%, 30%, 40%, 50%, or 60% higher than the level of linear counterpart of circular polyribonucleotide after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, or 30 days after providing the second composition of the circular polyribonucleotide.

In some embodiments, a third level of the circular polyribonucleotide is at least 30%, 40%, 50%, 60%, 70%, 80%, 90%, 100%, 110%, 120%, or 130% of the highest level of circular polyribonucleotide one day after providing the first composition for at least 1, 2, 3, 4, 5, 6, 7, 8, 9, 10, 12, 15, 20, 25, or 30 days after providing the third composition.

In some embodiments, the first level of circular polyribonucleotide is maintained after providing the third composition of circular polyribonucleotide for at least 6 hours, 1 day, 2 days, 3 days, 5 days, 7 days, 14 days, 21 days, 28 days or 30 days. In some embodiments, the third level of circular polyribonucleotide in the cell or subject after providing the third composition is at least 5%, 10%, 20%, 30%, 40%, 50%, or 60% higher than the first level of circular polyribonucleotide in the plurality after providing the first composition. In some embodiments, the third level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of circular polyribonucleotide after providing the first composition.

In some embodiments, the protein (e.g., erthypoietin) induces a response (e.g., production of reticulocytes) in the subject. In some embodiments, the protein is an antigen (e.g., viral antigen, bacterial antigen, tumor antigen).

In a tenth aspect, the invention features a method of binding a target in a cell or subject comprising: providing a first composition comprising a circular polyribonucleotide that comprises a binding site for a target, to the cell or subject, wherein the target binds to the binding site at a first level; and providing a second composition comprising the circular polyribonucleotide that comprises a binding site for a target to the cell or subject, wherein the target binds to the binding site at a second level and (i) the second level is at least as much as the first level, or (ii) the second level varies by no more than 20% of the first level;

thereby maintaining binding of the target in the cell or subject at least at the first level of binding.

In an eleventh aspect, the invention features a method of binding a target in a cell or subject after providing a first composition and a second composition of a circular polyribonucleotide to the cell or subject compared to a level of binding to the target in the cell or subject after providing a first composition and second composition of a linear counterpart of the circular polyribonucleotide, comprising: (a) providing a first composition of the circular polyribonucleotide comprising binding site to the cell or subject, wherein the cell or subject comprises the level of the binding to the target after providing the first composition of the circular polyribonucleotide; and (b) providing the second composition of the circular polyribonucleotide after the first composition to the cell or subject, wherein the cell or subject comprises (i) at least the level of the binding to the target after providing the second composition of the circular polyribonucleotide, or (ii) a level of the binding to a target that varies by no more than 20% of the level after providing the second composition of the circular polyribonucleotide; thereby maintaining the level of the binding to the target in the cell or subject after providing the first composition and the second composition of the circular polyribonucleotide compared to the level of the binding to the target in the cell or subject after providing the first composition and the second composition of the linear counterpart of the circular polyribonucleotide.

In some embodiments, wherein providing the second composition occurs after providing the first composition and before the first level of binding by the first composition is substantially undetectable in the cell or subject. In some embodiments, wherein providing the second composition occurs after providing the first composition and before the first level of binding by the first composition decreases by more than 50% in the cell or subject. In some embodiments, wherein providing the second composition occurs after providing the first composition and before the first level of binding by the first composition decreases by 25%-75% in the cell or subject.

In some embodiments, the method further comprises providing a third composition of the circular polyribonucleotide to the cell or subject after the second composition, thereby maintaining binding of the target in the cell or subject at least at the first level of binding. In some embodiments, providing the third composition occurs after providing the second composition and before the second level of the binding of the target in the cell or subject by the first and second composition is substantially undetectable in the cell or subject.

In some embodiments, providing the third composition occurs after providing the second composition and before the second level of the binding by the first and second composition in the cell or subject decreases by more than 50%.

In some embodiments, the method further comprises providing a fourth, fifth, sixth, seventh, eighth, ninth, or tenth composition of the circular polyribonucleotide.

In some embodiments, providing the second composition of circular polyribonucleotide occurs after the first composition and after the level of binding by the first composition is substantially undetectable. In some embodiments, the second composition is provided to the cell or subject at least 6 hours, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of binding by the first composition is substantially undetectable. In some embodiments, the second composition is provided to the cell or subject at 14 days after the first composition and no more than 90 days after the first composition.

In some embodiments, the first level of binding is the highest level of binding one day after providing the first composition. In some embodiments, the first level of the binding is 40%, 50%, 60%, 70%, 80%, or 90% of the highest level of binding one day after providing the first composition. In some embodiments, the second level of binding is at least 30%, 40%, 50%, 60%, 70%, 80%, 90%, 100%, 110%, 120%, or 130% of a highest level of binding one day after providing the first composition for at least 1, 2, 3, 4, 5, 6, 7, 8, 9, 10, 12, 15, 20, 25, 30, 35, 40, or 45 days after providing the second composition. In some embodiments, the third level of binding is at least 30%, 40%, 50%, 60%, 70%, 80%, 90%, 100%, 110%, 120%, or 130% of a highest level of binding one day after providing the first composition for at least 1, 2, 3, 4, 5, 6, 7, 8, 9, 10, 12, 15, 20, 25, or 30 days after providing the third composition. In some embodiments, for each subsequent composition provided after the first composition, a subsequent level of binding after each subsequent composition is at least 30%, 40%, 50%, 60%, 70%, 80%, 90%, 100%, 110%, 120%, or 130% of the highest level of binding one day after providing the first composition for at least 1, 2, 3, 4, 5, 6, 7, 8, 9, 10, 12, 15, 20, 25, or 30 days after providing each subsequent composition. In some embodiments, an average level of binding after providing the second composition is at least 40%, 50%, 60%, 70%, 80%, 90%, 100%, or 110% of the first level, wherein the average level of binding is measured from one day after providing the second composition to the day when the binding is substantially undetectable. In some embodiments, an average level of binding after providing each subsequent composition after the first composition is at least 40%, 50%, 60%, 70%, 80%, 90%, 100%, or 110% of the first level, wherein the average level of binding is measured from one day after providing each subsequent composition to the day when the binding is substantially undetectable. In some embodiments, the first level of the binding is maintained after providing the first composition and the second composition of the circular polyribonucleotide for at least 6 hours, 1 day, 2 days, 3 days, 5 days, 7 days, 14 days, 21 days, 28 days, or 30 days after providing the first composition In some embodiments, the second level of binding in the cell or subject after providing the second composition is at least 1%, 5%, 10%, 20%, 30%, 40%, 50%, or 60% higher than the first level of binding in the cell or subject after providing the first composition. In some embodiments, the second level of binding 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the second composition of the circular polyribonucleotide is at least 1%, 5%, 10%, 20%, 30%, 40%, 50%, or 60% higher than the first level of the binding after providing the first composition.

In some embodiments, the level of binding in the cell or subject after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days. In some embodiments, the level of binding in the cell or subject after providing the first composition and the second composition of the circular polyribonucleotide is at least 5%, 10%, 20%, 30%, 40%, 50%, or 60% higher than the level of binding in the cell or subject after providing the first composition and the second composition of the linear counterpart of the circular polyribonucleotide In some embodiments, the level of binding in the cell or subject after providing the first composition and the second composition of the circular polyribonucleotide is at least 5%, 10%, 20%, 30%, 40%, 50%, or 60% higher than the level of binding in the cell or subject after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, or 30 days after providing the second composition of the circular polyribonucleotide.

In some embodiments, the first level of the binding is maintained after providing the first composition, second composition, and third composition of the circular polyribonucleotide for at least 6 hours, 1 day, 2 days, 3 days, 5 days, 7 days, 14 days, 21 days, 28 days, or 30 days after providing the first composition In some embodiments, a third level of binding in the cell or subject after providing the third composition is at least 5%, 10%, 20%, 30%, 40%, 50%, or 60% higher than the first level of binding after providing the first composition. In some embodiments, a third level of binding 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of the binding after providing the first composition.

In some embodiments, the circular polyribonucleotide of the first composition and the circular polyribonucleotide of the second composition are the same. In some embodiments, the circular polyribonucleotide of the first composition and the circular polyribonucleotide of the second composition are different.

In some embodiments, the first composition and the second composition comprise about the same amount of the circular polyribonucleotide. In some embodiments, the first composition comprises a higher amount of the circular polyribonucleotides than the second composition. In some embodiments, the first composition comprises a higher amount of the circular polyribonucleotides than a third, fourth, fifth, sixth, seventh, eighth, ninth, or tenth composition. In some embodiments, an amount of circular polyribonucleotide of the second composition varies by no more than 1%, 5%, 10%, 15%, 20%, or 25% of an amount of circular polyribonucleotide of the first composition. In some embodiments, an amount of circular polyribonucleotide of the second composition is no more than 1%, 5%, 10%, 15%, 20%, or 25% less than an amount of circular polyribonucleotide of the first composition In some embodiments, the first composition further comprises a pharmaceutically acceptable carrier or excipient. In some embodiments, the second composition further comprises a pharmaceutically acceptable carrier or excipient. In some embodiments, the third composition further comprises a pharmaceutically acceptable carrier or excipient. In some embodiments, the cell is an animal cell (e.g., a mammalian cell, e.g., a human cell). In some embodiments, the cell is a plurality of cells in a subject. In some embodiments, the first composition and/or the second composition comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules. In some embodiments, the first composition and/or the second composition comprises at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to the total ribonucleotide molecules in the first composition and/or the second composition. In some embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) of total ribonucleotide molecules in first composition and/or the second composition are circular polyribonucleotide molecules.

In some embodiments, the subject is an animal (e.g., a mammal). In some embodiments, the subject is a human. In some embodiments, the protein is an antigen (e.g., tumor antigen, bacterial antigen, viral antigen).

In a twelfth aspect, the invention generally features a method of producing a circular polyribonucleotide in a cell comprising: providing a first composition comprising the circular polyribonucleotide to the cell, wherein the cell comprises a first level of the circular polyribonucleotide after providing the first composition; and providing a second composition of the circular polyribonucleotides to the cell, wherein the cell comprises a second level of the circular polyribonucleotide and the second level of circular polyribonucleotide is at least as much as the first level after providing the second composition; thereby maintaining the circular polyribonucleotide in the cell at least at the first level.

In an thirteenth aspect, the invention generally features a method of producing a circular polyribonucleotide in a mammal comprising: providing a first composition comprising the circular polyribonucleotide to the mammal, wherein the mammal comprises a first level of the circular polyribonucleotide after providing the first composition; and providing a second composition of the circular polyribonucleotides to the mammal, wherein the mammal comprises a second level of the circular polyribonucleotide and the second level of circular polyribonucleotide is at least as much as the first level after providing the second composition; thereby maintaining the circular polyribonucleotide in the mammal at least at the first level.

In a fourteenth aspect, the invention generally features a method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprising: providing a first composition of the circular polyribonucleotide to the cell, wherein the cell comprises the level of the circular polyribonucleotide after providing the first composition; and providing the second composition of the circular polyribonucleotide to the cell, wherein the cell comprises at least the level of the circular polyribonucleotide after providing the second composition; thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide.

In a fifteenth aspect, the invention generally features a method of producing a level of a circular polyribonucleotide in a mammal after providing a first composition and a second composition of the circular polyribonucleotide to the mammal compared to a level of a linear counterpart of the circular polyribonucleotide in the mammal after providing a first composition and second composition of the linear counterpart of the circular polyribonucleotide, comprising: providing a first composition of the circular polyribonucleotide to the mammal, wherein the mammal comprises the level of the circular polyribonucleotide after providing the first composition; and providing the second composition of the circular polyribonucleotide to the mammal, wherein the mammal comprises at least the level of the circular polyribonucleotide after providing the second composition; thereby maintaining the level of the circular polyribonucleotide in the mammal after providing the first composition and the second composition of the circular polyribonucleotide compared to the level of the linear counterpart in the mammal after providing the first composition and the second composition of the linear counterpart of the circular polyribonucleotide.

In a sixteenth aspect, the invention generally features a method of binding at target in a cell comprising: providing a first composition comprising the circular polyribonucleotide to the cell, wherein the cell comprises a first level of binding after providing the first composition; and providing a second composition of the circular polyribonucleotides to the cell, wherein the cell comprises a second level of binding and the second level of binding is at least as much as the first level of binding after providing the second composition; thereby maintaining the binding in the cell at least at the first level.

In a seventeenth aspect, the invention generally features a method of binding at target in a mammal comprising: providing a first composition comprising the circular polyribonucleotide to the mammal, wherein the mammal comprises a first level of binding after providing the first composition; and providing a second composition of the circular polyribonucleotides to the mammal, wherein the mammal comprises a second level of binding and the second level of binding is at least as much as the first level of binding after providing the second composition; thereby maintaining the binding in the mammal at least at the first level.

In a eighteenth aspect, the invention generally features a method of binding a target in a cell after providing a first composition and a second composition of the circular polyribonucleotide to the cell compared to a level of binding in the cell after providing a first composition and second composition of the linear counterpart of the circular polyribonucleotide, comprising: providing a first composition of the circular polyribonucleotide to the cell, wherein the cell comprises the level of binding after providing the first composition; and providing the second composition of the circular polyribonucleotide to the cell, wherein the cell comprises at least the level of binding after providing the second composition; thereby maintaining the level of binding in the cell after providing the first composition and the second composition of the circular polyribonucleotide compared to the level of binding in the cell after providing the first composition and the second composition of the linear counterpart of the circular polyribonucleotide.

In a nineteenth aspect, the invention generally features a method of binding a target in a mammal after providing a first composition and a second composition of the circular polyribonucleotide to the mammal compared to a level of binding in the mammal after providing a first composition and second composition of the linear counterpart of the circular polyribonucleotide, comprising: providing a first composition of the circular polyribonucleotide to the mammal, wherein the mammal comprises the level of binding after providing the first composition; and providing the second composition of the circular polyribonucleotide to the mammal, wherein the mammal comprises at least the level of binding after providing the second composition; thereby maintaining the level of binding in the mammal after providing the first composition and the second composition of the circular polyribonucleotide compared to the level of binding in the mammal after providing the first composition and the second composition of the linear counterpart of the circular polyribonucleotide.

### Definitions

The present invention will be described with respect to particular embodiments and with reference to certain figures but the invention is not limited thereto but only by the claims. Terms as set forth hereinafter are generally to be understood in their common sense unless indicated otherwise.

As used herein, the terms “circRNA” or “circular polyribonucleotide” or “circular RNA” are used interchangeably and mean a polyribonucleotide molecule that has a structure having no free ends (i.e., no free 3′ and/or 5′ ends), for example a polyribonucleotide molecule that forms a circular or end-less structure through covalent or non-covalent bonds.

As used herein, the term “aptamer sequence” is a non-naturally occurring or synthetic oligonucleotide that specifically binds to a target molecule. Typically an aptamer is from 20 to 500 nucleotides. Typically an aptamer binds to its target through secondary structure rather than sequence homology.

As used herein, the term “encryptogen” is a nucleic acid sequence or structure of the circular polyribonucleotide that aids in reducing, evading, and/or avoiding detection by an immune cell and/or reduces induction of an immune response against the circular polyribonucleotide.

As used herein, the term “expression sequence” is a nucleic acid sequence that encodes a product, e.g., a peptide or polypeptide, or a regulatory nucleic acid. An exemplary expression sequence that codes for a peptide or polypeptide comprises a plurality of nucleotide triads, each of which code for an amino acid and is termed as a “codon”.

As used herein the term “exogenous”, when used with reference to a biomolecule (such as a circular RNA) means that the biomolecule was introduced into a host genome, cell or organism by the hand of man. For example, a circular RNA that is added into an existing genome, cell, tissue or subject using recombinant DNA techniques and/or methods for internalizing a biomolecule into a cell, is exogenous to the existing nucleic acid sequence, cell, tissue or subject, and any progeny of the nucleic acid sequence, cell, tissue or subject that retain the biomolecule.

As used herein, the term “immunoprotein binding site” is a nucleotide sequence that binds to an immunoprotein. In some embodiments, the immunoprotein binding site aids in masking the circular polyribonucleotide as exogenous, for example, the immunoprotein binding site is bound by a protein (e.g., a competitive inhibitor) that prevents the circular polyribonucleotide from being recognized and bound by an immunoprotein, thereby reducing or avoiding an immune response against the circular polyribonucleotide.

As used herein, the term “immunoprotein” is any protein or peptide that is associated with an immune response, e.g., such as against an immunogen, e.g., the circular polyribonucleotide. Non-limiting examples of immunoprotein include T cell receptors (TCRs), antibodies (immunoglobulins), major histocompatibility complex (MHC) proteins, complement proteins, and RNA binding proteins.

As used herein, the term “modified ribonucleotide” means any ribonucleotide analog or derivative that has one or more chemical modifications to the chemical composition of an unmodified natural ribonucleotide, such as a natural unmodified nucleotide adenosine (A), uridine (U), guanine (G), cytidine (C). In some embodiments, the chemical modifications of the modified ribonucleotide are modifications to any one or more functional groups of the ribonucleotide, such as, the sugar the nucleobase, or the internucleoside linkage (e.g. to a linking phosphate/to a phosphodiester linkage/to the phosphodiester backbone).

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory element” is a moiety, such as a nucleic acid sequence, that modifies expression of an expression sequence within the circular polyribonucleotide.

As used herein, the term “repetitive nucleotide sequence” is a repetitive nucleic acid sequence within a stretch of DNA or RNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motifs useful for replication or that initiate transcription of the circular polyribonucleotide.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPG P, where x=any amino acid. In some embodiments, the stagger element may include a chemical moiety, such as glycerol, a non nucleic acid linking moiety, a chemical modification, a modified nucleic acid, or any combination thereof.

As used herein, the term “substantially resistant” means one that has at least 50%, 55%, 60%, 65%, 70%, 75%, 80%, 85%, 90%, 95%, 96%, 97%, 98% or 99% resistance as compared to a reference.

As used herein, the term “stoichiometric translation” means a substantially equivalent production of expression products translated from the circular polyribonucleotide. For example, for a circular polyribonucleotide having two expression sequences, stoichiometric translation of the circular polyribonucleotide can mean that the expression products of the two expression sequences can have substantially equivalent amounts, e.g., amount difference between the two expression sequences (e.g., molar difference) can be about 0, or less than 1%, 2%, 3%, 4%, 5%, 6%, 7%, 8%, 9%, 10%, 15%, or 20%.

As used herein, the term “translation initiation sequence” is a nucleic acid sequence that initiates translation of an expression sequence in the circular polyribonucleotide.

As used herein, the term “termination element” is a moiety, such as a nucleic acid sequence, that terminates translation of the expression sequence in the circular polyribonucleotide.

As used herein, the term “translation efficiency” means a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “circularization efficiency” is a measurement of resultant circular polyribonucleotide versus its starting material.

As used herein, the term “immunogenic” is a potential to induce an immune response to a substance. In some embodiments, an immune response may be induced when an immune system of an organism or a certain type of immune cells is exposed to an immunogenic substance. The term “non-immunogenic” is a lack of or absence of an immune response above a detectable threshold to a substance. In some embodiments, no immune response is detected when an immune system of an organism or a certain type of immune cells is exposed to a non-immunogenic substance. In some embodiments, a non-immunogenic circular polyribonucleotide as provided herein, does not induce an immune response above a pre-determined threshold when measured by an immunogenicity assay. For example, when an immunogenicity assay is used to measure an innate immune response against a circular polyribonucleotide (such as measuring inflammatory markers), a non-immunogenic polyribonucleotide as provided herein can lead to production of an innate immune response at a level lower than a predetermined threshold. The predetermined threshold can be, for instance, at most 1.5 times, 2 times, 3 times, 4 times, 5 times, 6 times, 7 times, 8 times, 9 times, or 10 times the level of a marker(s) produced by an innate immune response for a control reference.

As used herein, the term “substantially undetectable” can refer to the level of the circular polyribonucleotide or the protein expressed from the circular polyribonucleotide that is lower than the level detectable by a relevant detection technique (e.g., chromatography (column, paper, gel, HPLC, UHPLC, IC, SEC, etc.), electrophoresis (UREA PAGE, chip-based, polyacrylamide gel, RNA, capillary, c-IEF, etc.), fluorescence-based detection techniques, etc.).

As used herein, the term “linear counterpart” is a polyribonucleotide molecule (and its fragments) having the same or similar nucleotide sequence (e.g., 100%, 95%, 90%, 85%, 80%, 75%, or any percentage therebetween of sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween of sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “conjugation moiety” refers to a modified nucleotide comprising a functional group for use in a method of conjugation.

As used herein, the term “carrier” means a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier.

The term “diluent” means a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the term “response” or “response level” is any measurable change or any level of the measurable change resulting from exposure to a stimulus. For example, a measurable change is a shift or change in a phenotype (e.g., cellular phenotype, physical phenotype, molecular phenotype) or any characteristic that informs that a stimulus is working, and includes, for example, a change in cell morphology, an increase or decrease on production of a cell type, an increase or decrease in muscle mass, after exposure to the stimulus. As a further example, a stimulus is a protein (e.g., erythropoietin expressed from a circular polyribonucleotide) or a circular polyribonucleotide comprising a binding site, and the response or response level is a measurable shift in a phenotype (e.g., increased production or level of reticulocytes in the subject) after exposure to the protein or circular polyribonucleotide comprising a binding site in the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 shows that after injection into mice, circular RNA was detected at higher levels than linear RNA in livers of mice at 3, 4, and 7 days post-injection.

FIG. 2A and FIG. 2B show that after injection of circular RNA or linear RNA expressing Gaussia Luciferase into mice, Gaussia Luciferase activity was detected in plasma at 1, 2, 7, 11, 16, and 23 days post-dosing of circular RNA, while its activity was only detected in plasma at 1, and 2 days post-dosing of modified linear RNA.

FIG. 3 show that after injection of RNA, circular RNA but not linear RNA, was detected in the liver and spleen at 16 days post-administration of RNA.

FIG. 4 show that after injection of RNA, linear RNA but not circular RNA, showed immunogenicity as assessed by RIG-I, MDA-5, IFN-B and OAS.

FIG. 5 shows experimental data demonstrating increased persistence of Gaussia luciferase expression in mice following redosing with a circular polyribonucleotide (“Endless”) as compared to a linear polyribonucleotide counterpart (“Linear”).

FIG. 6 shows experimental data demonstrating increased persistence of Gaussia luciferase expression in mice following staggered dosing with a circular polyribonucleotide (“Endless 3 doses”) as compared to staggered dosing a linear polyribonucleotide counterpart (“Linear 3 doses”), or a single dose with the circular polyribonucleotide (“Endless”), or a single dose with a linear polyribonucleotide counterpart (“Linear”).

FIG. 7 shows experimental data demonstrating increased persistence of Gaussia luciferase expression in mice following a single dose of a circular polyribonucleotide (“Endless RNA”) as compared to a single dose of a linear polyribonucleotide counterpart (“Linear RNA”), staggered dosing with a linear polyribonucleotide counterpart (“3 doses Linear RNA”) as compared to a single dose (“Linear RNA”), or staggered dosing with a circular polyribonucleotide (“3 doses Endless RNA”) as compared a single dose (“Endless RNA”).

FIG. 8 shows circular polyribonucleotide administered intravenously, with carrier (TransIT) and without carrier (Unformulated), expressed protein in vivo for prolong periods for time, with levels of protein activity in the plasma at multiple days post injection.

FIG. 9 shows circular polyribonucleotide administered intramuscularly, without a carrier, expressed protein in vivo for prolonged periods of time, with levels of protein activity in the plasma at multiple days post injection.

FIG. 10 shows circular polyribonucleotide administered intravenously, expressed protein in vivo for prolonged periods of time, with levels of protein activity in the plasma at multiple days post injection and could be redosed at least 5 times.

FIG. 11 shows circular polyribonucleotide expressed protein in vivo for prolonged periods of time with increased levels of protein activity in the plasma after multiple continuous injections.

FIG. 12 shows an increased number of reticulocytes was detected in whole blood at 3, 5, 7, 14, 21 and 28 days post-dosing of the first dose of unformulated RNA, after which reticulocyte counts were back to the normal range of 3-5% in our mouse population.

FIG. 13 shows an increased number of reticulocytes was detected in whole blood at 3, 5, 7, 14, 21 and 28 days post-dosing of the first dose of TransIT-formulated RNA, after which reticulocyte counts were back to the normal range of 3-5% in our mouse population.

FIG. 14 shows an increase in reticulocyte count was detected for circular RNA dosing and mRNA dosing when unformulated compared to the vehicle only control.

FIG. 15 shows an increase in reticulocyte count was detected for circular RNA dosing and mRNA dosing when TransIT-formulated compared to the vehicle only control.

FIG. 16 shows a schematic of an exemplary in vitro production process of a circular RNA that contains a start-codon, an ORF (open reading frame) coding for GFP, a stagger element (2A), an encryptogen, and an IRES (internal ribosome entry site).

FIG. 17 shows a schematic of an exemplary in vivo production process of a circular RNA.

FIG. 18 shows design of an exemplary circular RNA that comprises a start-codon, an ORF coding for GFP, a stagger element (2A), and an encryptogen.

FIG. 19A and FIG. 19B are schematics demonstrating in vivo stoichiometric protein expression of two different circular RNAs.

FIG. 20 is a graph showing qRT-PCR analysis of immune related genes from 293T cells transfected with circular RNA or linear RNA.

FIG. 21 is a schematic demonstrating in vivo protein expression in mouse model from exemplary circular RNAs.

FIG. 22 is a schematic demonstrating in vivo biodistribution of an exemplary circular RNA in a mouse model.

FIG. 23 is a schematic demonstrating in vivo protein expression in mouse model from an exemplary circular RNA that harbors an encryptogen (intron).

FIG. 24 is a denaturing PAGE gel image demonstrating exemplary circular RNA after an exemplary purification process.

FIG. 25 is a Western blot image demonstrating expression of Flag protein (˜15 kDa) by an exemplary circular RNA that lacks IRES, cap, 5′ and 3′ UTRs.

### DETAILED DESCRIPTION

This invention relates generally to methods of dosing circular polyribonucleotides. The methods of dosing as disclosed herein generally relate to expressing a level of a protein or producing a level of a circular polyribonucleotide in cell after providing at least two compositions of circular polyribonucleotides, wherein the circular polyribonucleotide encodes the protein. The methods of dosing as disclosed herein also generally relate to binding of a target in a cell after providing at least two compositions of circular polyribonucleotides, wherein the circular polyribonucleotide encodes the protein. In some embodiments, the circular polyribonucleotide is an exogenous, synthetic circular polyribonucleotide.

In some aspects, the invention relates to a method of expressing a protein in a cell comprising: providing a first composition comprising a circular polyribonucleotide that encodes the protein to the cell, wherein the cell expresses a first level of the protein; and providing a second composition comprising the circular polyribonucleotide to the cell, wherein the cell expresses a second level of the protein and the second level is at least as much as the first level; thereby maintaining expression of the protein in the cell at least at the first level of the protein. In some aspects, a method of expressing a protein in a cell comprises: providing a first composition comprising a circular polyribonucleotide that encodes the protein to the cell, wherein the cell expresses a first level of the protein; and providing a second composition comprising the circular polyribonucleotide to the cell, wherein the cell expresses a second level of the protein and the second level varies by no more than 20% of the first level; thereby maintaining expression of the protein in the cell at least at the first level of the protein. In some aspects, a method of producing a circular polyribonucleotide in a cell comprises: providing a first composition comprising the circular polyribonucleotide to the cell, wherein the cell comprises a first level of the circular polyribonucleotide after providing the first composition; and providing a second composition of the circular polyribonucleotide to the cell, wherein the cell comprises a second level of the circular polyribonucleotide and the second level of circular polyribonucleotide is at least as much as the first level; thereby maintaining the circular polyribonucleotide in the cell at least at the first level. In some aspects, a method of producing a circular polyribonucleotide in a cell comprises: providing a first composition comprising the circular polyribonucleotide to the cell, wherein the cell comprises a first level of the circular polyribonucleotide after providing the first composition; and providing a second composition of the circular polyribonucleotides to the cell, wherein the cell comprises a second level of the circular polyribonucleotide and the second level of circular polyribonucleotide varies by no more than 20% of the first level after providing the second composition; thereby maintaining the circular polyribonucleotide in the cell at least at the first level. In some aspects, a method of producing a circular polyribonucleotide in a cell comprises: providing a first composition comprising the circular polyribonucleotide to the cell, wherein the cell comprises a first level of the circular polyribonucleotide after providing the first composition; and providing a second composition of the circular polyribonucleotides to the cell, wherein the cell comprises a second level of the circular polyribonucleotide and the second level of circular polyribonucleotide varies by no more than 20% of the first level after providing the second composition; thereby maintaining the circular polyribonucleotide in the cell at least at the first level. In some embodiments, providing the second composition occurs after providing the first composition and before the first level of protein expressed by the first composition is substantially undetectable in the cell.

In some aspects, the invention relates to a method of expressing a level of a protein in a cell after providing a first composition and a second composition of a circular polyribonucleotide to the cell compared to a level of the protein in the cell after providing a first composition and a second composition of a linear counterpart of the circular polyribonucleotide, comprising: providing a first composition of the circular polyribonucleotide encoding the protein to the cell, wherein the cell comprises the level of the protein after providing the first composition of the circular polyribonucleotide; and providing the second composition of the circular polyribonucleotide after the first composition to the cell, wherein the cell comprises at least the level of the protein after providing the second composition of the circular polyribonucleotide; thereby maintaining expression of the level of the protein in the cell after providing the first composition and the second composition of the circular polyribonucleotide compared to the level of the protein in the cell after providing the first composition and the second composition of the linear counterpart of the circular polyribonucleotide. In some aspects, a method of expressing a level of a protein in a cell after providing a first composition and a second composition of a circular polyribonucleotide to the cell compared to a level of the protein in the cell after providing a first composition and second composition of a linear counterpart of the circular polyribonucleotide, comprises: providing a first composition of the circular polyribonucleotide encoding the protein to the cell, wherein the cell comprises the level of the protein after providing the first composition of the circular polyribonucleotide; and providing the second composition of the circular polyribonucleotide after the first composition to the cell, wherein the cell comprises a level of the protein that varies by no more than 20% of the level after providing the second composition of the circular polyribonucleotide; thereby maintaining expression of the level of the protein in the cell after providing the first composition and the second composition of the circular polyribonucleotide compared to the level of the protein in the cell after providing the first composition and the second composition of the linear counterpart of the circular polyribonucleotide. In some aspects, a method of producing a level of a circular polyribonucleotide in a cell after providing a first composition and a second composition of the circular polyribonucleotide to the cell compared to a level of a linear counterpart of the circular polyribonucleotide in the cell after providing a first composition and a second composition of the linear counterpart of the circular polyribonucleotide, comprises: providing a first composition of the circular polyribonucleotide to the cell, wherein the cell comprises the level of the circular polyribonucleotide after providing the first composition; and providing the second composition of the circular polyribonucleotide to the cell, wherein the cell comprises at least the level of the circular polyribonucleotide after providing the second composition; thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide. In some aspects, a method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprises: providing a first composition of the circular polyribonucleotide to the cell, wherein the cell comprises a level of the circular polyribonucleotide after providing the first composition; and providing the second composition of the circular polyribonucleotide to the cell, wherein the cell comprises a level of the protein after providing the second composition that varies by no more than 20% of the level of the circular polyribonucleotide after providing the first composition; thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide. In some embodiments, providing the second composition of the circular polyribonucleotide occurs after the first composition and after the level of protein in the cell expressed by the first composition is substantially undetectable. The circular polyribonucleotides used in the methods described herein may comprise one or more expression sequences. In some embodiments, at least one of the expression sequences encodes a protein. The protein may be an intracellular protein, a membrane protein, or a secreted protein. The protein may be a therapeutic protein. In some embodiments the therapeutic protein may have an activity, for example has antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity.

The methods described herein may be therapeutic or veterinary methods for treating a subject. The methods described herein may be used to treat a disease in the plurality of cells. In some embodiments, the methods described herein are used to treat a disease resulting from a non-functional, poorly functional, or poorly expressed protein or gene product. In some embodiments, the methods described herein are used to treat a genetic disease (e.g., a mutation, a substitution, a deletion, an expansion, or a recombination), a cancer, a neurodegenerative disease, a cardiovascular disease, a pulmonary disease, a renal disease, a liver disease, a genetic disease, a vascular disease, ophthalmic disease, musculoskeletal disease, lymphatic disease, auditory and inner ear disease, a metabolic disease, an inflammatory disease, an autoimmune disease, or an infectious disease.

### Methods of Dosing

A method of dosing to produce a level of circular polyribonucleotide or express a level of a protein in a cell after providing the cell with at least two doses or compositions of circular polyribonucleotide is disclosed herein. A method of dosing to produce a level of circular polyribonucleotide or express a level of a protein in a subject (e.g., a mammal, e.g., a human) after providing (e.g., administering to) the subject with at least two doses or compositions of circular polyribonucleotide is disclosed herein. The composition can comprise a circular polyribonucleotide encoding a protein. The composition can comprise a circular polyribonucleotide comprises a binding site. A method of dosing can be redosing of a composition of circular polyribonucleotides in two or more doses, generally over an extended period of time. A method of dosing can be a staggered dosing of a composition over a short time interval. In some embodiments, the composition further comprises a pharmaceutically acceptable carrier or excipient. The protein from the circular polyribonucleotide can be expressed in a cell.

In some embodiments, the method comprises providing (e.g., administering) at least a first composition and a second composition to the cells or subject (e.g., a mammal, e.g., a human). In some embodiments, the method further comprises providing (e.g., administering) a third composition, fourth composition, fifth composition, sixth composition, seventh composition, eighth composition, ninth composition, tenth composition, or more. In some embodiments, additional compositions are provided for the duration of the life of the cell. In some embodiments, additional compositions are provided (e.g., administered) while the cell or subject obtains a benefit from the composition. In some embodiments, a plurality of compositions are provided (e.g., administered) in a staggered dosing regimen in which any composition provided (e.g., administered) after a previous composition is provided (e.g., administered) before the level of the protein or the circular polyribonucleotide from the previous composition in the plurality is substantially undetectable in the cell or subject (e.g., a mammal). For example, for providing a first composition and second composition in a staggered regimen, the second composition is provided (e.g., administered) after the first composition is provided (e.g., administered) and before the level of the protein or the circular polyribonucleotide from the first composition in the plurality is substantially undetectable in the cell or subject (e.g., mammal). In some embodiments, a plurality of compositions are provided in a redosing regimen in which any composition provided (e.g., administered) after a previous composition, is provided (e.g., administered) after the level of the protein or the circular polyribonucleotide from the previous composition in the plurality is substantially undetectable in the cell or subject (e.g., mammal). For example, for providing a first composition and a second composition in a redosing regimen, the second composition is provided (e.g., administered) after the first composition is provided (e.g., administered) and after the level of the protein or the circular polyribonucleotide from the first composition in the cell or subject is substantially undetectable in the cell or subject.

In some embodiments, a first composition in a staggered regimen or redosing regimen comprises a first amount of a circular polyribonucleotide. In some embodiments, a second composition in a staggered regimen or redosing regimen comprises a second amount of a circular polyribonucleotide. In some embodiments, a third composition, a fourth composition, a fifth composition, a sixth composition, a seventh composition, an eighth composition, a ninth composition, a tenth composition, or more in a staggered regimen or redosing regimen comprises a third, fourth, fifth, sixth, seventh, eighth, ninth, tenth or more amount of a circular polyribonucleotide. In some embodiments, the second amount of the circular polyribonucleotide is the same as the first amount of the circular polyribonucleotide. In some embodiments, the third amount of the circular polyribonucleotide is the same as the first amount of the circular polyribonucleotide. In some embodiments, the fourth, fifth, sixth, seventh, eighth, ninth, tenth, or more amount of the circular polyribonucleotide is the same as the first amount of the circular polyribonucleotide. In some embodiments, the second amount of the circular polyribonucleotide is less than the first amount of the circular polyribonucleotide. In some embodiments, the third amount of the circular polyribonucleotide is less than the first amount of the circular polyribonucleotide. In some embodiments, the fourth, fifth, sixth, seventh, eighth, ninth, tenth, or more amount of the circular polyribonucleotide is less than the first amount of the circular polyribonucleotide. In some embodiments, the second amount of the circular polyribonucleotide is greater than the first amount of the circular polyribonucleotide. In some embodiments, the third amount of the circular polyribonucleotide is greater than the first amount of the circular polyribonucleotide. In some embodiments, the fourth, fifth, sixth, seventh, eighth, ninth, tenth, or more amount of the circular polyribonucleotide is greater than the first amount of the circular polyribonucleotide. In some embodiments, an amount of circular polyribonucleotide of the second composition varies by no more than 1%, 5%, 10%, 15%, 20%, or 25% of an amount of circular polyribonucleotide of the first composition. In some embodiments, an amount of circular polyribonucleotide of the second composition is no more than 1%, 5%, 10%, 15%, 20%, or 25% less than an amount of circular polyribonucleotide of the first composition. In some embodiments, an amount of circular polyribonucleotide of a second composition is from 0.1-fold to 1000-fold higher than an amount of circular polyribonucleotide of a first composition. In some embodiments, an amount of circular polyribonucleotide of a second composition is 0.1-fold, 1-fold, 5-fold, 10-fold, 100-fold, or 1000-fold higher than an amount of circular polyribonucleotide of a first composition. In some embodiments, an amount of circular polyribonucleotide of a subsequent composition (e.g., a composition administered after a first composition) is 0.1-fold, 1-fold, 5-fold, 10-fold, 100-fold, or 1000-fold higher than an amount of circular polyribonucleotide of a first composition. In some embodiments, an amount of circular polyribonucleotide of a second composition is from 0.1-fold to 1000-fold lower than an amount of circular polyribonucleotide of a first composition. In some embodiments, an amount of circular polyribonucleotide of a second composition is 0.1-fold, 1-fold, 5-fold, 10-fold, 100-fold, or 1000-fold lower than an amount of circular polyribonucleotide of a first composition. In some embodiments, an amount of circular polyribonucleotide of a subsequent composition (e.g., a composition administered after a first composition) is 0.1-fold, 1-fold, 5-fold, 10-fold, 100-fold, or 1000-fold lower than an amount of circular polyribonucleotide of a first composition. In some embodiments, an amount of circular polyribonucleotide of a subsequent composition (e.g., after a first composition of an amount of circular polyribonucleotide) is from 0.1-fold to 1000-fold higher or lower than an amount of circular polyribonucleotide of a first composition. In some embodiments, an amount of circular polyribonucleotide of a subsequent composition (e.g., after a first composition of an amount of circular polyribonucleotide) is 0.1-fold, 1-fold, 5-fold, 10-fold, 100-fold, or 1000-fold higher or lower than an amount of circular polyribonucleotide of a first composition. For example a first composition comprises 1-fold circular polyribonucleotide, a second composition comprises 5-fold circular polyribonucleotide compared to the first composition, and a third composition comprises 0.2-fold circular polyribonucleotide compared to the first composition. In some embodiments, the second composition comprises at least 5-fold circular polyribonucleotide compared to an amount of circular polyribonucleotide of a first composition.

In some embodiments, the first composition comprises a higher amount of the circular polyribonucleotide than the second composition. In some embodiments, the first composition comprises a higher amount of the circular polyribonucleotides than the third, fourth, fifth, sixth, seventh, eighth, ninth, or tenth composition.

In some embodiments, the first composition further comprises a pharmaceutically acceptable carrier or excipient. In some embodiments, the second composition further comprises a pharmaceutically acceptable carrier or excipient. In some embodiments, the third composition, fourth composition, fifth composition, sixth composition, seventh composition, eighth composition, ninth composition, tenth composition, or more further comprises a pharmaceutically acceptable carrier or excipient.

In some embodiments, the first composition further comprises a pharmaceutically acceptable excipient and is free of any carrier. In some embodiments, the second composition further comprises a pharmaceutically acceptable excipient and is free of any carrier. In some embodiments, the third composition, fourth composition, fifth composition, sixth composition, seventh composition, eighth composition, ninth composition, tenth composition, or more further comprises a pharmaceutically acceptable excipient and is free of any carrier.

In some embodiments, the composition as described herein (e.g., a first composition, a second composition, a third composition, etc.) is delivered to a subject (e.g., a mammal). For example, a method of delivering a composition (e.g., a first composition, a second composition, a third composition, etc.) as described herein comprises parenterally administering to a subject in need thereof, the composition (e.g., a first composition, a second composition, a third composition, etc.) as described herein to the subject in need thereof. As another example, a method of delivering a composition (e.g., a first composition, a second composition, a third composition, etc.) to a subject, comprises administering parenterally the composition to the subject. In some embodiments, the composition (e.g., a first composition, a second composition, a third composition, etc.) as described herein comprises a carrier. In some embodiments the composition (e.g., a first composition, a second composition, a third composition, etc.) as described herein comprises a diluent and is free of any carrier. In some embodiments, parenteral administration is intravenously, intramuscularly, ophthalmically, or topically.

In some embodiments, the composition (e.g., a first composition, a second composition, a third composition, etc.) is administered orally. In some embodiments, the composition (e.g., a first composition, a second composition, a third composition, etc.) is administered nasally. In some embodiments, the composition (e.g., a first composition, a second composition, a third composition, etc.) is administered by inhalation. In some embodiments, the composition (e.g., a first composition, a second composition, a third composition, etc.) is administered topically. In some embodiments, the composition is administered opthalmically. In some embodiments, the composition (e.g., a first composition, a second composition, a third composition, etc.) is administered rectally. In some embodiments, the composition (e.g., a first composition, a second composition, a third composition, etc.) is administered by injection. In some embodiments, the composition (e.g., a first composition a second composition, a third composition, etc.) is administered by infusion. The administration can be systemic administration or local administration. In some embodiments, the composition (e.g., a first composition, a second composition, a third composition, etc.) is administered parenterally. In some embodiments, the composition (e.g., a first composition, a second composition, a third composition, etc.) is administered intravenously, intraarterially, intraperotoneally, intradermally, intracranially, intrathecally, intralymphaticly, subcutaneously, or intramuscularly. In some embodiments, the composition (e.g., a first composition, a second composition, a third composition, etc.) is administered via intraocular administration, intracochlear (inner ear) administration, or intratracheal administration. In some embodiments, any of the methods of delivery as described herein are performed with a carrier. In some embodiments, any methods of delivery as described herein are performed without the aid of a carrier.

A composition of a circular polyribonucleotide as described herein can induce a response in a subject. In some embodiments, a method of inducing a response in a subject comprises providing (e.g., administering) a composition that comprises a circular polyribonucleotide comprising a binding site and/or encoding a protein, for inducing a response level in the subject. In a particular embodiment, a method of inducing a response comprises providing (e.g., administering) a circular polyribonucleotide encoding erythropoietin to a subject, wherein expression of the erythropoietin from the circular polyribonucleotide in the subject induces production of reticulocytes in the subject. In some embodiments, a method of inducing a response level in a subject comprises (a) providing (e.g., administering) a first composition comprising a circular polyribonucleotide as described herein that induces the response, and from 14 days to 90 days following step (a), providing (e.g., administering) a second composition comprising a circular polyribonucleotide as described herein, to the subject, thereby inducing the response level in the subject after providing the first composition and the second composition. In some embodiments, a composition of a circular polyribonucleotide encodes a therapeutic protein for inducing a response or a response level in a subject after administration. In some embodiments, a composition of a circular polyribonucleotide encoding erthyropoietin is provided to a subject for inducing production of reticulocytes in the subject. In some embodiments, a method of inducing reticulocytes in a subject comprises (a) providing (e.g., administering) a first composition comprising a circular polyribonucleotide that encodes erthyropoietin to the subject; and (b) from 6 hours to 90 days after step (a), providing (e.g., administering) a second composition comprising a circular polyribonucleotide that encodes erthyropoietin to the subject, thereby inducing production of reticulocytes in the subject. In some embodiments, the method comprises providing (e.g., administering) the second composition from 6 hours to 30 days after step (a). In some embodiments, the method comprises providing (e.g., administering) the second composition from 14 days to 90 days after step (a). In some embodiments, the response level from providing a circular polyribonucleotide comprising a binding site or encoding a protein is greater than the response level from a linear counterpart polyribonucleotide. In some embodiments, a method of inducing a response level in a subject after providing a first composition and a second composition of a circular polyribonucleotide to the subject compared to a response level in the subject after providing a first composition and second composition of a linear counterpart of the circular polyribonucleotide, comprises: providing (e.g., administering) a first composition of the circular polyribonucleotide encoding a protein that induces a response level, to the subject, wherein the subject comprises a response level after the first composition of the circular polyribonucleotide is provided; and providing (e.g., administering) a second composition of the circular polyribonucleotide encoding a protein after the first composition to the subject, wherein the subject comprises at least the response level after the second composition of the circular polyribonucleotide is provided; thereby maintaining the response level in the subject after the first composition and the second composition of the circular polyribonucleotide are provided (e.g., administered) compared to the response level in the subject after the first composition and the second composition of the linear counterpart of the circular polyribonucleotide are provided (e.g., administered). For example, a method of inducing a level of reticulocyte production in a subject after providing a first composition and a second composition of a circular polyribonucleotide to the subject compared to a level of reticulocyte production in the subject after providing a first composition and second composition of a linear counterpart of the circular polyribonucleotide, comprises: providing (e.g., administering) a first composition of the circular polyribonucleotide encoding a erthyropoietin to the subject, wherein the subject comprises a level of reticulocyte production after the first composition of the circular polyribonucleotide is provided (e.g., administered); and providing (e.g., administering) the second composition of the circular polyribonucleotide encoding erthyropoietin after the first composition to the subject, wherein the subject comprises at least the level of reticulocyte production after the second composition of the circular polyribonucleotide is provided (e.g., administered); thereby maintaining the level of reticulocyte production in the subject after the first composition and the second composition of the circular polyribonucleotide are provided (e.g., administered) compared to the level of reticulocyte production in the subject after the first composition and the second composition of the linear counterpart of the circular polyribonucleotide are provided (e.g., administered).

In some embodiments, a composition of the circular polyribonucleotide as described here (e.g., a first composition, a second composition, a third composition, etc.) used for a method of dosing (e.g., staggered dosing or redosing) comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules. In some embodiments, a composition of the circular polyribonucleotide as described here (e.g., a first composition, a second composition, a third composition, etc.) comprises at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to the total ribonucleotide molecules in the composition of circular polyribonucleotides (e.g., a pharmaceutical composition as described herein). In some embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) of total ribonucleotide molecules in the composition as described herein are circular polyribonucleotide molecules.

### Staggered Dosing

A method of staggered dosing to produce a level of circular polyribonucleotide, express a level of a protein, or produce a level of binding to a target in a plurality of cells after providing the plurality of cells with at least two compositions of circular polyribonucleotide is disclosed herein. A method of staggered dosing to produce a level of circular polyribonucleotide, express a level of a protein, or produce a level of binding to a target in a subject (e.g., a mammal, e.g., a human) after providing (e.g., administering to) the subject (e.g., a mammal) with at least two compositions of circular polyribonucleotide is disclosed herein. In some embodiments, the at least two compositions of circular polyribonucleotide are the same compositions. In some embodiments, the at least two compositions of circular polyribonucleotide are different compositions. In some embodiments, the same compositions comprise circular polyribonucleotides encoding the same proteins or comprising the same binding sites. In some embodiments, the different compositions comprise circular polyribonucleotides encoding different proteins or comprising different binding sites, or a combination thereof.

In some embodiments, a method of maintaining expression of a protein in a mammal (e.g., a human), comprises: (a) providing (e.g., administering) a first composition comprising a circular polyribonucleotide that encodes the protein to the mammal (e.g., a human); and (b) from 6 hours to 90 days following step (a), providing (e.g., administering) a second composition comprising a circular polyribonucleotide that encodes the protein, to the mammal, thereby maintaining expression of the protein in the mammal.

In some embodiments a method of maintaining expression of an antigen in a mammal (e.g., a human), comprises: (a) providing (e.g., administering) a first composition comprising a circular polyribonucleotide that encodes the antigen to the mammal; and (b) from 6 hours to 90 days following step (a), providing (e.g., administering) a second composition comprising a circular polyribonucleotide that encodes the antigen, to the mammal, thereby maintaining expression of the protein in the mammal.

In some embodiments, the circular polyribonucleotide is an exogenous, synthetic circular polyribonucleotide. In some embodiments, the circular polyribonucleotide lacks a poly-A sequence, a replication element, or both.

In some embodiments, providing (e.g., administering) the second composition is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, or 90 days, or any time therebetween, after step (a). In some embodiments, providing (e.g., administering) the second composition is from 6 hours to 45 days, after step (a). In some embodiments, providing (e.g., administering) the second composition is from 6 hours to 30 days, after step (a). In some embodiments, providing (e.g., administering) the second composition is from 6 hours to 30 days plus the half-life of the protein encoded by the circular polyribonucleotide, after step (a). In some embodiments, providing (e.g., administering) the second composition is from 14 days to 30 days plus the half-life of the protein encoded by the circular polyribonucleotide, after step (a). In some embodiments, providing (e.g., administering) the second composition is from 14 days to 65 days plus the half-life of the protein encoded by the circular polyribonucleotide, after step (a). In some embodiments, providing (e.g., administering) the second composition is from 21 days to 41 days plus the half-life of the protein encoded by the circular polyribonucleotide, after step (a).

In some embodiments, the first composition comprises a first circular polyribonucleotide and the second compositions comprises a second circular polyribonucleotide, wherein the first circular polyribonucleotide and the second circular polyribonucleotide are the same. In some embodiments, the first composition comprises a first circular polyribonucleotide and the second compositions comprises a second circular polyribonucleotide, wherein the first circular polyribonucleotide and the second circular polyribonucleotide are different. In some embodiments, the first composition comprises a first circular polyribonucleotide encoding a first protein and the second compositions comprises a second circular polyribonucleotide encoding a second protein, wherein the first protein and the second protein are the same protein. In some embodiments, the first composition comprises a first circular polyribonucleotide encoding a first protein and the second compositions comprises a second circular polyribonucleotide encoding a second protein, wherein the first protein and the second protein are different proteins. In some embodiments, the first composition comprises a first circular polyribonucleotide comprising a first binding site and the second compositions comprises a second circular polyribonucleotide comprising a second binding site, wherein the first binding site and the second binding site are the same binding site. In some embodiments, the first composition comprises a first circular polyribonucleotide comprising a first binding site and the second compositions comprises a second circular polyribonucleotide comprising a second binding site, wherein the first binding site and the second binding site are different binding sites. In some embodiments, the first composition comprises a first circular polyribonucleotide encoding a protein and a second circular polyribonucleotide comprising a binding site.

In some embodiments, providing the second composition occurs after providing the first composition and before a first level of protein expressed by the first composition is substantially undetectable in the mammal (e.g., a human). In some embodiments, providing (e.g., administering) the second composition occurs after the first composition is provided (e.g., administered) and before a first level of protein expressed by the first composition decreases by more than 50% in the mammal. In some embodiments, providing (e.g., administering) the second composition occurs after the first composition is provided (e.g., administered) and before a first level of protein expressed by the first composition is substantially undetectable in the mammal. In some embodiments, providing (e.g., administering) the second composition occurs after the first composition is provided (e.g., administered) and before a first level of protein expressed by the first composition decreases 25% to 75% in the mammal. In some embodiments, the method further comprise providing (e.g., administering) a third composition of the circular polyribonucleotide to the mammal after the second composition, thereby maintaining expression of the protein in the mammal. In some embodiments, providing (e.g., administering) the third composition occurs after the second composition is provided (e.g., administered) and before a second level of the protein expressed by the first and second composition is substantially undetectable in the mammal. In some embodiments, providing (e.g., administering) the third composition occurs after the second composition is provided (e.g., administered) and before a second level of the protein expressed by the first and second composition in the mammal decreases by 25% to 75%.

In some embodiments, a method of producing a circular polyribonucleotide in a subject (e.g., mammal, e.g., a human) comprises: providing (e.g., administering) a first composition comprising the circular polyribonucleotide to the subject (e.g., mammal), wherein the subject (e.g., mammal) comprises a first level of the circular polyribonucleotide after the first composition is provided (e.g., administered); and providing (e.g., administering) a second composition of the circular polyribonucleotide to the subject (e.g., mammal), wherein the subject (e.g., mammal) comprises a second level of the circular polyribonucleotide and the second level of circular polyribonucleotide is at least as much as the first level; thereby maintaining the circular polyribonucleotide in the subject (e.g., mammal) at least at the first level. In some embodiments, a method of producing a circular polyribonucleotide in a subject (e.g., mammal, e.g., a human) comprises: providing (e.g., administering) a first composition comprising the circular polyribonucleotide to the subject (e.g., mammal), wherein the subject (e.g., mammal) comprises a first level of the circular polyribonucleotide after the first composition is provided (e.g., administered); and providing (e.g., administering) a second composition of the circular polyribonucleotides to the subject (e.g., mammal), wherein the subject (e.g., mammal) comprises a second level of the circular polyribonucleotide and the second level of circular polyribonucleotide varies by no more than 20% of the first level after the second composition is provided (e.g., administered); thereby maintaining the circular polyribonucleotide in the subject (e.g., mammal) at least at the first level. In some embodiments, a method of producing a circular polyribonucleotide in a subject (e.g., mammal, e.g., a human) comprises: providing (e.g., administering) a first composition comprising the circular polyribonucleotide to the subject (e.g., mammal), wherein the subject (e.g., mammal) comprises a first level of the circular polyribonucleotide after the first composition is provided (e.g., administered); and providing (e.g., administering) a second composition of the circular polyribonucleotides to the subject (e.g., mammal), wherein the subject (e.g., mammal) comprises a second level of the circular polyribonucleotide and the second level of circular polyribonucleotide varies by no more than 1%, 5%, 15%, 20%, 25%, 30%, 35%, 40%, or 50% of the first level after the second composition is provided (e.g., administered); thereby maintaining the circular polyribonucleotide in the subject (e.g., mammal) at least at the first level.

In some embodiments, the a first level of the protein is maintained after providing the first composition and the second composition for from 6 hours to 90 days after the first composition is provided. In some embodiments, a first level of the protein is maintained after providing the first composition, the second composition, and the third composition of the circular polyribonucleotide for from 6 hours to 270 days after the first composition is provided. In some embodiments, a first level of the protein is substantially undetectable after the first composition and the second composition are provided for 6 hours to 35 days after the first composition is provided.

Furthermore, the second composition can be provided after providing the first composition and before the level of circular polyribonucleotide from the first composition in the subject (e.g., mammal) is substantially undetectable in the subject (e.g., mammal). In some embodiments, providing the second composition occurs after the first composition is provided and before the first level of circular polyribonucleotide produced by the first composition decreases by more than 50% in the subject (e.g., mammal). In some embodiments, providing the second composition occurs after the first composition is provided and before the first level of circular polyribonucleotide produced by the first composition decreases by 25% to 75% in the subject (e.g., mammal). In some embodiments, providing the second composition occurs after the first composition is provided and before the first level of circular polyribonucleotide produced by the first composition decreases by more than 1%, 5%, 15%, 20%, 25%, 30%, 35%, 40%, or 50% in the subject (e.g., mammal).

In some embodiments, a method of producing a circular polyribonucleotide in a mammal (e.g., a human) comprises: providing (e.g., administering) a first composition comprising the circular polyribonucleotide to the mammal, wherein the mammal comprises a first level of the circular polyribonucleotide after the first composition is provided (e.g., administered); and providing (e.g., administering) a second composition of the circular polyribonucleotide to the mammal, wherein the mammal comprises a second level of the circular polyribonucleotide and the second level of circular polyribonucleotide is at least as much as the first level; thereby maintaining the circular polyribonucleotide in the mammal at least at the first level. In some embodiments, a method of producing a circular polyribonucleotide in a mammal (e.g., a human) comprises: providing (e.g., administering) a first composition comprising the circular polyribonucleotide to the mammal, wherein the mammal comprises a first level of the circular polyribonucleotide after the first composition is provided; and providing (e.g., administering) a second composition of the circular polyribonucleotides to the mammal, wherein the mammal comprises a second level of the circular polyribonucleotide and the second level of circular polyribonucleotide varies by no more than 20% of the first level after the second composition is provided (e.g., administered); thereby maintaining the circular polyribonucleotide in the mammal at least at the first level. In some embodiments, a method of producing a circular polyribonucleotide in a mammal (e.g., a human) comprises: providing (e.g., administering) a first composition comprising the circular polyribonucleotide to the mammal, wherein the mammal comprises a first level of the circular polyribonucleotide after the first composition is provided (e.g., administered); and providing (e.g., administering) a second composition of the circular polyribonucleotides to the mammal, wherein the mammal comprises a second level of the circular polyribonucleotide and the second level of circular polyribonucleotide varies by no more than 1%, 5%, 15%, 20%, 25%, 30%, 35%, 40%, or 50% of the first level after the second composition is provided (e.g., administered); thereby maintaining the circular polyribonucleotide in the mammal at least at the first level.

Furthermore, the second composition can be provided after the first composition is provided and before the level of circular polyribonucleotide from the first composition in the mammal is substantially undetectable in the mammal. In some embodiments, providing (e.g., administering) the second composition occurs after the first composition is provided (e.g., administered) and before the first level of circular polyribonucleotide produced by the first composition decreases by more than 50% in the mammal. In some embodiments, providing (e.g., administering) the second composition occurs after the first composition is administered) and before the first level of circular polyribonucleotide produced by the first composition decreases 25% to 75% in the mammal. In some embodiments, providing (e.g, administering) the second composition occurs after the first composition is provided (e.g., administered) and before the first level of circular polyribonucleotide produced by the first composition decreases by more than 1%, 5%, 15%, 20%, 25%, 30%, 35%, 40%, or 50% in the mammal.

In some embodiments, a method of expressing a protein in a subject (e.g., mammal, e.g., a human) comprises: providing (e.g., administering) a first composition comprising a circular polyribonucleotide that encodes the protein to the subject (e.g., mammal), wherein the subject (e.g., mammal) expresses a first level of an encoded protein; and providing (e.g., administering) a second composition comprising the circular polyribonucleotide to the subject (e.g., mammal), wherein the subject (e.g., mammal) expresses a second level of an encoded protein and the second level is at least as much as the first level; thereby maintaining expression of encoded protein in the subject (e.g., mammal) at least at the first level of encoded protein. In some embodiments, a method of expressing a protein in a subject (e.g., mammal, e.g., a human) comprises: providing (e.g., administering) a first composition comprising a circular polyribonucleotide that encodes the protein to the subject (e.g., mammal), wherein the subject (e.g., mammal) expresses a first level of an encoded protein; and providing (e.g., administering) a second composition comprising the circular polyribonucleotide to the subject (e.g., mammal), wherein the subject (e.g., mammal) expresses a second level of an encoded protein and the second level is at least as much as the first level; thereby maintaining expression of encoded protein in the subject (e.g., mammal) at a similar level compared to the first level of encoded protein. In some embodiments, a method of expressing a protein in a subject (e.g., mammal, e.g., a human) comprises: providing (e.g., administering) a first composition comprising a circular polyribonucleotide that encodes the protein to the subject (e.g., mammal), wherein the subject (e.g., mammal) expresses a first level of the protein; and providing (e.g., administering) a second composition comprising the circular polyribonucleotide to the subject (e.g., mammal), wherein the subject (e.g., mammal) expresses a second level of the protein and the second level varies by no more than 20% of the first level; thereby maintaining expression of the protein in the subject (e.g., mammal) at least at the first level of the protein. In some aspects, a method of expressing a protein in a subject (e.g., mammal, e.g., a human) comprises: providing (e.g., administering) a first composition comprising a circular polyribonucleotide that encodes the protein to the subject (e.g., mammal), wherein the subject (e.g., mammal) expresses a first level of the protein; and providing (e.g., administering) a second composition comprising the circular polyribonucleotide to the subject (e.g., mammal), wherein the subject (e.g., mammal) expresses a second level of the protein and the second level varies by no more than 1%, 5%, 15%, 20%, 25%, 30%, 35%, 40%, or 50% of the first level; thereby maintaining expression of the protein in the subject (e.g., mammal) at least at the first level of the protein. Furthermore, the second composition can be provided (e.g., administered) after the first composition is provided (e.g., administered) and before the level of protein produced by the first composition is substantially undetectable in the subject (e.g., mammal). In some embodiments, the second composition is provided (e.g., administered) after the first composition is provided (e.g., administered) and before the first level of protein expressed by the first composition decreases by more than 50% in the subject (e.g., mammal). In some embodiments, the second composition is provided (e.g., administered) after the first composition is provided (e.g., administered) and before the first level of protein expressed by the first composition decreases by 25% to 75% in the subject (e.g., mammal). In some embodiments, providing (e.g., administering) the second composition occurs after the first composition is provided (e.g., administered) and before the first level of protein expressed by the first composition decreases by more than 1%, 5%, 15%, 20%, 25%, 30%, 35%, 40%, or 50% in the subject (e.g., mammal).

In some embodiments, a method of expressing a protein in a mammal (e.g., a human) comprises: providing (e.g., administering) a first composition comprising a circular polyribonucleotide that encodes the protein to the mammal, wherein the mammal expresses a first level of the protein; and providing (e.g., administering) a second composition comprising the circular polyribonucleotide to the mammal, wherein the mammal expresses a second level of the protein and the second level is at least as much as the first level; thereby maintaining expression of the protein in the mammal at least at the first level of the protein. In some aspects, a method of expressing a protein in a mammal (e.g., a human) comprises: providing (e.g., administering) a first composition comprising a circular polyribonucleotide that encodes the protein to the mammal, wherein the mammal expresses a first level of the protein; and providing (e.g., administering) a second composition comprising the circular polyribonucleotide to the mammal, wherein the mammal expresses a second level of the protein and the second level varies by no more than 20% of the first level; thereby maintaining expression of the protein in the mammal at least at the first level of the protein. In some aspects, a method of expressing a protein in a mammal (e.g., a human) comprises: providing (e.g., administering) a first composition comprising a circular polyribonucleotide that encodes the protein to the mammal, wherein the mammal expresses a first level of the protein; and providing (e.g., administering) a second composition comprising the circular polyribonucleotide to the mammal, wherein the mammal expresses a second level of the protein and the second level varies by no more than 1%, 5%, 15%, 20%, 25%, 30%, 35%, 40%, or 50% of the first level; thereby maintaining expression of the protein in the mammal at least at the first level of the protein. Furthermore, the second composition can be provided (e.g., administered) after the first composition is provided (e.g., administered) and before the level of protein produced by the first composition is substantially undetectable in the mammal. In some embodiments, the second composition is provided (e.g., administered) after the first composition is provided (e.g., administered) and before the first level of protein expressed by the first composition decreases by more than 50% in the mammal. In some embodiments, the second composition is provided (e.g., administered) after the first composition is provided (e.g., administered) and before the first level of protein expressed by the first composition decreases by 25% to 75% in the mammal. In some embodiments, providing (e.g., administering) the second composition occurs after the first composition is provided (e.g., administered) and before the first level of protein expressed by the first composition decreases by more than 1%, 5%, 15%, 20%, 25%, 30%, 35%, 40%, or 50% in the mammal.

In some embodiments, a method of binding a target in a cell comprises: providing a first composition comprising the circular polyribonucleotide to the cell, wherein the cell comprises a first level of binding after the first composition is provided; and providing a second composition of the circular polyribonucleotide to the cell, wherein the cell comprises a second level of binding and the second level of binding is at least as much as the first level; thereby maintaining the binding to the target in the cell at least at the first level. In some embodiments, a method of binding to a target in a cell comprises: providing a first composition comprising the circular polyribonucleotide to the cell, wherein the cell comprises a first level of binding after the first composition is provided; and providing a second composition of the circular polyribonucleotides to the cell, wherein the cell comprises a second level of binding and the second level of binding varies by no more than 20% of the first level after providing the second composition; thereby maintaining the binding of the target in the cell at least at the first level. In some embodiments, a method of binding to a target in a cell comprises: providing a first composition comprising the circular polyribonucleotide to the cell, wherein the cell comprises a first level of the binding after the first composition is provided; and providing a second composition of the circular polyribonucleotides to the cell, wherein the cell comprises a second level of binding and the second level of binding varies by no more than 1%, 5%, 15%, 20%, 25%, 30%, 35%, 40%, or 50% of the first level after providing the second composition; thereby maintaining the binding to a target in the cell at least at the first level.

Furthermore, the second composition can be provided after the first composition is provided and before the level of binding from the first composition in the cell is substantially undetectable in the cell. In some embodiments, providing the second composition occurs after the first composition is provided and before the first level of binding produced by the first composition decreases by more than 50% in the cell. In some embodiments, providing the second composition occurs after the first composition is provided and before the first level of binding produced by the first composition decreases by 25% to 75% in the cell. In some embodiments, providing the second composition occurs after the first composition is provided and before the first level of binding produced by the first composition decreases by more than 1%, 5%, 15%, 20%, 25%, 30%, 35%, 40%, or 50% in the cell. In some embodiments, a method of binding a target in a cell comprises: providing a first composition comprising the circular polyribonucleotide to the cell, wherein the cell comprises a first level of binding after the first composition is provided; and providing a second composition of the circular polyribonucleotide to the cell, wherein the cell comprises a second level of binding and the second level of binding is at least as much as the first level; thereby maintaining the binding to the target in the cell at least at the first level. In some embodiments, a method of binding to a target in a cell comprises: providing a first composition comprising the circular polyribonucleotide to the cell, wherein the cell comprises a first level of binding after the first composition is provided; and providing a second composition of the circular polyribonucleotides to the cell, wherein the cell comprises a second level of binding and the second level of binding varies by no more than 20% of the first level after providing the second composition; thereby maintaining the binding of the target in the cell at least at the first level. In some embodiments, a method of binding to a target in a cell comprises: providing a first composition comprising the circular polyribonucleotide to the cell, wherein the cell comprises a first level of the binding after the first composition is provided; and providing a second composition of the circular polyribonucleotides to the cell, wherein the cell comprises a second level of binding and the second level of binding varies by no more than 1%, 5%, 15%, 20%, 25%, 30%, 35%, 40%, or 50% of the first level after providing the second composition; thereby maintaining the binding to a target in the cell at least at the first level.

Furthermore, the second composition can be provided after the first composition is provided and before the level of binding from the first composition in the cell is substantially undetectable in the cell. In some embodiments, providing the second composition occurs after the first composition is provided and before the first level of binding produced by the first composition decreases by more than 50% in the cell. In some embodiments, providing the second composition occurs after the first composition is provided and before the first level of binding produced by the first composition decreases by 25% to 75% in the cell. In some embodiments, providing the second composition occurs after the first composition is provided and before the first level of binding produced by the first composition decreases by more than 1%, 5%, 15%, 20%, 25%, 30%, 35%, 40%, or 50% in the cell.

In some embodiments, a method of binding a target in a subject (e.g., mammal, e.g., a human) comprises: providing (e.g., administering) a first composition comprising the circular polyribonucleotide to the subject (e.g., mammal), wherein the subject (e.g., mammal) comprises a first level of binding after the first composition is provided (e.g., administered); and providing (e.g., administering) a second composition of the circular polyribonucleotide to the subject (e.g., mammal), wherein the subject (e.g., mammal) comprises a second level of binding and the second level of binding is at least as much as the first level; thereby maintaining the binding to the target in the subject (e.g., mammal) at least at the first level. In some embodiments, a method of binding to a target in a subject (e.g., mammal, e.g., a human) comprises: providing (e.g., administering) a first composition comprising the circular polyribonucleotide to the subject (e.g., mammal), wherein the subject (e.g., mammal) comprises a first level of binding after the first composition is provided (e.g., administered); and providing (e.g., administering) a second composition of the circular polyribonucleotides to the subject (e.g., mammal), wherein the subject (e.g., mammal) comprises a second level of binding and the second level of binding varies by no more than 20% of the first level after the second composition is provided (e.g., administered); thereby maintaining the binding of the target in the subject (e.g., mammal) at least at the first level. In some embodiments, a method of binding to a target in a subject (e.g., mammal, e.g., a human) comprises: providing (e.g., administering) a first composition comprising the circular polyribonucleotide to the subject (e.g., mammal), wherein the subject (e.g., mammal) comprises a first level of the binding after the first composition is provided (e.g., administered); and providing (e.g., administering) a second composition of the circular polyribonucleotides to the subject (e.g., mammal), wherein the subject (e.g., mammal) comprises a second level of binding and the second level of binding varies by no more than 1%, 5%, 15%, 20%, 25%, 30%, 35%, 40%, or 50% of the first level after the second composition is provided (e.g., administered); thereby maintaining the binding to a target in the subject (e.g., mammal) at least at the first level.

Furthermore, the second composition can be provided (e.g., administered) after the first composition is provided (e.g., administered) and before the level of binding from the first composition in the subject (e.g., mammal) is substantially undetectable in the subject (e.g., mammal). In some embodiments, providing (e.g., administering) the second composition occurs after the first composition is provided (e.g., administered) and before the first level of binding produced by the first composition decreases by more than 50% in the subject (e.g., mammal). In some embodiments, providing (e.g., administering) the second composition occurs after the first composition is provided (e.g., administered) and before the first level of binding produced by the first composition decreases by 25% to 75% in the subject (e.g., mammal). In some embodiments, providing (e.g., administering) the second composition occurs after the first composition is provided (e.g., administered) and before the first level of binding produced by the first composition decreases by more than 1%, 5%, 15%, 20%, 25%, 30%, 35%, 40%, or 50% in the subject (e.g., mammal). In some embodiments, a method of binding a target in a subject (e.g., mammal, e.g., a human) comprises: providing (e.g., administering) a first composition comprising the circular polyribonucleotide to the subject (e.g., mammal), wherein the subject (e.g., mammal) comprises a first level of binding after the first composition is provided (e.g., administered); and providing (e.g., administering) a second composition of the circular polyribonucleotide to the subject (e.g., mammal), wherein the subject (e.g., mammal) comprises a second level of binding and the second level of binding is at least as much as the first level; thereby maintaining the binding to the target in the subject (e.g., mammal) at least at the first level. In some embodiments, a method of binding to a target in a subject (e.g., mammal, e.g., a human) comprises: providing (e.g., administering) a first composition comprising the circular polyribonucleotide to the subject (e.g., mammal), wherein the subject (e.g., mammal) comprises a first level of binding after the first composition is provided (e.g., administered); and providing (e.g., administering) a second composition of the circular polyribonucleotides to the subject (e.g., mammal), wherein the subject (e.g., mammal) comprises a second level of binding and the second level of binding varies by no more than 20% of the first level after the second composition is provided (e.g., administered); thereby maintaining the binding of the target in the subject (e.g., mammal) at least at the first level. In some embodiments, a method of binding to a target in a subject (e.g., mammal) comprises: providing (e.g., administering) a first composition comprising the circular polyribonucleotide to the subject (e.g., mammal), wherein the subject (e.g., mammal) comprises a first level of the binding after the first composition is provided (e.g., administered); and providing (e.g., administering) a second composition of the circular polyribonucleotides to the subject (e.g., mammal), wherein the subject (e.g., mammal) comprises a second level of binding and the second level of binding varies by no more than 1%, 5%, 15%, 20%, 25%, 30%, 35%, 40%, or 50% of the first level after the second composition is provided (e.g., administered); thereby maintaining the binding to a target in the subject (e.g., mammal) at least at the first level.

Furthermore, the second composition can be provided (e.g., administered) after the first composition is provided (e.g., administered) and before the level of binding from the first composition in the subject (e.g., mammal) is substantially undetectable in the subject (e.g., mammal). In some embodiments, providing (e.g., administering) the second composition occurs after the first composition is provided (e.g., administered) and before the first level of binding produced by the first composition decreases by more than 50% in the subject (e.g., mammal). In some embodiments, providing (e.g., administering) the second composition occurs after the first composition is provided (e.g., administered) and before the first level of binding produced by the first composition decreases by 25% to 75% in the subject (e.g., mammal). In some embodiments, providing (e.g., administering) the second composition occurs after the first composition is provided (e.g., administered) and before the first level of binding produced by the first composition decreases by more than 1%, 5%, 15%, 20%, 25%, 30%, 35%, 40%, or 50% in the subject (e.g., mammal).

In some embodiments, the first composition and second composition in the staggered dosing regimen or method may be followed by one or more additional composition of the circular polyribonucleotide. In some embodiments, the one or more additional compositions comprise a third, a fourth, a fifth, a sixth, a seventh, an eighth, a ninth, a tenth or more compositions.

In some embodiments, a third composition of the circular polyribonucleotide is provided (e.g., administered) to the cell or subject (e.g., mammal, e.g., a human) after the second composition, thereby maintaining the level of the circular polyribonucleotide, protein, or binding after the third composition is provided at least at the first level. In some embodiments, the third composition is provided (e.g., administered) after the second composition and before the level of the circular polyribonucleotide, protein, or binding produced by the first and second composition in the cell or subject (e.g., mammal) is substantially undetectable in the cell or subject (e.g., mammal). In some embodiments, the third composition is provided (e.g., administered) after the first composition is provided (e.g., administered) and before the first level of circular polyribonucleotide produced by, protein expressed by, or binding produced by the first composition decreases by more than 50% in the cell or subject (e.g., mammal). In some embodiments, the third composition is provided (e.g., administered) after the first composition is provided (e.g., administered) and before the first level of circular polyribonucleotide produced by, protein expressed by, or binding produced by the first composition decreases by 25% to 75% in the cell or subject (e.g., mammal). In some embodiments, the third composition is provided (e.g., administered) after the first composition is provided (e.g., administered) and before the first level of circular polyribonucleotide produced by, protein expressed by, or binding produced by the first composition decreases by more than 1%, 5%, 15%, 20%, 25%, 30%, 35%, 40%, or 50% in the cell or subject (e.g., mammal). For staggered dosing, the one or more additional compositions can be provided after providing a previous composition and before the level of circular polyribonucleotide, binding, or protein produced by the previous composition(s) is substantially undetectable in the cell or subject (e.g., mammal). In some embodiments, the one or more additional compositions is provided (e.g., administered) after a previous composition is provided (e.g., administered) and before the level of circular polyribonucleotide, binding, or protein produced by the previous composition(s) decreases by more than 50% in the cell or subject (e.g., mammal). In some embodiments, the one or more additional compositions is provided (e.g., administered) after a previous composition is provided (e.g., administered) and before the level of circular polyribonucleotide, binding, or protein produced by the previous composition(s) decreases by 25% to 75% in the cell or subject (e.g., mammal). In some embodiments, the one or more additional compositions is provided (e.g., administered) after a previous composition is provided (e.g., administered) and before the level of circular polyribonucleotide, binding, or protein produced by the previous composition(s) decreases by more than 1%, 5%, 15%, 20%, 25%, 30%, 35%, 40%, or 50% in the cell or subject (e.g., mammal).

In some embodiments, the second composition is administered to or provided to the cell or subject (e.g., mammal) before a level of the protein in the cell or subject (e.g., mammal) returns to about the level of the protein before administering or providing the first composition. In some embodiments, the second composition is administered to or provided to the cell or subject (e.g., mammal) before a level of the protein in the cell or subject (e.g., mammal) returns to about the level of the protein before administering or providing the first composition. In some embodiments, the third composition of the one or more additional doses is administered to or provided to the cell or subject (e.g., mammal) before the level of the protein in the cell or subject (e.g., mammal) returns to about the level of the protein before administering or providing the first composition. In some embodiments, the fourth, the fifth, the sixth, the seventh, the eighth, the ninth, the tenth or more compositions of the one or more additional compositions are administered to or provided to the cell or subject (e.g., mammal) before the level of the protein in the cell or subject (e.g., mammal) returns to about the level of the protein before administering or providing the first composition.

In some embodiments, a composition is administered to or provided to a cell or subject (e.g., mammal) after a time interval following administering or providing a preceding composition to the cell or subject (e.g., mammal). For example, a second composition may be administered to or provided to a cell or subject (e.g., mammal) after a first time interval following administering or providing a first composition to the cell or subject (e.g., mammal); a third composition may be administered to or provided to the cell or subject (e.g., mammal) after a second time interval following administering or providing the second composition to the cell or subject (e.g., mammal); a fourth composition may be administered to or provided to cell or subject (e.g., mammal) after a third time interval following administering or providing the third composition to the cell or subject (e.g., mammal); or a fifth, sixth, seventh, eighth, ninth, or more composition may be administered to or provided to the cell or subject (e.g., mammal) after a fourth, fifth, sixth, seventh, eighth, ninth, or more time interval following administering or providing the fourth, fifth, sixth, seventh, eighth, ninth, or more composition to the cell or subject (e.g., mammal). The first time interval may be shorter than the amount of time required for the level of the protein in cell or subject (e.g., mammal) to return to about the level of the protein before administering or providing the first composition.

In some embodiments, the second time interval is longer than the first time interval. In some embodiments, the third time interval is longer than the first time interval. In some embodiments, the fourth time interval is longer than the first time interval. In some embodiments, the fifth, sixth, seventh, eighth, ninth, or more time interval is longer than the first time interval. In some embodiments, the second time interval is the same as the first time interval. In some embodiments, the third time interval is the same as the first time interval. In some embodiments, the fourth time interval is the same as the first time interval. In some embodiments, the fifth, sixth, seventh, eighth, ninth, or more time interval is the same as the first time interval. In some embodiments, the second time interval is shorter than the first time interval. In some embodiments, the third time interval is shorter than the first time interval. In some embodiments, the fourth time interval is shorter than the first time interval. In some embodiments, the fifth, sixth, seventh, eighth, ninth, or more time interval is shorter than the first time interval. In some embodiments, the second time interval is longer than the first time interval. In some embodiments, the third time interval is longer than the second time interval. In some embodiments, the fourth time interval is longer than the third time interval.

In some embodiments, the first level of the circular polyribonucleotide is the highest level of the circular polyribonucleotide 1-2 days after providing the first composition. The highest level of circular polyribonucleotide 1-2 days after providing the first composition, for example, the peak amount of circular polyribonucleotide from 24 hours to 48 hours (e.g., 1-2 days) after providing the first composition. In some embodiments, the first level of the circular polyribonucleotide is 40%, 50%, 60%, 70%, 80%, or 90% of the highest level of the circular polyribonucleotide 1-2 days after providing the first composition. In some embodiments, the second level of the circular polyribonucleotide is at least 30%, 40%, 50%, 60%, 70%, 80%, 90%, 100%, 110%, 120%, 130%, 140%, 150%, 160%, 170%, 180%, 190%, or 200% of the highest level of the circular polyribonucleotide 1-2 days after providing the first composition for at least 1, 2, 3, 4, 5, 6, 7, 8, 9, 10, 12, 15, 20, 25, 30, 35, or 40 days after providing the second composition. In some embodiments, the second level of the circular polyribonucleotide is at least 1-fold, 5-fold, 10-fold, 100-fold, or 1000-fold higher than the highest level of the circular polyribonucleotide 1-2 days after providing the first composition for at least 1, 2, 3, 4, 5, 6, 7, 8, 9, 10, 12, 15, 20, 25, 30, 35, or 40 days after providing the second composition. In some embodiments, the third level of the circular polyribonucleotide is at least 30%, 40%, 50%, 60%, 70%, 80%, 90%, 100%, 110%, 120%, 130%, 140%, 150%, 160%, 170%, 180%, 190%, or 200% of the highest level of the circular polyribonucleotide 1-2 days after providing the first composition for at least 1, 2, 3, 4, 5, 6, 7, 8, 9, 10, 12, 15, 20, 25, 30, 35, or 40 days after providing the third composition. In some embodiments, the third level of the circular polyribonucleotide is least 1-fold, 5-fold, 10-fold, 100-fold, or 1000-fold higher than the highest level of the circular polyribonucleotide 1-2 days after providing the first composition for at least 1, 2, 3, 4, 5, 6, 7, 8, 9, 10, 12, 15, 20, 25, 30, 35, or 40 days after providing the third composition. In some embodiments, for each subsequent composition provided after the first composition, a subsequent level of the circular polyribonucleotide expressed after each subsequent composition is at least 30%, 40%, 50%, 60%, 70%, 80%, 90%, 100%, 110%, 120%, 130%, 140%, 150%, 160%, 170%, 180%, 190%, or 200% of the highest level of the circular polyribonucleotide 1-2 days after providing the first composition for at least 1, 2, 3, 4, 5, 6, 7, 8, 9, 10, 12, 15, 20, 25, 30, 35, or 40 days after providing each subsequent composition. In some embodiments, for each subsequent composition provided after the first composition, a subsequent level of the circular polyribonucleotide expressed after each subsequent composition is at least 1-fold, 5-fold, 10-fold, 100-fold, or 1000-fold higher than the highest level of the circular polyribonucleotide 1-2 days after providing the first composition for at least 1, 2, 3, 4, 5, 6, 7, 8, 9, 10, 12, 15, 20, 25, 30, 35, or 40 days after providing each subsequent composition.

In some embodiments, an average level of the circular polyribonucleotide after providing the second composition is at least 40%, 50%, 60%, 70%, 80%, or 90% of the first level, wherein the average level of the circular polyribonucleotide is measured from one day after providing the second composition to the day when the circular polyribonucleotide is substantially undetectable. In some embodiments, an average level of the circular polyribonucleotide after providing each subsequent composition after the first composition is at least 40%, 50%, 60%, 70%, 80%, or 90% of the first level, wherein the average level of the circular polyribonucleotide is measured from one day after providing each subsequent composition to the day when the circular polyribonucleotide is substantially undetectable.

In some embodiments, the first level of the circular polyribonucleotide is maintained after providing the second composition of the circular polyribonucleotide for at least 6 hours, 1 day, 2 days, 3 days, 5 days, 7 days, 14 days, 21 days, 28 days, or 35 days. In some embodiments, the first level of the circular polyribonucleotide is maintained after providing the second composition of the circular polyribonucleotide for at least 6 hours, 1 day, 2 days, 3 days, 5 days, 7 days, 14 days, 21 days, 28 days, or 35 days. In some embodiments, the first level of the circular polyribonucleotide is maintained after providing the third composition of the circular polyribonucleotide for at least 6 hours, 1 day, 2 days, 3 days, 5 days, 7 days, 14 days, 21 days, 28 days, or 35 days. In some embodiments, the second level of circular polyribonucleotide in the cell or subject (e.g., mammal) after providing the second composition is at least 1%, 5%, 10%, 20%, 30%, 40%, 50%, or 60% higher than the first level of circular polyribonucleotide in the cell or subject (e.g., mammal) after providing the first composition. In some embodiments, the third level of circular polyribonucleotide in the cell or subject (e.g., mammal) after providing the third composition is at least 5%, 10%, 20%, 30%, 40%, 50%, or 60% higher than the first level of circular polyribonucleotide in the plurality after providing the first composition.

In some embodiments, the second level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the circular polyribonucleotide after providing the first composition. In some embodiments, the third level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the circular polyribonucleotide after providing the first composition.

In some embodiments, the first level of the binding is maintained after providing the second composition of the circular polyribonucleotide for at least 6 hours, 1 day, 2 days, 3 days, 5 days, 7 days, 14 days, 21 days, 28 days, or 35 days. In some embodiments, the first level of binding is maintained after providing the third composition of the circular polyribonucleotide for at least 6 hours, 1 day, 2 days, 3 days, 5 days, 7 days, 14 days, 21 days, 28 days, or 35 days. In some embodiments, the second level of binding in the cell or subject (e.g., mammal) after providing the second composition is at least 1%, 5%, 10%, 20%, 30%, 40%, 50%, or 60% higher than the first level of binding in the cell or subject (e.g., mammal) after providing the first composition. In some embodiments, the third level of binding in the cell or subject (e.g., mammal) after providing the third composition is at least 5%, 10%, 20%, 30%, 40%, 50%, or 60% higher than the first level of binding in the plurality after providing the first composition.

In some embodiments, the first level of the protein is the highest level of the protein 1-2 days after providing the first composition. The highest level of protein 1-2 days after providing the first composition, for example, the peak amount of protein expressed from the circular polyribonucleotide from 24 hours to 48 hours (e.g., 1-2 days) after providing the first composition. In some embodiments, the first level of the protein is 40%, 50%, 60%, 70%, 80%, or 90% of the highest level of the protein 1-2 days after providing the first composition. In some embodiments, the second level of the protein is at least 30%, 40%, 50%, 60%, 70%, 80%, 90%, 100%, 110%, 120%, 130%, 140%, 150%, 160%, 170%, 180%, 190%, or 200% of the highest level of the protein 1-2 days after providing the first composition for at least 1, 2, 3, 4, 5, 6, 7, 8, 9, 10, 12, 15, 20, 25, 30, 35, or 40 days after providing the second composition. In some embodiments, the second level of the protein is at least 1-fold, 5-fold, 10-fold, 100-fold, or 1000-fold higher than the highest level of the protein 1-2 days after providing the first composition for at least 1, 2, 3, 4, 5, 6, 7, 8, 9, 10, 12, 15, 20, 25, 30, 35, or 40 days after providing the second composition. In some embodiments, the third level of the protein is at least 30%, 40%, 50%, 60%, 70%, 80%, 90%, 100%, 110%, 120%, 130%, 140%, 150%, 160%, 170%, 180%, 190%, or 200% of the highest level of the protein 1-2 days after providing the first composition for at least 1, 2, 3, 4, 5, 6, 7, 8, 9, 10, 12, 15, 20, 25, 30, 35, or 40 days after providing the third composition. In some embodiments, the third level of the protein is at least 1-fold, 5-fold, 10-fold, 100-fold, or 1000-fold higher than the highest level of the protein 1-2 days after providing the first composition for at least 1, 2, 3, 4, 5, 6, 7, 8, 9, 10, 12, 15, 20, 25, 30, 35, or 40 days after providing the third composition. In some embodiments, for each subsequent composition provided after the first composition, a subsequent level of the protein expressed after each subsequent composition is at least 30%, 40%, 50%, 60%, 70%, 80%, 90%, 100%, 110%, 120%, 130%, 140%, 150%, 160%, 170%, 180%, 190%, or 200% of the highest level of the protein 1-2 days after providing the first composition for at least 1, 2, 3, 4, 5, 6, 7, 8, 9, 10, 12, 15, 20, 25, 30, 35, or 40 days after providing each subsequent composition. In some embodiments, for each subsequent composition provided after the first composition, a subsequent level of the protein expressed after each subsequent composition is at least 1-fold, 5-fold, 10-fold, 100-fold, or 1000-fold higher than the highest level of the protein 1-2 days after providing the first composition for at least 1, 2, 3, 4, 5, 6, 7, 8, 9, 10, 12, 15, 20, 25, 30, 35, or 40 days after providing each subsequent composition.

In some embodiments, an average level of the protein after providing the second composition is at least 40%, 50%, 60%, 70%, 80%, or 90% of the first level, wherein the average level of the protein is measured from one day after providing the second composition to the day when the protein is substantially undetectable. In some embodiments, an average level of the protein after providing each subsequent composition after the first composition is at least 40%, 50%, 60%, 70%, 80%, or 90% of the first level, wherein the average level of the protein is measured from one day after providing each subsequent composition to the day when the protein is substantially undetectable.

In some embodiments, the first level of the protein is maintained after providing the first composition and the second composition of the circular polyribonucleotide for at least 6 hours, 1 day, 2 days, 3 days, 5 days, 7 days, 14 days, 21 days, 28 days, or 35 days after the first composition is provided. In some embodiments, the first level of the protein is maintained after providing the first composition, second composition, and third composition of the circular polyribonucleotide for at least 6 hours, 1 day, 2 days, 3 days, 5 days, 7 days, 14 days, 21 days, 28 days, or 35 days after the first composition is provided.

In some embodiments, the second level of protein in the cell or subject (e.g., mammal) after providing the second composition is at least 1%, 5%, 10%, 20%, 30%, 40%, 50%, or 60% higher than the first level of protein in the cell or subject (e.g., mammal) after the first composition is provided. In some embodiments, the third level of protein in the cell or subject (e.g., mammal) after providing the third composition is at least 5%, 10%, 20%, 30%, 40%, 50%, or 60% higher than the first level of protein in the plurality after the first composition is provided.

In some embodiments, the second level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the protein after the first composition is provided. In some embodiments, the third level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the protein after the first composition is provided.

In some embodiments, the level of the protein of the first composition is maintained after providing the second composition of the circular polyribonucleotide for at least 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days. In some embodiments, the level of the protein of the first composition is maintained after providing the third composition of the circular polyribonucleotide for at least 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days. In some embodiments, the level of the protein of the first composition is maintained after providing the fourth composition, fifth composition, sixth composition, seventh composition, eighth composition, nine composition, tenth composition or more of the circular polyribonucleotide for at least 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days. The level of the protein that is maintained is the level of the protein in cell or subject (e.g., mammal) at day 1 after the first composition is provided. In some embodiments, the level of the circular polyribonucleotide in the cell or subject (e.g., mammal) after the first composition is maintained after providing the second composition of the circular polyribonucleotide for at least 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days. In some embodiments, the level of the circular polyribonucleotide in the cell or subject (e.g., mammal) after the first composition is maintained after providing the third composition of the circular polyribonucleotide for at least 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days. In some embodiments, the level of the circular polyribonucleotide in the cell or subject (e.g., mammal) after the first composition is maintained after providing the fourth composition, fifth composition, sixth composition, seventh composition, eighth composition, nine composition, tenth composition or more of the circular polyribonucleotide for at least 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days.

In some embodiments, the level of protein in the cell or subject (e.g., mammal) after providing the second composition of the circular polyribonucleotide is from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% higher than the level of protein in cell or subject (e.g., mammal) after providing the first composition. In some embodiments, the level of circular polyribonucleotide in cell or subject (e.g., mammal) after providing the second composition of the circular polyribonucleotide is from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% higher than the level of circular polyribonucleotide in the cell or subject (e.g., mammal) after providing the first composition.

### Redosing

A method of redosing to produce a level of circular polyribonucleotide, a level of binding, or express protein in a cell after providing the cell with at least two compositions of circular polyribonucleotide is disclosed herein. A method of redosing to produce a level of circular polyribonucleotide, a level of binding, or express protein in a subject (e.g., a mammal, e.g., a human) after providing the cell with at least two compositions of circular polyribonucleotide is disclosed herein.

In some embodiments, the at least two compositions of circular polyribonucleotide are the same compositions. In some embodiments, the at least two compositions of circular polyribonucleotide are different compositions. In some embodiments, the same compositions comprise circular polyribonucleotides encoding the same proteins or comprising the same binding sites. In some embodiments, the different compositions comprise circular polyribonucleotides encoding different proteins or comprising different binding sites. In some embodiments, the first composition comprises a first circular polyribonucleotide encoding a first protein and the second compositions comprises a second circular polyribonucleotide encoding a second protein, wherein the first protein and the second protein are the same protein. In some embodiments, the first composition comprises a first circular polyribonucleotide encoding a first protein and the second compositions comprises a second circular polyribonucleotide encoding a second protein, wherein the first protein and the second protein are different proteins. In some embodiments, the first composition comprises a first circular polyribonucleotide comprising a first binding site and the second compositions comprises a second circular polyribonucleotide comprising a second binding site, wherein the first binding site and the second binding site are the same binding site. In some embodiments, the first composition comprises a first circular polyribonucleotide comprising a first binding site and the second compositions comprises a second circular polyribonucleotide comprising a second binding site, wherein the first binding site and the second binding site are different binding sites. In some embodiments, the first composition comprises a first circular polyribonucleotide encoding a protein and the second compositions comprises a second circular polyribonucleotide comprising a binding site.

In some embodiments, a method of maintaining expression of a protein in a mammal (e.g., a human), comprises: (a) providing (e.g., administering) a first composition comprising a circular polyribonucleotide that encodes the protein to the mammal; and (b) from 6 hours to 90 days following step (a), providing (e.g., administering) a second composition comprising a circular polyribonucleotide that encodes the protein, to the mammal, thereby maintaining expression of the protein in the mammal. In some embodiments, providing (e.g., administering) the second composition occurs after the first composition is provided (e.g., administered) and after the first level of the protein expressed by the first composition is substantially undectable in the mammal. In some embodiments, the method further comprises providing (e.g., administering) a third composition of the circular polyribonucleotide to the mammal after the second composition, thereby restoring the protein in the mammal.

In some embodiments a method of maintaining expression of an antigen in a mammal (e.g., a human), comprises: (a) providing (e.g., administering) a first composition comprising a circular polyribonucleotide that encodes the antigen to the mammal; and (b) from 6 hours to 90 days following step (a), providing (e.g., administering) a second composition comprising a circular polyribonucleotide that encodes the antigen, to the mammal, thereby maintaining expression of the protein in the mammal.

In some embodiments, providing (e.g., administering) the second composition is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, or 90 days, any time therebetween, after step (a). In some embodiments, providing (e.g., administering) the second composition is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, or 90 months, or any time therebetween, after step (a). In some embodiments, providing (e.g., administering) the second composition is 1, 2, 3, 4, 5, 6, 7, 8, 9, 10, 11, 12, 13, 14, 15, 16, 17, 18, 19, or 20 years, or any time therebetween, after step (a). In some embodiments, providing (e.g., administering) the second composition is from 6 hours to 45 days, after step (a). In some embodiments, providing (e.g., administering) the second composition is from 6 hours to 30 days, after step (a). In some embodiments, providing (e.g., administering) the second composition is from 6 hours to 65 days, after step (a). In some embodiments, providing (e.g., administering) the second composition is from 30 days to 45 days, after step (a). In some embodiments, providing (e.g., administering) the second composition is from 14 days to 30 days, after step (a). In some embodiments, providing (e.g., administering) the second composition is from 14 days to 45 days, after step (a). In some embodiments, providing (e.g., administering) the second composition is from 30 days to 65 days, after step (a). In some embodiments, providing (e.g, administering) the second composition is from 30 days to 90 days, after step (a).

In some embodiments, the a first level of the protein is maintained after providing (e.g., administering) the first composition and the second composition for from 6 hours to 90 days after the first composition is provided (e.g., administered). In some embodiments, a first level of the protein is maintained after providing (e.g., administering) the first composition, the second composition, and the third composition of the circular polyribonucleotide for from 6 hours to 270 days after the first composition is provided (e.g., administered). In some embodiments, a first level of the protein is substantially undetectable after providing (e.g., administering) the first composition and the second composition for 6 hours to 35 days after the first composition is provided (e.g., administered).

In some embodiments, a method of expressing a level of a protein in a cell after providing a first composition and a second composition of a circular polyribonucleotide to a cell compared to a level of the protein in a cell after providing a first composition and second composition of a linear counterpart of the circular polyribonucleotide, comprises: providing a first composition of a circular polyribonucleotide encoding a protein to a cell, wherein the cell comprises a level of the protein after the first composition of the circular polyribonucleotide is provided; and providing a second composition of circular polyribonucleotide after the first composition to the cell, wherein the cell comprises at least the level of the protein after the second composition of the circular polyribonucleotide is provided; thereby maintaining expression of the level of the protein in the cell after the first composition and the second composition of the circular polyribonucleotide are provided compared to the level of the protein in the cell after the first composition and the second composition of a linear counterpart of the circular polyribonucleotide are provided. In some embodiments, a method of expressing a level of a protein in a cell after a first composition and a second composition of circular polyribonucleotide are provided to a cell compared to a level of the protein in a cell after a first composition and second composition of a linear counterpart of the circular polyribonucleotide are provided, comprises: providing a first composition of circular polyribonucleotide encoding a protein to a cell, wherein the cell comprises a level of the protein after the first composition of circular polyribonucleotide is provided; and providing the second composition of circular polyribonucleotide after the first composition to a cell, wherein the cell comprises a level of the protein that varies by no more than 20% of the level after the second composition of circular polyribonucleotide is provided; thereby maintaining expression of the level of protein in a cell after the first composition is provided and the second composition of circular polyribonucleotide compared to a level of the protein in a cell after the first composition is provided and the second composition of linear counterpart of circular polyribonucleotide.

In some embodiments, a method of expressing a level of a protein in a subject (e.g., mammal) after providing a first composition and a second composition of a circular polyribonucleotide to the subject (e.g., mammal) compared to a level of the protein in the subject (e.g., mammal) after providing a first composition and second composition of a linear counterpart of the circular polyribonucleotide, comprises: providing (e.g., administering) a first composition of circular polyribonucleotide encoding a protein to the subject (e.g., mammal), wherein the subject (e.g., mammal) comprises a level of the protein after the first composition of the circular polyribonucleotide is provided (e.g., administered); and providing (e.g., administering) the second composition of the circular polyribonucleotide after the first composition to the subject (e.g., mammal), wherein the subject (e.g., mammal) comprises at least the level of the protein after the second composition of the circular polyribonucleotide is provided (e.g., administered); thereby maintaining expression of the level of the protein in the subject (e.g., mammal) after the first composition and the second composition of the circular polyribonucleotide are provided (e.g., administered) compared to the level of the protein in the subject (e.g., mammal) after the first composition and the second composition of the linear counterpart of the circular polyribonucleotide are provided (e.g., administered). In some embodiments, a method of expressing a level of a protein in a subject (e.g., mammal) after providing a first composition and a second composition of a circular polyribonucleotide to the subject (e.g., mammal) compared to a level of the protein in the subject (e.g., mammal) after providing a first composition and second composition of a linear counterpart of the circular polyribonucleotide, comprises: providing (e.g., administering) a first composition of the circular polyribonucleotide encoding the protein to the subject (e.g., mammal), wherein the subject (e.g., mammal) comprises a level of the protein after the first composition of the circular polyribonucleotide is provided (e.g., administered); and providing (e.g., administering) the second composition of the circular polyribonucleotide after the first composition to the subject (e.g., mammal), wherein the subject (e.g., mammal) comprises a level of the protein that varies by no more than 20% of the level after the second composition of the circular polyribonucleotide is provided (e.g., administered); thereby maintaining expression of the level of the protein in the subject (e.g., mammal) after the first composition and the second composition of the circular polyribonucleotide are provided (e.g., administered) compared to the level of the protein in the subject (e.g., mammal) after the first composition and the second composition of the linear counterpart of the circular polyribonucleotide are provided (e.g., administered).

In some embodiments, a method of expressing a level of a protein in a cell after providing a first composition and a second composition of a circular polyribonucleotide to the cell compared to a level of the protein in the cell after providing a first composition and second composition of a linear counterpart of the circular polyribonucleotide, comprises: providing a first composition of the circular polyribonucleotide encoding the protein to the cell, wherein the cell comprises the level of the protein after the first composition of the circular polyribonucleotide is provided; and providing the second composition of the circular polyribonucleotide after the first composition to the cell, wherein the cell comprises a level of the protein that varies by no more than 1%, 5%, 15%, 20%, 25%, 30%, 35%, 40%, or 50% of the level after the second composition of the circular polyribonucleotide is provided; thereby maintaining expression of the level of the protein in the cell after providing the first composition and the second composition of the circular polyribonucleotide compared to the level of the protein in the cell after providing the first composition and the second composition of the linear counterpart of the circular polyribonucleotide.

In some embodiments, a method of expressing a level of a protein in a subject (e.g., mammal) after providing a first composition and a second composition of a circular polyribonucleotide to the subject (e.g., mammal) compared to a level of the protein in the subject (e.g., mammal) after providing a first composition and second composition of a linear counterpart of the circular polyribonucleotide, comprises: providing (e.g., administering) a first composition of the circular polyribonucleotide encoding the protein to the subject (e.g., mammal), wherein the subject (e.g., mammal) comprises a level of the protein after the first composition of the circular polyribonucleotide is provided (e.g., administered); and providing (e.g., administering) the second composition of the circular polyribonucleotide after the first composition to the subject (e.g., mammal), wherein the subject (e.g., mammal) comprises a level of the protein that varies by no more than 1%, 5%, 15%, 20%, 25%, 30%, 35%, 40%, or 50% of the level after the second composition of the circular polyribonucleotide is provided (e.g., administered); thereby maintaining expression of the level of the protein in the subject (e.g., mammal) after the first composition and the second composition of the circular polyribonucleotide are provided (e.g., administered) compared to the level of the protein in the subject (e.g., mammal) after the first composition and the second composition of the linear counterpart of the circular polyribonucleotide are provided (e.g., administered).

Furthermore, the second composition can be provided after providing the first composition and after the level of protein produced by the first composition is substantially undetectable in the cell or subject (e.g., mammal). In some embodiments, the second composition is provided to the cell or subject (e.g., mammal) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the level of protein in the cell or subject (e.g., mammal) produced by the first composition is substantially undetectable. In some embodiments, the second composition is provided to the cell or subject (e.g., mammal) from 1 minute to 20 years, or any time therebetween, after the level of protein in the cell or subject (e.g., mammal) produced by the first composition is substantially undetectable. In some embodiments, the second composition is provided to the cell or subject (e.g., mammal) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the first composition and less than 20 years, 15 years, or 10 years after the first composition. In some embodiments, the second composition is provided to the cell or subject (e.g., mammal) from 1 minute to 20 years, or any time therebetween.

In some embodiments, a method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprises: providing a first composition of the circular polyribonucleotide to the cell, wherein the cell comprises the level of the circular polyribonucleotide after providing the first composition; and providing the second composition of the circular polyribonucleotide to the cell, wherein the cell comprises at least the level of the circular polyribonucleotide after providing the second composition; thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide. In some embodiments, a method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprises: providing a first composition of the circular polyribonucleotide to the cell, wherein the cell comprises a level of the circular polyribonucleotide after the first composition is provided; and providing the second composition of the circular polyribonucleotide to the cell, wherein the cell comprises a level of the protein after providing the second composition that varies by no more than 20% of the level of the circular polyribonucleotide; thereby maintaining the level of the circular polyribonucleotide in the cell after the first composition and the second composition of the circular polyribonucleotide are provided compared to the level of the linear counterpart in the cell after the first composition and the second composition of the linear counterpart of the circular polyribonucleotide are provided.

In some embodiments, a method of producing a level of a circular polyribonucleotide in a subject (e.g., mammal) after providing a first composition and a second composition of the circular polyribonucleotide to the subject (e.g., mammal) compared to a level of a linear counterpart of the circular polyribonucleotide in the subject (e.g., mammal) after providing a first composition and second composition of the linear counterpart of the circular polyribonucleotide, comprises: providing (e.g., administering) a first composition of the circular polyribonucleotide to the subject (e.g., mammal), wherein the subject (e.g., mammal) comprises a level of the circular polyribonucleotide after the first composition is provided (e.g., administered); and providing (e.g., administering) the second composition of the circular polyribonucleotide to the subject (e.g., mammal), wherein the subject (e.g., mammal) comprises at least the level of the circular polyribonucleotide after the second composition is provided (e.g., administered); thereby maintaining the level of the circular polyribonucleotide in the subject (e.g., mammal) after the first composition and the second composition of the circular polyribonucleotide are provided (e.g., administered) compared to the level of the linear counterpart in the subject (e.g., mammal) after the first composition and the second composition of the linear counterpart of the circular polyribonucleotide are provided (e.g., administered). In some embodiments, a method of producing a level of a circular polyribonucleotide in a subject (e.g., mammal) after providing a first composition and a second composition of the circular polyribonucleotide to the subject (e.g., mammal) compared to a level of a linear counterpart of the circular polyribonucleotide in the subject (e.g., mammal) after providing a first composition and second composition of the linear counterpart of the circular polyribonucleotide, comprises: providing (e.g., administering) a first composition of the circular polyribonucleotide to the subject (e.g., mammal, e.g., a human), wherein the subject (e.g., mammal) comprises a level of the circular polyribonucleotide after the first composition is provided (e.g., administered); and providing (e.g., administering) the second composition of the circular polyribonucleotide to the subject (e.g., mammal), wherein the subject (e.g., mammal) comprises a level of the protein after the second composition is provided (e.g., administered) that varies by no more than 20% of the level of the circular polyribonucleotide; thereby maintaining the level of the circular polyribonucleotide in the subject (e.g., mammal) after the first composition and the second composition of the circular polyribonucleotide are provided (e.g., administered) compared to the level of the linear counterpart in the subject (e.g., mammal) after the first composition and the second composition of the linear counterpart of the circular polyribonucleotide are provided (e.g., administered).

In some embodiments, a method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprises: providing a first composition of the circular polyribonucleotide to the cell, wherein the cell comprises a level of the circular polyribonucleotide after the first composition is provided; and providing the second composition of the circular polyribonucleotide to the cell, wherein the cell comprises a level of the protein after the second composition is provided that varies by no more than 1%, 5%, 15%, 20%, 25%, 30%, 35%, 40%, or 50% of the level of the circular polyribonucleotide; thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide.

In some embodiments, a method of producing a level of a circular polyribonucleotide in a subject (e.g., mammal, e.g., a human) after providing a first composition and a second composition of the circular polyribonucleotide to the subject (e.g., mammal) compared to a level of a linear counterpart of the circular polyribonucleotide in the subject (e.g., mammal) after providing a first composition and second composition of the linear counterpart of the circular polyribonucleotide, comprises: providing (e.g., administering) a first composition of the circular polyribonucleotide to the subject (e.g., mammal), wherein the subject (e.g., mammal) comprises a level of the circular polyribonucleotide after the first composition is provided (e.g., administered); and providing (e.g., administering) the second composition of the circular polyribonucleotide to the subject (e.g., mammal), wherein the subject (e.g., mammal) comprises a level of the protein after the second composition is provided (e.g., administered) that varies by no more than 1%, 5%, 15%, 20%, 25%, 30%, 35%, 40%, or 50% of the level of the circular polyribonucleotide; thereby maintaining the level of the circular polyribonucleotide in the subject (e.g., mammal) after providing the first composition and the second composition of the circular polyribonucleotide compared to the level of the linear counterpart in the subject (e.g., mammal) after providing the first composition and the second composition of the linear counterpart of the circular polyribonucleotide.

In some embodiments, the circular polyribonucleotide is an exogenous, synthetic circular polyribonucleotide. In some embodiments, the circular polyribonucleotide lacks a poly-A sequence, a replication element, or both.

In some embodiments, the first composition comprises a first circular polyribonucleotide and the second compositions comprises a second circular polyribonucleotide, wherein the first circular polyribonucleotide and the second circular polyribonucleotide are the same. In some embodiments, the first composition comprises a first circular polyribonucleotide and the second compositions comprises a second circular polyribonucleotide, wherein the first circular polyribonucleotide and the second circular polyribonucleotide are different.

Furthermore, the second composition can be provided after providing the first composition and before the level of circular polyribonucleotide in the cell or subject (e.g., mammal, e.g., a human) from the first composition is substantially undetectable in the cell or subject (e.g., mammal, e.g., a human). In some embodiments, the second composition is provided (e.g., administered) to the cell or subject (e.g., mammal, e.g., a human) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the level of the circular polyribonucleotide in the cell or subject (e.g., mammal) produced by the first composition is substantially undetectable. In some embodiments, the second composition is provided (e.g., administered) to the cell or subject (e.g., mammal, e.g., a human) from 1 minute to 20 years, or any time therebetween, after the level of circular polyribonucleotide in the cell or subject (e.g., mammal) produced by the first composition is substantially undetectable. In some embodiments, the second composition is provided to the cell or subject (e.g., mammal, e.g., a human) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the first composition and less than 20 years, 15 years, or 10 years after the first composition. In some embodiments, the second composition is provided (e.g., administered) to the cell or subject (e.g., mammal, e.g., a human) from 1 minute to 20 years, or any time therebetween.

In some embodiments, a method binding a target in a cell after providing a first composition and a second composition of a circular polyribonucleotide to the cell compared to a level of binding in the cell after providing a first composition and second composition of a linear counterpart of the circular polyribonucleotide, comprises: providing a first composition of the circular polyribonucleotide encoding the protein to the cell, wherein the cell comprises the level of binding after providing the first composition of the circular polyribonucleotide; and providing the second composition of the circular polyribonucleotide after the first composition to the cell, wherein the cell comprises at least the level of binding after providing the second composition of the circular polyribonucleotide; thereby maintaining expression of the level of binding in the cell after providing the first composition and the second composition of the circular polyribonucleotide compared to the level of binding in the cell after providing the first composition and the second composition of the linear counterpart of the circular polyribonucleotide. In some embodiments, a method of binding to a target in a cell after providing a first composition and a second composition of a circular polyribonucleotide to the cell compared to a level of the protein in the cell after providing a first composition and second composition of a linear counterpart of the circular polyribonucleotide, comprises: providing a first composition of the circular polyribonucleotide encoding the protein to the cell, wherein the cell comprises the level of the protein after providing the first composition of the circular polyribonucleotide; and providing the second composition of the circular polyribonucleotide after the first composition to the cell, wherein the cell comprises a level of binding that varies by no more than 20% of the level after providing the second composition of the circular polyribonucleotide; thereby maintaining the level of binding in the cell after providing the first composition and the second composition of the circular polyribonucleotide compared to the level of the protein in the cell after providing the first composition and the second composition of the linear counterpart of the circular polyribonucleotide.

In some embodiments, a method binding a target in a subject (e.g., mammal, e.g., a human) after providing a first composition and a second composition of a circular polyribonucleotide to the subject (e.g., mammal) compared to a level of binding in the subject (e.g., mammal) after providing a first composition and second composition of a linear counterpart of the circular polyribonucleotide, comprises: providing (e.g., administering) a first composition of the circular polyribonucleotide encoding the protein to the subject (e.g., mammal, e.g., a human), wherein the subject (e.g., mammal) comprises a level of binding after the first composition of the circular polyribonucleotide is provided (e.g., administered); and providing (e.g., administering) the second composition of the circular polyribonucleotide after the first composition to the subject (e.g., mammal), wherein the subject (e.g., mammal) comprises at least the level of binding after providing the second composition of the circular polyribonucleotide; thereby maintaining expression of the level of binding in the subject (e.g., mammal) after the first composition and the second composition of the circular polyribonucleotide are provided (e.g., administered) compared to the level of binding in the subject (e.g., mammal) after the first composition and the second composition of the linear counterpart of the circular polyribonucleotide are provided (e.g., administered). In some embodiments, a method of binding to a target in a subject (e.g., mammal) after providing a first composition and a second composition of a circular polyribonucleotide to the subject (e.g., mammal) compared to a level of the protein in the subject (e.g., mammal) after providing a first composition and second composition of a linear counterpart of the circular polyribonucleotide, comprises: providing (e.g., administering) a first composition of the circular polyribonucleotide encoding the protein to the subject (e.g., mammal, e.g., human), wherein the subject (e.g., mammal) comprises a level of the protein after the first composition of the circular polyribonucleotide is provided (e.g., administered); and providing (e.g., administering) the second composition of the circular polyribonucleotide after the first composition to the subject (e.g., mammal), wherein the subject (e.g., mammal) comprises a level of binding that varies by no more than 20% of the level after the second composition of the circular polyribonucleotide is provided (e.g., administered); thereby maintaining the level of binding in the subject (e.g., mammal) after providing (e.g., administering) the first composition and the second composition of the circular polyribonucleotide compared to the level of the protein in the subject (e.g., mammal) after providing (e.g., administering) the first composition and the second composition of the linear counterpart of the circular polyribonucleotide.

In some embodiments, a method for producing a level of binding in a cell after providing a first composition and a second composition of a circular polyribonucleotide to the cell compared to a level of binding in the cell after providing a first composition and second composition of a linear counterpart of the circular polyribonucleotide, comprises: providing a first composition of the circular polyribonucleotide comprising a binding site to the cell, wherein the cell comprises a level of binding after the first composition of the circular polyribonucleotide is provided; and providing the second composition of the circular polyribonucleotide after the first composition to the cell, wherein the cell comprises a level of binding that varies by no more than 1%, 5%, 15%, 20%, 25%, 30%, 35%, 40%, or 50% of the level after the second composition of the circular polyribonucleotide is provided; thereby maintaining expression of the level of binding in the cell after providing the first composition and the second composition of the circular polyribonucleotide compared to the level of binding in the cell after providing the first composition and the second composition of the linear counterpart of the circular polyribonucleotide.

In some embodiments, a method binding a target in a subject (e.g., mammal, e.g., human) after providing a first composition and a second composition of a circular polyribonucleotide to the subject (e.g., mammal) compared to a level of binding in the subject (e.g., mammal) after providing a first composition and second composition of a linear counterpart of the circular polyribonucleotide, comprises: providing (e.g., administering) a first composition of the circular polyribonucleotide encoding the protein to the subject (e.g., mammal, e.g., a human), wherein the subject (e.g., mammal) comprises a level of binding after the first composition of the circular polyribonucleotide is provided (e.g., administered); and providing (e.g., administering) the second composition of the circular polyribonucleotide after the first composition to the subject (e.g., mammal), wherein the subject (e.g., mammal) comprises at least the level of binding after the second composition of the circular polyribonucleotide is provided (e.g., administered); thereby maintaining expression of the level of binding in the subject (e.g., mammal) after providing the first composition and the second composition of the circular polyribonucleotide compared to the level of binding in the subject (e.g., mammal) after providing the first composition and the second composition of the linear counterpart of the circular polyribonucleotide. In some embodiments, a method of binding to a target in a subject (e.g., mammal) after providing a first composition and a second composition of a circular polyribonucleotide to the subject (e.g., mammal) compared to a level of the protein in the subject (e.g., mammal) after providing a first composition and second composition of a linear counterpart of the circular polyribonucleotide, comprises: providing (e.g., administering) a first composition of the circular polyribonucleotide encoding the protein to the subject (e.g., mammal), wherein the subject (e.g., mammal) comprises a level of the protein after the first composition of the circular polyribonucleotide is provided (e.g., administered); and providing (e.g., administering) the second composition of the circular polyribonucleotide after the first composition to the subject (e.g., mammal), wherein the subject (e.g., mammal) comprises a level of binding that varies by no more than 20% of the level after the second composition of the circular polyribonucleotide is provided (e.g., administered); thereby maintaining the level of binding in the subject (e.g., mammal) after providing the first composition and the second composition of the circular polyribonucleotide compared to the level of the protein in the subject (e.g., mammal) after providing the first composition and the second composition of the linear counterpart of the circular polyribonucleotide.

Furthermore, the second composition can be provided after providing the first composition and after the level of protein produced by the first composition is substantially undetectable in the cell or subject (e.g., mammal). In some embodiments, the second composition is provided (e.g., administered) to the cell or subject (e.g., mammal, e.g., a human) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the level of binding in the cell or subject (e.g., mammal) produced by the first composition is substantially undetectable. In some embodiments, the second composition is provided (e.g., administered) to the cell or subject (e.g., mammal, e.g. a human) from 1 minute to 20 years, or any time therebetween, after the level of binding in the cell or subject (e.g., mammal) produced by the first composition is substantially undetectable. In some embodiments, the second composition is provided (e.g., administered) to the cell or subject (e.g., mammal, e.g., a human) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the first composition and less than 20 years, 15 years, or 10 years after the first composition. In some embodiments, the second composition is provided (e.g., administered) to the cell or subject (e.g., mammal, e.g., a human) from 1 minute to 20 years, or any time therebetween.

In some embodiments, the first composition and second composition in the redosing regimen or method may be followed by one or more additional compositions of the circular polyribonucleotide. In some embodiments, the one or more additional compositions comprise a third, a fourth, a fifth, a sixth, a seventh, an eighth, a ninth, a tenth or more compositions. For redosing, the one or more additional compositions can be provided after providing a previous composition and after the level of circular polyribonucleotide, binding or protein produced by the previous composition(s) is substantially undetectable in the plurality of cells (e.g., in a subject). For example, the one or more additional compositions is provided to the plurality at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the level of circular polyribonucleotide produced by, binding produced by, or protein expressed by the previous composition is substantially undetectable. For redosing, the one or more additional compositions can be provided after providing a previous composition. For example, the one or more additional compositions is provided to cell or subject (e.g., a mammal) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after providing the previous composition(s). In some embodiments, the one or more additional compositions is provided to cell or subject (e.g., a mammal) from 1 minute to 20 years after providing the previous composition(s). In some embodiments, the one or more additional compositions is provided to cell or subject (e.g., a mammal) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year, but no more than 20 years, 15 years, or 10 years after providing the previous composition(s).

In some embodiments, the second composition is administered to or provided to the cell or subject (e.g., mammal, e.g., a human) after a level of the protein in the cell or subject (e.g., mammal) returns to about the level of the protein before administering or providing the first composition. In some embodiments, the second composition is provided to the cell or subject (e.g., mammal) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after a level of the protein in cell or subject (e.g., mammal) returns to about the level of the protein before administering or providing the first composition. In some embodiments, the third composition of the one or more additional compositions is administered to or provided to the cell or subject (e.g., mammal) after the level of the protein in the cell or subject (e.g., mammal) returns to about the level of the protein before administering or providing the first composition. In some embodiments, the third composition of the one or more additional compositions is administered to or provided to the cell or subject (e.g., mammal) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after a level of the protein in the cell or subject (e.g., mammal) returns to about the level of the protein before administering or providing the first composition. In some embodiments, the fourth, the fifth, the sixth, the seventh, the eighth, the ninth, the tenth or more compositions of the one or more additional compositions are administered to or provided to the cell or subject (e.g., mammal) after the level of the protein in the cell or subject (e.g., mammal) returns to about the level of the protein before administering or providing the first composition. In some embodiments, the fourth, the fifth, the sixth, the seventh, the eighth, the ninth, the tenth or more compositions of the one or more additional compositions are administered to or provided to the cell or subject (e.g., mammal) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after a level of the protein in the cell or subject (e.g., mammal) returns to about the level of the protein before administering or providing the first composition. In some embodiments, the compositions as described above are administered or provided to the cell or subject (e.g., mammal) from 1 minute to 20 years after a level of the protein in the cell or subject (e.g., mammal) returns to about the level of the protein before administering or providing the first composition.

In some embodiments, the second composition is administered to or provided to the cell after providing the first composition. In some embodiments, the second composition is provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after providing the first composition. In some embodiments, the third composition of the one or more additional compositions is administered to or provided to the cell after providing the first composition. In some embodiments, the third composition of the one or more additional compositions is administered to or provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after providing the first composition. In some embodiments, the fourth, the fifth, the sixth, the seventh, the eighth, the ninth, the tenth or more compositions of the one or more additional compositions are administered to or provided to the cell after providing the first composition. In some embodiments, the fourth, the fifth, the sixth, the seventh, the eighth, the ninth, the tenth or more compositions of the one or more additional composition are administered to or provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after providing the first composition. In some embodiments, the compositions as described above are administered or provided to the cell or subject (e.g., mammal) from 1 minute to 20 years after providing the first composition.

In some embodiments, a composition is administered to or provided to a cell or subject (e.g., mammal) after a time interval following administering or providing a preceding composition to the cell or subject (e.g., mammal). For example, a second composition may be administered to or provided to a cell or subject (e.g., mammal) after a first time interval following administering or providing a first composition to the cell or subject (e.g., mammal); a third composition may be administered to or provided to the cell or subject (e.g., mammal) after a second time interval following administering or providing the second composition to the cell or subject (e.g., mammal); a fourth composition may be administered to or provided to the cell or subject (e.g., mammal) after a third time interval following administering or providing the third composition to the cell or subject (e.g., mammal); or a fifth, sixth, seventh, eighth, ninth, or more composition may be administered to or provided to the cell or subject (e.g., mammal) after a fourth, fifth, sixth, seventh, eighth, ninth, or more time interval following administering or providing the fourth, fifth, sixth, seventh, eighth, ninth, or more composition to the cell or subject (e.g., mammal). The first time interval may be longer than the amount of time required for the level of the protein in the cell or subject (e.g., mammal) returns to about the level of the protein before administering or providing the first composition.

In some embodiments, the second time interval is longer than the first time interval. In some embodiments, the third time interval is longer than the first time interval. In some embodiments, the fourth time interval is longer than the first time interval. In some embodiments, the fifth, sixth, seventh, eighth, ninth, or more time interval is longer than the first time interval. In some embodiments, the second time interval is the same as the first time interval. In some embodiments, the third time interval is the same as the first time interval. In some embodiments, the fourth time interval is the same as the first time interval. In some embodiments, the fifth, sixth, seventh, eighth, ninth, or more time interval is the same as the first time interval. In some embodiments, the second time interval is shorter than the first time interval. In some embodiments, the third time interval is shorter than the first time interval. In some embodiments, the fourth time interval is shorter than the first time interval. In some embodiments, the fifth, sixth, seventh, eighth, ninth, or more time interval is shorter than the first time interval. In some embodiments, the second time interval is longer than the first time interval. In some embodiments, the third time interval is longer than the second time interval. In some embodiments, the fourth time interval is longer than the third time interval.

In some embodiments, the level of the protein in the cell or subject (e.g., mammal) after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.

In some embodiments, the level of the protein in the cell or subject (e.g., mammal) after providing the first composition and the second composition of the circular polyribonucleotide is at least 5%, 10%, 20%, 30%, 40%, 50%, or 60% higher than the level of the protein in the cell or subject (e.g., mammal) after providing the first composition and the second composition of the linear counterpart of the circular polyribonucleotide.

In some embodiments, the level of the protein in the cell or subject (e.g., mammal) after providing the first composition and the second composition of the circular polyribonucleotide is at least 5%, 10%, 20%, 30%, 40%, 50%, or 60% higher than the level of the protein in the cell or subject (e.g., mammal) after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.

In some embodiments, the level of the circular polyribonucleotide in the cell or subject (e.g., mammal) after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.

In some embodiments, the level of the circular polyribonucleotide in the cell or subject (e.g., mammal) after providing the first composition and the second composition of the circular polyribonucleotide is at least 5%, 10%, 20%, 30%, 40%, 50%, or 60% higher than the level of the linear counterpart of the circular polyribonucleotide in the cell or subject (e.g., mammal) after providing the first composition and the second composition of the linear counterpart of the circular polyribonucleotide.

In some embodiments, the level of the circular polyribonucleotide in the plurality after providing the first composition and the second composition of the circular polyribonucleotide is at least 5%, 10%, 20%, 30%, 40%, 50%, or 60% higher than the level of the linear counterpart of the circular polyribonucleotide in the plurality after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.

In some embodiments, the level of binding in the cell or subject (e.g., mammal) after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.

In some embodiments, the level of binding in the cell or subject (e.g., mammal) after providing the first composition and the second composition of the circular polyribonucleotide is at least 5%, 10%, 20%, 30%, 40%, 50%, or 60% higher than the level of binding in the cell or subject (e.g., mammal) after providing the first composition and the second composition of the linear counterpart of the circular polyribonucleotide.

In some embodiments, the level of the circular polyribonucleotide in the plurality after providing the first composition and the second composition of the circular polyribonucleotide is at least 5%, 10%, 20%, 30%, 40%, 50%, or 60% higher than the level of binding in the plurality after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.

### Circular Polyribonucleotides

The circular polyribonucleotides described herein and compositions or pharmaceutical compositions thereof may be used in therapeutic and veterinary methods of dosing to produce a level of circular polyribonucleotide, a level of binding to a target, or a level of protein in a plurality of cells after providing the plurality with at least two doses of circular polyribonucleotide. In some embodiments, the circular polyribonucleotide is non-immunogenic in a mammal, e.g., a human. In some embodiments, the circular polyribonucleotide is capable of replicating or replicates in a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof. In some embodiments, the invention includes a cell comprising the circular polyribonucleotide described herein, wherein the cell is a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, a cultured cell, a primary cell or a cell line, a stem cell, a progenitor cell, a differentiated cell, a germ cell, a cancer cell (e.g., tumorigenic, metastic), a non-tumorigenic cell (normal cells), a fetal cell, an embryonic cell, an adult cell, a mitotic cell, a non-mitotic cell, or any combination thereof. In some embodiments, the cell is modified to comprise the circular polyribonucleotide.

In some embodiments, the circular polyribonucleotide includes sequences for expression products. In some embodiments, the circular polyribonucleotide comprises a binding site for binding to a target. In some embodiments, the circular polyribonucleotide is provided to a plurality of cells via any of the dosing, staggered dosing, or redosing methods described herein. In some embodiments, the circular polyribonucleotide as described herein induces a response or response level in a subject. In some embodiments, the expression products encoded by the sequences included in the circular polyribonucleotide are expressed in one or more of cells in the plurality of cells.

In some embodiments, the circular polyribonucleotide has a half-life of at least that of a linear counterpart, e.g., linear expression sequence, or linear circular polyribonucleotide. In some embodiments, the circular polyribonucleotide has a half-life that is increased over that of a linear counterpart. In some embodiments, the half-life is greater by about 5%, 10%, 15%, 20%, 25%, 30%, 35%, 40%, 45%, 50%, or greater. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween. In some embodiments, the circular polyribonucleotide has a half-life or persistence in a cell while the cell is dividing. In some embodiments, the circular polyribonucleotide has a half-life or persistence in a cell post division. In certain embodiments, the circular polyribonucleotide has a half-life or persistence in a dividing cell for greater than about 10 minutes to about 30 days, or at least about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the circular polyribonucleotide modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the circular polyribonucleotide may be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, or at least 100 nucleotides may be useful.

In some embodiments, the circular polyribonucleotide comprises one or more expression sequences and is configured for persistent expression in a cell of a subject in vivo. In some embodiments, the circular polyribonucleotide is configured such that expression of the one or more expression sequences in the cell at a later time point is equal to or higher than an earlier time point. In such embodiments, the expression of the one or more expression sequences can be either maintained at a relatively stable level or can increase overtime. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

Expression Sequences

Disclosed herein is are dosing methods that produce a level of protein from an expression sequence in a cell after providing the cell with at least two compositions of circular polyribonucleotide wherein the circular polyribonucleotide encode the protein.

In some embodiments, the circular polyribonucleotide comprises at least one expression sequence that encodes a peptide or polypeptide. Such peptide may include, but is not limited to, small peptide, peptidomimetic (e.g., peptoid), amino acids, and amino acid analogs. The peptide may be linear or branched. Such peptide may have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such peptide may include, but is not limited to, a neurotransmitter, a hormone, a drug, a toxin, a viral or microbial particle, a synthetic molecule, and agonists or antagonists thereof.

The polypeptide may be linear or branched. The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Some examples of a peptide or polypeptide include, but are not limited to, fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, an intra-organellar antigen.

In some embodiments, the circular polyribonucleotide comprises one or more RNA expression sequences, each of which may encode a polypeptide. The polypeptide may be produced in substantial amounts. As such, the polypeptide may be any proteinaceous molecule that can be produced. A polypeptide can be a polypeptide that can be secreted from a cell, or localized to the cytoplasm, nucleus or membrane compartment of a cell. Some polypeptides include, but are not limited to, at least a portion of a viral envelope protein, metabolic regulatory enzymes (e.g., that regulate lipid or steroid production), an antigen, a toleragen, a cytokine, a toxin, enzymes whose absence is associated with a disease, and polypeptides that are not active in an animal until cleaved (e.g., in the gut of an animal), and a hormone.

In some embodiments, the circular polyribonucleotide includes an expression sequence encoding a protein e.g., a therapeutic protein. In some embodiments, the expression product of the expression sequence is a protein, e.g., a therapeutic protein. In some embodiments, therapeutic proteins that can be expressed from the circular polyribonucleotide disclosed herein have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins may include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof. In some embodiments, the therapeutic protein is an antigen. In some embodiments, an antigen is a tumor antigen, a bacterial antigen, or a viral antigen.

In some embodiments, exemplary proteins that can be expressed from the circular polyribonucleotide disclosed herein include human proteins, for instance, receptor binding protein, hormone, growth factor, growth factor receptor modulator, and regenerative protein (e.g., proteins implicated in proliferation and differentiation, e.g., therapeutic protein, for wound healing). In some embodiments, exemplary proteins that can be expressed from the circular polyribonucleotide disclosed herein include EGF (epithelial growth factor). In some embodiments, exemplary proteins that can be expressed from the circular polyribonucleotide disclosed herein include enzymes, for instance, oxidoreductase enzymes, metabolic enzymes, mitochondrial enzymes, oxygenases, dehydrogenases, ATP-independent enzyme, and desaturases. In some embodiments, exemplary proteins that can be expressed from the circular polyribonucleotide disclosed herein include an intracellular protein or cytosolic protein. In some embodiments, the circular polyribonucleotide expresses a phenylalanine hydroxylase. In some embodiments, the circular polyribonucleotide expresses a NanoLuc® luciferase (nLuc). In some embodiments, exemplary proteins that can be expressed from the circular polyribonucleotide disclosed herein include a secreted protein, for instance, a secretary enzyme. In some embodiments, the circular polyribonucleotide expresses an erythropoietin. In some cases, the circular polyribonucleotide expresses a secretary protein that can have a short half-life therapeutic in the blood, or can be a protein with a subcellular localization signal, or protein with secretory signal peptide. In some embodiments, the circular polyribonucleotide expresses a Gaussia Luciferase (gLuc). In some embodiments, exemplary proteins that can be expressed from the circular polyribonucleotide disclosed herein include a membrane protein, or a transmembrane protein. In some embodiments, the circular polyribonucleotide expresses a transmembrane receptor, e.g., a G-protein-coupled receptor (GPCR), a receptor tyrosine kinase (RTK), an antigen receptor, or a chimeric antigen receptor. In some cases, the circular polyribonucleotide expresses a non-human protein, for instance, a fluorescent protein, an energy-transfer acceptor, or a protein-tag like Flag, Myc, or His. In some embodiments, exemplary proteins that can be expressed from the circular polyribonucleotide include a GFP. In some embodiments, the circular polyribonucleotide expresses tagged proteins, e.g., fusion proteins or engineered proteins containing a protein tag, e.g., chitin binding protein (CBP), maltose binding protein (MBP), Fc tag, glutathione-S-transferase (GST), AviTag (GLNDIFEAQKIEWHE), Calmodulin-tag (KRRWKKNFIAVSAANRFKKISSSGAL); polyglutamate tag (EEEEEE); E-tag (GAPVPYPDPLEPR); FLAG-tag (DYKDDDDK), HA-tag (YPYDVPDYA); His-tag (HHHHHH); Myc-tag (EQKLISEEDL); NE-tag (TKENPRSNQEESYDDNES); S-tag (KETAAAKFERQHMDS); SBP-tag (MDEKTTGWRGGHVVEGLAGELEQLRARLEHHPQGQREP); Softag 1 (SLAELLNAGLGGS); Softag 3 (TQDPSRVG); Spot-tag (PDRVRAVSHWSS); Strep-tag (Strep-tag II. WSHPQFEK); TC tag (CCPGCC); Ty tag (EVHTNQDPLD); V5 tag (GKPIPNPLLGLDST); VSV-tag (YTDIEMNRLGK); or Xpress tag (DLYDDDDK).

In some embodiments, the circular polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can comprise more than one expression sequence, each of which expresses a portion of an antibody, and the sum of which can constitute the antibody. In some cases, the circular polyribonucleotide comprises one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

Disclosed herein is are dosing methods that produce a level of protein in a cell after providing the cell with at least two compositions of circular polyribonucleotide wherein the circular polyribonucleotide encode the protein.

A protein can be an intracellular protein, a membrane protein, or a secreted protein. A protein can be a polypeptide that can be secreted from a cell, or localized to the cytoplasm, nucleus or membrane compartment of a cell. A protein can include, but is not limited to, at least a portion of a viral envelope protein, metabolic regulatory enzymes (e.g., that regulate lipid or steroid production), an antigen, a toleragen, a cytokine, a toxin, enzymes whose absence is associated with a disease, and polypeptides that are not active in an animal until cleaved (e.g., in the gut of an animal), and a hormone.

In some embodiments, the protein is a therapeutic protein. The therapeutic protein can have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins may include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof. In some embodiments, the therapeutic protein is Human Factor VIII, Human Factor IX, REP1, adenosine deaminase, human NGF, nuclear-encoded ND4, SECRA2a, SUMO1, VEGF, PDE6A, p53, PBFD, ARSA, ABCD1, APOE4, RPGR, DCLRE1C, VEGF 165, PDGF-B, gamma-sarcoglycan, dystrophin, LAMP2B, CNGB3, Retinitis Pigmentosa GTPase Regulator, or CLN6.

In some embodiments, the protein includes human proteins, for instance, receptor binding protein, hormone, growth factor, growth factor receptor modulator, and regenerative protein (e.g., proteins implicated in proliferation and differentiation, e.g., therapeutic protein, for wound healing). In some embodiments, the protein is EGF (epithelial growth factor). In some embodiments, an exemplary protein is an enzyme, for instance, oxidoreductase enzyme, metabolic enzyme, mitochondrial enzyme, oxygenase, dehydrogenase, ATP-independent enzyme, and desaturase. In some embodiments, exemplary proteins disclosed herein include an intracellular protein or cytosolic protein. In some embodiments, exemplary proteins include a secretory protein, for instance, a secretory enzyme. In some cases, a secretory protein can have a short half-life therapeutic in the blood, or can be a protein with a subcellular localization signal, or protein with secretory signal peptide. In some cases, the protein is a non-human protein, for instance, a fluorescent protein, an energy-transfer acceptor, or a protein-tag like Flag, Myc, or His. In some embodiments, the protein is a tagged protein, e.g., fusion protein or engineered protein containing a protein tag, e.g., chitin binding protein (CBP), maltose binding protein (MBP), Fc tag, glutathione-S-transferase (GST), AviTag (GLNDIFEAQKIEWHE), Calmodulin-tag (KRRWKKNFIAVSAANRFKKISSSGAL); polyglutamate tag (EEEEEE); E-tag (GAPVPYPDPLEPR); FLAG-tag (DYKDDDDK), HA-tag (YPYDVPDYA); His-tag (HHHHHH); Myc-tag (EQKLISEEDL); NE-tag (TKENPRSNQEESYDDNES); S-tag (KETAAAKFERQHMDS); SBP-tag (MDEKTTGWRGGHVVEGLAGELEQLRARLEHHPQGQREP); Softag 1 (SLAELLNAGLGGS); Softag 3 (TQDPSRVG); Spot-tag (PDRVRAVSHWSS); Strep-tag (Strep-tag II. WSHPQFEK); TC tag (CCPGCC); Ty tag (EVHTNQDPLD); V5 tag (GKPIPNPLLGLDST); VSV-tag (YTDIEMNRLGK); or Xpress tag (DLYDDDDK).

In some embodiments, protein is an antibody, e.g., an antibody fragment, or a portion thereof. The antibody can be of any isotype, such as IgA, IgD, IgE, IgG, IgM. In some embodiments, the protein is a portion of an antibody, such as a light chain, a heavy chain, a Fc fragment, a CDR (complementary determining region), a Fv fragment, or a Fab fragment, a further portion thereof. In some embodiments, protein is one or more portions of an antibody. For instance, the circular polyribonucleotide can comprise more than one expression sequence, each of which expresses a portion of an antibody, and the sum of which can constitute the antibody. In some cases, the circular polyribonucleotide comprises one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

The present invention includes a method for protein expression, comprising translating at least a region of the circular polyribonucleotide provided herein. Protein expression may occur in one or more cells, for example a cell after providing a first composition, a second composition, a third composition, a fourth composition, a fifth composition, a sixth composition, a seventh composition, an eighth composition, a ninth composition, or a tenth composition to the cell.

In some embodiments, the methods for protein expression comprises translation of at least 10%, at least 20%, at least 30%, at least 40%, at least 50%, at least 60%, at least 70%, at least 80%, at least 90%, or at least 95% of the total length of the circular polyribonucleotide into polypeptides. In some embodiments, the methods for protein expression comprises translation of the circular polyribonucleotide into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression comprises translation of the circular polyribonucleotide into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods comprise translation of the circular polyribonucleotide into continuous polypeptides as provided herein, discrete polypeptides as provided herein, or both.

In some embodiments, the translation of the at least a region of the circular polyribonucleotide takes place in vitro, such as rabbit reticulocyte lysate. In some embodiments, the translation of the at least a region of the circular polyribonucleotide takes place in vivo, for instance, after transfection of a eukaryotic cell, or transformation of a prokaryotic cell such as a bacteria. In some embodiments, the translation takes place in one or more cells, for example after providing a composition of the circular polyribonucleotide to a cell.

In some aspects, the present disclosure provides methods of in vivo expression of one or more expression sequences in a subject, comprising: administering a circular polyribonucleotide to a cell of the subject wherein the circular polyribonucleotide comprises the one or more expression sequences; and expressing the one or more expression sequences from the circular polyribonucleotide in the cell. In some embodiments, the circular polyribonucleotide is configured such that expression of the one or more expression sequences in the cell at a later time point is equal to or higher than an earlier time point. In some embodiments, the circular polyribonucleotide expresses of the one or more expression sequences in the cell over a time period of at least 7, 8, 9, 10, 12, 14, 16, 18, 20, 22, 23 or more days does not decrease by greater than about 40%. In some embodiments, the circular polyribonucleotide expresses of the one or more expression sequences in the cell is maintained at a level that does not vary by more than about 40% for at least 7, 8, 9, 10, 12, 14, 16, 18, 20, 22, 23 or more days. In some embodiments, the administration of the circular polyribonucleotide is conducted using any delivery method described herein. In some embodiments, the circular polyribonucleotide is administered to the subject via intravenous injection. In some embodiments, the administration of the circular polyribonucleotide includes, but is not limited to, prenatal administration, neonatal administration, postnatal administration, oral, by injection (e.g., intravenous, intraarterial, intraperotoneal, intradermal, intracranial, intrathecal, intralymphatic, subcutaneous and intramuscular), by ophthalmic administration, by intracochlear (inner ear) administration, by intranasal administration, by intratracheal administration, and through inhaled administration.

In some embodiments, the methods for protein expression comprise modification, folding, or other post-translation modification of the translation product. In some embodiments, the methods for protein expression comprise post-translation modification in vivo or in a cell, e.g., via cellular machinery.

Binding Site

In some embodiments, the circular polyribonucleotide encodes at least one binding site. The at least one binding site can bind a target, such as protein, RNA, or DNA. The at least one binding site be a protein binding site, an RNA binding site, or a DNA binding site. The at least one binding site confers at least one therapeutic characteristic to the cell. In some embodiments, the at least one binding site confers nucleic acid (e.g., the circular polyribonucleotide as described herein) localization to a cell. In some embodiments, the at least one binding site confers nucleic acid activity (e.g., is a miRNA binding site that results in nucleic acid degradation in cells comprising the miRNA) to the cell comprising the circular polyribonucleotide. In some embodiments, the at least one binding site binds to a cell receptor on a surface of a cell. In some embodiments, a circular polyribonucleotide is internalized into the cell as described herein when the at least one binding site binds to a cell receptor on the surface of the cell. In some embodiments, the at least binding site hybridizes to a linear polynucleotide that aids in internalization of the circular polyribonucleotide into a cell. For example, the linear polynucleotide comprises a region that hybridizes to the at least one binding site of the circular polyribonucleotide and a region that binds to a cell receptor on the surface of the cell. In some embodiments, the region of the linear polyribonucleotide that binds to the cell receptor results in internalization of the linear polyribonucleotide hybridized to the circular polyribonucleotide after binding.

In some instances, a circRNA comprises a binding site. A binding site can comprise an aptamer. In some instances, a circRNA comprises at least two binding sites. For example, a circRNA can comprise 2, 3, 4, 5, 6, 7, 8, 9, 10, 11, 12, 13, 14, 15, 16, 17, 18, 19, 20, or more binding sites. In some embodiments, circRNA described herein is a molecular scaffold that binds one or more targets, or one or more binding moieties of one or more targets. Each target may be, but is not limited to, a different or the same nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, cells, cellular moieties, any fragments thereof, and any combination thereof. In some embodiments, the one or more binding sites binds to the same target. In some embodiments, the one or more binding sites bind to one or more binding moieties of the same target. In some embodiments, the one or more binding sites bind to one or more different targets. In some embodiments, the one or more binding sites bind to one or more binding moieties of different targets. In some embodiments, a circRNA acts as a scaffold for one or more binding one or more targets. In some embodiments, a circRNA acts as a scaffold for one or more binding moieties of one or more targets. In some embodiments, a circRNA modulates cellular processes by specifically binding to one or more one or more targets. In some embodiments, a circRNA modulates cellular processes by specifically binding to one or more binding moieties of one or more targets. In some embodiments, a circRNA modulates cellular processes by specifically binding to one or more targets. In some embodiments, a circRNA described herein includes binding sites for one or more specific targets of interest. In some embodiments, circRNA includes multiple binding sites or a combination of binding sites for each target of interest. In some embodiments, circRNA includes multiple binding sites or a combination of binding sites for each binding moiety of interest. For example, a circRNA can include one or more binding sites for a polypeptide target. In some embodiments, a circRNA includes one or more binding sites for a polynucleotide target, such as a DNA or RNA, an mRNA target, an rRNA target, a tRNA target, or a genomic DNA target.

In some instances, a circRNA comprises a binding site for a single-stranded DNA. In some instances, a circRNA comprises a binding site for double-stranded DNA. In some instances, a circRNA comprises a binding site for an antibody. In some instances, a circRNA comprises a binding site for a virus particle. In some instances, a circRNA comprises a binding site for a small molecule. In some instances, a circRNA comprises a binding site that binds in or on a cell. In some instances, a circRNA comprises a binding site for a RNA-DNA hybrid. In some instances, a circRNA comprises a binding site for a methylated polynucleotide. In some instances, a circRNA comprises a binding site for an unmethylated polynucleotide. In some instances, a circRNA comprises a binding site for an aptamer. In some instances, a circRNA comprises a binding site for a polypeptide. In some instances, a circRNA comprises a binding site for a polypeptide, a protein, a protein fragment, a tagged protein, an antibody, an antibody fragment, a small molecule, a virus particle (e.g., a virus particle comprising a transmembrane protein), or a cell. In some instances, a circRNA comprises a binding site for a binding moiety on a single-stranded DNA. In some instances, a circRNA comprises a binding site for a binding moiety on a double-stranded DNA. In some instances, a circRNA comprises a binding site for a binding moiety on an antibody. In some instances, a circRNA comprises a binding site for a binding moiety on a virus particle. In some instances, a circRNA comprises a binding site for a binding moiety on a small molecule. In some instances, a circRNA comprises a binding site for a binding moiety in or on a cell. In some instances, a circRNA comprises a binding site for a binding moiety on a RNA-DNA hybrid. In some instances, a circRNA comprises a binding site for a binding moiety on a methylated polynucleotide. In some instances, a circRNA comprises a binding site for a binding moiety on an unmethylated polynucleotide. In some instances, a circRNA comprises a binding site for a binding moiety on an aptamer. In some instances, a circRNA comprises a binding site for a binding moiety on a polypeptide. In some instances, a circRNA comprises a binding site for a binding moiety on a polypeptide, a protein, a protein fragment, a tagged protein, an antibody, an antibody fragment, a small molecule, a virus particle (e.g., a virus particle comprising a transmembrane protein), or a cell.

In some instances, a binding site binds to a portion of a target comprising at least two amide bonds. In some instances, a binding site does not bind to a portion of a target comprising a phosphodiester linkage. In some instances, a portion of the target is not DNA or RNA. In some instances, a binding moiety comprises at least two amide bonds. In some instances, a binding moiety does not comprise a phosphodiester linkage. In some instances, a binding moiety is not DNA or RNA.

The circRNAs provided herein can include one or more binding sites for binding moieties on a complex. The circRNAs provided herein can include one or more binding sites for targets to form a complex. For example, the circRNAs provided herein can act as a scaffold to form a complex between a circRNA and a target. In some embodiments, a circRNA forms a complex with a single target. In some embodiments, a circRNA forms a complex with two targets. In some embodiments, a circRNA forms a complex with three targets. In some embodiments, a circRNA forms a complex with four targets. In some embodiments, a circRNA forms a complex with five or more targets. In some embodiments, a circRNA forms a complex with a complex of two or more targets. In some embodiments, a circRNA forms a complex with a complex of three or more targets. In some embodiments, two or more circRNAs form a complex with a single target. In some embodiments, two or more circRNAs form a complex with two or more targets. In some embodiments, a first circRNA forms a complex with a first binding moiety of a first target and a second different binding moiety of a second target. In some embodiments, a first circRNA forms a complex with a first binding moiety of a first target and a second circRNA forms a complex with a second binding moiety of a second target.

In some embodiments, a circRNA can include a binding site for one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some embodiments, a circRNA can include a binding site for one or more binding moieties on one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some instances, a binding site binds to a polypeptide, protein, or fragment thereof. In some embodiments, a binding site binds to a domain, a fragment, an epitope, a region, or a portion of a polypeptide, protein, or fragment thereof of a target. For example, a binding site binds to a domain, a fragment, an epitope, a region, or a portion of an isolated polypeptide, a polypeptide of a cell, a purified polypeptide, or a recombinant polypeptide. For example, a binding site binds to a domain, a fragment, an epitope, a region, or a portion of an antibody or fragment thereof. For example, a binding site binds to a domain, a fragment, an epitope, a region, or a portion of a transcription factor. For example, a binding site binds to a domain, a fragment, an epitope, a region, or a portion of a receptor. For example, a binding site binds to a domain, a fragment, an epitope, a region, or a portion of a transmembrane receptor. Binding sites may bind to a domain, a fragment, an epitope, a region, or a portion of isolated, purified, and/or recombinant polypeptides. Binding sites can bind to a domain, a fragment, an epitope, a region, or a portion of a mixture of analytes (e.g., a lysate). For example, a binding site binds to a domain, a fragment, an epitope, a region, or a portion of from a plurality of cells or from a lysate of a single cell. A binding site can bind to a binding moiety of a target. In some instances, a binding moiety is on a polypeptide, protein, or fragment thereof. In some embodiments, a binding moiety comprises a domain, a fragment, an epitope, a region, or a portion of a polypeptide, protein, or fragment thereof. For example, a binding moiety comprises a domain, a fragment, an epitope, a region, or a portion of an isolated polypeptide, a polypeptide of a cell, a purified polypeptide, or a recombinant polypeptide. For example, a binding moiety comprises a domain, a fragment, an epitope, a region, or a portion of an antibody or fragment thereof. For example, a binding moiety comprises a domain, a fragment, an epitope, a region, or a portion of a transcription factor. For example, a binding moiety comprises a domain, a fragment, an epitope, a region, or a portion of a receptor. For example, a binding moiety comprises a domain, a fragment, an epitope, a region, or a portion of a transmembrane receptor. Binding moieties may be on or comprise a domain, a fragment, an epitope, a region, or a portion of isolated, purified, and/or recombinant polypeptides. Binding moieties include binding moieties on or a domain, a fragment, an epitope, a region, or a portion of a mixture of analytes (e.g., a lysate). For example, binding moieties are on or comprise a domain, a fragment, an epitope, a region, or a portion of from a plurality of cells or from a lysate of a single cell.

In some instances, a binding site binds to a domain, a fragment, an epitope, a region, or a portion of a chemical compound (e.g., small molecule). For example, a binding binds to a domain, a fragment, an epitope, a region, or a portion of a drug. For example, a binding site binds to a domain, a fragment, an epitope, a region, or a portion of a compound. For example, a binding moiety binds to a domain, a fragment, an epitope, a region, or a portion of an organic compound. In some instances, a binding site binds to a domain, a fragment, an epitope, a region, or a portion of a small molecule with a molecular weight of 900 Daltons or less. In some instances, a binding site binds to a domain, a fragment, an epitope, a region, or a portion of a small molecule with a molecular weight of 500 Daltons or more. The portion the small molecule that the binding site binds to may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference. A binding site can bind to a binding moiety of a small molecule. In some instances, a binding moiety is on or comprises a domain, a fragment, an epitope, a region, or a portion of a small molecule. For example, a binding moiety is on or comprises a domain, a fragment, an epitope, a region, or a portion of a drug. For example, a binding moiety is on or comprises a domain, a fragment, an epitope, a region, or a portion of a compound. For example, a binding moiety is on or comprises a domain, a fragment, an epitope, a region, or a portion of an organic compound. In some instances, a binding moiety is on or comprises a domain, a fragment, an epitope, a region, or a portion of a small molecule with a molecular weight of 900 Daltons or less. In some instances, a binding moiety is on or comprises a domain, a fragment, an epitope, a region, or a portion of a small molecule with a molecular weight of 500 Daltons or more. Binding moieties may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

A binding site can bind to a domain, a fragment, an epitope, a region, or a portion of a member of a specific binding pair (e.g., a ligand). A binding site can bind to a domain, a fragment, an epitope, a region, or a portion of monovalent (monoepitopic) or polyvalent (polyepitopic). A binding site can bind to an antigenic or haptenic portion of a target. A binding site can bind to a domain, a fragment, an epitope, a region, or a portion of a single molecule or a plurality of molecules that share at least one common epitope or determinant site. A binding site can bind to a domain, a fragment, an epitope, a region, or a portion of a part of a cell (e.g., a bacteria cell, a plant cell, or an animal cell). A binding site can bind to a target that is in a natural environment (e.g., tissue), a cultured cell, or a microorganism (e.g., a bacterium, fungus, protozoan, or virus), or a lysed cell. A binding site can bind to a portion of a target that is modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group. A binding site can bind to a binding moiety of a member of a specific binding pair. A binding moiety can be on or comprise a domain, a fragment, an epitope, a region, or a portion of a member of a specific binding pair (e.g., a ligand). A binding moiety can be on or comprise a domain, a fragment, an epitope, a region, or a portion of monovalent (monoepitopic) or polyvalent (polyepitopic). A binding moiety can be antigenic or haptenic. A binding moiety can be on or comprise a domain, a fragment, an epitope, a region, or a portion of a single molecule or a plurality of molecules that share at least one common epitope or determinant site. A binding moiety can be on or comprise a domain, a fragment, an epitope, a region, or a portion of a part of a cell (e.g., a bacteria cell, a plant cell, or an animal cell). A binding moiety can be either in a natural environment (e.g., tissue), a cultured cell, or a microorganism (e.g., a bacterium, fungus, protozoan, or virus), or a lysed cell. A binding moiety can be modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

In some instances, a binding site binds to a domain, a fragment, an epitope, a region, or a portion of a molecule found in a sample from a host. A binding site can bind to a binding moeity of a molecule found in a sample from a host. In some instances, a binding moiety is on or comprises a domain, a fragment, an epitope, a region, or a portion of a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render a binding moiety more readily detectible. Samples include a quantity of a substance from a living thing or formerly living things. A sample can be natural, recombinant, synthetic, or not naturally occurring. A binding site can bind to any of the above that is expressed from a cell naturally or recombinantly, in a cell lysate or cell culture medium, an in vitro translated sample, or an immunoprecipitation from a sample (e.g., a cell lysate). A binding moiety can be any of the above that is expressed from a cell naturally or recombinantly, in a cell lysate or cell culture medium, an in vitro translated sample, or an immunoprecipitation from a sample (e.g., a cell lysate).

In some instances, a binding site binds to a target expressed in a cell-free system or in vitro. For example, a binding site binds to a target in a cell extract. In some instances, a binding site binds to a target in a cell extract with a DNA template, and reagents for transcription and translation. A binding site can bind to a binding moiety of a target expressed in a cell-free system or in vitro. In some instances, a binding moiety of a target is expressed in a cell-free system or in vitro. For example, a binding moiety of a target is in a cell extract. In some instances, a binding moiety of a target is in a cell extract with a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express target polypeptides (e.g., to produce target polypeptides on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and μIP-microintaglio printing, and pMAC-protein microarray copying (See Kilb et al., Eng. Life Sci. 2014, 14, 352-364).

In some instances, a binding site binds to a target that is synthesized in situ (e.g., on a solid substrate of an array) from a DNA template. A binding site can bind to binding moiety of a target that is synthesized in situ. In some instances, a binding moiety of a target is synthesized in situ (e.g., on a solid substrate of an array) from a DNA template. In some instances, a plurality of binding moieties is synthesized in situ from a plurality of corresponding DNA templates in parallel or in a single reaction. Exemplary methods for in situ target polypeptide expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, a binding site binds to a nucleic acid target comprising a span of at least 6 nucleotides, for example, least 8, 9, 10, 12, 15, 20, 25, 30, 40, 50, or 100 nucleotides. In some instances, a binding site binds to a protein target comprising a contiguous stretch of nucleotides. In some instances, a binding site binds to a protein target comprising a non-contiguous stretch of nucleotides. In some instances, a binding site binds to a nucleic acid target comprising a site of a mutation or functional mutation, including a deletion, addition, swap, or truncation of the nucleotides in a nucleic acid sequence. A binding site can bind to a binding moiety of a nucleic acid target. In some instances, a binding moiety of a nucleic acid target comprises a span of at least 6 nucleotides, for example, least 8, 9, 10, 12, 15, 20, 25, 30, 40, 50, or 100 nucleotides. In some instances, a binding moiety of a protein target comprises a contiguous stretch of nucleotides. In some instances, a binding moiety of a protein target comprises a non-contiguous stretch of nucleotides. In some instances, a binding moiety of a nucleic acid target comprises a site of a mutation or functional mutation, including a deletion, addition, swap, or truncation of the nucleotides in a nucleic acid sequence.

In some instances, a binding site binds to a protein target comprising a span of at least 6 amino acids, for example, least 8, 9, 10, 12, 15, 20, 25, 30, 40, 50, or 100 amino acids. In some instances, a binding site binds to a protein target comprising a contiguous stretch of amino acids. In some instances, a binding site binds to a protein target comprising a non-contiguous stretch of amino acids. In some instances, a binding site binds to a protein target comprising a site of a mutation or functional mutation, including a deletion, addition, swap, or truncation of the amino acids in a polypeptide sequence. A binding site can bind to a binding moiety of a protein target. In some instances, a binding moiety of a protein target comprises a span of at least 6 amino acids, for example, least 8, 9, 10, 12, 15, 20, 25, 30, 40, 50, or 100 amino acids. In some instances, a binding moiety of a protein target comprises a contiguous stretch of amino acids. In some instances, a binding moiety of a protein target comprises a non-contiguous stretch of amino acids. In some instances, a binding moiety of a protein target comprises a site of a mutation or functional mutation, including a deletion, addition, swap, or truncation of the amino acids in a polypeptide sequence.

In some embodiments, a binding site binds to a domain, a fragment, an epitope, a region, or a portion of a membrane bound protein. A binding site can bind to a binding moiety of a membrane bound protein. In some embodiments, a binding moiety is on or comprises a domain, a fragment, an epitope, a region, or a portion of a membrane bound protein. Exemplary membrane bound proteins include, but are not limited to, GPCRs (e.g., adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), non-excitable and excitable channels, receptor tyrosine kinases, receptor serine/threonine kinases, receptor guanylate cyclases, growth factor and hormone receptors (e.g., epidermal growth factor (EGF) receptor), and others. The binding site can bind to a domain, a fragment, an epitope, a region, or a portion of a mutant or modified variants of membrane-bound proteins. The binding site can bind to a binding moiety of a mutant or modified variant of membrane-bound protein. The binding moiety may also be on or comprise a domain, a fragment, an epitope, a region, or a portion of a mutant or modified variants of membrane-bound proteins. For example, some single or multiple point mutations of GPCRs retain function and are involved in disease (See, e.g., Stadel et al., (1997) Trends in Pharmacological Review 18:430-37).

A binding site binds to, for example, a domain, a fragment, an epitope, a region, or a portion of a ubiquitin ligase. A binding site binds to, for example, a domain, a fragment, an epitope, a region, or a portion of a ubiquitin adaptor, proteasome adaptor, or proteasome protein. A binding site binds to, for example, a domain, a fragment, an epitope, a region, or a portion of a protein involved in endocytosis, phagocytosis, a lysosomal pathway, an autophagic pathway, macroautophagy, microautophagy, chaperone-mediated autophagy, the multivesicular body pathway, or a combination thereof.

RNA Binding Sites

In some embodiments, the circular polyribonucleotide comprises one or more RNA binding sites. In some embodiments, the circular polyribonucleotide includes RNA binding sites that modify expression of an endogenous gene and/or an exogenous gene. In some embodiments, the RNA binding site modulates expression of a host gene. The RNA binding site can include a sequence that hybridizes to an endogenous gene (e.g., a sequence for a miRNA, siRNA, mRNA, lncRNA, RNA, DNA, an antisense RNA, gRNA as described herein), a sequence that hybridizes to an exogenous nucleic acid such as a viral DNA or RNA, a sequence that hybridizes to an RNA, a sequence that interferes with gene transcription, a sequence that interferes with RNA translation, a sequence that stabilizes RNA or destabilizes RNA such as through targeting for degradation, or a sequence that modulates a DNA- or RNA-binding factor. In some embodiments, the circular polyribonucleotide comprises an aptamer sequence that binds to an RNA. The aptamer sequence can bind to an endogenous gene (e.g., a sequence for a miRNA, siRNA, mRNA, lncRNA, RNA, DNA, an antisense RNA, gRNA as described herein), to an exogenous nucleic acid such as a viral DNA or RNA, to an RNA, to a sequence that interferes with gene transcription, to a sequence that interferes with RNA translation, to a sequence that stabilizes RNA or destabilizes RNA such as through targeting for degradation, or to a sequence that modulates a DNA- or RNA-binding factor. The secondary structure of the aptamer sequence can bind to the RNA. The circular RNA can form a complex with the RNA by binding of the aptamer sequence to the RNA.

In some embodiments, the RNA binding site can be one of a tRNA, lncRNA, lincRNA, miRNA, rRNA, snRNA, microRNA, siRNA, piRNA, snoRNA, snRNA, exRNA, scaRNA, Y RNA, and hnRNA binding site. RNA binding sites are well-known to persons of ordinary skill in the art.

Certain RNA binding sites can inhibit gene expression through the biological process of RNA interference (RNAi). In some embodiments, the circular polyribonucleotides comprises an RNAi molecule with RNA or RNA-like structures typically having 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNA (siRNA), double-strand RNA (dsRNA), microRNA (miRNA), short hairpin RNA (shRNA), meroduplexes, and dicer substrates.

In some embodiments, the RNA binding site comprises an siRNA or an shRNA. siRNA and shRNA resemble intermediates in the processing pathway of the endogenous miRNA genes. In some embodiments, siRNA can function as miRNA and vice versa. MicroRNA, like siRNA, can use RISC to downregulate target genes, but unlike siRNA, most animal miRNA do not cleave the mRNA. Instead, miRNA reduce protein output through translational suppression or polyA removal and mRNA degradation. Known miRNA binding sites are within mRNA 3′-UTRs; miRNA seem to target sites with near-perfect complementarity to nucleotides 2-8 from the miRNA's 5′ end. This region is known as the seed region. Because siRNA and miRNA are interchangeable, exogenous siRNA can downregulate mRNA with seed complementarity to the siRNA. Multiple target sites within a 3′-UTR can give stronger downregulation.

MicroRNA (miRNA) are short noncoding RNA that bind to the 3′-UTR of nucleic acid molecules and down-regulate gene expression either by reducing nucleic acid molecule stability or by inhibiting translation. The circular polyribonucleotide can comprise one or more miRNA target sequences, miRNA sequences, or miRNA seeds. Such sequences can correspond to any miRNA.

A miRNA sequence comprises a “seed” region, i.e., a sequence in the region of positions 2-8 of the mature miRNA, which sequence has Watson-Crick complementarity to the miRNA target sequence. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA target is flanked by an adenine (A) opposed to miRNA position 1. In some embodiments, a miRNA seed can comprise 6 nucleotides (e.g., nucleotides 2-7 of the mature miRNA), wherein the seed-complementary site in the corresponding miRNA target is flanked by an adenine (A) opposed to miRNA at position 1.

The bases of the miRNA seed can be substantially complementary with the target sequence. By engineering miRNA target sequences into the circular polyribonucleotide, the circular polyribonucleotide can evade or be detected by the host's immune system, have modulated degradation, or modulated translation. This process can reduce the hazard of off target effects upon circular polyribonucleotide delivery.

The circular polyribonucleotide can include an miRNA sequence identical to about 5 to about 25 contiguous nucleotides of a target gene. In some embodiments, the miRNA sequence targets a mRNA and commences with the dinucleotide AA, comprises a GC-content of about 30%-70%, about 30%-60%, about 40%-60%, or about 45%-55%, and does not have a high percentage identity to any nucleotide sequence other than the target in the genome of the mammal in which it is to be introduced, for example, as determined by standard BLAST search.

Conversely, miRNA binding sites can be engineered out of (i.e., removed from) the circular polyribonucleotide to modulate protein expression in specific tissues. Regulation of expression in multiple tissues can be accomplished through introduction or removal or one or several miRNA binding sites (e.g., the miRNA binding site confers nucleic acid activity in a cell).

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). MiRNA can also regulate complex biological processes, such as angiogenesis (miR-132). In the circular polyribonucleotides described herein, binding sites for miRNA that are involved in such processes can be removed or introduced, in order to tailor the expression from the circular polyribonucleotide to biologically relevant cell types or to the context of relevant biological processes. In some embodiments, the miRNA binding site includes, e.g., miR-7.

Through an understanding of the expression patterns of miRNA in different cell types, the circular polyribonucleotide described herein can be engineered for more targeted expression in specific cell types or only under specific biological conditions. Through introduction of tissue-specific miRNA binding sites, the circular polyribonucleotide can be designed for optimal protein expression in a tissue or in the context of a biological condition.

In addition, miRNA seed sites can be incorporated into the circular polyribonucleotide to modulate expression in certain cells which results in a biological improvement. An example of this is incorporation of miR-142 sites. Incorporation of miR-142 sites into the circular polyribonucleotide described herein can modulate expression in hematopoietic cells, but also reduce or abolish immune responses to a protein encoded in the circular polyribonucleotide.

In some embodiments, the circular polyribonucleotide comprises at least one miRNA, e.g., 2, 3, 4, 5, 6, or more. In some embodiments, the circular polyribonucleotide comprises an miRNA having at least about 75%, about 80%, about 85%, about 90%, about 95%, about 96%, about 97%, about 98%, about 99%, or 100% nucleotide sequence identity to any one of the nucleotide sequences or a sequence that is complementary to a target sequence.

Lists of known miRNA sequences can be found in databases maintained by research organizations, for example, Wellcome Trust Sanger Institute, Penn Center for Bioinformatics, Memorial Sloan Kettering Cancer Center, and European Molecule Biology Laboratory. RNAi molecules can be readily designed and produced by technologies known in the art. In addition, computational tools can be used to determine effective and specific sequence motifs.

In some embodiments, a circular polyribonucleotide comprises a long non-coding RNA. Long non-coding RNA (lncRNA) include non-protein coding transcripts longer than 100 nucleotides. The longer length distinguishes lncRNA from small regulatory RNA, such as miRNA, siRNA, and other short RNA. In general, the majority (˜78%) of lncRNA are characterized as tissue-specific. Divergent lncRNA that are transcribed in the opposite direction to nearby protein-coding genes (comprise a significant proportion ˜20% of total lncRNA in mammalian genomes) can regulate the transcription of the nearby gene.

The length of the RNA binding site may be between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30, or more nucleotides. The degree of identity of the RNA binding site to a target of interest can be at least 75%, at least 80%, at least 85%, at least 90%, or at least 95%.

In some embodiments, the circular polyribonucleotide includes one or more large intergenic non-coding RNA (lincRNA) binding sites. LincRNA make up most of the long non-coding RNA. LincRNA are non-coding transcripts and, in some embodiments, are more than about 200 nucleotides long. In some embodiments, lincRNA have an exon-intron-exon structure, similar to protein-coding genes, but do not encompass open-reading frames and do not code for proteins. LincRNA expression can be strikingly tissue-specific compared to coding genes. LincRNA are typically co-expressed with their neighboring genes to a similar extent to that of pairs of neighboring protein-coding genes. In some embodiments, the circular polyribonucleotide comprises a circularized lincRNA.

In some embodiments, the circular polyribonucleotides disclosed herein include one or more lincRNA, for example, FIRRE, LINC00969, PVT1, LINC01608, JPX, LINC01572, LINC00355, Clorf132, C3orf35, RP11-734, LINC01608, CC-499B15.5, CASC15, LINC00937, and RP11-191.

Lists of known lincRNA and lncRNA sequences can be found in databases maintained by research organizations, for example, Institute of Genomics and Integrative Biology, Diamantina Institute at the University of Queensland, Ghent University, and Sun Yat-sen University. LincRNA and lncRNA molecules can be readily designed and produced by technologies known in the art. In addition, computational tools can be used to determine effective and specific sequence motifs.

The RNA binding site can comprise a sequence that is substantially complementary, or fully complementary, to all or a fragment of an endogenous gene or gene product (e.g., mRNA). The complementary sequence can complement sequences at the boundary between introns and exons to prevent the maturation of newly-generated nuclear RNA transcripts of specific genes into mRNA for transcription. The complementary sequence may be specific to genes by hybridizing with the mRNA for that gene and prevent its translation. The RNA binding site can comprise a sequence that is antisense or substantially antisense to all or a fragment of an endogenous gene or gene product, such as DNA, RNA, or a derivative or hybrid thereof.

The RNA binding site can comprise a sequence that is substantially complementary, or fully complementary, to all or a fragment of an endogenous gene or gene product (e.g., mRNA). The complementary sequence can complement sequences at the boundary between introns and exons to prevent the maturation of newly-generated nuclear RNA transcripts of specific genes into mRNA for transcription. The complementary sequence may be specific to genes by hybridizing with the mRNA for that gene and prevent its translation. The RNA binding site can comprise a sequence that is antisense or substantially antisense to all or a fragment of an endogenous gene or gene product, such as DNA, RNA, or a derivative or hybrid thereof.

The RNA binding site can comprise a sequence that is substantially complementary, or fully complementary, to a region of a linear polyribonucleotide. The complementary sequence may be specific to the region of the linear polyribonucleotide for hybridization of the circular polyribonucleotide to the linear polyribonucleotide. In some embodiments, the linear polyribonucleotide also comprises a region for binding to a protein, such as a receptor, on a cell. In some embodiments, the region of the linear polyribonucleotide that binds to a cell receptor results in internalization of the linear polyribonucleotide hybridized to the circular polyribonucleotide into the cell after binding.

In some embodiments, the circular polyribonucleotide comprises a RNA binding site that has an RNA or RNA-like structure typically between about 5-5000 base pairs (depending on the specific RNA structure, e.g., miRNA 5-30 bps, lncRNA 200-500 bps) and has a nucleobase sequence identical (complementary) or nearly identical (substantially complementary) to a coding sequence in an expressed target gene within the cell.

DNA Binding Sites

In some embodiments, the circular polyribonucleotide comprises a DNA binding site, such as a sequence for a guide RNA (gRNA). In some embodiments, the circular polyribonucleotide comprises a guide RNA or a complement to a gRNA sequence. A gRNA short synthetic RNA composed of a “scaffold” sequence necessary for binding to the incomplete effector moiety and a user-defined ˜20 nucleotide targeting sequence for a genomic target. Guide RNA sequences can have a length of between 17-24 nucleotides (e.g., 19, 20, or 21 nucleotides) and complementary to the targeted nucleic acid sequence. Custom gRNA generators and algorithms can be used in the design of effective guide RNA. Gene editing can be achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNA can be effective in genome editing.

The gRNA can recognize specific DNA sequences (e.g., sequences adjacent to or within a promoter, enhancer, silencer, or repressor of a gene).

In some embodiments, the gRNA is part of a CRISPR system for gene editing. For gene editing, the circular polyribonucleotide can be designed to include one or multiple guide RNA sequences corresponding to a desired target DNA sequence. The gRNA sequences may include at least 10, 11, 12, 13, 14, 15, 16, 17, 18, 19, 20, 21, 22, 23, 24, 25, 26, 27, 28, 29, 30 or more nucleotides for interaction with Cas9 or other exonuclease to cleave DNA, e.g., Cpf1 interacts with at least about 16 nucleotides of gRNA sequence for detectable DNA cleavage.

In some embodiments, the circular polyribonucleotide comprises an aptamer sequence that can bind to DNA. The secondary structure of the aptamer sequence can bind to DNA. In some embodiments, the circular polyribonucleotide forms a complex with the DNA by binding of the aptamer sequence to the DNA.

In some embodiments, the circular polyribonucleotide includes sequences that bind a major groove of in duplex DNA. In one such instance, the specificity and stability of a triplex structure created by the circular polyribonucleotide and duplex DNA is afforded via Hoogsteen hydrogen bonds, which are different from those formed in classical Watson-Crick base pairing in duplex DNA. In one instance, the circular polyribonucleotide binds to the purine-rich strand of a target duplex through the major groove.

In some embodiments, triplex formation occurs in two motifs, distinguished by the orientation of the circular polyribonucleotide with respect to the purine-rich strand of the target duplex. In some instances, polypyrimidine sequence stretches in a circular polyribonucleotides bind to the polypurine sequence stretches of a duplex DNA via Hoogsteen hydrogen bonding in a parallel fashion (i.e., in the same 5′ to 3′, orientation as the purine-rich strand of the duplex), whereas the polypurine stretches (R) bind in an antiparallel fashion to the purine strand of the duplex via reverse-Hoogsteen hydrogen bonds. In the antiparallel, a purine motif comprises triplets of G:G-C, A:A-T, or T:A-T; whereas in the parallel, a pyrimidine motif comprises canonical triples of C+:G-C or T:A-T triplets (where C+ represents a protonated cytosine on the N3 position). Antiparallel GA and GT sequences in a circular polyribonucleotide may form stable triplexes at neutral pH, while parallel CT sequences in a circular polyribonucleotide may bind at acidic pH. N3 on cytosine in the circular polyribonucleotide may be protonated. Substitution of C with 5-methyl-C may permit binding of CT sequences in the circular polyribonucleotide at physiological pH as 5-methyl-C has a higher pK than does cytosine. For both purine and pyrimidine motifs, contiguous homopurine-homopyrimidine sequence stretches of at least 10 base pairs aid circular polyribonucleotide binding to duplex DNA, since shorter triplexes may be unstable under physiological conditions, and interruptions in sequences can destabilize the triplex structure. In some embodiments, the DNA duplex target for triplex formation includes consecutive purine bases in one strand. In some embodiments, a target for triplex formation comprises a homopurine sequence in one strand of the DNA duplex and a homopyrimidine sequence in the complementary strand.

In some embodiments, a triplex comprising a circular polyribonucleotide is a stable structure. In some embodiments, a triplex comprising a circular polyribonucleotide exhibits an increased half-life, e.g., increased by about 5%, 10%, 15%, 20%, 25%, 30%, 35%, 40%, 45%, 50%, or greater, e.g., persistence for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between.

Protein Binding Sites

In some embodiments, the circular polyribonucleotide includes one or more protein binding sites. In some embodiments, a protein binding site comprises an aptamer sequence. In one embodiment, the circular polyribonucleotide includes a protein binding site to reduce an immune response from the host as compared to the response triggered by a reference compound, e.g., a circular polyribonucleotide lacking the protein binding site, e.g., linear RNA.

In some embodiments, circular polyribonucleotides disclosed herein include one or more protein binding sites to bind a protein, e.g., a ribosome. By engineering protein binding sites, e.g., ribosome binding sites, into the circular polyribonucleotide, the circular polyribonucleotide can evade or have reduced detection by the host's immune system, have modulated degradation, or modulated translation.

In some embodiments, the circular polyribonucleotide comprises at least one immunoprotein binding site, for example, to mask the circular polyribonucleotide from components of the host's immune system, e.g., evade CTL responses. In some embodiments, the immunoprotein binding site is a nucleotide sequence that binds to an immunoprotein and aids in masking the circular polyribonucleotide as non-endogenous.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) or translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

In some embodiments, circular polyribonucleotides disclosed herein comprise a protein binding sequence that binds to a protein. In some embodiments, the protein binding sequence targets or localizes a circular polyribonucleotide to a specific target. In some embodiments, the protein binding sequence specifically binds an arginine-rich region of a protein.

In some embodiments, circular polyribonucleotides disclosed herein include one or more protein binding sites that each bind a target protein, e.g., acting as a scaffold to bring two or more proteins in close proximity. In some embodiments, circular polynucleotides disclosed herein comprise two protein binding sites that each bind a target protein, thereby bringing the target proteins into close proximity. In some embodiments, circular polynucleotides disclosed herein comprise three protein binding sites that each bind a target protein, thereby bringing the three target proteins into close proximity. In some embodiments, circular polynucleotides disclosed herein comprise four protein binding sites that each bind a target protein, thereby bringing the four target proteins into close proximity. In some embodiments, circular polynucleotides disclosed herein comprise five or more protein binding sites that each bind a target protein, thereby bringing five or more target proteins into close proximity. In some embodiments, the target proteins are the same. In some embodiments, the target proteins are different. In some embodiments, bringing target proteins into close proximity promotes formation of a protein complex. For example, a circular polyribonucleotide of the disclosure can act as a scaffold to promote the formation of a complex comprising one, two, three, four, five, six, seven, eight, nine, or ten target proteins, or more. In some embodiments, bringing two or more target proteins into close proximity promotes interaction of the two or more target proteins. In some embodiments, bringing two or more target proteins into close proximity modulates, promotes, or inhibits of an enzymatic reaction. In some embodiments, bringing two or more target proteins into close proximity modulates, promotes, or inhibits a signal transduction pathway.

In some embodiments, the protein binding site includes, but is not limited to, a binding site to the protein, such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, p53, PCBP2, POLR2A, PRPF8, PTBP1, RBFOX1, RBFOX2, RBFOX3, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

In some embodiments, a protein binding site is a nucleic acid sequence that binds to a protein, e.g., a sequence that can bind a transcription factor, enhancer, repressor, polymerase, nuclease, histone, or any other protein that binds DNA. In some embodiments, a protein binding site is an aptamer sequence that binds to a protein. In some embodiments, the secondary structure of the aptamer sequence binds the protein. In some embodiments, the circular RNA forms a complex with the protein by binding of the aptamer sequence to the protein.

In some embodiments, a circular RNA is conjugated to a small molecule or a part thereof, wherein the small molecule or part thereof binds to a target such as a protein. A small molecule can be conjugated to a circular RNA via a modified nucleotide, e.g., by click chemistry. Examples of small molecules that can bind to proteins include, but are not limited to 4-hydroxytamoxifen (4-OHT), AC220, Afatinib, an aminopyrazole analog, an AR antagonist, BI-7273, Bosutinib, Ceritinib, Chloroalkane, Dasatinib, Foretinib, Gefitinib, a HIF-1α-derived (R)-hydroxyproline, HJB97, a hydroxyproline-based ligand, IACS-7e, Ibrutinib, an ibrutinib derivative, JQ1, Lapatinib, an LCL161 derivative, Lenalidomide, a nutlin small molecule, OTX015, a PDE4 inhibitor, Pomalidomide, a ripk2 inhibitor, RN486, Sirt2 inhibitor 3b, SNS-032, Steel factor, a TBK1 inhibitor, Thalidomide, a thalidomide derivative, a Thiazolidinedione-based ligand, a VH032 derivative, VHL ligand 2, VHL-1, VL-269, and derivatives thereof.

In some embodiments, a circular RNA is conjugated to more than one small molecule, for instance, 2, 3, 4, 5, 6, 7, 8, 9, 10, or more small molecules. In some embodiments, a circular RNA is conjugated to more than one different small molecules, for instance, 2, 3, 4, 5, 6, 7, 8, 9, 10, or more different small molecules. In some embodiments, the more than one small molecule conjugated to the circular RNA are configured to recruit their respective target proteins into proximity, which can lead to interaction between the target proteins, and/or other molecular and cellular changes. For instance, a circular RNA can be conjugated to both JQ1 and thalidomide, or derivative thereof, which can thus recruit a target protein of JQ1, e.g., BET family proteins, and a target protein of thalidomide, e.g., E3 ligase. In some cases, the circular RNA conjugated with JQ1 and thalidomide recruits a BET family protein via JQ1, or derivative thereof, tags the BET family protein with ubiquitin by E3 ligase that is recruited through thalidomide or derivative thereof, and thus leads to degradation of the tagged BET family protein.

Other Binding Sites

In some embodiments, the circular polyribonucleotide comprises one or more binding sites to a non-RNA or non-DNA target. In some embodiments, the binding site can be one of a small molecule, an aptamer, a lipid, a carbohydrate, a virus particle, a membrane, a multi-component complex, a cell, a cellular moiety, or any fragment thereof binding site. In some embodiments, the circular polyribonucleotide comprises one or more binding sites to a lipid. In some embodiments, the circular polyribonucleotide comprises one or more binding sites to a carbohydrate. In some embodiments, the circular polyribonucleotide comprises one or more binding sites to a carbohydrate. In some embodiments, the circular polyribonucleotide comprises one or more binding sites to a membrane. In some embodiments, the circular polyribonucleotide comprises one or more binding sites to a multi-component complex, e.g., ribosome, nucleosome, transcription machinery, etc.

In some embodiments, the circular polyribonucleotide comprises an aptamer sequence. The aptamer sequence can bind to any target as described herein (e.g., a nucleic acid molecule, a small molecule, a protein, a carbohydrate, a lipid, etc.). The aptamer sequence has a secondary structure that can bind the target. In some embodiments, the aptamer sequence has a tertiary structure that can bind the target. In some embodiments, the aptamer sequence has a quaternary structure that can bind the target. The circular polyribonucleotide can bind to the target via the aptamer sequence to form a complex. In some embodiments, the complex is detectable for at least 5 days. In some embodiments, the complex is detectable for at least 2 days, 3, days, 4 days, 5 days, 6 days, 7 days, 8 days, 9 days, 10 days, 11 days, 12 days, 13 days, 14 days, 15 days, 16 days.

Targets

The least one binding site can bind to a target. The at least one binding site can comprise at least one aptamer sequence that binds to a target. In some embodiments, the circRNA comprises one or more binding sites for one or more targets. Targets include, but are not limited to, nucleic acids (e.g., RNAs, DNAs, RNA-DNA hybrids), small molecules (e.g., drugs, fluorophores, metabolites), aptamers, polypeptides, proteins, lipids, carbohydrates, antibodies, viruses, virus particles, membranes, multi-component complexes, organelles, cells, other cellular moieties, any fragments thereof, and any combination thereof. (See, e.g., Fredriksson et al., (2002) Nat Biotech 20:473-77; Gullberg et al., (2004) PNAS, 101:8420-24). For example, a target is a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof.

In some embodiments, a target is a polypeptide, a protein, or any fragment thereof. For example, a target can be a purified polypeptide, an isolated polypeptide, a fusion tagged polypeptide, a polypeptide attached to or spanning the membrane of a cell or a virus or virion, a cytoplasmic protein, an intracellular protein, an extracellular protein, a kinase, a tyrosine kinase, a serine/threonine kinase, a phosphatase, an aromatase, a phosphodiesterase, a cyclase, a helicase, a protease, an oxidoreductase, a reductase, a transferase, a hydrolase, a lyase, an isomerase, a glycosylase, a extracellular matrix protein, a ligase, a ubiquitin ligase, any ligase that affects post-translational modification, an ion transporter, a channel, a pore, an apoptotic protein, a cell adhesion protein, a pathogenic protein, an aberrantly expressed protein, a transcription factor, a transcription regulator, a translation protein, an epigenetic factor, an epigenetic regulator, a chromatin regulator, a chaperone, a secreted protein, a ligand, a hormone, a cytokine, a chemokine, a nuclear protein, a receptor, a transmembrane receptor, a receptor tyrosine kinase, a G-protein coupled receptor, a growth factor receptor, a nuclear receptor, a hormone receptor, a signal transducer, an antibody, a membrane protein, an integral membrane protein, a peripheral membrane protein, a cell wall protein, a globular protein, a fibrous protein, a glycoprotein, a lipoprotein, a chromosomal protein, a proto-oncogene, an oncogene, a tumor-suppressor gene, any fragment thereof, or any combination thereof. In some embodiments, a target is a heterologous polypeptide. In some embodiments, a target is a protein overexpressed in a cell using molecular techniques, such as transfection. In some embodiments, a target is a recombinant polypeptide. For example, a target is in a sample produced from bacterial (e.g., E. coli), yeast, mammalian, or insect cells (e.g., proteins overexpressed by the organisms). In some embodiments, a target is a polypeptide with a mutation, insertion, deletion, or polymorphism. In some embodiments, a target is a polypeptide naturally expressed by a cell (e.g., a healthy cell or a cell associated with a disease or condition). In some embodiments, a target is an antigen, such as a polypeptide used to immunize an organism or to generate an immune response in an organism, such as for antibody production.

In some embodiments, a target is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can comprise three domains, C_{H1}, C_{H2}, and C_{H3}. Each light chain can comprise a light chain variable region (V_{L}) and a light chain constant region. The light chain constant region can comprise one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., lgG₁, lgG₂, lgG₃, lgG₄, lgA₁ and lgA₂), subclass or modified version thereof. Antibodies may include a complete immunoglobulin or fragments thereof. An antibody fragment can refer to one or more fragments of an antibody that retain the ability to specifically bind to a binding moiety, such as an antigen. In addition, aggregates, polymers, and conjugates of immunoglobulins or their fragments are also included so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. An antibody can be a polyvalent antibody, for example, bivalent, trivalent, tetravalent, pentavalent, hexavalanet, heptavalent, or octavalent antibodies. An antibody can be a multi-specific antibody. For example, bispecific, trispecific, tetraspecific, pentaspecific, hexaspecific, heptaspecific, or octaspecific antibodies can be generated, e.g., by recombinantly joining a combination of any two or more antigen binding agents (e.g., Fab, F(ab)₂, scFv, Fv, IgG). Multi-specific antibodies can be used to bring two or more targets into close proximitiy, e.g., degradation machinery and a target substrate to degrade, or a ubiquitin ligase and a substrate to ubiquitinate. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal.

In some embodiments, a target is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a polynucleotide target is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a target is a recombinant polynucleotide. In some embodiments, a target is a heterologous polynucleotide. For example, a target is a polynucleotide produced from bacterial (e.g., E. coli), yeast, mammalian, or insect cells (e.g., polynucleotides heterologous to the organisms). In some embodiments, a target is a polynucleotide with a mutation, insertion, deletion, or polymorphism.

In some embodiments, a target is an aptamer. An aptamer is an isolated nucleic acid molecule that binds with high specificity and affinity to a binding moiety or target molecule, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is fortuitous and is not thought to play a role in the binding of an aptamer to its cognate target. Aptamers are differentiated from naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids (e.g., nucleic acid-binding proteins). Aptamers on the other hand non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any partner of interest including small molecules, carbohydrates, peptides, etc. For most partners, even proteins, a naturally occurring nucleic acid sequence to which it binds does not exist. For those partners that do have such a sequence, e.g., nucleic acid-binding proteins, such sequences will differ from aptamers as a result of the relatively low binding affinity used in nature as compared to tightly binding aptamers. Aptamers are capable of specifically binding to selected partners and modulating the partner's activity or binding interactions, e.g., through binding, aptamers may block their partner's ability to function. The functional property of specific binding to a partner is an inherent property an aptamer. An aptamer can be 6-35 kDa. An aptamer can be from 20 to 500 nucleotides. An aptamer can bind its partner with micromolar to sub-nanomolar affinity, and may discriminate against closely related targets (e.g., aptamers may selectively bind related proteins from the same gene family). In some cases, an aptamer only binds one molecule. In some cases, an aptamer binds family members of a molecule of interest. An aptamer, in some cases, binds to multiple different molecules. Aptamers are capable of using commonly seen intermolecular interactions such as hydrogen bonding, electrostatic complementarities, hydrophobic contacts, and steric exclusion to bind with a specific partner. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides. An aptamer can be a linear ribonucleic acid (e.g., linear aptamer) comprising an aptamer sequence or a circular polyribonucleic acid comprising an aptamer sequence (e.g., a circular aptamer).

In some embodiments, a target is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from −0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, circRNA comprises a binding site to a single target or a plurality of (e.g., two or more) targets. In one embodiment, the single circRNA comprises 2, 3, 4, 5, 6, 7, 8, 9, 10, or more different binding sites for a single target. In one embodiment, the single circRNA comprises 2, 3, 4, 5, 6, 7, 8, 9, 10, or more of the same binding sites for a single target. In one embodiment, the single circRNA comprises 2, 3, 4, 5, 6, 7, 8, 9, 10, or more different binding sites for one or more different targets. In one embodiment, two or more targets are in a sample, such as a mixture or library of targets, and the sample comprises circRNA comprising two or more binding sites that bind to the two or more targets.

In some embodiments, a single target or a plurality of (e.g., two or more) targets have a plurality of binding moieties. In one embodiment, the single target may have 2, 3, 4, 5, 6, 7, 8, 9, 10, or more binding moieties. In one embodiment, two or more targets are in a sample, such as a mixture or library of targets, and the sample comprises two or more binding moieties. In some embodiments, a single target or a plurality of targets comprise a plurality of different binding moieties. For example, a plurality may include at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 binding moieties.

A target can comprise a plurality of binding moieties comprising at least 2 different binding moieties. For example, a binding moiety can comprise a plurality of binding moieties comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different binding moieties.

Circular Polyribonucleotide Elements

In some embodiments, the circular polyribonucleotide comprises one or more of the elements as described herein in addition to comprising a sequence encoding a protein (e.g., a therapeutic protein) and/or at least one binding site. In some embodiments, the circular polyribonucleotide lacks a poly-A tail. In some embodiments, the circular polyribonucleotide lacks a replication element. In some embodiments, the circular polyribonucleotide lacks an IRES. In some embodiments, the circular polyribonucleotide lacks a cap. In some embodiments, the circular polyribonucleotide comprises any feature or any combination of features as disclosed in WO2019/118919, which is hereby incorporated by reference in its entirety.

For example, the circular polyribonucleotide comprises sequences encoding one or more polypeptides or peptides in addition to those disclosed above. Some examples include, but are not limited to, fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides. In some embodiments, the circular polyribonucleotide further comprises an expression sequence encoding an additional therapeutic protein as described herein. Further examples of regulatory elements are described in paragraphs [0151]-[0153] of WO2019/118919, which is hereby incorporated by reference in its entirety.

For example, the circular polyribonucleotide comprises a regulatory element, e.g., a sequence that modifies expression of an expression sequence within the circular polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. In addition, one regulatory element can increase an amount of products expressed for multiple expression sequences attached in tandem. Hence, one regulatory element can enhance the expression of one or more expression sequences. Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the circular polyribonucleotide, for instance, certain riboswitch aptazymes. A regulatory element can also include a selective degradation sequence. As used herein, the term “selective degradation sequence” refers to a nucleic acid sequence that initiates degradation of the circular polyribonucleotide, or an expression product of the circular polyribonucleotide. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element. Further examples of regulatory elements are described in paragraphs [0154]-[0161] of WO2019/118919, which is hereby incorporated by reference in its entirety.

In some embodiments, the circular polyribonucleotide comprises a sequence encoding a protein (e.g., a therapeutic protein) and/or at least one binding site, and comprises a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide. Further examples of translation initiation sequences are described in paragraphs [0163]-[0165] of WO2019/118919, which is hereby incorporated by reference in its entirety.

In some embodiments, a circular polyribonucleotide described herein comprises an internal ribosome entry site (IRES) element. A suitable IRES element to include in a circular polyribonucleotide can be an RNA sequence capable of engaging an eukaryotic ribosome. Further examples of an IRES are described in paragraphs [0166]-[0168] of WO2019/118919, which is hereby incorporated by reference in its entirety.

A circular polyribonucleotide can include one or more expression sequences (e.g., a therapeutic protein), and each expression sequence may or may not have a termination element. Further examples of termination elements are described in paragraphs [0169]-[0170] of WO2019/118919, which is hereby incorporated by reference in its entirety.

A circular polyribonucleotide of the disclosure can comprise a stagger element. The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPGP, where x=any amino acid. In some embodiments, the stagger element may include a chemical moiety, such as glycerol, a non nucleic acid linking moiety, a chemical modification, a modified nucleic acid, or any combination thereof.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and/or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

Examples of stagger elements are described in paragraphs [0172]-[0175] of WO2019/118919, which is hereby incorporated by reference in its entirety.

In some embodiments, the circular polyribonucleotide comprises one or more regulatory nucleic acid sequences or comprises one or more expression sequences that encode regulatory nucleic acid, e.g., a nucleic acid that modifies expression of an endogenous gene and/or an exogenous gene. In some embodiments, the expression sequence of a circular polyribonucleotide as provided herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

Exemplary regulatory nucleic acids are described in paragraphs [0177]-[0194] of WO2019/118919, which is hereby incorporated by reference in its entirety.

In some embodiments, the translation efficiency of a circular polyribonucleotide as provided herein is greater than a reference, e.g., a linear counterpart, a linear expression sequence, or a linear circular polyribonucleotide. In some embodiments, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some embodiments, a circular polyribonucleotide has a translation efficiency 10% greater than that of a linear counterpart. In some embodiments, a circular polyribonucleotide has a translation efficiency 300% greater than that of a linear counterpart.

In some embodiments, the circular polyribonucleotide produces stoichiometric ratios of expression products. Rolling circle translation continuously produces expression products at substantially equivalent ratios. In some embodiments, the circular polyribonucleotide has a stoichiometric translation efficiency, such that expression products are produced at substantially equivalent ratios. In some embodiments, the circular polyribonucleotide has a stoichiometric translation efficiency of multiple expression products, e.g., products from 2, 3, 4, 5, 6, 7, 8, 9, 10, 11, 12, or more expression sequences.

In some embodiments, once translation of the circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the circular polyribonucleotide.

In some embodiments, the rolling circle translation of the circular polyribonucleotide leads to generation of polypeptide product that is translated from more than one round of translation of the circular polyribonucleotide (“continuous” expression product). In some embodiments, the circular polyribonucleotide comprises a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the amount ratio of the discrete products over the total polypeptides is tested in an in vitro translation system. In some embodiments, the in vitro translation system used for the test of amount ratio comprises rabbit reticulocyte lysate. In some embodiments, the amount ratio is tested in an in vivo translation system, such as a eukaryotic cell or a prokaryotic cell, a cultured cell or a cell in an organism.

In some embodiments, the circular polyribonucleotide comprises untranslated regions (UTRs). UTRs of a genomic region comprising a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full-length human intron, e.g., ZKSCAN1.

Exemplary untranslated regions are described in paragraphs [0197]-[201] of WO2019/118919, which is hereby incorporated by reference in its entirety.

In some embodiments, the circular polyribonucleotide may include a poly-A sequence. Exemplary poly-A sequences are described in paragraphs [0202]-[0205] of WO2019/118919, which is hereby incorporated by reference in its entirety. In some embodiments, the circular polyribonucleotide lacks a poly-A sequence.

In some embodiments, the circular polyribonucleotide comprises one or more riboswitches. Exemplary riboswitches are described in paragraphs [0232]-[0252] of WO2019/118919, which is hereby incorporated by reference in its entirety.

In some embodiments, the circular polyribonucleotide comprises an aptazyme. Exemplary aptazymes are described in paragraphs [0253]-[0259] of WO2019/118919, which is hereby incorporated by reference in its entirety.

In some embodiments, the circular polyribonucleotide comprises one or more RNA binding sites. microRNAs (or miRNA) can be short noncoding RNAs that bind to the 3′UTR of nucleic acid molecules and down-regulate gene expression either by reducing nucleic acid molecule stability or by inhibiting translation. The circular polyribonucleotide may comprise one or more microRNA target sequences, microRNA sequences, or microRNA seeds. Such sequences may correspond to any known microRNA, such as those taught in US Publication US2005/0261218 and US Publication US2005/0059005, the contents of which are incorporated herein by reference in their entirety. Further examples of RNA binding sites are described in paragraphs [0206]-[0215] of WO2019/118919, which is hereby incorporated by reference in its entirety.

In some embodiments, the circular polyribonucleotide includes one or more protein binding sites that enable a protein, e.g., a ribosome, to bind to an internal site in the RNA sequence. Further examples of protein binding sites are described in paragraphs [0218]-[0221] of WO2019/118919, which is hereby incorporated by reference in its entirety.

In some embodiments, the circular polyribonucleotide comprises an encryptogen to reduce, evade or avoid the innate immune response of a cell. In one aspect, provided herein are circular polyribonucleotide which when delivered to cells (e.g., contacting), results in a reduced immune response from the host as compared to the response triggered by a reference compound, e.g. a linear polynucleotide corresponding to the described circular polyribonucleotide or a circular polyribonucleotide lacking an encryptogen. In some embodiments, the circular polyribonucleotide has less immunogenicity than a counterpart lacking an encryptogen.

In some embodiments, an encryptogen enhances stability. There is growing body of evidence about the regulatory roles played by the UTRs in terms of stability of a nucleic acid molecule and translation. The regulatory features of a UTR may be included in the encryptogen to enhance the stability of the circular polyribonucleotide.

In some embodiments, 5′ or 3′UTRs can constitute encryptogens in a circular polyribonucleotide. For example, removal or modification of UTR AU rich elements (AREs) may be useful to modulate the stability or immunogenicity of the circular polyribonucleotide.

In some embodiments, removal of modification of AU rich elements (AREs) in expression sequence, e.g., translatable regions, can be useful to modulate the stability or immunogenicity of the circular polyribonucleotide

In some embodiments, an encryptogen comprises miRNA binding site or binding site to any other non-coding RNAs. For example, incorporation of miR-142 sites into the circular polyribonucleotide described herein may not only modulate expression in hematopoietic cells, but also reduce or abolish immune responses to a protein encoded in the circular polyribonucleotide.

In some embodiments, an encyptogen comprises one or more protein binding sites that enable a protein, e.g., an immunoprotein, to bind to the RNA sequence. By engineering protein binding sites into the circular polyribonucleotide, the circular polyribonucleotide may evade or have reduced detection by the host's immune system, have modulated degradation, or modulated translation, by masking the circular polyribonucleotide from components of the host's immune system. In some embodiments, the circular polyribonucleotide comprises at least one immunoprotein binding site, for example to evade immune responses, e.g., CTL responses. In some embodiments, the immunoprotein binding site is a nucleotide sequence that binds to an immunoprotein and aids in masking the circular polyribonucleotide as exogenous.

In some embodiments, an encryptogen comprises one or more modified nucleotides. Exemplary modifications can include any modification to the sugar, the nucleobase, the internucleoside linkage (e.g. to a linking phosphate/to a phosphodiester linkage/to the phosphodiester backbone), and any combination thereof that can prevent or reduce immune response against the circular polyribonucleotide. Some of the exemplary modifications provided herein are described in details below.

In some embodiments, the circular polyribonucleotide includes one or more modifications as described elsewhere herein to reduce an immune response from the host as compared to the response triggered by a reference compound, e.g. a circular polyribonucleotide lacking the modifications. In particular, the addition of one or more inosine has been shown to discriminate RNA as endogenous versus viral. See for example, Yu, Z. et al. (2015) RNA editing by ADAR1 marks dsRNA as “self”. Cell Res. 25, 1283-1284, which is incorporated by reference in its entirety.

In some embodiments, the circular polyribonucleotide includes one or more expression sequences for shRNA or an RNA sequence that can be processed into siRNA, and the shRNA or siRNA targets RIG-I and reduces expression of RIG-I. RIG-I can sense foreign circular RNA and leads to degradation of foreign circular RNA. Therefore, a circular polynucleotide harboring sequences for RIG-I-targeting shRNA, siRNA or any other regulatory nucleic acids can reduce immunity, e.g., host cell immunity, against the circular polyribonucleotide.

In some embodiments, the circular polyribonucleotide lacks a sequence, element or structure, that aids the circular polyribonucleotide in reducing, evading or avoiding an innate immune response of a cell. In some such embodiments, the circular polyribonucleotide may lack a polyA sequence, a 5′ end, a 3′ end, phosphate group, hydroxyl group, or any combination thereof.

In some embodiments, the circular polyribonucleotide comprises a spacer sequence. In some embodiments, elements of a polyribonucleotide may be separated from one another by a spacer sequence or linker. Exemplary of spacer sequences are described in paragraphs [0293]-[0302] of WO2019/118919, which is hereby incorporated by reference in its entirety.

The circular polyribonucleotide described herein may also comprise a non-nucleic acid linker. Exemplary non-nucleic acid linkers are described in paragraphs [0303]-[0307] of WO2019/118919, which is hereby incorporated by reference in its entirety.

In some embodiments, the circular polyribonucleotide further includes another nucleic acid sequence. In some embodiments, the circular polyribonucleotide may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In some embodiments, the circular polyribonucleotide includes an siRNA to target a different locus of the same gene expression product as the circular polyribonucleotide. In some embodiments, the circular polyribonucleotide includes an siRNA to target a different gene expression product than a gene expression product that is present in the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein In some embodiments, the circular polyribonucleotide lacks a 5′ cap.

In some embodiments, the circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide comprises one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

As a result of its circularization, the circular polyribonucleotide may include certain characteristics that distinguish it from linear RNA. For example, the circular polyribonucleotide is less susceptible to degradation by exonuclease as compared to linear RNA. As such, the circular polyribonucleotide can be more stable than a linear RNA, especially when incubated in the presence of an exonuclease. The increased stability of the circular polyribonucleotide compared with linear RNA can make the circular polyribonucleotide more useful as a cell transforming reagent to produce polypeptides (e.g., antigens and/or epitopes to elicit antibody responses). The increased stability of the circular polyribonucleotide compared with linear RNA can make the circular polyribonucleotide easier to store for long than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide can be less susceptible to dephosphorylation when the circular polyribonucleotide is incubated with phosphatase, such as calf intestine phosphatase.

In some embodiments, the circular polyribonucleotide comprises particular sequence characteristics. For example, the circular polyribonucleotide may comprise a particular nucleotide composition. In some such embodiments, the circular polyribonucleotide may include one or more purine (adenine and/or guanosine) rich regions. In some such embodiments, the circular polyribonucleotide may include one or more purine poor regions. In some embodiments, the circular polyribonucleotide may include one or more AU rich regions or elements (AREs). In some embodiments, the circular polyribonucleotide may include one or more adenine rich regions.

In some embodiments, the circular polyribonucleotide may include one or more repetitive elements described elsewhere herein. In some embodiments, the circular polyribonucleotide comprises one or more modifications described elsewhere herein.

A circular polyribonucleotide may include one or more substitutions, insertions and/or additions, deletions, and covalent modifications with respect to reference sequences. For example, circular polyribonucleotides with one or more insertions, additions, deletions, and/or covalent modifications relative to a parent polyribonucleotide are included within the scope of this disclosure. Exemplary modifications are described in paragraphs [0310]-[0325] of WO2019/118919, which is hereby incorporated by reference in its entirety.

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure.

In some embodiments, one or more sequences of the circular polyribonucleotide include substantially single stranded vs double stranded regions. In some embodiments, the ratio of single stranded to double stranded may influence the functionality of the circular polyribonucleotide.

In some embodiments, one or more sequences of the circular polyribonucleotide that are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, the circular polyribonucleotide sequences that are substantially double stranded. In some embodiments, one or more sequences of the circular polyribonucleotide that are substantially double stranded may include a conformational recognition site, e.g., a riboswitch or aptazyme. In some embodiments, the circular polyribonucleotide sequences that are substantially double stranded may be conformationally rigid. In some such instances, the conformationally rigid sequence may sterically hinder the circular polyribonucleotide from binding a protein or a nucleic acid. In some embodiments, the sequence of the circular polyribonucleotide is purposefully engineered to include such secondary structures to avoid or reduce protein or nucleic acid binding.

There are 16 possible base-pairings, however of these, six (AU, GU, GC, UA, UG, CG) may form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the circular polyribonucleotide comprises a tertiary structure with one or more motifs, e.g., a pseudoknot, a g-quadruplex, a helix, and coaxial stacking.

Further examples of structure of circular polyribonucleotides as disclosed herein are described in paragraphs [0326]-[0333] of WO2019/118919, which is hereby incorporated by reference in its entirety.

In some embodiments, a circular polyribonucleotide as disclosed herein comprises a conjugation moiety for conjugation of the circular polyribonucleotide to, for example, to a chemical compound (e.g., a small molecule), an antibody or fragment thereof, a peptide, a protein, an aptamer, a drug, or a combination thereof. In some embodiments, a small molecule can be conjugated to a circRNA, thereby generating a circRNA comprising a small molecule. In some embodiments, the circRNA comprises at least two conjugation moieties, e.g., a first conjugation moiety that binds to a first small molecule (e.g., JQ1) and a second conjugation molecule that binds to a second small molecule (e.g., thalidomide). In some embodiments, the circRNA comprises a conjugation moiety that binds to a small molecule (e.g., thalidomide) and a binding site that binds to a protein (e.g., BRD4).

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the circular polyribonucleotide may be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least t 400 nucleotides, at least 300 nucleotides, at least 200 nucleotides, at least 100 nucleotides may be useful.

In some embodiments, the circular polyribonucleotide is capable of replicating or replicates in a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof. In some embodiments, the invention includes a cell comprising the circular polyribonucleotide described herein, wherein the cell is a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, a cultured cell, a primary cell or a cell line, a stem cell, a progenitor cell, a differentiated cell, a germ cell, a cancer cell (e.g., tumorigenic, metastic), a non-tumorigenic cell (normal cells), a fetal cell, an embryonic cell, an adult cell, a mitotic cell, a non-mitotic cell, or any combination thereof.

Stability and Half Life

In some embodiments, a circular polyribonucleotide provided herein has increased half-life over a reference, e.g., a linear polyribonucleotide having the same nucleotide sequence that is not circularized (linear counterpart). In some embodiments, the circular polyribonucleotide is substantially resistant to degradation, e.g., exonuclease degradation. In some embodiments, the circular polyribonucleotide is resistant to self-degradation. In some embodiments, the circular polyribonucleotide lacks an enzymatic cleavage site, e.g., a dicer cleavage site. Further examples of stability and half life of circular polyribonucleotides as disclosed herein are described in paragraphs [0308]-[0309] of WO2019/118919, which is hereby incorporated by reference in its entirety.

In some embodiments, the circular polyribonucleotide has a half-life of at least that of a linear counterpart, e.g., linear expression sequence, or linear circular polyribonucleotide. In some embodiments, the circular polyribonucleotide has a half-life that is increased over that of a linear counterpart. In some embodiments, the half-life is increased by about 5%, 10%, 15%, 20%, 25%, 30%, 35%, 40%, 45%, 50%, or greater. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween. In some embodiments, the circular polyribonucleotide has a half-life or persistence in a cell while the cell is dividing. In some embodiments, the circular polyribonucleotide has a half-life or persistence in a cell post division. In certain embodiments, the circular polyribonucleotide has a half-life or persistence in a dividing cell for greater than about 10 minutes to about 30 days, or at least about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, at least about 10%, 20%, 30%, 40%, 50%, 60%, 70%, 80%, 90%, or 95% of an amount of the circular polyribonucleotide persists for a time period of at least about 3, 4, 5, 6, 7, 8, 9, 10, 12, 14, or 16 days in a cell.

In some embodiments, the circular polyribonucleotide is non-immunogenic in a mammal, e.g., a human.

Production Methods

In some embodiments, the circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins, such as fusion proteins comprising multiple antigens and/or epitopes). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

In some embodiments, the circular polyribonucleotides is purified, e.g., free ribonucleic acids, linear or nicked RNA, DNA, proteins, etc are removed. In some embodiments, the circular polyribonucleotides may be purified by any known method commonly used in the art. Examples of nonlimiting purification methods include, column chromatography, gel excision, size exclusion, etc.

Circularization

In some embodiments, a linear circular polyribonucleotide may be cyclized, or concatemerized. In some embodiments, the linear circular polyribonucleotide may be cyclized in vitro prior to formulation and/or delivery. In some embodiments, the linear circular polyribonucleotide may be cyclized within a cell.

Extracellular Circularization

In some embodiments, the linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a DNA or RNA ligase may be used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphorodiester linkage. In an example reaction, a linear circular polyribonucleotide is incubated at 37° C. for 1 hour with 1-10 units of T4 RNA ligase (New England Biolabs, Ipswich, Mass.) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase may be used in the synthesis of the circular polynucleotides. As a non-limiting example, the ligase may be a circ ligase or circular ligase.

In some embodiments, either the 5′- or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, a linear circular polyribonucleotide may be cyclized or concatermerized by using at least one non-nucleic acid moiety. In one aspect, the at least one non-nucleic acid moiety may react with regions or features near the 5′ terminus and/or near the 3′ terminus of the linear circular polyribonucleotide in order to cyclize or concatermerize the linear circular polyribonucleotide. In another aspect, the at least one non-nucleic acid moiety may be located in or linked to or near the 5′ terminus and/or the 3′ terminus of the linear circular polyribonucleotide. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an apatamer or a non-nucleic acid linker as described herein.

In some embodiments, a linear circular polyribonucleotide may be cyclized or concatermerized due to a non-nucleic acid moiety that causes an attraction between atoms, molecular surfaces at, near or linked to the 5′ and 3′ ends of the linear circular polyribonucleotide. As a non-limiting example, one or more linear circular polyribonucleotides may be cyclized or concatermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, the linear circular polyribonucleotide may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear circular polyribonucleotide to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, the linear circular polyribonucleotide may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate may occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

In some embodiment, the circular polyribonucleotide includes at least one splicing element. Exemplary splicing elements are described in paragraphs [0270]-[0275] of WO2019/118919, which is hereby incorporated by reference in its entirety.

Other Circularization Methods

In some embodiments, linear circular polyribonucleotides may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motifs useful for replication. Exemplary replication elements are described in paragraphs [0280]-[0286] of WO2019/118919, which is hereby incorporated by reference in its entirety. In some embodiments, the circular polyribonucleotide as disclosed herein lacks a replication element.

In some embodiments, the circular polyribonucleotide lacks a poly-A sequence and a replication element.

It is within the scope of this disclosure to use any of the circular polyribonucleotides described herein in a method administration, wherein the method comprises providing a first does of a circular polyribonucleotide to a plurality of cells, followed by providing a second dose of the circular polyribonucleotide to the plurality of cells. It is also within the scope of this disclosure to use any of the circular polyribonucleotides described herein in a composition administration. The circular polyribonucleotide may comprise one or more of an expression sequence, a regulatory element, or an untranslated region. The circular polyribonucleotide may be competent for rolling circle translation. In some embodiments, the circular polyribonucleotide lacks a termination element. The circular polyribonucleotide may comprise a stagger element at the 3′ end of at least one of the expression sequences. In some embodiments, the stagger element stalls a ribosome during rolling circle translation. The stagger element may encode a sequence with a C-terminal consensus sequence that is D(V/I)ExNPGP, wherein x represents any amino acid. In some embodiments, the circular polyribonucleotide lacks an internal ribosomal entry site. In some embodiments, one or more of the expression sequences comprise a Kozak initiation sequence. The circular polyribonucleotide may comprise a termination element, e.g., a stop codon. In some embodiments, the circular polyribonucleotide comprises one or more of an encryptogen, a regulatory element, at replication element, or a quasi-double-stranded secondary structure. The circular polyribonucleotide may comprise one or more functional characteristics, e.g., greater translation efficiency than a linear counterpart, a stoichiometric translation efficiency of multiple translation products, less immunogenicity than a counterpart lacking an encryptogen, increased half-life over a linear counterpart, or persistence during cell division. The circular polyribonucleotide may comprise a replication domain, enabling self-replication of the circular polyribonucleotide.

Pharmaceutical Compositions

The method of the present invention comprises providing or administering compositions in combination with one or more pharmaceutically acceptable excipients. A composition of a circular polyribonucleotide may be used or administered as a pharmaceutical composition, using any of the dosing, redosing, or staggered dosing methods described herein. The circular polyribonucleotide compositions described herein may be provided or administered in a variety of different dosages and at a variety of different concentrations. The circular polyribonucleotide composition may be provided or administered as a pharmaceutical composition. The pharmaceutical composition may comprise one or more pharmaceutically relevant carriers or excipients. The pharmaceutical composition may comprise a circular polyribonucleotide and one or more pharmaceutically acceptable carriers or excipients.

A pharmaceutically acceptable excipient can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Pharmaceutical compositions described herein can be used in therapeutic and veterinary. In some embodiments, pharmaceutical compositions (e.g., comprising a circular polyribonucleotide as described herein) provided herein are suitable for administration to a subject, wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, goats, cats, dogs, mice, and/or rats; and/or birds, including commercially relevant birds such as parrots, poultry, chickens, ducks, geese, hens or roosters and/or turkeys; zoo animals, e.g., a feline; non-mammal animals, e.g., reptiles, fish, amphibians, etc.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the pharmaceutically acceptable carrier or excipient is a sugar (e.g., sucrose, lactose, mannitol, maltose, sorbitol or fructose), a neutral salt (e.g., sodium chloride, magnesium sulfate, magnesium chloride, potassium sulfate, sodium carbonate, sodium sulfite, potassium acid phosphate, or sodium acetate), an acidic component (e.g., fumaric acid, maleic acid, adipic acid, citric acid or ascorbic acid), an alkaline component (e.g., tris(hydroxymethyl) aminomethane (TRIS), meglumine, tribasic or dibasic phosphates of sodium or potassium), or an amino acid (e.g., glycine or arginine).

The circular polyribonucleotide described herein may also be included in pharmaceutical compositions with a delivery carrier.

Pharmaceutical compositions described herein may be formulated for example to include a pharmaceutical excipient or carrier. A pharmaceutical carrier can be a membrane, lipid biylar, and/or a polymeric carrier, e.g., a liposome, such as a nanoparticle, e.g., a lipid nanoparticle, and delivered by known methods, such as via partial or full encapsulation of the modified circular polyribonucleotide, to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi: 10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

In some embodiments, the circular polyribonucleotide or pharmaceutical composition is delivered as a naked delivery formulation. A naked delivery formulation delivers a circular polyribonucleotide as disclosed herein to a cell without the aid of a carrier and without covalent modification or partial or complete encapsulation of the circular polyribonucleotide.

A naked delivery formulation is a formulation that is free from a carrier and wherein the circular polyribonucleotide as described herein is without a covalent modification that binds a moiety that aids in delivery to a cell or without partial or complete encapsulation of the circular polyribonucleotide. In some embodiments, a circular polyribonucleotide without covalent modification bound to a moiety that aids in delivery to a cell is not covalently bound to a protein, small molecule, a particle, a polymer, or a biopolymer that aids in delivery to a cell.

In some embodiments, a naked delivery formulation may be free of any or all of: transfection reagents, cationic carriers, carbohydrate carriers, nanoparticle carriers, or protein carriers. For example, a naked delivery formulation may be free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

A naked delivery formulation may comprise a non-carrier excipient. In some embodiments, a non-carrier excipient may comprise an inactive ingredient. In some embodiments, a non-carrier excipient may comprise a buffer, for example PBS. In some embodiments, a non-carrier excipient may be a solvent, a non-aqueous solvent, a diluent (e.g., a parenterally acceptable diluent), a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may comprise a diluent (e.g., a parenterally acceptable diluent). A diluent may be a liquid diluent or a solid diluent. In some embodiments, a diluent may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

The invention is further directed to a host or host cell comprising the circular polyribonucleotide described herein. In some embodiments, the host or host cell is a plant, insect, bacteria, fungus, vertebrate, mammal (e.g., human), or other organism or cell.

In some embodiments, the circular polyribonucleotide is non-immunogenic in the host. In some embodiments, the circular polyribonucleotide has a decreased or fails to produce a response by the host's immune system as compared to the response triggered by a reference compound, e.g., a linear polynucleotide corresponding to the described circular polyribonucleotide or a circular polyribonucleotide lacking an encryptogen. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., delivered to or administered to) the circular polyribonucleotide. In some embodiments, the host is a mammal, such as a human. The amount of the circular polyribonucleotide, expression product, or both in the host can be measured at any time after administration.

### Cell

The cell in the method of present invention can be a eukaryotic cell. In some embodiments, the cell is an animal cell. In some embodiments, the cell is a mammalian cell. In some embodiments, the cell is a human cell. In some embodiments, the cell is a cell from an aquaculture animal (fish, crabs, shrimp, oysters etc.), a mammal, e.g., from a pet or zoo animal (cats, dogs, lizards, birds, lions, tigers and bears etc.), from a farm or working animal (horses, cows, pigs, chickens etc.), a human, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof.

In some embodiments, a cell is from an organ, a tissue, or an organism. The cell can be removed from a subject prior to use in the methods disclosed herein, e.g., excised surgically, by venipuncture, etc. The cell can be from a cell culture. The methods disclosed herein can be used on cell in a subject, e.g., a composition as disclosed herein is administered to a subject comprising a cell. A subject comprising a cell can be an aquaculture animal (fish, crabs, shrimp, oysters etc.), a mammal, e.g., from a pet or zoo animal (cats, dogs, lizards, birds, lions, tigers and bears etc.), a farm or working animal (horses, cows, pigs, chickens etc.), or a human. In some embodiments, the subject is a subject in need thereof and the protein produced by the circular polyribonucleotide of the method disclosed herein treats the subject.

In some embodiments, the cell is a plurality of cells. The plurality of cells in the method of present invention can be a plurality of eukaryotic cells. In some embodiments, the plurality of cells a plurality of animal cells. In some embodiments, the plurality of cells is a plurality of mammalian cells. In some embodiments, the plurality of cells is a plurality of human cells. In some embodiments, the plurality of cells is a plurality of cell from an aquaculture animal (fish, crabs, shrimp, oysters etc.), a mammal, e.g., from a pet or zoo animal (cats, dogs, lizards, birds, lions, tigers and bears etc.), from a farm or working animal (horses, cows, pigs, chickens etc.), a human, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof. The cell can be a plurality of cells in a subject. A subject can be an animal. A subject can be a mammal. A subject can be a human.

In some embodiments, a plurality of cells are cells from an organ, a tissue, or an organism. The plurality of cells can be removed from a subject prior to use in the methods disclosed herein, e.g., excised surgically, by venipuncture, etc. The plurality of cells can be from a cell culture. The methods disclosed herein can be used on a plurality of cells in a subject, e.g., a dose as disclosed herein is administered to a subject comprising a plurality of cells. A subject comprising a plurality of cells can be an aquaculture animal (fish, crabs, shrimp, oysters etc.), a mammal, e.g., from a pet or zoo animal (cats, dogs, lizards, birds, lions, tigers and bears etc.), a farm or working animal (horses, cows, pigs, chickens etc.), or a human. In some embodiments, the subject is a subject in need thereof and the protein produced by the circular polyribonucleotide of the method disclosed herein treats the subject.

### Subject

The subject in the method of present invention can be an animal. In some embodiments, the subject is an animal cell. In some embodiments, the subject is a mammal. In some embodiments, the subject is a human. In some embodiments, the subject is an aquaculture animal (fish, crabs, shrimp, oysters etc.), a mammal, e.g., from a pet or zoo animal (cats, dogs, lizards, birds (e.g., parrots), lions, tigers and bears etc.), from a farm or working animal (horses, cows (e.g., dairy and beef cattle) pigs, chickens, turkeys, hens or roosters, goats, sheep, etc.), or a human.

In some embodiments, the a cell as disclosed herein is in a subject as disclosed herein.

In some embodiments, the subject is a subject in need thereof and the protein produced by the circular polyribonucleotide of the method disclosed herein treats the subject.

### Numbered Embodiments #1

[1] A method of expressing a protein in a cell comprising:
   providing a first composition comprising a circular polyribonucleotide that encodes the protein to the cell, wherein the cell expresses a first level of the protein; and
   providing a second composition comprising the circular polyribonucleotide to the cell, wherein the cell expresses a second level of the protein and the second level is at least as much as the first level;
   thereby maintaining expression of the protein in the cell at least at the first level of the protein.
[2] A method of expressing a protein in a cell comprising:
   providing a first composition comprising a circular polyribonucleotide that encodes the protein to the cell, wherein the cell expresses a first level of the protein; and
   providing a second composition comprising the circular polyribonucleotide to the cell, wherein the cell expresses a second level of the protein and the second level varies by no more than 20% of the first level;
   thereby maintaining expression of the protein in the cell at least at the first level of the protein.
[3] A method of producing a circular polyribonucleotide in a cell comprising:
   providing a first composition comprising the circular polyribonucleotide to the cell, wherein the cell comprises a first level of the circular polyribonucleotide after providing the first composition; and
   providing a second composition of the circular polyribonucleotide to the cell, wherein the cell comprises a second level of the circular polyribonucleotide and the second level of circular polyribonucleotide is at least as much as the first level;
   thereby maintaining the circular polyribonucleotide in the cell at least at the first level.
[4] A method of producing a circular polyribonucleotide in a cell comprising:
   providing a first composition comprising the circular polyribonucleotide to the cell, wherein the cell comprises a first level of the circular polyribonucleotide after providing the first composition; and
   providing a second composition of the circular polyribonucleotides to the cell, wherein the cell comprises a second level of the circular polyribonucleotide and the second level of circular polyribonucleotide varies by no more than 20% of the first level after providing the second composition;
   thereby maintaining the circular polyribonucleotide in the cell at least at the first level.
[5] A method of expressing a level of a protein in a cell after providing a first composition and a second composition of a circular polyribonucleotide to the cell compared to a level of the protein in the cell after providing a first composition and second composition of a linear counterpart of the circular polyribonucleotide, comprising:
   providing a first composition of the circular polyribonucleotide encoding the protein to the cell, wherein the cell comprises the level of the protein after providing the first composition of the circular polyribonucleotide; and
   providing the second composition of the circular polyribonucleotide after the first composition to the cell, wherein the cell comprises at least the level of the protein after providing the second composition of the circular polyribonucleotide;
   thereby maintaining expression of the level of the protein in the cell after providing the first composition and the second composition of the circular polyribonucleotide compared to the level of the protein in the cell after providing the first composition and the second composition of the linear counterpart of the circular polyribonucleotide.
[6] A method of expressing a level of a protein in a cell after providing a first composition and a second composition of a circular polyribonucleotide to the cell compared to a level of the protein in the cell after providing a first composition and second composition of a linear counterpart of the circular polyribonucleotide, comprising:
   providing a first composition of the circular polyribonucleotide encoding the protein to the cell, wherein the cell comprises the level of the protein after providing the first composition of the circular polyribonucleotide; and
   providing the second composition of the circular polyribonucleotide after the first composition to the cell, wherein the cell comprises a level of the protein that varies by no more than 20% of the level after providing the second composition of the circular polyribonucleotide;
   thereby maintaining expression of the level of the protein in the cell after providing the first composition and the second composition of the circular polyribonucleotide compared to the level of the protein in the cell after providing the first composition and the second composition of the linear counterpart of the circular polyribonucleotide.
[7] A method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprising:
   providing a first composition of the circular polyribonucleotide to the cell, wherein the cell comprises the level of the circular polyribonucleotide after providing the first composition; and
   providing the second composition of the circular polyribonucleotide to the cell, wherein the cell comprises at least the level of the circular polyribonucleotide after providing the second composition;
   thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide.
[8] A method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprising:
   providing a first composition of the circular polyribonucleotide to the cell, wherein the cell comprises the level of the circular polyribonucleotide after providing the first composition; and
   providing the second composition of the circular polyribonucleotide to the cell, wherein the cell comprises a level of the protein after providing the second composition that varies by no more than 20% of the level of the circular polyribonucleotide;
   thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide.
[9] The method of any one of the preceding embodiments, wherein providing the second composition occurs after providing the first composition and before the first level of protein expressed by the first composition is substantially undetectable in the cell.
[10] The method of any one of the preceding embodiments, wherein providing the second composition occurs after providing the first composition and before the first level of protein expressed by the first composition decreases by more than 50% in the cell.
[11] The method of any one of the preceding embodiments further comprising providing a third composition of the circular polyribonucleotide to the cell after the second composition, thereby maintaining expression of the protein in the cell at least at the first level of protein.
[12] The method of any one of the preceding embodiments, wherein providing the third composition occurs after providing the second composition and before the second level of the protein expressed by the first and second composition is substantially undetectable in the cell.
[13] The method of any one of the preceding embodiments, wherein providing the third composition occurs after providing the second composition and before the second level of the protein expressed by the first and second composition in the cell decreases by more than 50%.
[14] The method of any one of the preceding embodiments further comprising providing a fourth, fifth, sixth, seventh, eighth, ninth, or tenth composition of the circular polyribonucleotide.
[15] The method of any one of the preceding embodiments, wherein providing the second composition occurs after providing the first composition and before the level of the circular polyribonucleotide produced by providing the first composition is substantially undetectable in the cell.
[16] The method of any one of the preceding embodiments further comprising providing a third composition of the circular polyribonucleotide to the cell after the second composition, thereby maintaining the level of the circular polyribonucleotide after providing the third composition at least at the first level.
[17] The method of any one of the preceding embodiments, wherein providing the third composition occurs after providing the second composition and before the level of the circular polyribonucleotide produced by the first and second composition in the cell is substantially undetectable in the cell.
[18] The method of any one of the preceding embodiments, wherein providing the third composition occurs after providing the second composition and before the level of the circular polyribonucleotide produced by the first and second composition in the cell decreases by more than 50%.
[19] The method of any one of the preceding embodiments further comprising providing a fourth, fifth, sixth, seventh, eighth, ninth, or tenth composition of the circular polyribonucleotide to the cell.
[20] The method of any one of the preceding embodiments, wherein providing the second composition of the circular polyribonucleotide occurs after the first composition and after the level of protein in the cell expressed by the first composition is substantially undetectable.
[21] The method of any one of the preceding embodiments, wherein the second composition is provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the level of protein in the cell expressed by the first composition is substantially undetectable.
[22] The method of any one of the preceding embodiments, wherein providing the second composition of the circular polyribonucleotide occurs after the first composition and after the level of the circular polyribonucleotide in the cell produced by the first composition is substantially undetectable.
[23] The method of any one of the preceding embodiments, wherein the second composition is provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 10 months, or 1 year after the level of the circular polyribonucleotide in the plurality produced by the first composition is substantially undetectable.
[24] The method of any one of the preceding embodiments, wherein the first composition further comprises a pharmaceutically acceptable carrier or excipient.
[25] The method of any one of the preceding embodiments, wherein the second composition further comprises a pharmaceutically acceptable carrier or excipient.
[26] The method of any one of the preceding embodiments, wherein the third composition further comprises a pharmaceutically acceptable carrier or excipient.
[27] The method of any one of the preceding embodiments, wherein the first composition and the second composition comprise about the same amount of the circular polyribonucleotide.
[28] The method of any one of the preceding embodiments, wherein the first composition comprises a higher amount of the circular polyribonucleotides than the second composition.
[29] The method of any one of the preceding embodiments, wherein the first composition comprises a higher amount of the circular polyribonucleotides than the third, fourth, fifth, sixth, seventh, eighth, ninth, or tenth composition.
[30] The method of any one of the preceding embodiments, wherein an amount of circular polyribonucleotide of the second composition varies by no more than 1%, 5%, 10%, 15%, 20%, or 25% of an amount of circular polyribonucleotide of the first composition.
[31] The method of any one of the preceding embodiments, wherein an amount of circular polyribonucleotide of the second composition is no more than 1%, 5%, 10%, 15%, 20%, or 25% less than an amount of circular polyribonucleotide of the first composition.
[32] The method of any one of the preceding embodiments, wherein the first level of the protein is the highest level of the protein one day after providing the first composition.
[33] The method of any one of the preceding embodiments, wherein the first level of the protein is 40%, 50%, 60%, 70%, 80%, or 90% of the highest level of the protein one day after providing the first composition.
[34] The method of any one of the preceding embodiments, wherein the second level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, 30, 35, or 40 days after providing the second composition.
[35] The method of any one of the preceding embodiments, wherein the third level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, 30, 35, or 40 days after providing the third composition.
[36] The method of any one of the preceding embodiments, wherein for each subsequent composition provided after the first composition, a subsequent level of the protein expressed after each subsequent composition is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, 30, 35, or 40 days after providing each subsequent composition.
[37] The method of any one of the preceding embodiments, wherein an average level of the protein after providing the second composition is at least 40%, 50%, 60%, 70%, 80%, or 90% of the first level, wherein the average level of the protein is measured from one day after providing the second composition to the day when the protein is substantially undetectable.
[38] The method of any one of the preceding embodiments, wherein an average level of the protein after providing each subsequent composition after the first composition is at least 40%, 50%, 60%, 70%, 80%, or 90% of the first level, wherein the average level of the protein is measured from one day after providing each subsequent composition to the day when the protein is substantially undetectable.
[39] The method of any one of the preceding embodiments, wherein the first level of the protein is maintained after providing the first composition and the second composition of the circular polyribonucleotide for at least 6 hours, 1 day, 2 days, 3 days, 5 days, 7 days, 14 days, 21 days, 28 days, or 35 days after providing the first composition.
[40] The method of any one of the preceding embodiments, wherein the first level of the protein is maintained after providing the first composition, second composition, and third composition of the circular polyribonucleotide for at least 6 hours, 1 day, 2 days, 3 days, 5 days, 7 days, 14 days, 21 days, 28 days, or 35 days after providing the first composition.
[41] The method of any one of the preceding embodiments, wherein the second level of protein in the cell after providing the second composition is at least 1%, 5%, 10%, 20%, 30%, 40%, 50%, or 60% higher than the first level of protein in the cell after providing the first composition.
[42] The method of any one of the preceding embodiments, wherein the third level of protein in the cell after providing the third composition is at least 5%, 10%, 20%, 30%, 40%, 50%, or 60% higher than the first level of protein in the plurality after providing the first composition.
[43] The method of any one of the preceding embodiments, wherein the second level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the protein after providing the first composition.
[44] The method of any one of the preceding embodiments, wherein the third level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the protein after providing the first composition.
[45] The method of any one of the preceding embodiments, wherein the first level of the circular polyribonucleotide is the highest level of the circular polyribonucleotide one day after providing the first composition.
[46] The method of any one of the preceding embodiments, wherein the first level of the circular polyribonucleotide is 40%, 50%, 60%, 70%, 80%, or 90% of the highest level of the circular polyribonucleotide one day after providing the first composition.
[47] The method of any one of the preceding embodiments, wherein the second level of the circular polyribonucleotide is at least 30%, 40%, 50%, 60%, 70%, 80%, 90%, 100%, 110%, 120%, or 130% of the highest level of the circular polyribonucleotide one day after providing the first composition for at least 1, 2, 3, 4, 5, 6, 7, 8, 9, 10, 12, 15, 20, 25, 30, 35, or 40 days after providing the second composition.
[48] The method of any one of the preceding embodiments, wherein the third level of the circular polyribonucleotide is at least 30%, 40%, 50%, 60%, 70%, 80%, 90%, 100%, 110%, 120%, or 130% of the highest level of the circular polyribonucleotide one day after providing the first composition for at least 1, 2, 3, 4, 5, 6, 7, 8, 9, 10, 12, 15, 20, 25, 30, 35, or 40 days after providing the third composition.
[49] The method of any one of the preceding embodiments, wherein for each subsequent composition provided after the first composition, a subsequent level of the circular polyribonucleotide expressed after each subsequent composition is at least 30%, 40%, 50%, 60%, 70%, 80%, 90%, 100%, 110%, 120%, or 130% of the highest level of the circular polyribonucleotide one day after providing the first composition for at least 1, 2, 3, 4, 5, 6, 7, 8, 9, 10, 12, 15, 20, 25, 30, 35, or 40 days after providing each subsequent composition.
[50] The method of any one of the preceding embodiments, wherein an average level of the circular polyribonucleotide after providing the second composition is at least 40%, 50%, 60%, 70%, 80%, or 90% of the first level, wherein the average level of the circular polyribonucleotide is measured from one day after providing the second composition to the day when the circular polyribonucleotide is substantially undetectable.
[51] The method of any one of the preceding embodiments, wherein an average level of the circular polyribonucleotide after providing each subsequent composition after the first composition is at least 40%, 50%, 60%, 70%, 80%, or 90% of the first level, wherein the average level of the circular polyribonucleotide is measured from one day after providing each subsequent composition to the day when the circular polyribonucleotide is substantially undetectable.
[52] The method of any one of the preceding embodiments, wherein the first level of the circular polyribonucleotide is maintained after providing the second composition of the circular polyribonucleotide for at least 6 hours, 1 day, 2 days, 3 days, 5 days, 7 days, 14 days, 21 days, 28 days, or 35 days.
[53] The method of any one of the preceding embodiments, wherein the first level of the circular polyribonucleotide is maintained after providing the third composition of the circular polyribonucleotide for at least 6 hours, 1 day, 2 days, 3 days, 5 days, 7 days, 14 days, 21 days, 28 days, or 35 days.
[54] The method of any one of the preceding embodiments, wherein the second level of circular polyribonucleotide in the cell after providing the second composition is at least 1%, 5%, 10%, 20%, 30%, 40%, 50%, or 60% higher than the first level of circular polyribonucleotide in the cell after providing the first composition.
[55] The method of any one of the preceding embodiments, wherein the third level of circular polyribonucleotide in the cell after providing the third composition is at least 5%, 10%, 20%, 30%, 40%, 50%, or 60% higher than the first level of circular polyribonucleotide in the plurality after providing the first composition.
[56] The method of any one of the preceding embodiments, wherein the second level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the circular polyribonucleotide after providing the first composition.
[57] The method of any one of the preceding embodiments, wherein the third level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the circular polyribonucleotide after providing the first composition.
[58] The method of any one of the preceding embodiments, wherein the level of the protein in the cell after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.
[59] The method of any one of the preceding embodiments, wherein the level of the protein in the cell after providing the first composition and the second composition of the circular polyribonucleotide is at least 5%, 10%, 20%, 30%, 40%, 50%, or 60% higher than the level of the protein in the cell after providing the first composition and the second composition of the linear counterpart of the circular polyribonucleotide.
[60] The method of any one of the preceding embodiments, wherein the level of the protein in the cell after providing the first composition and the second composition of the circular polyribonucleotide is at least 5%, 10%, 20%, 30%, 40%, 50%, or 60% higher than the level of the protein in the cell after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.
[61] The method of any one of the preceding embodiments, wherein the level of the circular polyribonucleotide in the cell after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.
[62] The method of any one of the preceding embodiments, wherein the level of the circular polyribonucleotide in the cell after providing the first composition and the second composition of the circular polyribonucleotide is at least 5%, 10%, 20%, 30%, 40%, 50%, or 60% higher than the level of the linear counterpart of the circular polyribonucleotide in the cell after providing the first composition and the second composition of the linear counterpart of the circular polyribonucleotide.
[63] The method of any one of the preceding embodiments, wherein the level of the circular polyribonucleotide in the plurality after providing the first composition and the second composition of the circular polyribonucleotide is at least 5%, 10%, 20%, 30%, 40%, 50%, or 60% higher than the level of the linear counterpart of the circular polyribonucleotide in the plurality after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.
[64] The method of any one of the preceding embodiments, wherein the protein is a therapeutic protein.
[65] The method of any one of the preceding embodiments, wherein the protein is an intracellular protein, a membrane protein, or a secreted protein.
[66] The method of any one of the preceding embodiments, wherein the therapeutic protein has antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity.
[67] The method of any one of the preceding embodiments, wherein the therapeutic protein is Human Factor VIII, Human Factor IX, REP1, adenosine deaminase, human NGF, nuclear-encoded ND4, SECRA2a, SUMO1, VEGF, PDE6A, p53, PBFD, ARSA, ABCD1, APOE4, RPGR, DCLRE1C, VEGF 165, PDGF-B, gamma-sarcoglycan, dystrophin, LAMP2B, CNGB3, Retinitis Pigmentosa GTPase Regulator, or CLN6.
[68] The method of any one of the preceding embodiments, wherein the cell is a eukaryotic cell.
[69] The method of any one of the preceding embodiments, wherein the cell is an animal cell.
[70] The method of any one of the preceding embodiments, wherein the cell is a mammalian cell.
[71] The method of any one of the preceding embodiments, wherein the cell is a human cell.
[72] The method of any one of the preceding embodiments, wherein the cell is a plurality of cells in a subject.
[73] The method of any one of the preceding embodiments, wherein the subject is an animal.
[74] The method of any one of the preceding embodiments, wherein the subject is a mammal.
[75] The method of any one of the preceding embodiments, wherein the subject is a human.
[76] The method of any one of the preceding embodiments, wherein the circular polyribonucleotide further comprises a stagger element at a 3′ end of an expression sequence, and lacks a termination element.
[77] The method of embodiment [76], wherein the stagger element stalls a ribosome during the rolling circle translation of the circular polyribonucleotide.
[78] The method of embodiment [76] or [77], wherein the stagger element encodes a sequence with a C-terminal consensus sequence that is D(V/I)ExNPGP, where x=any amino acid.
[79] The method of any one of the preceding embodiments, wherein the circular polyribonucleotide lacks an internal ribosomal entry site.
[80] The method of any one of the preceding embodiments, wherein the one or more expression sequences comprise a Kozak initiation sequence.
[81] The method of any one of the preceding embodiments, wherein the circular polyribonucleotide further comprises at least one structural element selected from:
   (a) an encryptogen;
   (b) a regulatory element;
   (c) a replication element; and
   (d) quasi-double-stranded secondary structure.
[82] The method of any one of the preceding embodiments, wherein the circular polyribonucleotide comprises at least one functional characteristic selected from:
   (i) greater translation efficiency than a linear counterpart;
   (ii) a stoichiometric translation efficiency of multiple translation products;
   (iii) less immunogenicity than a counterpart lacking an encryptogen;
   (iv) increased half-life over a linear counterpart; and
   (v) persistence during cell division.
[83] The method of embodiment [76], wherein the termination element comprises a stop codon.
[84] The method of any one of the preceding embodiments, wherein the circular polyribonucleotide further comprises a replication domain configured to mediate self-replication of the circular polyribonucleotide.
[85] The method of any one of the preceding embodiments, wherein the circular polyribonucleotide persists during cell division.

### Numbered Embodiments #2

[1] A method of expressing a protein in a cell comprising:
   providing a first composition comprising a circular polyribonucleotide that encodes the protein to the cell, wherein the cell expresses a first level of the protein; and
   providing a second composition comprising the circular polyribonucleotide to the cell, wherein the cell expresses a second level of the protein and the second level is at least as much as the first level;
   thereby maintaining expression of the protein in the cell at least at the first level of the protein.
[2] A method of expressing a protein in a cell comprising:
   providing a first composition comprising a circular polyribonucleotide that encodes the protein to the cell, wherein the cell expresses a first level of the protein; and
   providing a second composition comprising the circular polyribonucleotide to the cell, wherein the cell expresses a second level of the protein and the second level varies by no more than 20% of the first level;
   thereby maintaining expression of the protein in the cell at least at the first level of the protein.
[3] A method of expressing a level of a protein in a cell after providing a first composition and a second composition of a circular polyribonucleotide to the cell compared to a level of the protein in the cell after providing a first composition and second composition of a linear counterpart of the circular polyribonucleotide, comprising:
   providing a first composition of the circular polyribonucleotide encoding the protein to the cell, wherein the cell comprises the level of the protein after providing the first composition of the circular polyribonucleotide; and
   providing the second composition of the circular polyribonucleotide after the first composition to the cell, wherein the cell comprises at least the level of the protein after providing the second composition of the circular polyribonucleotide;
   thereby maintaining expression of the level of the protein in the cell after providing the first composition and the second composition of the circular polyribonucleotide compared to the level of the protein in the cell after providing the first composition and the second composition of the linear counterpart of the circular polyribonucleotide.
[4] A method of expressing a level of a protein in a cell after providing a first composition and a second composition of a circular polyribonucleotide to the cell compared to a level of the protein in the cell after providing a first composition and second composition of a linear counterpart of the circular polyribonucleotide, comprising:
   providing a first composition of the circular polyribonucleotide encoding the protein to the cell, wherein the cell comprises the level of the protein after providing the first composition of the circular polyribonucleotide; and
   providing the second composition of the circular polyribonucleotide after the first composition to the cell, wherein the cell comprises a level of the protein that varies by no more than 20% of the level after providing the second composition of the circular polyribonucleotide;
   thereby maintaining expression of the level of the protein in the cell after providing the first composition and the second composition of the circular polyribonucleotide compared to the level of the protein in the cell after providing the first composition and the second composition of the linear counterpart of the circular polyribonucleotide.
[5] The method of any one of embodiments [1] or [2], wherein providing the second composition occurs after providing the first composition and before the first level of protein expressed by the first composition is substantially undetectable in the cell.
[6] The method of any one of embodiments [1]-[2], wherein providing the second composition occurs after providing the first composition and before the first level of protein expressed by the first composition decreases by more than 50% in the cell.
[7] The method of any one of the embodiments [1]-[2] or [5]-[6], further comprising providing a third composition of the circular polyribonucleotide to the cell after the second composition, thereby maintaining expression of the protein in the cell at least at the first level of protein.
[8] The method of embodiment [7], wherein providing the third composition occurs after providing the second composition and before the second level of the protein expressed by the first and second composition is substantially undetectable in the cell.
[9] The method of embodiment [7], wherein providing the third composition occurs after providing the second composition and before the second level of the protein expressed by the first and second composition in the cell decreases by more than 50%.
[10] The method of any one of the preceding embodiments further comprising providing a fourth, fifth, sixth, seventh, eighth, ninth, or tenth composition of the circular polyribonucleotide.
[11] The method of any one of embodiments [3] or [4], wherein the second composition is provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of protein in the cell expressed by the first composition is substantially undetectable.
[12] The method of any one of embodiments [1], [2], or [5]-[10], wherein the first level of the protein is a highest level of the protein one day after providing the first composition.
[13] The method of any one of embodiments [1], [2], or [5]-[10], wherein the first level of the protein is 40%, 50%, 60%, 70%, 80%, or 90% of a highest level of the protein one day after providing the first composition.
[14] The method of embodiment [13], wherein the second level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, 30, 35, or 40 days after providing the second composition.
[15] The method of any one of embodiments [13] or [14], wherein a third level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, 30, 35, or 40 days after providing the third composition.
[16] The method of embodiment [15], wherein for each subsequent composition provided after the first composition, a subsequent level of the protein expressed after each subsequent composition is at least 30%, 40%, 50%, 60%, 70%, 80%, 90%, 100%, 110%, 120%, or 130% of a highest level of the protein one day after providing the first composition for at least 1, 2, 3, 4, 5, 6, 7, 8, 9, 10, 12, 15, 20, 25, 30, 35, or 40 days after providing each subsequent composition.
[17] The method of any one of embodiments [1], [2], or [5]4101, wherein an average level of the protein after providing the second composition is at least 40%, 50%, 60%, 70%, 80%, or 90% of the first level, wherein the average level of the protein is measured from one day after providing the second composition to the day when the protein is substantially undetectable.
[18] The method of any one of embodiments [1], [2], or [5]4101, wherein an average level of the protein after providing each subsequent composition after the first composition is at least 40%, 50%, 60%, 70%, 80%, or 90% of the first level, wherein the average level of the protein is measured from one day after providing each subsequent composition to the day when the protein is substantially undetectable.
[19] The method of any one of embodiments [1], [2], [5], [7], or [10], wherein the first level of the protein is maintained after providing the first composition and the second composition of the circular polyribonucleotide for at least 6 hours, 1 day, 2 days, 3 days, 5 days, 7 days, 14 days, 21 days, 28 days, or 35 days after providing the first composition.
[20] The method of any one of embodiments [7], [8], or [10], wherein the first level of the protein is maintained after providing the first composition, the second composition, and the third composition of the circular polyribonucleotide for at least 6 hours, 1 day, 2 days, 3 days, 5 days, 7 days, 14 days, 21 days, 28 days, or 35 days after providing the first composition.
[21] The method of any one of embodiments [1], [2], [5], [7], [8], or [10], wherein the second level of protein in the cell after providing the second composition is at least 1%, 5%, 10%, 20%, 30%, 40%, 50%, or 60% higher than the first level of protein in the cell after providing the first composition.
[22] The method of any one of embodiments [7], [8] or [10], wherein a third level of protein produced in the cell after providing the third composition is at least 5%, 10%, 20%, 30%, 40%, 50%, or 60% higher than the first level of protein in the plurality after providing the first composition.
[23] The method of any one of embodiments [1], [2], [5], [7], [8], or [10], wherein the second level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the protein after providing the first composition.
[24] The method of any one of embodiments [7], [8], or [10], wherein the third level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the protein after providing the first composition.
[25] The method of any one of embodiments [3], [4], [10], or [11], wherein the level of the protein in the cell after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.
[26] The method of any one of embodiments [3], [4], [10], or [11], wherein the level of the protein in the cell after providing the first composition and the second composition of the circular polyribonucleotide is at least 5%, 10%, 20%, 30%, 40%, 50%, or 60% higher than the level of the protein in the cell after providing the first composition and the second composition of the linear counterpart of the circular polyribonucleotide.
[27] The method of any one of embodiments [3], [4], [10], or [11], wherein the level of the protein in the cell after providing the first composition and the second composition of the circular polyribonucleotide is at least 5%, 10%, 20%, 30%, 40%, 50%, or 60% higher than the level of the protein in the cell after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.
[28] The method of any one of the preceding embodiments, wherein the protein is a therapeutic protein.
[29] The method of any one of the preceding embodiments, wherein the protein is an intracellular protein, a membrane protein, or a secreted protein.
[30] The method of any one embodiments [28] or [29], wherein the therapeutic protein has a) antioxidant activity, binding, cargo receptor activity, catalytic activity, molecular carrier activity, molecular transducer activity, nutrient reservoir activity, structural molecule activity, toxin activity, transcription regulator activity, translation regulator activity, or transporter activity; b) is a molecular function regulator; or c) functions as a protein tag.
[31] The method of any one embodiments [28]-[30], wherein the therapeutic protein is Human Factor VIII, Human Factor IX, REP1, adenosine deaminase, human NGF, nuclear-encoded ND4, SECRA2a, SUMO1, VEGF, PDE6A, p53, PBFD, ARSA, ABCD1, APOE4, RPGR, DCLRE1C, VEGF 165, PDGF-B, gamma-sarcoglycan, dystrophin, LAMP2B, CNGB3, Retinitis Pigmentosa GTPase Regulator, or CLN6.
[32] A method of producing a circular polyribonucleotide in a cell comprising:
   providing a first composition comprising the circular polyribonucleotide to the cell, wherein the cell comprises a first level of the circular polyribonucleotide after providing the first composition; and
   providing a second composition of the circular polyribonucleotide to the cell, wherein the cell comprises a second level of the circular polyribonucleotide and the second level of circular polyribonucleotide is at least as much as the first level;
   thereby maintaining the circular polyribonucleotide in the cell at least at the first level.
[33] A method of producing a circular polyribonucleotide in a cell comprising:
   providing a first composition comprising the circular polyribonucleotide to the cell, wherein the cell comprises a first level of the circular polyribonucleotide after providing the first composition; and
   providing a second composition of the circular polyribonucleotides to the cell, wherein the cell comprises a second level of the circular polyribonucleotide and the second level of circular polyribonucleotide varies by no more than 20% of the first level after providing the second composition;
   thereby maintaining the circular polyribonucleotide in the cell at least at the first level.
[34] A method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprising:
   providing a first composition of the circular polyribonucleotide to the cell, wherein the cell comprises the level of the circular polyribonucleotide after providing the first composition; and
   providing the second composition of the circular polyribonucleotide to the cell, wherein the cell comprises at least the level of the circular polyribonucleotide after providing the second composition;
   thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide.
[35] A method of producing a level of a circular polyribonucleotide in a cell after providing a first composition and a second composition of the circular polyribonucleotide to the cell compared to a level of a linear counterpart of the circular polyribonucleotide in the cell after providing a first composition and second composition of the linear counterpart of the circular polyribonucleotide, comprising:
   providing a first composition of the circular polyribonucleotide to the cell, wherein the cell comprises the level of the circular polyribonucleotide after providing the first composition; and
   providing the second composition of the circular polyribonucleotide to the cell, wherein the cell comprises a level of the protein after providing the second composition that varies by no more than 20% of the level of the circular polyribonucleotide;
   thereby maintaining the level of the circular polyribonucleotide in the cell after providing the first composition and the second composition of the circular polyribonucleotide compared to the level of the linear counterpart in the cell after providing the first composition and the second composition of the linear counterpart of the circular polyribonucleotide.
[36] The method of any one of embodiments [32] or [33], wherein providing the second composition occurs after providing the first composition and before the level of the circular polyribonucleotide produced by providing the first composition is substantially undetectable in the cell.
[37] The method of any one of embodiments [32], [33], or [36], wherein providing the second composition of the circular polyribonucleotide occurs after the first composition and after the level of the circular polyribonucleotide in the cell produced by the first composition is substantially undetectable.
[38] The method of any one of embodiments [32], [33], [36] or [37], wherein the second composition is provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of the circular polyribonucleotide in the plurality produced by the first composition is substantially undetectable.
[39] The method of any one of embodiments [32], [33], or [36]-[38], further comprising providing a third composition of the circular polyribonucleotide to the cell after the second composition, thereby maintaining the level of the circular polyribonucleotide after providing the third composition at least at the first level.
[40] The method of any one of embodiments [32], [33], or [36]-[39], wherein providing the third composition occurs after providing the second composition and before the level of the circular polyribonucleotide produced by the first and second composition in the cell is substantially undetectable in the cell.
[41] The method of embodiments [32], 3[3], or [36]-[40], wherein providing the third composition occurs after providing the second composition and before the level of the circular polyribonucleotide produced by the first and second composition in the cell decreases by more than 50%.
[42] The method of any one of embodiments [32]-[41], further comprising providing a fourth, fifth, sixth, seventh, eighth, ninth, or tenth composition of the circular polyribonucleotide to the cell.
[43] The method of any one of embodiments [34] or [35], wherein providing the second composition of the circular polyribonucleotide occurs after the first composition and after the level of protein in the cell expressed by the first composition is substantially undetectable.
[44] The method of any one of embodiments [32], [33], or [36]-[42], wherein the first level of the circular polyribonucleotide is a highest level of the circular polyribonucleotide one day after providing the first composition.
[45] The method of any one of embodiments [32], [33], or [36]-[42], wherein the level of the circular polyribonucleotide produced by the first composition is 40%, 50%, 60%, 70%, 80%, or 90% of a highest level of the circular polyribonucleotide one day after providing the first composition.
[46] The method of any one of embodiments [32], [33], or [36]-[42], wherein the level of the circular polyribonucleotide produced by the second composition is at least 30%, 40%, 50%, 60%, 70%, 80%, 90%, 100%, 110%, 120%, or 130% of the highest level of the circular polyribonucleotide one day after providing the first composition for at least 1, 2, 3, 4, 5, 6, 7, 8, 9, 10, 12, 15, 20, 25, 30, 35, or 40 days after providing the second composition.
[47] The method of any one of embodiments [39]-[42], wherein the third level of the circular polyribonucleotide is at least 30%, 40%, 50%, 60%, 70%, 80%, 90%, 100%, 110%, 120%, or 130% of the highest level of the circular polyribonucleotide one day after providing the first composition for at least 1, 2, 3, 4, 5, 6, 7, 8, 9, 10, 12, 15, 20, 25, 30, 35, or 40 days after providing the third composition.
[48] The method of any one of embodiments [32], [33], or [36]-[42], wherein for each subsequent composition provided after the first composition, a subsequent level of the circular polyribonucleotide expressed after each subsequent composition is at least 30%, 40%, 50%, 60%, 70%, 80%, 90%, 100%, 110%, 120%, or 130% of the highest level of the circular polyribonucleotide one day after providing the first composition for at least 1, 2, 3, 4, 5, 6, 7, 8, 9, 10, 12, 15, 20, 25, 30, 35, or 40 days after providing each subsequent composition.
[49] The method of any one of embodiments [32], [33], or [36]-[42], wherein an average level of the circular polyribonucleotide after providing the second composition is at least 40%, 50%, 60%, 70%, 80%, or 90% of the first level, wherein the average level of the circular polyribonucleotide is measured from one day after providing the second composition to the day when the circular polyribonucleotide is substantially undetectable.
[50] The method of any one of embodiments [32], [33], or [36]-[42], wherein an average level of the circular polyribonucleotide after providing each subsequent composition after the first composition is at least 40%, 50%, 60%, 70%, 80%, or 90% of the first level, wherein the average level of the circular polyribonucleotide is measured from one day after providing each subsequent composition to the day when the circular polyribonucleotide is substantially undetectable.
[51] The method of any one of embodiments [32], [33], or [36]-[42], wherein the first level of the circular polyribonucleotide is maintained after providing the second composition of the circular polyribonucleotide for at least 6 hours, 1 day, 2 days, 3 days, 5 days, 7 days, 14 days, 21 days, 28 days, or 35 days.
[52] The method of any one of embodiments [39]-[42], wherein the first level of the circular polyribonucleotide is maintained after providing the third composition of the circular polyribonucleotide for at least 6 hours, 1 day, 2 days, 3 days, 5 days, 7 days, 14 days, 21 days, 28 days, or 35 days.
[53] The method of any one of embodiments [32], [33], or [36]-[42], wherein the second level of circular polyribonucleotide in the cell after providing the second composition is at least 1%, 5%, 10%, 20%, 30%, 40%, 50%, or 60% higher than the first level of circular polyribonucleotide in the cell after providing the first composition.
[54] The method of any one of embodiments [39]-[42], wherein the third level of circular polyribonucleotide in the cell after providing the third composition is at least 5%, 10%, 20%, 30%, 40%, 50%, or 60% higher than the first level of circular polyribonucleotide in the plurality after providing the first composition.
[55] The method of any one of embodiments [32], [33], or [36]-[42], wherein the second level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the circular polyribonucleotide after providing the first composition.
[56] The method of any one of embodiments [39]-[42], wherein the third level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the circular polyribonucleotide after providing the first composition.
[57] The method of any one of embodiments [34], [35], or [43], wherein the level of the circular polyribonucleotide in the cell after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.
[58] The method of any one of embodiments [34], [35], or [43], wherein the level of the circular polyribonucleotide in the cell after providing the first composition and the second composition of the circular polyribonucleotide is at least 5%, 10%, 20%, 30%, 40%, 50%, or 60% higher than the level of the linear counterpart of the circular polyribonucleotide in the cell after providing the first composition and the second composition of the linear counterpart of the circular polyribonucleotide.
[59] The method of any one of embodiments [34], [35], or [43], wherein the level of the circular polyribonucleotide in the plurality after providing the first composition and the second composition of the circular polyribonucleotide is at least 5%, 10%, 20%, 30%, 40%, 50%, or 60% higher than the level of the linear counterpart of the circular polyribonucleotide in the plurality after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.
[60] The method of any one of embodiments [32]-[59], wherein the circular polyribonucleotide comprises a binding site for a target, encodes a protein, or both.
[61] A method of binding a target in a cell comprising:
   providing a first composition comprising a circular polyribonucleotide that comprises a binding site, to the cell, wherein the target binds to the binding site at a first level; and
   providing a second composition comprising the circular polyribonucleotide to the cell, wherein the target binds to the binding site at a second level and the second level is at least as much as the first level;
   thereby maintaining binding of the target in the cell at least at the first level of binding.
[62] A method of binding a target in a cell comprising:
   providing a first composition comprising a circular polyribonucleotide comprises a binding site to the cell, wherein the target binds to the binding site at a first level; and
   providing a second composition comprising the circular polyribonucleotide to the cell, wherein the target binds to the binding site at a second level and the second level varies by no more than 20% of the first level;
   thereby maintaining binding of the target in the cell at least at the first level of binding.
[63] A method of binding a target in a cell after providing a first composition and a second composition of a circular polyribonucleotide to the cell compared to a level of binding to the target in the cell after providing a first composition and second composition of a linear counterpart of the circular polyribonucleotide, comprising:
   providing a first composition of the circular polyribonucleotide comprising binding site to the cell, wherein the cell comprises the level of the binding to the target after providing the first composition of the circular polyribonucleotide; and providing the second composition of the circular polyribonucleotide after the first composition to the cell, wherein the cell comprises at least the level of the binding to the target after providing the second composition of the circular polyribonucleotide;
   thereby maintaining the level of the binding to the target in the cell after providing the first composition and the second composition of the circular polyribonucleotide compared to the level of the binding to the target in the cell after providing the first composition and the second composition of the linear counterpart of the circular polyribonucleotide.
[64] A method of binding a target in a cell after providing a first composition and a second composition of a circular polyribonucleotide to the cell compared to a level of binding to the target in the cell after providing a first composition and second composition of a linear counterpart of the circular polyribonucleotide, comprising:
   providing a first composition of the circular polyribonucleotide comprising binding site to the cell, wherein the cell comprises the level of the binding to the target after providing the first composition of the circular polyribonucleotide; and providing the second composition of the circular polyribonucleotide after the first composition to the cell, wherein the cell comprises a level of the binding to a target that varies by no more than 20% of the level after providing the second composition of the circular polyribonucleotide;
   thereby maintaining the level of the binding to the target in the cell after providing the first composition and the second composition of the circular polyribonucleotide compared to the level of the binding to the target in the cell after providing the first composition and the second composition of the linear counterpart of the circular polyribonucleotide.
[65] The method of any one of embodiments [61] or [62], wherein providing the second composition occurs after providing the first composition and before the first level of binding by the first composition is substantially undetectable in the cell.
[66] The method of any one of embodiments [61], [62], or [65], wherein providing the second composition occurs after providing the first composition and before the first level of binding by the first composition decreases by more than 50% in the cell.
[67] The method of any one of embodiments [61], [62], [65], or [66], further comprising providing a third composition of the circular polyribonucleotide to the cell after the second composition, thereby maintaining binding of the target in the cell at least at the first level of binding.
[68] The method of embodiment [67], wherein providing the third composition occurs after providing the second composition and before the second level of the binding of the target in the cell by the first and second composition is substantially undetectable in the cell.
[69] The method of any one of embodiments [67] or [68], wherein providing the third composition occurs after providing the second composition and before the second level of the binding by the first and second composition in the cell decreases by more than 50%.
[70] The method of any one of embodiments [61]-[69] further comprising providing a fourth, fifth, sixth, seventh, eighth, ninth, or tenth composition of the circular polyribonucleotide.
[71] The method of any one of embodiments [63], [64], or [70], wherein providing the second composition of the circular polyribonucleotide occurs after the first composition and after the level of binding by the first composition is substantially undetectable.
[72] The method of any one of embodiments [63], [64], [70], or [71], wherein the second composition is provided to the cell at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of binding by the first composition is substantially undetectable.
[73] The method of any one of embodiments [61], [62], or [65]-[70], wherein the first level of binding is the highest level of the binding one day after providing the first composition.
[74] The method of any one of embodiments [61], [62], or [65]-[70], wherein the first level of the binding is 40%, 50%, 60%, 70%, 80%, or 90% of the highest level of the binding one day after providing the first composition.
[75] The method of any one of embodiments [61], [62], or [65]-[70], wherein the second level of binding is at least 30%, 40%, 50%, 60%, 70%, 80%, 90%, 100%, 110%, 120%, or 130% of the highest level of binding one day after providing the first composition for at least 1, 2, 3, 4, 5, 6, 7, 8, 9, 10, 12, 15, 20, 25, 30, 35, or 40 days after providing the second composition.
[76] The method of any one of embodiments [67]-[70], wherein the third level of the binding is at least 30%, 40%, 50%, 60%, 70%, 80%, 90%, 100%, 110%, 120%, or 130% of the highest level of binding one day after providing the first composition for at least 1, 2, 3, 4, 5, 6, 7, 8, 9, 10, 12, 15, 20, 25, 30, 35, or 40 days after providing the third composition.
[77] The method of any one of embodiments [61], [62], or [65]-[70], wherein for each subsequent composition provided after the first composition, a subsequent level of binding after each subsequent composition is at least 30%, 40%, 50%, 60%, 70%, 80%, 90%, 100%, 110%, 120%, or 130% of the highest level of binding one day after providing the first composition for at least 1, 2, 3, 4, 5, 6, 7, 8, 9, 10, 12, 15, 20, 25, 30, 35, or 40 days after providing each subsequent composition.
[78] The method of any one of embodiments [61], [62], or [65]-[70], wherein an average level of binding after providing the second composition is at least 40%, 50%, 60%, 70%, 80%, or 90% of the first level, wherein the average level of binding is measured from one day after providing the second composition to the day when the binding is substantially undetectable.
[79] The method of any one of embodiments [61], [62], or [65]-[70], wherein an average level of binding after providing each subsequent composition after the first composition is at least 40%, 50%, 60%, 70%, 80%, or 90% of the first level, wherein the average level of binding is measured from one day after providing each subsequent composition to the day when the binding is substantially undetectable.
[80] The method of any one of embodiments [61], [62], or [65]-[70], wherein the first level of the binding is maintained after providing the first composition and the second composition of the circular polyribonucleotide for at least 6 hours, 1 day, 2 days, 3 days, 5 days, 7 days, 14 days, 21 days, 28 days, or 35 days after providing the first composition.
[81] The method of any one of embodiments [67]-[70], wherein the first level of the binding is maintained after providing the first composition, second composition, and third composition of the circular polyribonucleotide for at least 6 hours, 1 day, 2 days, 3 days, 5 days, 7 days, 14 days, 21 days, 28 days, or 35 days after providing the first composition.
[82] The method of any one of embodiments [61], [62], or [65]-[70], wherein the second level of binding in the cell after providing the second composition is at least 1%, 5%, 10%, 20%, 30%, 40%, 50%, or 60% higher than the first level of binding in the cell after providing the first composition.
[83] The method of any one of embodiments [67]-[70], wherein a third level of binding in the cell after providing the third composition is at least 5%, 10%, 20%, 30%, 40%, 50%, or 60% higher than the first level of binding in the plurality after providing the first composition.
[84] The method of any one of embodiments [61], [62], or [65]-[70], wherein the second level of binding 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the binding after providing the first composition.
[85] The method of any one of embodiments [67]-[70], wherein a third level of binding 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the third composition of the circular polyribonucleotide is at least 1%, 5%, 10%, 20%, 30%, 40%, 50%, or 60% higher than the first level of the binding after providing the first composition.
[86] The method of any one of embodiments [63], [64], or [70]-[72], wherein the level of binding in the cell after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days.
[87] The method of any one of the embodiments [63], [64], or [70]-[472], wherein the level of binding in the cell after providing the first composition and the second composition of the circular polyribonucleotide is at least 5%, 10%, 20%, 30%, 40%, 50%, or 60% higher than the level of binding in the cell after providing the first composition and the second composition of the linear counterpart of the circular polyribonucleotide.
[88] The method of any one of embodiments [63], [64], or [70]-[72], wherein the level of binding in the cell after providing the first composition and the second composition of the circular polyribonucleotide is at least 5%, 10%, 20%, 30%, 40%, 50%, or 60% higher than the level of binding in the cell after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide.
[89] The method of any one of embodiments [1]-[88], wherein the first composition and the second composition comprise about the same amount of the circular polyribonucleotide.
[90] The method of any one of embodiments [1]-[89], wherein the first composition comprises a higher amount of the circular polyribonucleotides than the second composition.
[91] The method of any one of embodiments [1]-[90], wherein the first composition comprises a higher amount of the circular polyribonucleotides than a third, fourth, fifth, sixth, seventh, eighth, ninth, or tenth composition.
[92] The method of any one of embodiments [1]-[91], wherein an amount of circular polyribonucleotide of the second composition varies by no more than 1%, 5%, 10%, 15%, 20%, or 25% of an amount of circular polyribonucleotide of the first composition.
[93] The method of any one of embodiments [1]-[92], wherein an amount of circular polyribonucleotide of the second composition is no more than 1%, 5%, 10%, 15%, 20%, or 25% less than an amount of circular polyribonucleotide of the first composition.
[94] The method of any one of embodiments [1]-[93], wherein the first composition further comprises a pharmaceutically acceptable carrier or excipient.
[95] The method of any one of embodiments [1]-[94], wherein the second composition further comprises a pharmaceutically acceptable carrier or excipient.
[96] The method of any one of embodiments [1]-[95], wherein the third composition further comprises a pharmaceutically acceptable carrier or excipient.
[97] The method of any one of embodiments [1]-[96], wherein the cell is a eukaryotic cell.
[98] The method of any one of embodiments [1]-[97], wherein the cell is an animal cell.
[99] The method of any one of embodiments [1]-[98], wherein the cell is a mammalian cell.
[100] The method of any one of embodiments [1]-[99], wherein the cell is a human cell.
[101] The method of any one of embodiments [1]-[100], wherein the cell is a plurality of cells in a subject.
[102] The method of any one of embodiments [1]-[101], wherein the subject is an animal.
[103] The method of any one of embodiments [1]-[102], wherein the subject is a mammal.
[104] The method of any one of embodiments [1]-[103], wherein the subject is a human.
[105] The method of any one of embodiments [1]-[104], wherein the circular polyribonucleotide further comprises a stagger element at a 3′ end of an expression sequence, and lacks a termination element.
[106] The method of embodiment [105], wherein the stagger element stalls a ribosome during the rolling circle translation of the circular polyribonucleotide.
[107] The method of embodiment [105] or [106], wherein the stagger element encodes a sequence with a C-terminal consensus sequence that is D(V/I)ExNPGP, where x=any amino acid.
[108] The method of any one of embodiments [1]-[107], wherein the circular polyribonucleotide lacks an internal ribosomal entry site, a poly-A tail, a replication element, or any combination thereof.
[109] The method of any one of embodiments [1]-[108], wherein the one or more expression sequences comprise a Kozak initiation sequence.
[110] The method of any one of embodiments [1]-[109], wherein the circular polyribonucleotide further comprises at least one structural element selected from:
   (a) an encryptogen;
   (b) a regulatory element;
   (c) a replication element; and
   (d) quasi-double-stranded secondary structure.
[111] The method of any one of embodiments [1]-[110], wherein the circular polyribonucleotide comprises at least one functional characteristic selected from:
   (i) greater translation efficiency than a linear counterpart;
   (ii) a stoichiometric translation efficiency of multiple translation products;
   (iii) less immunogenicity than a counterpart lacking an encryptogen;
   (iv) increased half-life over a linear counterpart; and
   (v) persistence during cell division.
[112] The method of embodiment [105], wherein the termination element comprises a stop codon.
[113] The method of any one of embodiments [1]-[112], wherein the circular polyribonucleotide further comprises a replication domain configured to mediate self-replication of the circular polyribonucleotide.
[114] The method of any one of embodiments [1]-[113], wherein the circular polyribonucleotide persists during cell division.
[115] A method of inducing a response level in a subject after providing a first composition and a second composition of a circular polyribonucleotide to the subject compared to a response level in the subject after providing a first composition and second composition of a linear counterpart of the circular polyribonucleotide, comprising:
   providing a first composition of the circular polyribonucleotide encoding a protein and/or comprising a binding site that induces the response level, to the subject, wherein the subject comprises the response level after providing the first composition of the circular polyribonucleotide; and
   providing the second composition of the circular polyribonucleotide encoding a protein and/or comprising binding site after the first composition to the subject, wherein the subject comprises at least the response level after providing the second composition of the circular polyribonucleotide;
[116] thereby maintaining expression of the response level in the subject after providing the first composition and the second composition of the circular polyribonucleotide compared to the response level in the subject after providing the first composition and the second composition of the linear counterpart of the circular polyribonucleotide.A method of inducing a response level in a subject, comprising:
   (a) providing a first composition comprising a circular polyribonucleotide that encodes a protein and/or comprising binding site that induces the response to the subject; and
   (b) from 6 hours to 90 days following step (a), providing a second composition comprising a circular polyribonucleotide that encodes the protein and/or comprising binding site, to the subject,
   thereby inducing the response in the subject after providing the first composition and after providing the second composition.
[117] A method of inducing a response in a subject, comprising:
   (a) providing a first composition comprising a circular polyribonucleotide that encodes a protein and/or comprising binding site that induces the response to the subject, wherein the response is at a first level; and
   (b) from 6 hours to 90 days following step (a), providing a second composition comprising a circular polyribonucleotide that encodes the protein and/or comprising binding site that induces the response, to the subject.
[118] The method of any one of embodiments [115]-[117], wherein the protein is erthyropoietin.
[119] The method of any one of [115]-[118], wherein the response is to produce erythrocytes or the response level is a level of produced erthryocytes.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1: Circular RNA Administrated In Vivo and Displayed a Longer Half-Life/Increased Stability

This Example demonstrates the ability to deliver circular RNA and the increased stability of circular RNA compared to linear RNA in vivo.

For this Example, circular RNAs were designed to include an EMCV IRES with an ORF encoding Nanoluciferase (Nluc) and stagger sequence (EMCV 2A 3×FLAG Nluc 2A no stop and EMCV 2A 3×FLAG Nluc 2A stop). The circular RNA was generated in vitro.

Balb/c mice were injected with circular RNA with Nluc ORF, or linear RNA as a control, via intravenous (IV) tail vein administration. Animals received a single dose of 5 μg of RNA formulated in a lipid-based transfection reagent (Mirus) according to manufacturer's instructions.

Mice were sacrificed, and livers were collected at 3, 4, and 7 days post-dosing (n=2 mice/time point). The livers were collected and stored in an RNA stabilization reagent (Invitrogen). The tissue was homogenized in RIPA buffer with micro tube homogenizer (Fisher scientific) and RNA was extracted using a phenol-based RNA extraction reagent for cDNA synthesis. qPCR was used to measure the presence of both linear and circular RNA in the liver.

RNA detection in tissues was performed by qPCR. To detect linear and circular RNA primers that amplify the Nluc ORF were used. (F: AGATTTCGTTGGGGACTGGC, R: CACCGCTCAGGACAATCCTT). To detect only circular RNA, primers that amplified the 5′-3′ junction allowed for detection of circular but not linear RNA constructs (F: CTGGAGACGTGGAGGAGAAC, R: CCAAAAGACGGCAATATGGT).

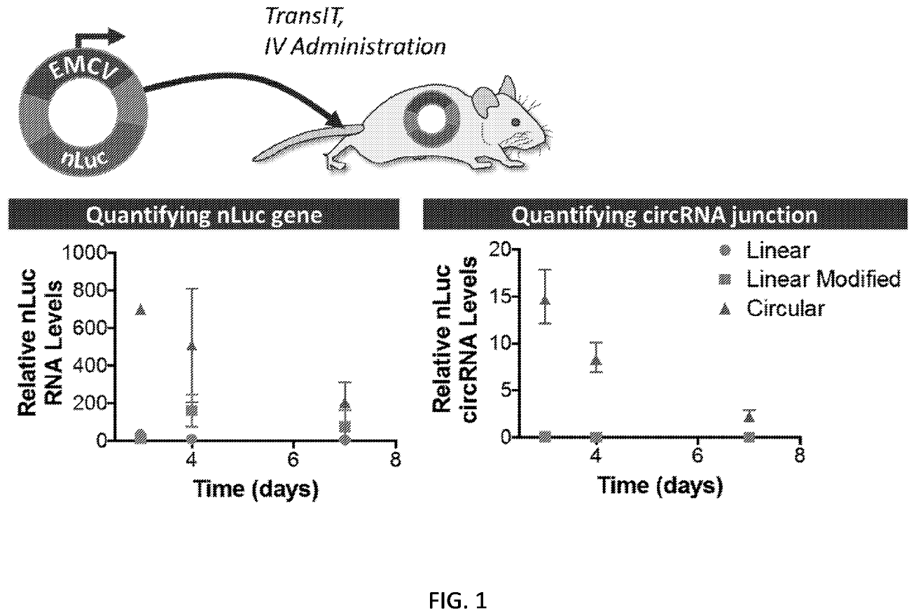

Circular RNA was detected at higher levels than linear RNA in livers of mice at 3, 4- and 7-days post-injection (FIG. 1). Therefore, circular RNA was administered and detectable in vivo for at least 7 days post administration.

### Example 2: In Vivo Expression, Half-Life, and Non-Immunogenicity of Circular RNA

This Example demonstrates the ability to drive expression from circular RNA in vivo. It demonstrates increased half-life of circular RNA compared to linear RNA. Finally, it demonstrates that circular RNA was engineered to be non-immunogenic in vivo

For this Example, circular RNAs included a CVB3 IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with an RNA cleanup kit (New England Biolabs, T2050), treated with RNA 5′-phosphohydrolase (RppH) (New England Biolabs, M0356) following the manufacturer's instructions, and purified again with an RNA purification column. RppH treated RNA was circularized using a splint DNA (GTCAACGGATTTTCCCAAGTCCGTAGCGTCTC) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNase free water.

Mice received a single tail vein injection dose of 2.5 μg of circular RNA with the Gaussia Luciferase ORF, or linear RNA as a control, both formulated in a lipid-based transfection reagent (Mirus) as a carrier.

Blood samples (50 μl) were collected from the tail-vein of each mouse into EDTA tubes, at 1, 2, 7, 11, 16, and 23 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μl of 1× GLuc substrate was added to 5 μl of plasma to carry out the GLuc luciferase activity assay. Plates were read right after mixing in a luminometer instrument (Promega).

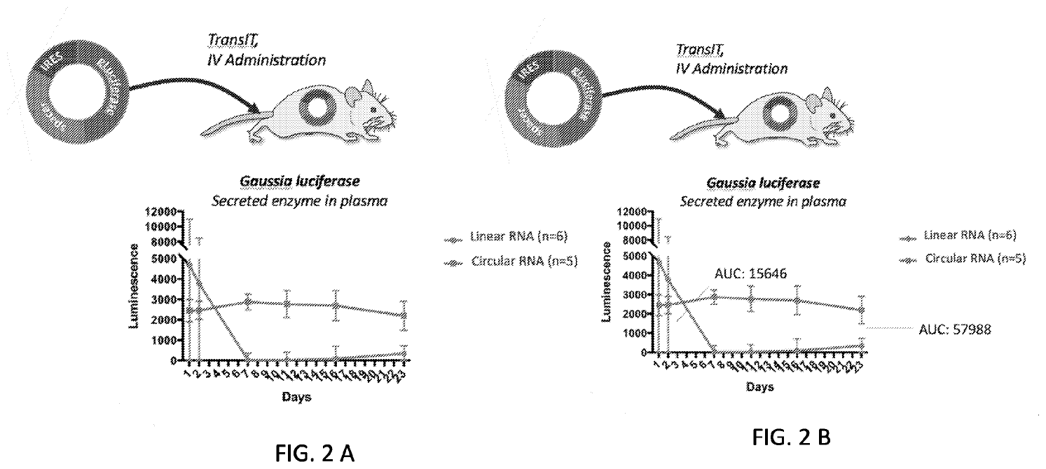

Gaussia Luciferase activity was detected in plasma at 1, 2, 7, 11, 16, and 23 days post-dosing of circular RNA (FIG. 2A and FIG. 2B).

In contrast, Gaussia Luciferase activity was only detected in plasma at 1, and 2 days post-dosing of modified linear RNA. Enzyme activity from linear RNA derived protein was not detected above background levels at day 6 or beyond (FIG. 2A and FIG. 2B).

At day 16, livers were dissected from three animals and total RNA was isolated from cells using a phenol-based extraction reagent (Invitrogen). Total RNA (500 ng) was subjected to reverse transcription to generate cDNA. qRT-PCR analysis was performed using a dye-based quantitative PCR mix (BioRad).

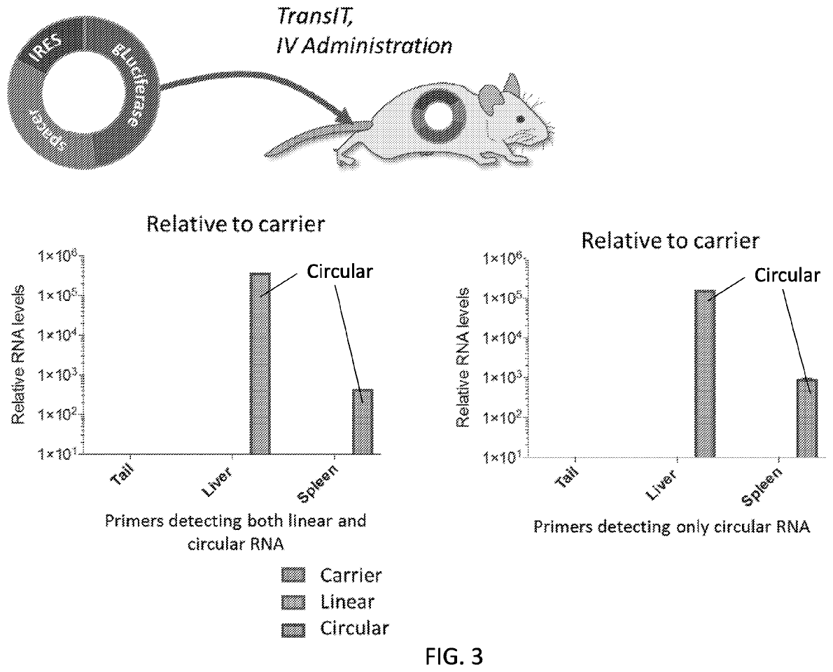

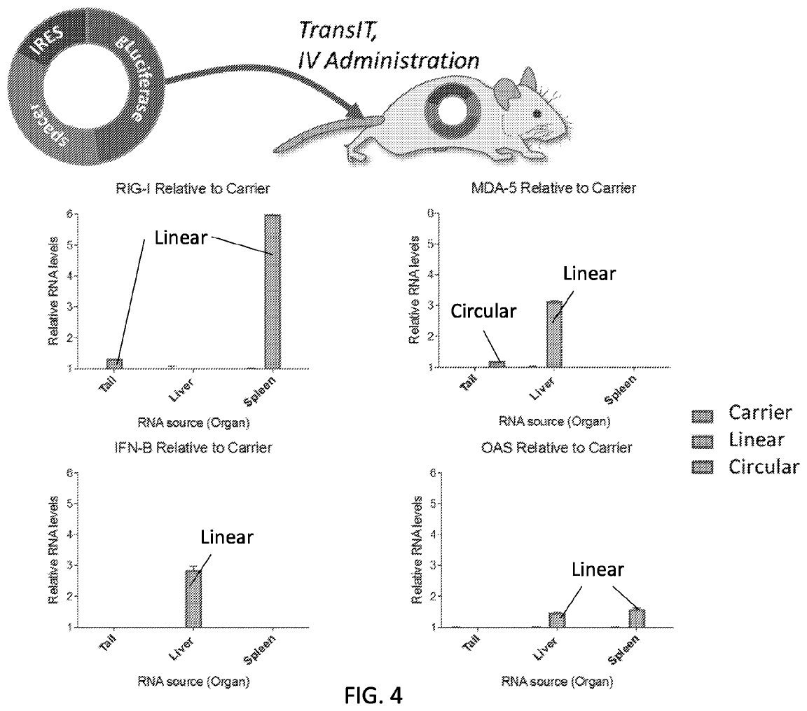

As shown in FIG. 3, qRT-PCR levels of circular RNA but not linear RNA were detected in both liver and spleen at day 16. As shown in FIG. 4, immune related genes from livers transfected with linear RNA showed increased expression of RIG-I, MDA5, IFN-B and OAS, while livers transfected with circular RNA did not show increased expression RIG-I, MDA5, PKR and IFN-beta of these markers as compared to carrier transfected animals at day 16. Thus, induction of immunogenic related genes in recipient cells was not present in circular RNA from transfected livers.

This Example demonstrated that circular RNA expressed protein in vivo for prolonged periods of time, with levels of protein activity in the plasma at multiple days post injection. Given the half-life of Gaussian Luciferase in mouse plasma is about 20 mins (see Tannous, Nat Protoc., 2009, 4(4):582-591), the similar levels of activity indicate continual expression from circular RNA. Further, circular RNA displayed a longer expression profile than its modified linear RNA counterpart without inducing immune related genes.

### Example 3: In Vivo Re-Dosing of Circular RNA

This Example demonstrates the ability to drive expression from circular RNA in vivo using two doses of circular RNA.

For this Example, circular RNAs included an EMCV IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with a Monarch RNA cleanup kit (New England Biolabs, T2050), treated with RNA 5′-phosphohydrolase (RppH) (New England Biolabs, M0356) following the manufacturer's instructions, and purified again with a Monarch RNA cleanup system. RppH treated RNA was circularized using a splint DNA (GTTTTTCGGCTATTCCCAATAGCCGTTTTG) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

Mice received a single tail vein injection dose of 0.25 μg of circular RNA with the Gaussia Luciferase ORF, or linear RNA as a control, both formulated in a lipid-based transfection reagent (Mirus) as a carrier at day 0, a second dose was administered at day 56.

Blood samples (50 μl) were collected from the tail-vein of each mouse into EDTA tubes, at 1, 2, 7, 11, 16, and 23 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μl of 1× GLuc substrate was added to 5 μl of plasma to carry out the GLuc luciferase activity assay. Plates were read right after mixing in a luminometer instrument (Promega).

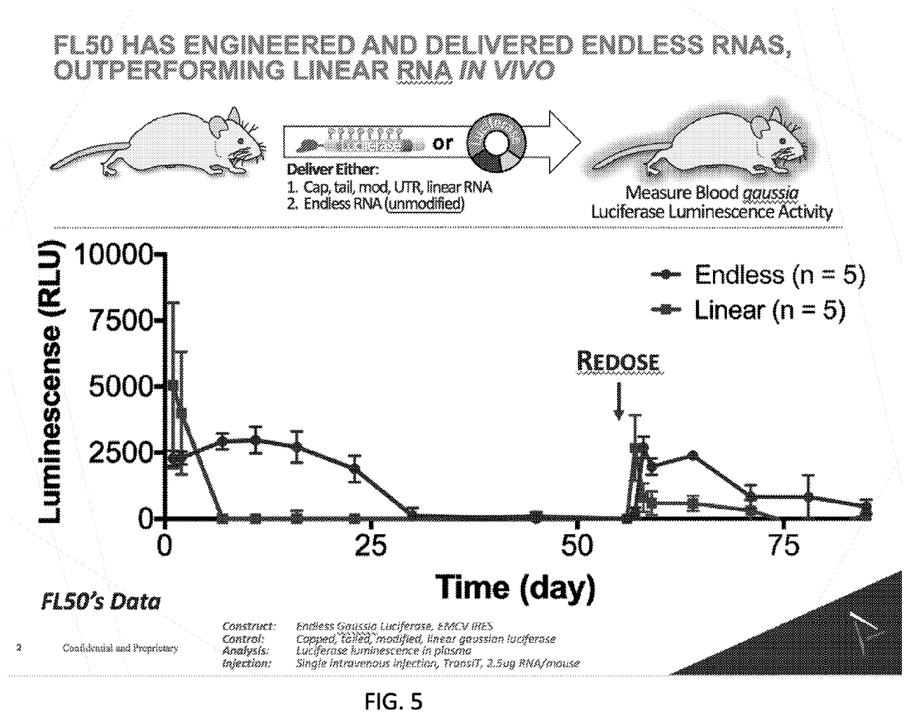

Gaussia Luciferase activity was detected in plasma at 1, 2, 7, 11, 16, and 23 days post-dosing of the first dose of circular RNA (FIG. 5).

In contrast, Gaussia Luciferase activity was only detected in plasma at 1, and 2 days post-dosing of modified linear RNA (FIG. 5).

Gaussia Luciferase activity was detected again in plasma at 2, 3, 8, and 15 days post-dosing of the second dose of circular RNA (FIG. 5).

In contrast, Gaussia Luciferase activity was only detected in plasma at 1, 2, 3 days post-dosing of modified linear RNA.

This Example demonstrated that circular RNA expressed protein in vivo for prolonged periods of time, with levels of protein activity in the plasma at multiple days post injection. Additionally, it demonstrates re-dosing of circular RNA results in a similar expression profile.

### Example 4: In Vivo Staggered Dosing of Circular RNA

This Example demonstrates the ability to drive higher expression from circular RNA in vivo using continuous staggered doses of circular RNA.

For this Example, circular RNAs included an EMCV IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with an RNA cleanup kit (New England Biolabs, T2050), treated with RNA 5′-phosphohydrolase (RppH) (New England Biolabs, M0356) following the manufacturer's instructions, and purified again with an RNA purification column. RppH treated RNA was circularized using a splint DNA (GTCAACGGATTTTCCCAAGTCCGTAGCGTCTC) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNase free water.

Mice received a tail vein injection dose of 0.25 μmol of circular RNA with the Gaussia Luciferase ORF, or linear RNA as a control, both formulated in a lipid-based transfection reagent (Mirus) as a carrier at day 0, day 2 and day 5.

Blood samples (50 μl) were collected from the tail-vein of each mouse into EDTA tubes, at 6 hours, 1, 2, 3, 5, 7, 14, 21, 28, 35, 42 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μl of 1× GLuc substrate was added to 5 μl of plasma to carry out the GLuc luciferase activity assay. Plates were read right after mixing in a luminometer instrument (Promega).

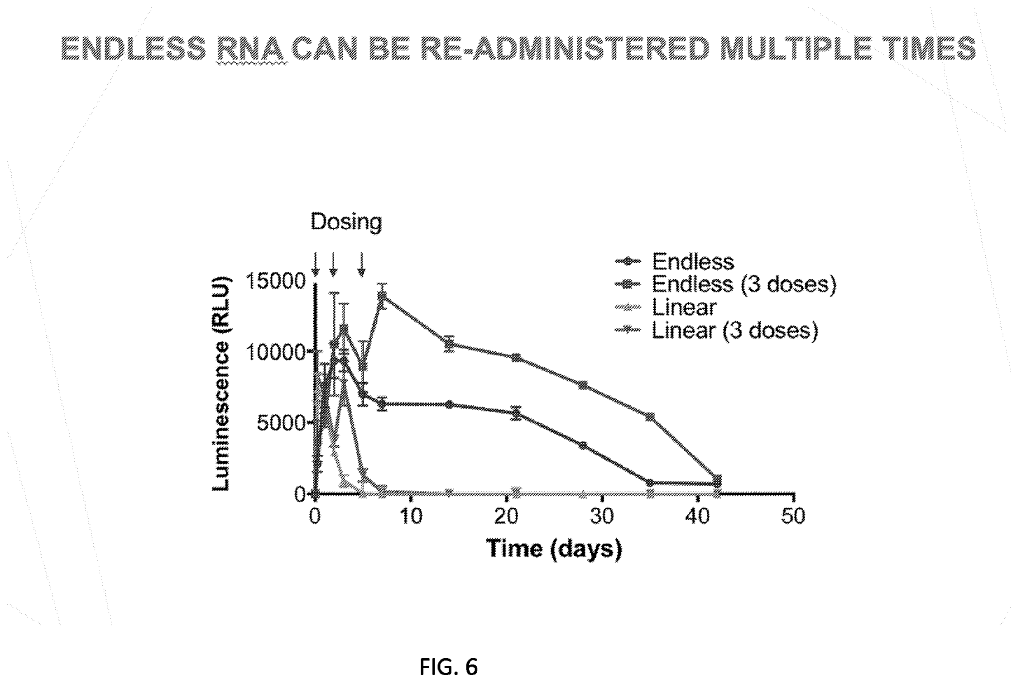

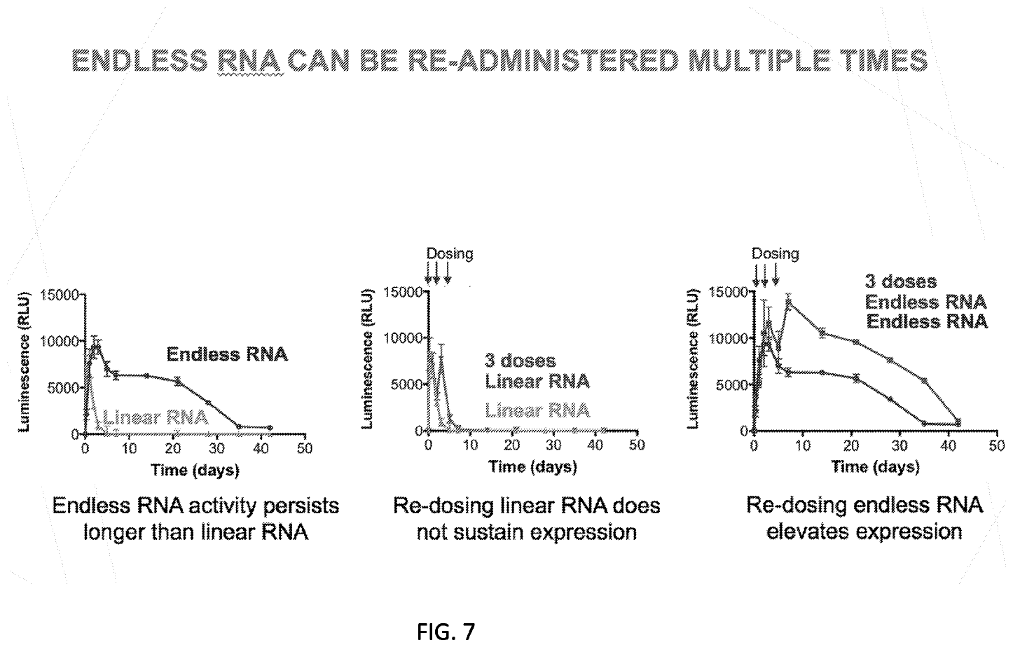

Gaussia Luciferase activity was detected in plasma at 6 hours, 1, 2, 3, 5, 7, 14, 21, 28 days post-dosing of a single dose of circular RNA (FIG. 6 and FIG. 7). Gaussia Luciferase activity was detected in plasma at 6 hours, 1, 2, 3, 5, 7, 14, 21, 28, 35 days post-dosing of the first dose of circular RNA when dosed with 3 doses (FIG. 6 and FIG. 7).

In contrast, Gaussia Luciferase activity was only detected in plasma at 6 hours, 1, 2, 3 days post-dosing of modified linear RNA and expression levels never increased beyond its initial dose. Enzyme activity from linear RNA derived protein was not detected above background levels at day x or beyond even though additional linear RNA was dosed (FIG. 6 and FIG. 7).

This Example demonstrated that circular RNA expressed protein in vivo for prolonged periods of time, with increased levels of protein activity in the plasma after multiple injections. Additionally, it demonstrates repeated dosing of circular RNA but not linear RNA results in expression.

### Example 5: Naked and Carrier Dose and Redose of Circular RNA Via Intravenous Delivery

This Example demonstrates the ability to drive expression from circular RNA in vivo using two doses of circular administered intravenously.

For this Example, circular RNAs included an EMCV IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with a Monarch RNA cleanup kit (New England Biolabs, T2050), treated with RNA 5′-phosphohydrolase (RppH) (New England Biolabs, M0356) following the manufacturer's instructions, and purified again with a Monarch RNA cleanup system. RppH-treated RNA was circularized using a splint DNA (GTTTTTCGGCTATTCCCAATAGCCGTTTTG) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

In this example, modified mRNA was custom synthesized by Trilink Biotechnologies and included all the motifs listed above. In this example, RNA was fully substituted with Pseudo-Uridine and 5-Methyl-C, capped with CleanCap™ AG and is polyadenylated (120A).

To generate unformulated RNA, circular RNA and mRNA were then diluted to a final concentration of 0.25 picomoles in 100 μL of PBS.

Circular RNA and mRNA were also formulated using a cationic lipid carrier. In this example, 15% TransIT (Mirus Bio) and 7.5% Boost were complexed with the RNA according to the manufacturer's instructions.

Mice received a single tail vein injection dose of 0.25 picomoles of circular RNA with the Gaussia Luciferase ORF in each formulation. Injections were performed at day 0, and a second dose was administered at day 49. Vehicle only was used as control.

Blood samples (50 μL) were collected by submental puncture into EDTA tubes, at 1, 2, 7, 11, 16, and 23 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μL of plasma to carry out the GLuc luciferase activity assay. Plates were read right after mixing in a luminometer instrument (Promega).

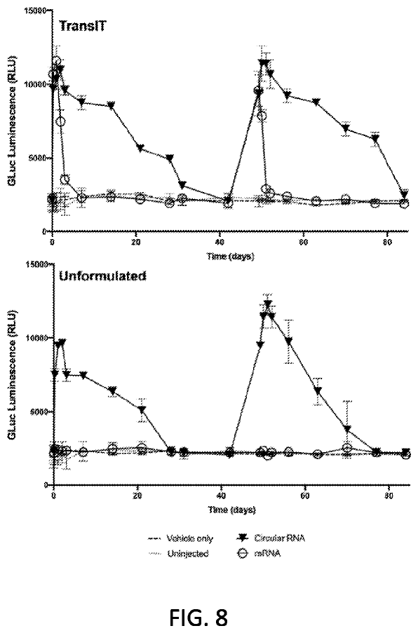

Gaussia Luciferase activity was detected in plasma at 1, 2, 7, 11, 16, and 23 days post-dosing of the first dose of both unformulated and TransIT-formulated circular RNA (FIG. 8).

In contrast, Gaussia Luciferase activity was only detected in plasma at 1, and 2 days post-dosing of both unformulated and TransIT-formulated modified mRNA (FIG. 8).

Gaussia Luciferase activity was detected again in plasma at 1, 2, 3, 8, 14 and 21 days post-dosing of the second dose of both unformulated and TransIT-formulated circular RNA (FIG. 8).

In contrast, Gaussia Luciferase activity was only detected in plasma at 1, 2, 3 days post-dosing of both unformulated and TransIT-formulated modified mRNA (FIG. 8).

In each case, Gaussia Luciferase activity was greater than the vehicle only control.

This Example demonstrated that circular RNA administered intravenously, with and without carrier, expressed protein in vivo for prolonged periods of time, with levels of protein activity in the plasma at multiple days post injection. Additionally, it demonstrates re-dosing of circular RNA results in a similar expression profile.

### Example 6: Naked Dose and Redose of Circular RNA Via Intramuscular Injection

This Example demonstrates the ability to drive expression from circular RNA in vivo using two doses of circular administered intramuscularly.

For this Example, circular RNAs included an EMCV IRES, an ORF encoding Gaussia Luciferase (GLuc), and two spacer elements flanking the IRES-ORF.

The circular RNA and mRNA were generated as described in Example 5.

To generate unformulated RNA, circular RNA and mRNA were then diluted to a final concentration of 2.5 picomoles in 100 μL of PBS.

Mice received a single intramuscular injection to the hind leg of dose of 2.5 picomoles of circular RNA with the Gaussia Luciferase ORF. Injections were performed at day 0, and a second dose was administered at day 49. Vehicle only was used as control.

Blood samples (50 μL) were collected by submental puncture into EDTA tubes, at 1, 2, 7, 11, 16, and 23 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μl of plasma to carry out the GLuc luciferase activity assay. Plates were read right after mixing in a luminometer instrument (Promega).

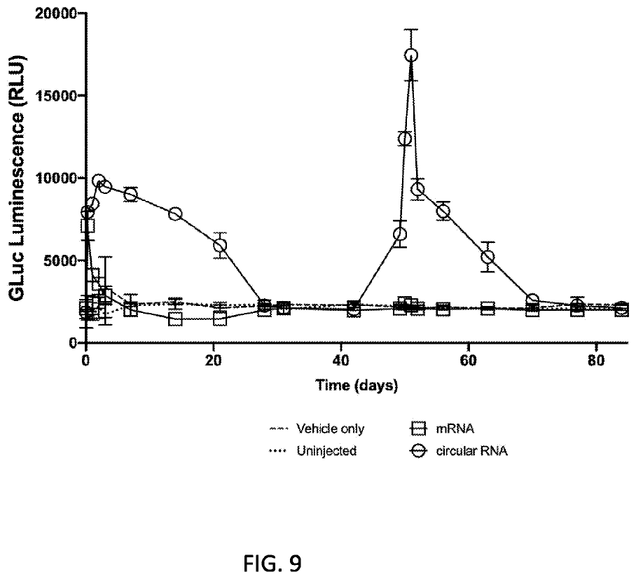

Gaussia Luciferase activity was detected in plasma at 1, 2, 7, 11, 16, and 23 days post-dosing of the first dose of unformulated circular RNA. (FIG. 9)

In contrast, Gaussia Luciferase activity was only detected in plasma at 1, and 2 days post-dosing of unformulated mRNA. (FIG. 9)

Gaussia Luciferase activity was detected again in plasma at 2, 3, 8, and 15 days post-dosing of the second dose of unformulated circular RNA. (FIG. 9)

In contrast, Gaussia Luciferase activity was only detected in plasma at 1, 2, 3 days post-dosing of unformulated modified mRNA. (FIG. 9)

In each case, Gaussia Luciferase activity was greater than the vehicle only control.

This Example demonstrated that circular RNA administered intramuscularly, without a carrier, expressed protein in vivo for prolonged periods of time, with levels of protein activity in the plasma at multiple days post injection. Additionally, it demonstrates re-dosing of circular RNA results in a similar expression profile.

### Example 7: Carrier Redose of Circular RNA Via Intravenous Injection Repeated Five Times, Results in Expression of Functional Protein

This Example demonstrates the ability to drive expression from circular RNA in vivo using five doses of circular RNA administered intravenously.

For this Example, circular RNAs included an EMCV IRES, an ORF encoding Gaussia Luciferase (GLuc), and two spacer elements flanking the IRES-ORF.

The circular RNA and mRNA were generated as described in Example 5.

Circular RNA and mRNA were formulated using a cationic lipid carrier. In this example, 10% TransIT (Mirus Bio) and 5% Boost were complexed with the RNA according to the manufacturer's instructions.

Mice received a single tail vein injection dose of 0.25 picomoles of circular RNA including the Gaussia Luciferase ORF. Injections were performed at: day 0, day 71, day 120, day 196, and day 359. Vehicle only was used as control.

Blood samples (50 μL) were collected submental puncture into EDTA tubes, at 0.25, 1, 2, 3, 7, 14, 21, 28 and 35 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μL of plasma to carry out the GLuc luciferase activity assay. Plates were read right after mixing in a luminometer instrument (Promega).

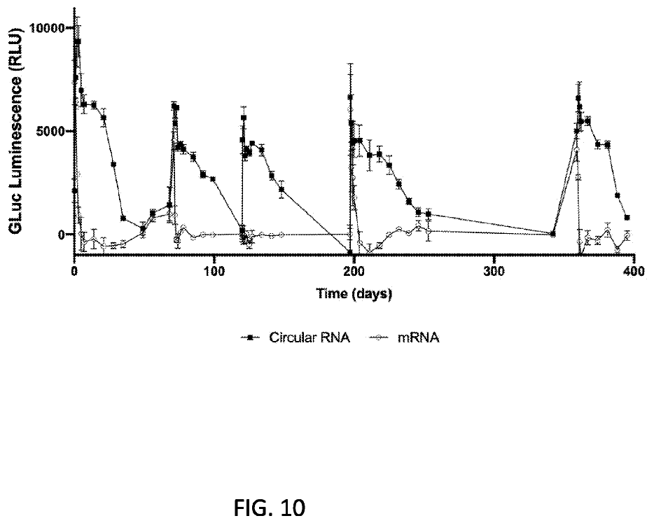

When dosed with Trans-IT formulated circular RNA, Gaussia Luciferase activity was detected in plasma at: days 1, 2, 3, 7, 14, 21 and 28 post-doing of the first dose; days 1, 2, 3, 7, 14 and 21 post-dosing of the second dose; 1, 2, 3, 7, 14 and 21 post-doing of the third dose; days 1, 2, 3, 7, 14, 21 and 28 post-doing of the fourth dose; and, days 1, 2, 3, 7, 14 and 21 post-doing of the fifth dose. (FIG. 10)

In contrast, when dosed with Trans-IT formulated modified mRNA, Gaussia Luciferase activity was detected in plasma at: days 0.25, 1 and 2 post-doing of the first dose; days 0.25, 1 and 2 post-dosing of the second dose; days 0.25, 1 and 2 post-doing of the third dose; days 0.25, 1 and 2 post-doing of the fourth dose; and, days 0.25, 1 and 2 post-doing of the fifth dose. (FIG. 10)

In each case, Gaussia Luciferase activity and thus expression was greater for circular RNA than for the mRNA.

This Example demonstrated that circular RNA administered intravenously, expressed protein in vivo for prolonged periods of time, with levels of protein activity in the plasma at multiple days post injection and could be redosed at least 5 times. Additionally, it demonstrates extended re-dosing of circular RNA results in a similar expression profile.

### Example 8: Carrier Redosing (5×) IV—Toxicity

This Example demonstrates the non-toxic effect of intravenous dosing of circular RNA five times over more than one year.

For this Example, circular RNAs included an EMCV IRES, an ORF encoding Gaussia Luciferase (GLuc), and two spacer elements flanking the IRES-ORF.

The circular RNA and mRNA were generated as described in Example 5.

Circular RNA and mRNA were formulated using a cationic lipid carrier. In this example, 10% TransIT (Mirus Bio) and 5% Boost were complexed with the RNA according to the manufacturer's instructions.

Mice received a single tail vein injection dose of 0.25 picomoles of circular RNA encoding Gaussia Luciferase in each formulation. Injections were performed at: day 0, day 71, day 120, day 196, and day 359. Vehicle only was used as control.

Mice were sacrificed at day 399 and liver, spleen, lung, and gallbladder were fixed in formalin. One slide per block was sectioned and stained with hematoxylin and eosin (H&E). Glass slides were evaluated by an ACVP board-certified veterinary pathologist using light microscopy. Histologic findings in each tissue were graded for severity 0-5, where 0=absent, 1=minimal, 2=mild, 3=moderate, 4=marked, 5=severe.

When dosed five times with Trans-IT formulated circular RNA, minimal to mild histologic changes were observed in the liver, spleen, lung, and gallbladder. Nearly all histopathologic findings (Table 1) were present in both control (carrier control and/or untreated control), circular RNA and mRNA injected animals. All findings were consistent with either background, perimortem, or non-specific changes not related to tested RNA toxicity

This Example demonstrated that circular RNA can be repeatedly dosed for more than one year with no signs of histopathological toxicity.

TABLE 1
When dosed five times with Trans-IT formulated circular
RNA, minimal to mild histologic changes were observed
in the liver, spleen, lung and gallbladder

| | | Liver/Gallbladder | | | | |
|---|---|---|---|---|---|---|
| | | Subacute | | | Spleen | |
| | | inflam- | | | Relative | Lung |
| | | mation | | | increase | Extra- |
| | | hepato- | Mixed | Extra- | extra- | medul- |
| | Ani- | cellular | infiltrate, | medullary | medullary | lary |
| | mal | necrosis, | peri- | hemato- | hemato- | hemato- |
| Group | # | random | biliary | poiesis | poiesis | poiesis |
| Vehicle | 1 | 1 | 0 | 0 | 0 | 0 |
| only | 2 | 0 | 0 | 0 | 0 | 0 |
| | 3 | 0 | 0 | 0 | 0 | 0 |
| Circular | 1 | 1 | 0 | 0 | 0 | 0 |
| RNA | 2 | 1 | 0 | 0 | 0 | 0 |
| | 3 | 0 | 1 | 2 | 3 | 1 |
| | 4 | 0 | 0 | 0 | 0 | 0 |
| mRNA | 1 | 0 | 0 | 0 | 0 | 0 |
| | 2 | 1 | 0 | 0 | 0 | 0 |
| | 3 | 1 | 0 | 0 | 0 | 0 |
| | 4 | 1 | 0 | 0 | 0 | 0 |
| Un- | 1 | 1 | 0 | 0 | 0 | 0 |
| treated | 2 | 1 | 1 | 0 | 0 | 0 |
| control | | | | | | |

### Example 9: Carrier Staggered Redosing (X2)—GLuc Activity

This Example demonstrates the ability to drive expression from circular RNA by redosing continuous staggered doses of circular RNA.

For this Example, circular RNAs included an EMCV IRES, an ORF encoding Gaussia Luciferase (GLuc), and two spacer elements flanking the IRES-ORF.

The circular RNA and mRNA were generated as described in Example 5.

Circular RNA and mRNA were also formulated using a cationic lipid carrier. In this example, 10% TransIT (Minis Bio) and 5% Boost were complexed with the RNA according to the manufacturer's instructions.

Mice received a single tail vein injection dose of 0.25 picomoles of the formulated circular RNA and modified mRNA with the Gaussia Luciferase ORF. Injections were performed in batches: a first batch at day 0, day 2, and day 5; and a second batch at day 71, day 73 and day 76. Vehicle only was used as control.

Blood samples (50 μL) were collected by submental puncture into EDTA tubes, at 0.25, 1, 2, 3, 7, 14, 21, 28 and 35 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μL of plasma to carry out the GLuc luciferase activity assay. Plates were read right after mixing in a luminometer instrument (Promega).

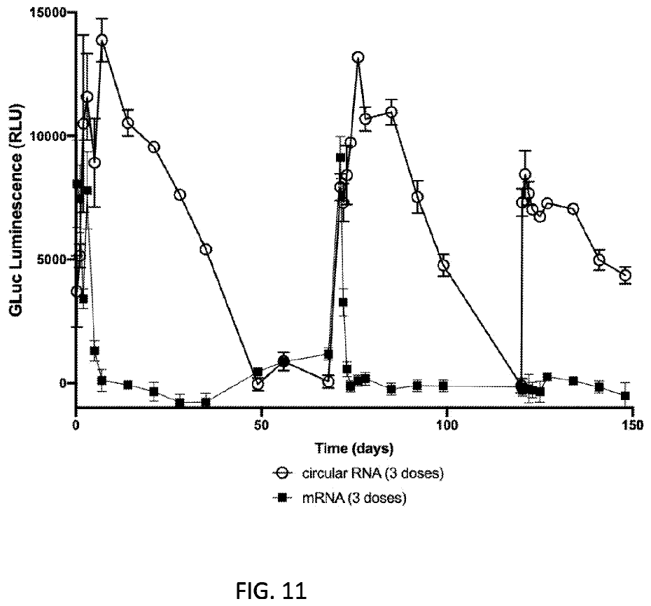

When dosed with Trans-IT formulated circular RNA, Gaussia Luciferase activity was detected in plasma at: days 1, 2, 3, 7, 14, 21, 28 and 35 of the first dose of batch 1; and then days 1, 2, 3, 7, 14, 21, 28 and 35 post-dosing of the first dose of batch 2 (FIG. 11).

In contrast, when dosed with Trans-IT formulated modified mRNA, Gaussia Luciferase activity was detected in plasma at: days 0.25, 1, 2 and 3 of the first dose of batch 1; and then days 0.25, 1 and 2 post-dosing of the first dose of batch 2 (FIG. 11).

Gaussia Luciferase activity was greater when expressed from circular RNA compared to the mRNA counterpart.

This Example demonstrated that circular RNA expressed protein in vivo for prolonged periods of time, with increased levels of protein activity in the plasma after multiple continuous injections. It demonstrates repeated dosing of circular RNA but not linear RNA results in expression. Additionally, it demonstrates extended re-dosing of circular RNA results in a similar expression profile.

### Example 10: TransIT Dose and Resdose of Therapeutically Relevant Circular Polvribonucleotides Administered IV

This Example demonstrates the ability to drive expression from circular RNA in vivo using two doses of a therapeutically relevant circular RNA administered intravenously.

For this Example, circular RNAs included an EMCV IRES, an ORF encoding Erythropoietin (EPO), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with a Monarch RNA cleanup kit (New England Biolabs, T2050), treated with RNA 5′-phosphohydrolase (RppH) (New England Biolabs, M0356) following the manufacturer's instructions, and purified again with a Monarch RNA cleanup system. RppH treated RNA was circularized using a splint DNA (5′-GTTTTTCGGCTATTCCCAATAGCCGTTTTG-3′) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

In this example, modified mRNA included a 5′ and 3′ Globin UTRs flanking the same EPO ORF nucleotide sequence encoded in the circular RNA. mRNA nucleotides were fully substituted with Pseudo-Uridine and 5-Methyl-C, capped with CleanCap™ AG and polyadenylated (90A).

In one formulation, circular RNA and mRNA were formulated using a cationic lipid carrier. In this example, 15% TransIT (Mirus Bio) and 7.5% Boost were complexed with 25 μmol RNA according to the manufacturer's instructions in a 100 uL final injection volume.

Unformulated circular RNA and mRNA were also tested. In this example, 25 μmol of each RNA was diluted to a final volume of 100 uL using PBS.

Mice received a single tail vein injection dose of 25 picomole at day 0, and a second dose was administered at day 79. Vehicle only was used as control.

Blood samples (40 uL) were collected from the mouse tail into EDTA tubes, at 1, 2, 3, 5, 7, 21, 28 and 35 days post first dosing and at 1, 2, 3, 5 and 7 days post second dose. Whole blood was stained with Retic-Count reagent (BD) for 30 min and acquired on a flow cytometer. Analysis was restricted to the red blood cell (RBC) population falling within a forward-scatter (FSC) versus side-scatter (SSC) gate for which 50000 events were acquired. Reticulocytes are reported as the percent of positively stained cells in the total RBC population.

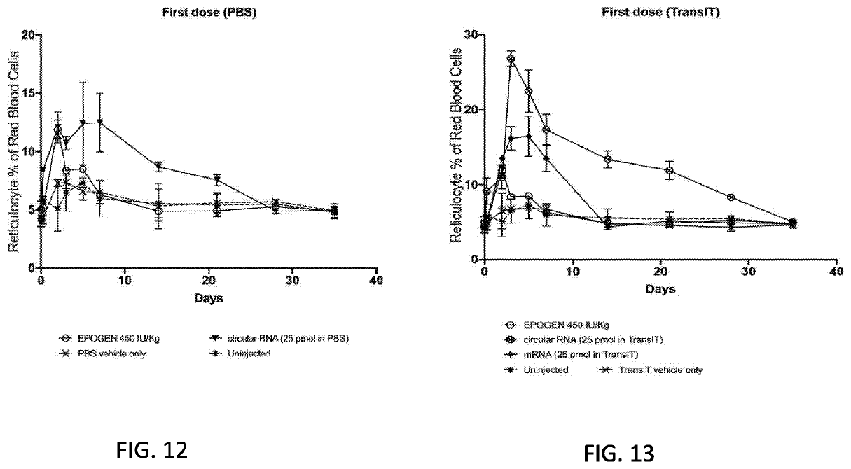

An increased number of reticulocytes was detected in whole blood at 3, 5, 7, 14, 21 and 28 days post-dosing of the first dose, after which reticulocyte counts were back to the normal range of 3-5% in our mouse population (FIG. 12 for unformulated and FIG. 13 for TransIT-formulated).

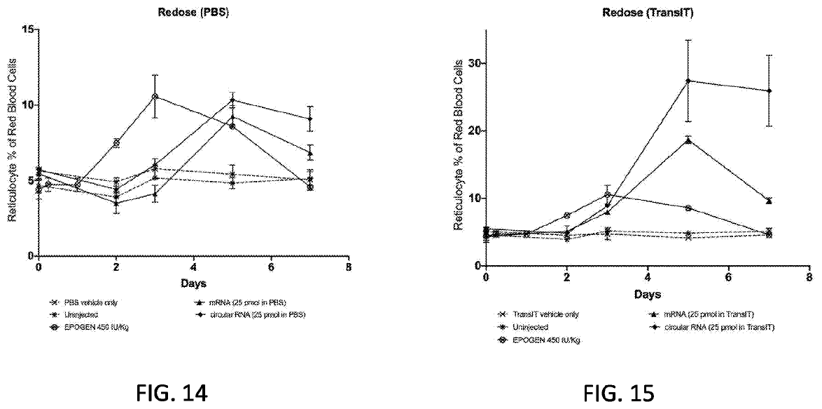

Post-dosing of the second dose, an increase in reticulocyte count was detected for circular RNA dosing and mRNA dosing when formulated with TransIT compared to the vehicle only control. This increase was greater and more sustained for both mRNA and circular RNA compared to a therapeutically relevant EPOGEN dose; and the increase was greater and more sustained for circular RNA than for mRNA dosing (FIG. 14).

Additionally, post-dosing of the second dose, an increase in reticulocyte count was detected for circular RNA dosing and mRNA dosing when unformulated compared to the vehicle only control. At days 5 and 7, reticulocyte count induced by circular RNA and mRNA was greater than the therapeutically relevant EPOGEN dose, demonstrating a more sustained therapeutic effect (FIG. 15). The therapeutic effect induced by circular RNA is greater than that of the mRNA.

This example shows circular RNA coding for hEPO elicits a biological effect above that seen for the equivalent dose of mRNA. The magnitude of response for each treatment is very similar between 1st and 2nd dose, approximately 25% for circular RNA, 18-20% for mRNA.

This Example demonstrates that circular RNA encoding a therapeutically relevant ORF, administered intravenously, with carrier, expressed protein in vivo for prolonged periods of time resulting on physiological effects observed for multiple days post injection. Additionally, it demonstrates re-dosing of circular RNA results in a similar expression profile.

### Example 11: In Vitro Circular RNA Production

This example describes in vitro production of a circular RNA.

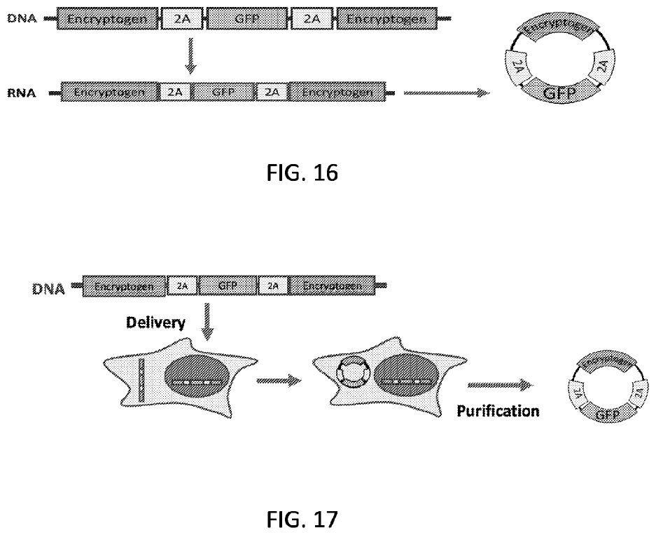

A circular RNA is designed with a start-codon (SEQ ID NO: 1), ORF(s) (SEQ ID NO:2), stagger element(s) (SEQ ID NO:3), encryptogen(s) (SEQ ID NO:4), and an IRES (SEQ ID NO:5), shown in FIG. 16. Circularization enables rolling circle translation, multiple open reading frames (ORFs) with alternating stagger elements for discrete ORF expression and controlled protein stoichiometry, encryptogen(s) to attenuate or mitigate RNA immunogenicity, and an optional IRES that targets RNA for ribosomal entry without poly-A sequence.

In this Example, the circular RNA is generated as follows. Unmodified linear RNA is synthesized by in vitro transcription using T7 RNA polymerase from a DNA segment having 5′- and 3′-ZKSCAN1 introns and an ORF encoding GFP linked to 2A sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), and the circular RNA is isolated following enrichment with RNase R treatment. RNA quality is assessed by agarose gel or through automated electrophoresis (Agilent).

### Example 12: In Vivo Circular RNA Production, Cell Culture

This example describes in vivo production of a circular RNA.

GFP (SEQ ID NO: 2) is cloned into an expression vector, e.g. pcDNA3.1(+) (Addgene) (SEQ ID NO: 6). This vector is mutagenized to induce circular RNA production in cells (SEQ ID NO: 6 and described by Kramer et al 2015), shown in FIG. 17.

HeLa cells are grown at 37° C. and 5% CO₂ in Dulbecco's modified Eagle's medium (DMEM) with high glucose (Life Technologies), supplemented with penicillin-streptomycin and 10% fetal bovine serum. One microgram of the above described expression plasmid is transfected using lipid transfection reagent (Life Technologies), and total RNA from the transfected cells is isolated using a phenol-based RNA isolation reagent (Life Technologies) as per the manufacturer's instructions between 1 hour and 20 days after transfection.

To measure GFP circular RNA and mRNA levels, qPCR reverse transcription using random hexamers is performed. In short, for RT-qPCR Hela cells' total RNA and RNase R-digested RNA from the same source are used as templates for the RT-PCR. To prepare the cDNAs of GFP mRNAs and circular GFP RNAs, the reverse transcription reactions are performed with a reverse transcriptase (Super-Script II: RNase H; Invitrogen) and random hexamers in accordance with the manufacturer's instruction. The amplified PCR products are analyzed using a 6% PAGE and visualized by ethidium bromide staining. To estimate the enrichment factor, the PCR products are quantified by densitometry (ImageQuant; Molecular Dynamics) and the concentrations of total RNA samples are measured by UV absorbance.

An additional RNA measurement is performed with northern blot analysis. Briefly, whole cell extract was obtained using a phenol based reagent (TRIzol) or nuclear and cytoplasmic protein extracts are obtained by fractionation of the cells with a commercial kit (CelLytic NuCLEAR Extraction Kit, Sigma). To inhibit RNA polymerase II transcription, cells are treated with flavopiridol (1 mM final concentration; Sigma) for 0-6 h at 37° C. For RNase R treatments, 10 mg of total RNA is treated with 20 U of RNase R (Epicentre) for 1 h at 37° C.

Northern blots using oligonucleotide probes are performed as follows. Oligonucleotide probes, PCR primers are designed using standard primer designing tools. T7 promoter sequence is added to the reverse primer to obtain an antisense probe in in vitro transcription reaction. In vitro transcription is performed using T7 RNA polymerase with a DIG-RNA labeling mix according to manufacturer's instruction. DNA templates are removed by DNAs I digestion and RNA probes purified by phenol chloroform extraction and subsequent precipitation. Probes are used at 50 ng/ml. Total RNA (2 μg-10 g) is denatured using Glyoxal load dye (Ambion) and resolved on 1.2% agarose gel in MOPS buffer. The gel is soaked in 1×TBE for 20 min and transferred to a Hybond-N+ membrane (GE Healthcare) for 1 h (15 V) using a semi-dry blotting system (Bio-Rad). Membranes are dried and UV-crosslinked (at 265 nm) 1× at 120,000 μJ cm-2. Pre-hybridization is done at 68° C. for 1 h and DIG-labelled in-vitro transcribed RNA probes are hybridized overnight. The membranes are washed three times in 2×SSC, 0.1% SDS at 68° C. for 30 min, followed by three 30 min washes in 0.2×SSC, 0.1% SDS at 68° C. The immunodetection is performed with anti-DIG directly-conjugated with alkaline phosphatase antibodies. Immunoreactive bands are visualized using chemiluminescent alkaline phosphatase substrate (CDP star reagent) and an image detection and quantification system (LAS-4000 detection system).

### Example 13: Preparation of Circular RNA and In Vitro Translation

This example describes gene expression and detection of the gene product from a circular RNA.

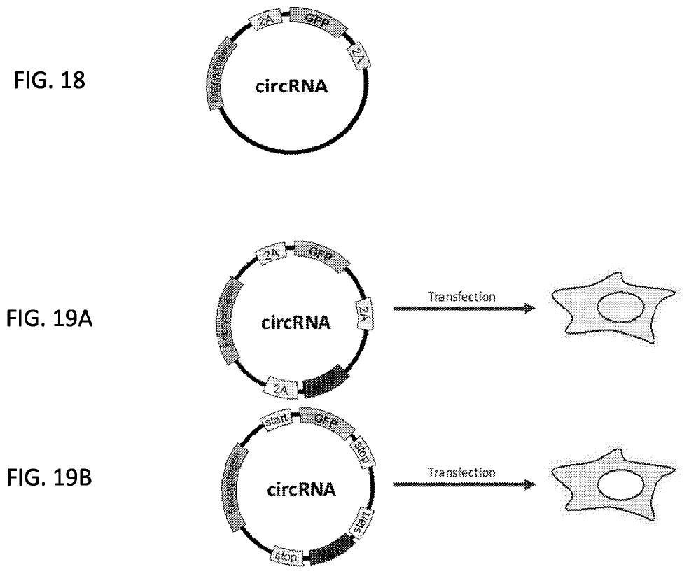

In this Example, the circular RNA is designed with a start-codon (SEQ ID NO: 1), a GFP ORF (SEQ ID NO:2), stagger element(s) (SEQ ID NO:3), human-derived encryptogen(s) (SEQ ID NO:4), and with or without an IRES (SEQ ID NO:5), see FIG. 18. In this Example, the circular RNA is generated either in vitro or in cells as described in Example 10 and 11.

The circular RNA is incubated for 5 h or overnight in rabbit reticulocyte lysate (Promega, Fitchburg, Wis., USA) at 30° C. The final composition of the reaction mixture includes 70% rabbit reticulocyte lysate, 10 μM methionine and leucine, 20 μM amino acids other than methionine and leucine, and 0.8 U/μL RNase inhibitor (Toyobo, Osaka, Japan). Aliquots are taken from the mixture and separated on 10-20% gradient polyacrylamide/sodium dodecyl sulfate (SDS) gels (Atto, Tokyo, Japan). The supernatant is removed and the pellet is dissolved in 2×SDS sample buffer (0.125 M Tris-HCl, pH 6.8, 4% SDS, 30% glycerol, 5% 2-mercaptoethanol, 0.01% bromophenol blue) at 70° C. for 15 min. The hemoglobin protein is removed during this process whereas proteins other than hemoglobin are concentrated.

After centrifugation at 1,400×g for 5 min, the supernatant is analyzed on 10-20% gradient polyacrylamide/SDS gels. A commercially available standard (BioRad) is used as the size marker. After being electrotransferred to a polyvinylidene fluoride (PVDF) membrane (Millipore) using a semi-dry method, the blot is visualized using a chemiluminescent kit (Rockland).

It is expected that the GFP protein is visualized in cell lysates and is detected in higher quantities in circular RNA than linear RNA, as a result of rolling circle translation.

### Example 14: Stoichiometric Protein Expression from Circular RNA

This example describes the ability of circular RNA to stoichiometrically express of proteins.

In this Example, one circular RNA is designed to include encryptogens (SEQ ID NO:4) and an ORF encoding GFP (SEQ ID NO: 2) and an ORF encoding RFP (SEQ ID NO:7) with stagger elements (SEQ ID NO: 3) flanking the GFP and RFP ORFs, see FIG. 19A Another circular RNA is designed similarly, however instead of flanking 2A sequences it will have a Stop and Start codon in between the GFP and RFP ORFs, see FIG. 19B. The circular RNAs are generated either in vitro or in cells as described in Example 11 and 12.

The circular RNAs are incubated for 5 h or overnight in rabbit reticulocyte lysate (Promega, Fitchburg, Wis., USA) at 30° C. The final composition of the reaction mixture includes 70% rabbit reticulocyte lysate, 10 μM methionine and leucine, 20 μM amino acids other than methionine and leucine, and 0.8 U/L RNase inhibitor (Toyobo, Osaka, Japan). Aliquots are taken from the mixture and separated on 10-20% gradient polyacrylamide/sodium dodecyl sulfate (SDS) gels (Atto, Tokyo, Japan). The supernatant is removed and the pellet is dissolved in 2×SDS sample buffer (0.125 M Tris-HCl, pH 6.8, 4% SDS, 30% glycerol, 5% 2-mercaptoethanol, 0.01% bromophenol blue) at 70° C. for 15 min. The hemoglobin protein is removed during this process whereas proteins other than hemoglobin are concentrated.

After centrifugation at 1,400×g for 5 min, the supernatant is analyzed on 10-20% gradient polyacrylamide/SDS gels. A commercially available standard (BioRad) is used as the size marker. After being electrotransferred to a polyvinylidene fluoride (PVDF) membrane (Millipore) using a semi-dry method, the blot is visualized using a chemiluminescent kit (Rockland).

It is expected that circular RNA with GFP and RFP ORFs not separated by a Stop and start codon will have equal amounts of either protein, while cells treated with the circular RNA including the start and stop codon in between the ORFs will have different amounts of either protein.

### Example 15: Synthetic Circular RNA Demonstrated Reduced Immunogenic Gene Expression in Cells

This Example demonstrates circular RNA engineered to have reduced immunogenicity as compared to a linear RNA.

Circular RNA that encoded a therapeutic protein provided a reduced induction of immunogenic related genes (RIG-I, MDA5, PKA and IFN-beta) in recipient cells, as compared to linear RNA. RIG-I can recognize short 5′ triphosphate uncapped double stranded or single stranded RNA, while MDA5 can recognize longer dsRNAs. RIG-I and MDA5 can both be involved in activating MAVS and triggering antiviral responses. PKR can be activated by dsRNA and induced by interferons, such as IFN-beta. As shown in the following Example, circular RNA was shown to have a reduced activation of an immune related genes in 293T cells than an analogous linear RNA, as assessed by expression of RIG-I, MDA5, PKR and IFN-beta by q-PCR.

The circular RNA and linear RNA were designed to encode either (1) a Kozak, 3×FLAG-EGF sequence with no termination element; (2) a Kozak, 3×FLAG-EGF, flanked by a termination element (stop codon); (3) a Kozak, 3×FLAG-EGF, flanked by a 2A sequence; or (4) a Kozak, 3×FLAG-EGF sequence flanked by a 2A sequence followed by a termination element (stop codon).

In this Example, the level of innate immune response genes were monitored in cells by plating 0.1×10⁶ cells into each well of a 12 well plate. After 1 day, 1 μg of linear or circular RNA was transfected into each well using a lipid-based transfection reagent (Invitrogen). Twenty-four hours after transfection, total RNA was isolated from cells using a phenol-based extraction reagent (Invitrogen). Total RNA (500 ng) was subjected to reverse transcription to generate cDNA. qRT-PCR analysis was performed using a dye-based quantitative PCR mix (BioRad).

Primer sequences used: Primers for GAPDH, F: AGGGCTGCTTTTAACTCTGGT, R: CCCCACTTGATTTTGGAGGGA; RIG-I, F: TGTGGGCAATGTCATCAAAA, R: GAAGCACTTGCTACCTCTTGC; MDA5, F: GGCACCATGGGAAGTGATT, R: ATTTGGTAAGGCCTGAGCTG; PKR, F: TCGCTGGTATCACTCGTCTG, R: GATTCTGAAGACCGCCAGAG; IFN-beta, F: CTCTCCTGTTGTGCTTCTCC, R: GTCAAAGTTCATCCTGTCCTTG.

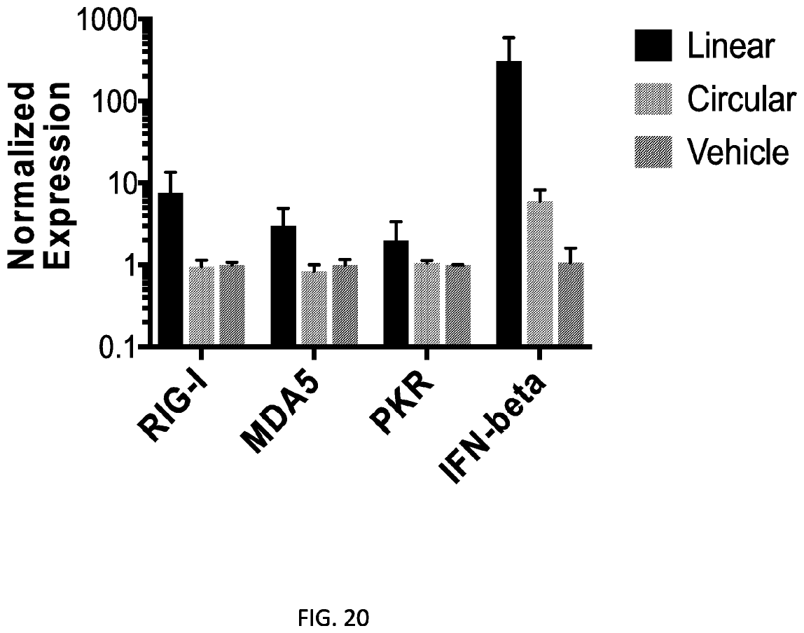

As shown in FIG. 20, qRT-PCR levels of immune related genes from 293T cells transfected with circular RNA showed reduction of RIG-I, MDA5, PKR and IFN-beta as compared to linear RNA transfected cells. Thus, induction of immunogenic related genes in recipient cells was reduced in circular RNA transfected cells, as compared to linear RNA transfected cells.

### Example 16: In Vivo Expression

This example describes the ability to express protein from a circular RNA in vivo.

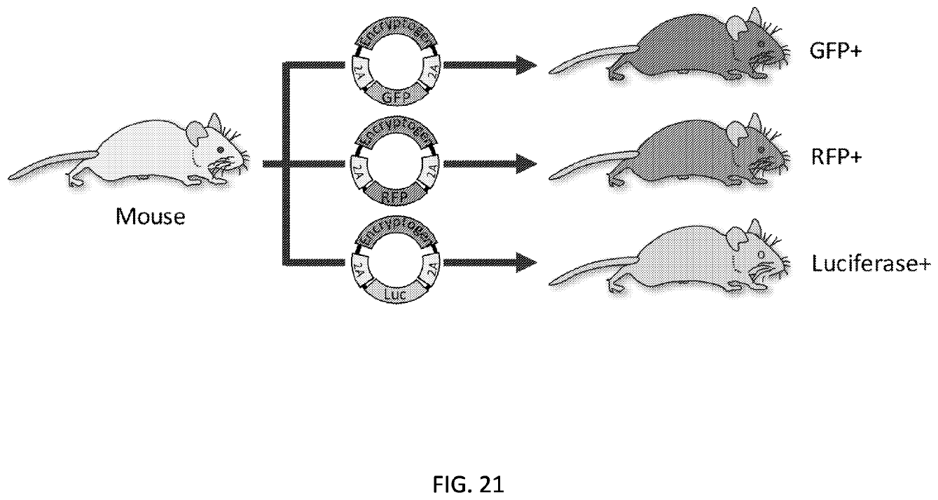

For this Example, circular RNAs are designed to include including encryptogen(s) (SEQ ID NO:4) and an ORF encoding GFP (SEQ ID NO:2) or RFP (SEQ ID NO:7) or Luciferase (SEQ ID NO:8) with stagger elements (SEQ ID NO:3) flanking the GFP, RFP or Luciferase ORF, see FIG. 21. The circular RNA is generated either in vitro or in cells as described in Example 11 and Example 12.

Male BALB/c mice 6-8 weeks old receive 300 mg/kg (6 mg) circular RNA (50 uL vol) with GFP, RFP, or luciferase ORFs, as described herein, or linear RNA as a control, via intradermal (ID), intramuscular (IM), oral (PO), intraperitoneal (IP), or intravenous (IV) administration. Animals receive a single dose or three injections (day 1, day 3, day 5).

Blood, heart, lung, spleen, kidney, liver, and skin injection sites are collected from non-dosed control mice and at 2, 4, 8, 24, 48, 72, 96 120, 168, and 264 hr post-dosing (n=4 mice/time point). Blood samples are collected from jugular venipuncture at study termination.

Circular RNA quantification for both serum and tissues is performed using quantification of branched DNA (bDNA) (Panomics/Affymetrix). A standard curve on each plate of known amounts of RNA (added to untreated tissue samples) is used to quantitate the RNA in treated tissues. The calculated amount in picograms (pg) is normalized to the amount of weighed tissue in the lysate applied to the plate. Protein expression (RFP or GFP) is evaluated by FACS or western blot in each tissue as described in a previous Example.

A separate group of mice dosed with luciferase circular RNA are injected with 3 mg luciferin at 6, 24, 48, 72, and 96 hr post-dosing and the animals are imaged on an in vivo imaging system (IVIS Spectrum, PerkinElmer). At 6 hr post-dosing, three animals are sacrificed and dissected, and the muscle, skin, draining lymph nodes, liver, and spleen are imaged ex vivo.

It is expected that mice express GFP, RFP, or luciferase in treated tissues.

### Example 17: In Vivo Biodistribution

This example describes the ability to control and measure biodistribution of circular RNA in vivo.

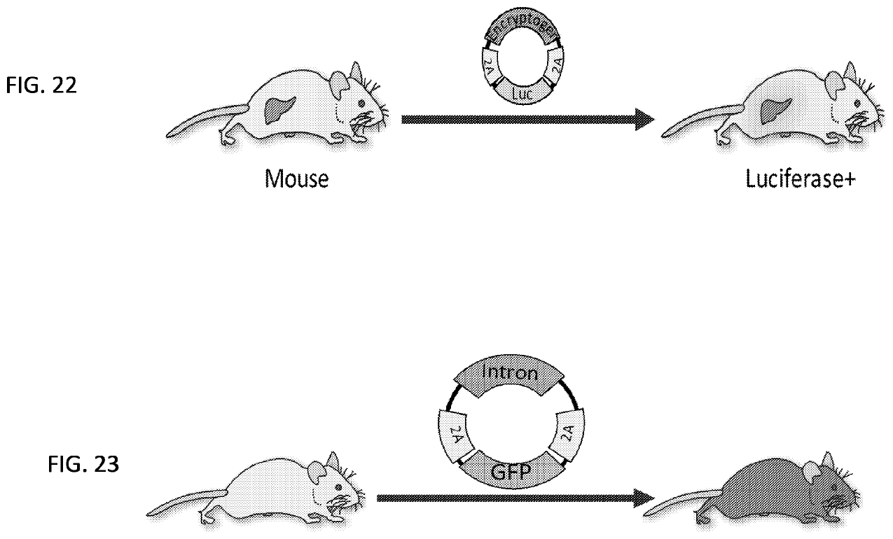

In this Example, mice are treated with the circular RNA encoding luciferase as described in Example 8. In short, circular RNAs designed to include including encryptogen(s) (SEQ ID NO:4) and an ORF encoding Luciferase (SEQ ID NO:8) with stagger elements (SEQ ID NO:3) flanking the Luciferase ORF, see FIG. 22. The circular RNA is generated either in vitro or in cells as described in Example 11 and 12.

Mice are dosed with luciferase circular RNA by injected with 3 mg luciferin, at 6, 24, 48, 72, and 96 hr post-dosing and the animals are imaged on an in vivo imaging system (IVIS Spectrum, PerkinElmer). At 6 hr post-dosing, three animals are sacrificed and dissected, and the muscle, skin, draining lymph nodes, liver, and spleen are imaged ex vivo

Circular RNA quantification for both serum and tissues is performed by using quantification of branched DNA (bDNA) (Panomics/Affymetrix). A standard curve on each plate of known amounts of RNA (added to untreated tissue samples) is used to quantitate the RNA in treated tissues. The calculated amount in picograms (pg) is normalized to the amount of weighed tissue in the lysate applied to the plate.

A separate group of male BALB/c mice 6-8 weeks old are dosed with luciferase circular RNA via IM or ID administration at four dose levels: 10, 2, 0.4, and 0.08 mg (n=6 per group). At 6, 24, 48, 72, and 96 hr post-dosing, animals are injected with 3 mg luciferin and imaged on an in vivo imaging system (IVIS Spectrum, PerkinElmer). At 6 hr post-dosing, three animals are sacrificed and dissected, and the muscle, skin, draining lymph nodes, liver, and spleen are imaged ex vivo. Tissues from the mice are also assessed for luciferase expression as described in Example 8 and tissue distribution of this expression is analyzed.

It is expected that mice show expression of luciferase in the treated tissues.

### Example 18: Non-Immunogenicity In Vivo

This example describes in vivo assessment of immunogenicity of the circular RNA after cell infection.

This Example describes quantification and comparison of the immune response after administrations of circular RNA harboring an encryptogen, see FIG. 23. In an embodiment, any of the circular RNA with an encryptogen, will have a reduced (e.g., reduced compared to administration of control RNA) immunogenic response following one or more administrations of the circular RNA compared to control.

A measure of immunogenicity for circular RNA are the cytokine levels in serum.

In this Example, cytokine serum levels are examined after one or more administrations of circular RNA. Circular RNA from any one of the previous Examples is administered via intradermal (ID), intramuscular (IM), oral (PO), intraperitoneal (IP), or intravenous (IV) into BALB/c mice 6-8 weeks old. Serum is drawn from the different cohorts: mice injected systemically and/or locally with injection(s) of circular RNA harboring an encryptogen and circular RNA without an encryptogen.

Collected serum samples are diluted 1-10× in PBS and analyzed for mouse IFN-α by enzyme-linked immunosorbent assay (PBL Biomedical Labs, Piscataway, N.J.) and TNF-α (R&D, Minneapolis, Minn.).

In addition to cytokine levels in serum, expression of inflammatory markers is another measure of immunogenicity. In this Example, spleen tissue from mice treated with vehicle (no circular RNA), linear RNA, or circular RNA will be harvested 1, 4, and 24 hours post administration. Samples will be analyzed using the following techniques qRT-PCR analysis, Northern blot or FACS analysis.

For qRT-PCR analysis mRNA levels for RIG-I, MDA5, OAS, OASL, TNF-alpha and PKR are quantified as described previously.

For Northern blot analysis. Samples are processed and analyzed for IFN-alpha 13, IFN-beta (Open Biosystems), TNF-alpha, or GAPDH (ATCC) as described above.

For FACS analysis, cells are stained with a directly conjugated antibodies against CD83 (Research Diagnostics Inc), HLA-DR, CD80 or CD86 and analyzed on a flow cytometer.

In an embodiment, circular RNA with an encryptogen will have decreased cytokine levels (as measured by ELISA, Northern blot, FACS and/or qRT-PCR) after one or multiple administrations, as compared control RNA.

### Example 19: Isolation and Purification of Circular RNA

This Example demonstrates circular RNA purification.

In certain embodiments, circular RNAs, as described in the previous Examples, may be isolated and purified before expression of the encoded protein products. This Example describes isolation using UREA gel separation. As shown in the following Example, circular RNA was isolated and purified.

CircRNA1 was designed to encode triple FLAG tagged EGF without stop codon (264nts). It has a Kozak sequence at the start codon for translation initiation (SEQ ID NO: 10). CirRNA2 has identical sequences with circular RNA1 except it has a termination element (triple stop codons) (273nts, SEQ ID NO: 11). Circular RNA3 was designed to encode triple FLAG tagged EGF flanked by a stagger element (2A sequence), without a termination element (stop codon) (330nts, SEQ ID NO: 9). CircRNA4 has identical sequences with circular RNA3 except it has a termination element (triple stop codon) (339nts). CircRNA5 was designed to encode FLAG tagged EGF flanked by a 2A sequence and followed by FLAG tagged nano luciferase (873nts, SEQ ID NO: 12). CircRNA6 has identical sequence with circular RNA5 except it included a termination element (triple stop codon) between the EGF and nano luciferase genes, and a termination element (triple stop codon) at the end of the nano luciferase sequence (762nts, SEQ ID NO: 13). CircRNA1, CircRNA2, CircRNA3, CircRNA4, CircRNA5, and CircRNA6 were isolated as described herein. CircRNA1, CircRNA2, CircRNA3, CircRNA4, CircRNA5, and CircRNA6 were isolated as described herein.

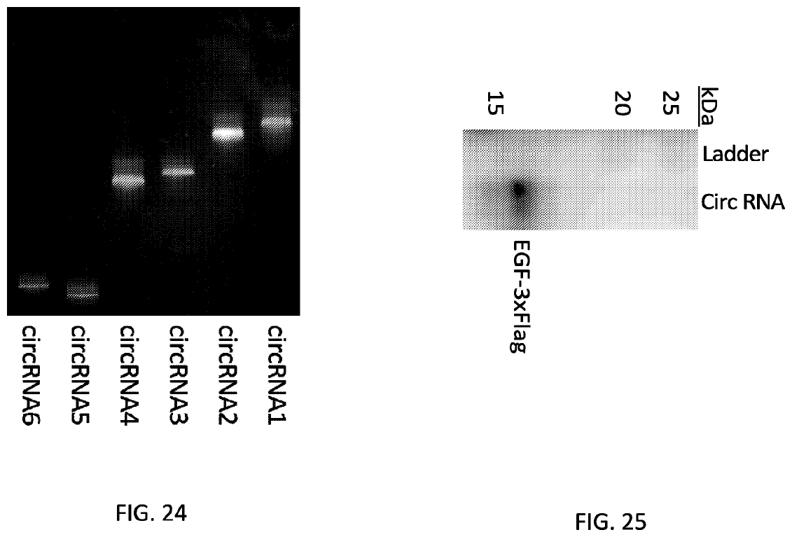

In this Example, linear and circular RNA were generated as described. To purify the circular RNAs, ligation mixtures were resolved on 6% denaturing PAGE and RNA bands corresponding to each of the circular RNAs were excised. Excised RNA gel fragments were crushed and RNA was eluted with 800 μl of 300 mM NaCl overnight. Gel debris was removed by centrifuge filters and RNA was precipitated with ethanol in the presence of 0.3 M sodium acetate. Eluted circular RNA was analyzed by 6% denaturing PAGE, see FIG. 24.

Single bands were visualized by PAGE for the circular RNAs having variable sizes.

### Example 20: Detection of Protein Expression

This Example demonstrates in vitro protein expression from a circular RNA.

Protein expression is the process of generating a specific protein from mRNA. This process includes the transcription of DNA into messenger RNA (mRNA), followed by the translation of mRNA into polypeptide chains, which are ultimately folded into functional proteins and may be targeted to specific subcellular or extracellular locations.

As shown in the following Example, a protein was expressed in vitro from a circular RNA sequence.

Circular RNA was designed to encode triple FLAG tagged EGF flanked by a 2A sequence without a termination element (stop codon) (330nts, SEQ ID NO: 9).

Linear or circular RNA was incubated for 5 hr in rabbit reticulocyte lysate at 30° C. in a volume of 25 μl. The final composition of the reaction mixture contained 70% rabbit reticulocyte lysate, 20 μM amino acids, 0.8 U/μl RNase inhibitor and 1p g of linear or circular RNA. After incubation, hemoglobin protein was removed by adding acetic acid (0.32μl) and water (300 μl) to the reaction mixture (16 μl) and centrifuging at 20,817×g for 10 min at 15° C. The supernatant was removed and the pellet was dissolved in 30° 1 of 2×SDS sample buffer and incubated at 70° C. for 15 min. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit (see FIG. 25) and western blot band intensity was measured by ImageJ.

Fluorescence was detected indicated expression product was present. Thus, circular RNA was shown to drive expression of a protein.

### SEQUENCE LISTING

(Start Codon)
SEQ ID NO: 1
AUG
(GFP)
SEQ ID NO: 2
EGFP:
atggtgagcaagggcgaggagctgttcaccggggtggtgcccatcctggtcgagctggacggcgacgtaaacggccacaagttcagcgtgtccggc
gagggcgagggcgatgccacctacggcaagctgaccctgaagttcatctgcaccaccggcaagctgcccgtgccctggcccaccctcgtgaccaccc
tgacctacggcgtgcagtgcttcagccgctaccccgaccacatgaagcagcacgacttcttcaagtccgccatgcccgaaggctacgtccaggagcgc
accatcttcttcaaggacgacggcaactacaagacccgcgccgaggtgaagttcgagggcgacaccctggtgaaccgcatcgagctgaagggcatcg
acttcaaggaggacggcaacatcctggggcacaagctggagtacaactacaacagccacaacgtctatatcatggccgacaagcagaagaacggcat
caaggtgaacttcaagatccgccacaacatcgaggacggcagcgtgcagctcgccgaccactaccagcagaacacccccatcggcgacggccccgt
gctgctgcccgacaaccactacctgagcacccagtccgccctgagcaaagaccccaacgagaagcgcgatcacatggtcctgctggagttcgtgacc
gccgccgggatcactctcggcatggacgagctgtacaag
(stagger element)
SEQ ID NO: 3
P2A: gctactaacttcagcctgctgaagcaggctggcgacgtggaggagaaccctggacct
T2A: gagggcaggggaagtctactaacatgcggggacgtggaggaaaatcccggccca
E2A: cagtgtactaattatgctctcttgaaattggctggagatgttgagagcaacccaggtccc
Others: F2A, BmCPV2A, BmIFV2A
ZKSCAN introns
SEQ ID NO: 4
GTAAAAAGAGGTGAAACCTATTATGTGTGAGCAGGGCACAGACGTTGAAACTGGAGCCAGG
AGAAGTATTGGCAGGCTTTAGGTTATTAGGTGGTTACTCTGTCTTAAAAATGTTCTGGCTTTC
TTCCTGCATCCACTGGCATACTCATGGTCTGTTTTTAAATATTTTAATTCCCATTTACAAAGT
GATTTACCCACAAGCCCAACCTGTCTGTCTTCAG
Or
GTAAGAAGCAAGGTTTCATTTAGGGGAAGGGAAATGATTCAGGACGAGAGTCTTTGTGCTG
CTGAGTGCCTGTGATGAAGAAGCATGTTAGTcctgggcaacgtagcgagaccccatctctacaaaaaatagaaaaattag
ccaggtatagtggcgcacacctgtgattccagctacgcaggaggctgaggtgggaggattgcttgagcccaggaggttgaggctgcagtgagctgtaa
tcatgccactactccaacctgggcaacacagcaaggaccctgtctcaaaaGCTACTTACAGAAAAGAATTAggctcggcacggtag
ctcacacctgtaatcccagcactttgggaggctgaggcgggcagatcacttgaggtcaggagtagagaccagcctggccaacatggtgaaaccttgtct
ctactaaaaatatgaaaattagccaggcatggtggcacattcctgtaatcccagctactcgggaggctgaggcaggagaatcacttgaacccaggaggt
ggaggttgcagtaagccgagatcgtaccactgtgctctagccttggtgacagagcgagactgtcttaaaaaaaaaaaaaaaaaaaaaagaattaattaaa
aatttaaaaaaaaatgaaaaaaaGCTGCATGCTTGTTTTTTGTTTTTAGTTATTCTACATTGTTGTCATTATT
ACCAAATATTGGGGAAAATACAACTTACAGACCAATCTCAGGAGTTAAATGTTACTACGAA
GGCAAATGAACTATGCGTAATGAACCTGGTAGGCATTA
(IRES)
IRES (EMCV):
SEQ ID NO: 5
Acgttactggccgaagccgcttggaataaggccggtgtgcgtttgtctatatgttattttccaccatattgccgtcttttggcaatgtgagggcccggaaacc
tggccctgtcttcttgacgagcattcctaggggtctttcccctctcgccaaaggaatgcaaggtctgttgaatgtcgtgaaggaagcagttcctctggaagct
tcttgaagacaaacaacgtctgtagcgaccctttgcaggcagcggaaccccccacctggcgacaggtgcctctgcggccaaaagccacgtgtataaga
tacacctgcaaaggcggcacaaccccagtgccacgttgtgagttggatagttgtggaaagagtcaaatggctctcctcaagcgtattcaacaaggggctg
aaggatgcccagaaggtaccccattgtatgggatctgatctggggcctcggtgcacatgctttacatgtgtttagtcgaggttaaaaaacgtctaggcccc
ccgaaccacggggacgtggttttcctttgaaaaacacgatgataata
(addgene p3.1 laccase)
pcDNA3.1(+) Laccase2 MCS Exon Vector sequence 6926 bps
SEQ ID NO: 6
GACGGATCGGGAGATCTCCCGATCCCCTATGGTGCACTCTCAGTACAATCTGCTCTGATGCC
GCATAGTTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTGCTTAGG
GTTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTGATGC
GGTTTTGGCAGTACATCAATGGGCGTGGATAGCGGTTTGACTCACGGGGATTTCCAAGTCTC
CACCCCATTGACGTCAATGGGAGTTTGTTTTGGCACCAAAATCAACGGGACTTTCCAAAATG
TCGTAACAACTCCGCCCCATTGACGCAAATGGGCGGTAGGCGTGTACGGTGGGAGGTCTATA
TAAGCAGAGCTCTCTGGCTAACTAGAGAACCCACTGCTTACTGGCTTATCGAAATTAATACG
ACTCACTATAGGGAGACCCAAGCTGGCTAGCGTTTAAACTTAAGCTTGGTACCGAGCTCGGA
TCCACTAGTCCAGTGTGGTGGAATTCCATTGAGAAATGACTGAGTTCCGGTGCTCTCAAGTC
ATTGATCTTTGTCGACTTTTATTTGGTCTCTGTAATAACGACTTCAAAAACATTAAATTCTGT
TGCGAAGCCAGTAAGCTACAAAAAGAAAaaacaagagagaatgctatagtcgtatagtatagtttcccgactatctgataccc
attacttatctagggggaatgcgaacccaaaatittatcagttttctcggatatcgatagatattggggaataaatttaaataaataaattttgggcgggttt
agggcgtggcaaaaagttttttggcaaatcgctagaaatttacaagacttataaaattatgaaaaaatacaacaaaatittaaacacgtgggcgtgacagttt
tggGcggttnagggcgttagagtaggcgaggacagggttacatcgactaggattgatcctgatcaagaatatatatactttataccgcttccttctacatgtt
acctatttttcaacgaatctagtatacctttttactgtacgatttatgggtataaTAATAAGCTAAATCGAGACTAAGttttattgttatatatatt
ttttttattttatGCAGAAATTAATTAAACCGGTCCTGCAGGTGATCAGGCGCGCCGGTTACCGGCCGG
CCCCGCGGAGCGTAAGTATTCAAAATTCCAAAATTTTTTACTAGAAATATTCGATTTTTTAAT
AGGCAGTTTCTATACTATTGTATACTATTGtagattcgttgaaaagtatgtaacaggaagaataaagcataccgaccatgtaa
agtatatatattcttaataaggatcaatagccgagtcgatctcgccatgtccgtctgtcttattGttttattaccgccgagacatcaggaactataaaagc
tagaaggatgagttttagcatacagattctagagacaaggacgcagagcaagtagttgatccatgctgccacgctttaactactcaaattgcccaaaactg
ccatgcccacatttttgaactattttcgaaattttttcataattgtattactcgtgtaaatttccatcaatttgccaaaaaactttttgtcacgcgttaac
gccctaaagccgccaatttggtcacgcccacactattgaGcaattatcaaattttttctcattttattccccaatatctatcgatatccccgattatgaaa
ttattaaatttcgcgttcgcattcacactagctgagtaacgagtatctgatagttggggaaatcgactTATTTTTTATATACAATGAAAATGAATTTAATC
ATATGAATATCGATTATAGCTTTTTATTTAATATGAATATTTATTTGGGCTTAAGGTGTAACC
TcctcgacataagactcacatggcgcaggcacattgaagacaaaaatactcaTTGTCGGGTCTCGCACCCTCCAGCAGCAC
CTAAAATTATGTCTTCAATTATTGCCAACATTGGAGACACAATTAGTCTGTGGCACCTCAGG
CGGCCGCTCGAGTCTAGAGGGCCCGTTTAAACCCGCTGATCAGCCTCGACTGTGCCTTCTAG
TTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCA
TGCTGGGGATGCGGTGGGCTCTATGGCTTCTGAGGCGGAAAGAACCAGCTGGGGCTCTAGG
GGGTATCCCCACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCTTCCCTTCCTTTCT
CGCCACGTTCGCCGGCTTTCCCCGTCAAGCTCTAAATCGGGGGCTCCCTTTAGGGTTCCGATT
TAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGAC
TCTTGTTCCAAACTGGAACAACACTCAACCCTATCTCGGTCTATTCTTTTGATTTATAAGGGA
TTTTGCCGATTTCGGCCTATTGGTTAAAAAATGAGCTGATTTAACAAAAATTTAACGCGAAT
TAATTCTGTGGAATGTGTGTCAGTTAGGGTGTGGAAAGTCCCCAGGCTCCCCAGCAGGCAGA
AGTATGCAAAGCATGCATCTCAATTAGTCAGCAACCAGGTGTGGAAAGTCCCCAGGCTCCCC
AGCAGGCAGAAGTATGCAAAGCATGCATCTCAATTAGTCAGCAACCATAGTCCCGCCCCTA
ACTCCGCCCATCCCGCCCCTAACTCCGCCCAGTTCCGCCCATTCTCCGCCCCATGGCTGACTA
ATTTTTTTTATTTATGCAGAGGCCGAGGCCGCCTCTGCCTCTGAGCTATTCCAGAAGTAGTGA
GGAGGCTTTTTTGGAGGCCTAGGCTTTTGCAAAAAGCTCCCGGGAGCTTGTATATCCATTTTC
GGATCTGATCAAGAGACAGGATGAGGATCGTTTCGCATGATTGAACAAGATGGATTGCACG
CAGGTTCTCCGGCCGCTTGGGTGGAGAGGCTATTCGGCTATGACTGGGCACAACAGACAATC
GGCTGCTCTGATGCCGCCGTGTTCCGGCTGTCAGCGCAGGGGCGCCCGGTTCTTTTTGTCAA
GACCGACCTGTCCGGTGCCCTGAATGAACTGCAGGACGAGGCAGCGCGGCTATCGTGGCTG
GCCACGACGGGCGTTCCTTGCGCAGCTGTGCTCGACGTTGTCACTGAAGCGGGAAGGGACTG
GCTGCTATTGGGCGAAGTGCCGGGGCAGGATCTCCTGTCATCTCACCTTGCTCCTGCCGAGA
AAGTATCCATCATGGCTGATGCAATGCGGCGGCTGCATACGCTTGATCCGGCTACCTGCCCA
TTCGACCACCAAGCGAAACATCGCATCGAGCGAGCACGTACTCGGATGGAAGCCGGTCTTG
TCGATCAGGATGATCTGGACGAAGAGCATCAGGGGCTCGCGCCAGCCGAACTGTTCGCCAG
GCTCAAGGCGCGCATGCCCGACGGCGAGGATCTCGTCGTGACCCATGGCGATGCCTGCTTGC
CGAATATCATGGTGGAAAATGGCCGCTTTTCTGGATTCATCGACTGTGGCCGGCTGGGTGTG
GCGGACCGCTATCAGGACATAGCGTTGGCTACCCGTGATATTGCTGAAGAGCTTGGCGGCGA
ATGGGCTGACCGCTTCCTCGTGCTTTACGGTATCGCCGCTCCCGATTCGCAGCGCATCGCCTT
CTATCGCCTTCTTGACGAGTTCTTCTGAGCGGGACTCTGGGGTTCGAAATGACCGACCAAGC
GACGCCCAACCTGCCATCACGAGATTTCGATTCCACCGCCGCCTTCTATGAAAGGTTGGGCT
TCGGAATCGTTTTCCGGGACGCCGGCTGGATGATCCTCCAGCGCGGGGATCTCATGCTGGAG
TTCTTCGCCCACCCCAACTTGTTTATTGCAGCTTATAATGGTTACAAATAAAGCAATAGCATC
ACAAATTTCACAAATAAAGCATTTTTTTCACTGCATTCTAGTTGTGGTTTGTCCAAACTCATC
AATGTATCTTATCATGTCTGTATACCGTCGACCTCTAGCTAGAGCTTGGCGTAATCATGGTCA
TAGCTGTTTCCTGTGTGAAATTGTTATCCGCTCACAATTCCACACAACATACGAGCCGGAAG
CATAAAGTGTAAAGCCTGGGGTGCCTAATGAGTGAGCTAACTCACATTAATTGCGTTGCGCT
CACTGCCCGCTTTCCAGTCGGGAAACCTGTCGTGCCAGCTGCATTAATGAATCGGCCAACGC
GCGGGGAGAGGCGGTTTGCGTATTGGGCGCTCTTCCGCTTCCTCGCTCACTGACTCGCTGCG
CTCGGTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCCAGGA
ACCGTAAAAAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCTCAGTT
CGGTGTAGGTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTATCGCCACT
GGCAGCAGCCACTGGTAACAGGATTAGCAGAGCGAGGTATGTAGGCGGTGCTACAGAGTTC
TTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCT
GGTAGCGGTTTTTTTGTTTGCAAGCAGCAGATTACGCGCAGAAAAAAAGGATCTCAAGAAG
ATCCTTTGATCTTTTCTACGGGGTCTGACGCTCAGTGGAACGAAAACTCACGTTAAGGGATT
TTGGTCATGAGATTATCAAAAAGGATCTTCACCTAGATCCTTTTAAATTAAAAATGAAGTTTT
AAATCAATCTAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATCGTGGT
GTCACGCTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGTCAGAA
GTAAGTTGGCCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGCCACATAG
CAGAACTTTAAAAGTGCTCATCATTGGAAAACGTTCTTCGGGGCGAAAACTCTCAAGGATCT
TACCGCTGTTGAGATCCAGTTCGATGTAACCCACTCGTGCACCCAACTGATCTTCAGCATCTT
TTACTTTCACCAGCGTTTCTGGGTGAGCAAAAACAGGAAGGCAAAATGCCGCAAAAAAGGG
AATAAGGGCGACACGGAAATGTTGAATACTCATACTCTTCCTTTTTCAATATTATTGAAGCA
TTTATCAGGGTTATTGTCTCATGAGCGGATACATATTTGAATGTATTTAGAAAAATAAACAA
ATAGGGGTTCCGCGCACATTTCCCCGAAAAGTGCCACCTGACGTC
(RFP)
mCherry:
SEQ ID NO: 7
atggtgagcaagggcgaggaggataacatggccatcatcaaggagttcatgcgcttcaaggtgcacatggagggctccgtgaacggccacgagttcg
agatcgagggcgagggcgagggccgcccctacgagggcacccagaccgccaagctgaaggtgaccaagggtggccccctgcccttcgcctggga
catcctgtcccctcagttcatgtacggctccaaggcctacgtgaagcaccccgccgacatccccgactacttgaagctgtccttccccgagggcttcaagt
gggagcgcgtgatgaacttcgaggacggcaacgtggtgaccgtgacccaggactcctccctgcaggacggcgagttcatctacaaggtgaagctgcg
cggcaccaacttcccctccgacggccccgtaatgcagaagaagaccatgggctgggaggcctcctccgagcggatgtaccccgaggacggcgccct
gaagggcgagatcaagcagaggctgaagctgaaggacaacggccactacgacgctgaggtcaagaccacctacaaggccaagaagcccgtgcag
ctgcccggcgcctacaacgtcaacatcaagttggacatcacctcccacaacgaggactacaccatcgtggaacagtacgaacgcgccgagggccgcc
actccaccggcggcatggacgagctgtacaag
(luciferase)
nLuc:
SEQ ID NO: 8
ATGGTCTTCACACTCGAAGATTTCGTTGGGGACTGGCGACAGACAGCCGGCTACAACCTGGA
CCAAGTCCTTGAACAGGGAGGTGTGTCCAGTTTGTTTCAGAATCTCGGGGTGTCCGTAACTC
CGATCCAAAGGATTGTCCTGAGCGGTGAAAATGGGCTGAAGATCGACATCCATGTCATCATC
CCGTATGAAGGTCTGAGCGGCGACCAAATGGGCCAGATCGAAAAAATTTTTAAGGTGGTGT
ACCCTGTGGATGATCATCACTTTAAGGTGATCCTGCACTATGGCACACTGGTAATCGACGGG
GTTACGCCGAACATGATCGACTATTTCGGACGGCCGTATGAAGGCATCGCCGTGTTCGACGG
CAAAAAGATCACTGTAACAGGGACCCTGTGGAACGGCAACAAAATTATCGACGAGCGCCTG
ATCAACCCCGACGGCTCCCTGCTGTTCCGAGTAACCATCAACGGAGTGACCGGCTGGCGGCT
GTGCGAACGCATTCTGGCGTAA
Kozak 3XFLAG-EGF P2A nostop (330 bps)
SEQ ID NO: 9
GGGAGCCACCATGGACTACAAGGACGACGACGACAAGATCATCGACTATAAAGACGACGAC
GATAAAGGTGGCGACTATAAGGACGACGACGACAAAGCCATTAATAGTGACTCTGAGTGTC
CCCTGTCCCACGACGGGTACTGCCTCCACGACGGTGTGTGCATGTATATTGAAGCATTGGAC
AAGTACGCCTGCAACTGTGTTGTTGGCTACATCGGGGAGCGCTGTCAGTACCGAGACCTGAA
GTGGTGGGAACTGCGCGGAAGCGGAGCTACTAACTTCAGCCTGCTGAAGCAGGCTGGAGAC
GTGGAGGAGAACCCTGGACCTCT
5-13: Kozak sequence
14-262: 3XFLAG-EGF
263-328: P2A
Kozak 3XFLAG-EGF nostop (264 bps)
SEQ ID NO: 10
GGGAGCCACCATGGACTACAAGGACGACGACGACAAGATCATCGACTATAAAGACGACGAC
GATAAAGGTGGCGACTATAAGGACGACGACGACAAAGCCATTAATAGTGACTCTGAGTGTC
CCCTGTCCCACGACGGGTACTGCCTCCACGACGGTGTGTGCATGTATATTGAAGCATTGGAC
AAGTACGCCTGCAACTGTGTTGTTGGCTACATCGGGGAGCGCTGTCAGTACCGAGACCTGAA
GTGGTGGGAACTGCGCCT
5-13: Kozak sequence
14-262: 3XFLAG-EGF
Kozak 3XFLAG-EGF stop (273 bps)
SEQ ID NO: 11
GGGAGCCACCATGGACTACAAGGACGACGACGACAAGATCATCGACTATAAAGACGACGAC
GATAAAGGTGGCGACTATAAGGACGACGACGACAAAGCCATTAATAGTGACTCTGAGTGTC
CCCTGTCCCACGACGGGTACTGCCTCCACGACGGTGTGTGCATGTATATTGAAGCATTGGAC
AAGTACGCCTGCAACTGTGTTGTTGGCTACATCGGGGAGCGCTGTCAGTACCGAGACCTGAA
GTGGTGGGAACTGCGCTGATAGTAACT
5-13: Kozak sequence
14-262: 3XFLAG-EGF
263-271: Triple stop codon
Kozak 1XFLAG-EGF T2A 1XFLAG-Nluc P2A nostop (873 bps)
SEQ ID NO: 12
GGGAGCCACCATGGACTACAAGGACGACGACGACAAGATCATCAATAGTGACTCTGAGTGT
CCCCTGTCCCACGACGGGTACTGCCTCCACGACGGTGTGTGCATGTATATTGAAGCATTGGA
CAAGTACGCCTGCAACTGTGTTGTTGGCTACATCGGGGAGCGCTGTCAGTACCGAGACCTGA
AGTGGTGGGAACTGCGCGGCTCCGGCGAGGGCAGGGGAAGTCTTCTAACATGCGGGGACGT
GGAGGAAAATCCCGGCCCAGACTATAAGGACGACGACGACAAAATCATCGTCTTCACACTC
GAAGATTTCGTTGGGGACTGGCGACAGACAGCCGGCTACAACCTGGACCAAGTCCTTGAAC
AGGGAGGTGTGTCCAGTTTGTTTCAGAATCTCGGGGTGTCCGTAACTCCGATCCAAAGGATT
GTCCTGAGCGGTGAAAATGGGCTGAAGATCGACATCCATGTCATCATCCCGTATGAAGGTCT
GAGCGGCGACCAAATGGGCCAGATCGAAAAAATTTTTAAGGTGGTGTACCCTGTGGATGAT
CATCACTTTAAGGTGATCCTGCACTATGGCACACTGGTAATCGACGGGGTTACGCCGAACAT
GATCGACTATTTCGGACGGCCGTATGAAGGCATCGCCGTGTTCGACGGCAAAAAGATCACTG
TAACAGGGACCCTGTGGAACGGCAACAAAATTATCGACGAGCGCCTGATCAACCCCGACGG
CTCCCTGCTGTTCCGAGTAACCATCAACGGAGTGACCGGCTGGCGGCTGTGCGAACGCATTC
TGGCGGGAAGCGGAGCTACTAACTTCAGCCTGCTGAAGCAGGCTGGAGACGTGGAGGAGAA
CCCTGGACCTCT
5-13: Kozak sequence
14-202: 1XFLAG-EGF
203-265: T2A
266-805: 1XFLAG-Nluc
806-871: P2A
Kozak 1XFLAG-EGF stop 1XFLAG-Nluc stop (762 bps)
SEQ ID NO: 13
GGGAGCCACCATGGACTACAAGGACGACGACGACAAGATCATCAATAGTGACTCTGAGTGT
CCCCTGTCCCACGACGGGTACTGCCTCCACGACGGTGTGTGCATGTATATTGAAGCATTGGA
CAAGTACGCCTGCAACTGTGTTGTTGGCTACATCGGGGAGCGCTGTCAGTACCGAGACCTGA
AGTGGTGGGAACTGCGCTGATAGTAAGACTATAAGGACGACGACGACAAAATCATCGTCTT
CACACTCGAAGATTTCGTTGGGGACTGGCGACAGACAGCCGGCTACAACCTGGACCAAGTC
CTTGAACAGGGAGGTGTGTCCAGTTTGTTTCAGAATCTCGGGGTGTCCGTAACTCCGATCCA
AAGGATTGTCCTGAGCGGTGAAAATGGGCTGAAGATCGACATCCATGTCATCATCCCGTATG
AAGGTCTGAGCGGCGACCAAATGGGCCAGATCGAAAAAATTTTTAAGGTGGTGTACCCTGT
GGATGATCATCACTTTAAGGTGATCCTGCACTATGGCACACTGGTAATCGACGGGGTTACGC
CGAACATGATCGACTATTTCGGACGGCCGTATGAAGGCATCGCCGTGTTCGACGGCAAAAA
GATCACTGTAACAGGGACCCTGTGGAACGGCAACAAAATTATCGACGAGCGCCTGATCAAC
CCCGACGGCTCCCTGCTGTTCCGAGTAACCATCAACGGAGTGACCGGCTGGCGGCTGTGCGA
ACGCATTCTGGCGTGATAGTAACT
5-13: Kozak sequence
14-202: 1XFLAG-EGF
203-211: Triple stop codon
212-751: 1XFLAG-Nluc
752-760: Triple stop codon
hEPO ORF
ATGGGAGTGCACGAGTGTCCCGCGTGGTTGTGGTTGCTGCTGTCGCTCTTGAGCCTCCCACT
GGGACTGCCTGTGCTGGGGGCACCACCCAGATTGATCTGCGACTCACGGGTACTTGAGAGGT
ACCTTCTTGAAGCCAAAGAAGCCGAAAACATCACAACCGGATGCGCCGAGCACTGCTCCCT
CAATGAGAACATTACTGTACCGGATACAAAGGTCAATTTCTATGCATGGAAGAGAATGGAA
GTAGGACAGCAGGCCGTCGAAGTGTGGCAGGGGCTCGCGCTTTTGTCGGAGGCGGTGTTGC
GGGGTCAGGCCCTCCTCGTCAACTCATCACAGCCGTGGGAGCCCCTCCAACTTCATGTCGAT
AAAGCGGTGTCGGGGCTCCGCAGCTTGACGACGTTGCTTCGGGCTCTGGGCGCACAAAAGG
AGGCTATTTCGCCGCCTGACGCGGCCTCCGCGGCACCCCTCCGAACGATCACCGCGGACACG
TTTAGGAAGCTTTTTAGAGTGTACAGCAATTTCCTCCGCGGAAAGCTGAAATTGTATACTGG
TGAAGCGTGTAGGACAGGGGATCGCTAA

What is claimed is:

1. A method of maintaining expression of a protein in a mammal, comprising:
(a) providing a first composition comprising a circular polyribonucleotide that encodes the protein to the mammal; and
(b) from 6 hours to 90 days following step (a), providing a second composition comprising a circular polyribonucleotide that encodes the protein, to the mammal, thereby maintaining expression of the protein in the mammal.

2. The method of claim 1, wherein providing the second composition occurs after providing the first composition and
(i) before a first level of protein expressed by the first composition is substantially undetectable in the mammal; or
(ii) after the first level of protein expressed by the first composition is substantially undetectable in the mammal; or
(iii) before a first level of protein expressed by the first composition decreases by more than 50% in the mammal.

3. The method of any one of claims 1-2, wherein the circular polyribonucleotide: (i) is an exogenous, synthetic circular polyribonucleotide; and/or (ii) lacks a poly-A sequence, a replication element, or both.

4. The method of any one of claims 1-3, wherein the first composition comprises a first circular polyribonucleotide and the second compositions comprises a second circular polyribonucleotide, wherein:
(i) the first circular polyribonucleotide and the second circular polyribonucleotide are the same; or
(ii) the first circular polyribonucleotide and the second circular polyribonucleotide are different.

5. The method of any one of the claims 1-4, further comprising:
(i) providing a third composition of the circular polyribonucleotide to the mammal after the second composition, thereby maintaining expression of the protein in the mammal, or
(ii) providing a third composition of the circular polyribonucleotide to the mammal after the second composition, thereby restoring expression of the protein in the mammal;
and optionally, wherein providing the third composition occurs after providing the second composition and (a) before a second level of the protein expressed by the first and second composition is substantially undetectable in the mammal; or (b) before a second level of the protein expressed by the first and second composition in the mammal decreases by more than 50%; and/or
(iii) providing a fourth, fifth, sixth, seventh, eighth, ninth, or tenth composition of a circular polyribonucleotide encoding the protein.

6. The method of any one of claims 1-5, wherein:
(i) a first level of the protein expressed by the first composition is a highest level of the protein 1-2 days after providing the first composition; or
(ii) a first level of the protein expressed by the first composition is 40%, 50%, 60%, 70%, 80%, or 90% of the highest level of the protein one day after providing the first composition; and optionally,
(iii) a second level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, or 30 days after providing the second composition; and optionally,
(iv) a third level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, or 30 days after providing the third composition; and/or
(v) for each subsequent composition provided after the first composition, a subsequent level of the protein expressed after each subsequent composition is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, or 30 days after providing each subsequent composition.

7. The method of any one of claims 1-6, wherein an average level of the protein after providing the second composition is at least 40%, 50%, 60%, 70%, 80%, 90%, 100%, or 110% of a first level of protein from the first composition, wherein the average level of the protein is measured from (i) one day after providing the second composition to the day when the protein is substantially undetectable; or (ii) after providing each subsequent composition to the day when the protein is substantially undetectable.

8. The method of any one of claims 1-7, wherein
(i) a first level of the protein is maintained after providing the first composition and the second composition for from 6 hours to 90 days after providing the first composition; and/or
(ii) a first level of the protein is maintained after providing the first composition, the second composition, and the third composition of the circular polyribonucleotide for from 6 hours to 270 days after providing the first composition; and/or
(iii) a first level of the protein is substantially undetectable after providing the first composition and the second composition for 6 hours to 35 days after providing the first composition.

9. The method of any one of claims 1-8, wherein:
(i) a second level of protein in the mammal after providing the second composition is at least 1%, 5%, 10%, 20%, 30%, 40%, 50%, or 60% higher than a first level of protein in the mammal after providing the first composition; and/or
(ii) a third level of protein produced in the mammal after providing the third composition is at least 5%, 10%, 20%, 30%, 40%, 50%, or 60% higher than a first level of protein after providing the first composition; and/or
(iii) a second level of protein 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the second composition of the circular polyribonucleotide is at least 1%, 5%, 10%, 20%, 30%, 40%, 50%, or 60% higher than a first level of the protein after providing the first composition; and/or
(iv) a third level of protein 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of the protein after providing the first composition.

10. The method of any one of claims 1-9, wherein the protein is a therapeutic protein, e.g., erythropoietin; and/or
wherein expression of the protein (e.g., erthyropoietin) induces a response (e.g., reticulocyte production) in the mammal.

11. A method of maintaining expression of a protein in a cell or subject, comprising:
(a) providing a first composition comprising a circular polyribonucleotide that encodes the protein to the cell or subject; and
(b) from 6 hours to 90 days following step (a), providing a second composition comprising a circular polyribonucleotide that encodes the protein, to the cell or subject, thereby maintaining expression of the protein in the cell or subject; optionally wherein providing the first composition is to a first cell in the subject and providing the second composition is to a second cell in the subject and wherein the first cell and second cell are the same cell or different cells.

12. A method of expressing protein in a cell or a subject comprising:
(a) providing a first composition comprising a circular polyribonucleotide that encodes a protein to the cell or the subject, wherein the cell or the subject expresses a first level of an encoded protein; and
(b) providing a second composition comprising a circular polyribonucleotide that encodes a protein to the cell or the subject, wherein the cell or the subject expresses a second level of an encoded protein and
(i) the second level is at least as much as the first level, or
(ii) the second level varies by no more than 20% of the first level;
thereby maintaining expression of encoded protein in the cell or the subject at least at the first level of the protein; optionally wherein providing the first composition is to a first cell in the subject and providing the second composition is to a second cell in the subject and wherein the first cell and second cell are the same cell or different cells.

13. A method of expressing a level of a protein in a cell or subject after providing a first composition and a second composition of a circular polyribonucleotide to the cell or subject compared to a level of the protein in the cell or subject after providing a first composition and second composition of a linear counterpart of the circular polyribonucleotide, comprising:
(a) providing a first composition of circular polyribonucleotide encoding a protein to a cell or subject, wherein the cell or subject comprises a level of the protein after providing the first composition of the circular polyribonucleotide; and
(b) providing a second composition of circular polyribonucleotide after the first composition to the cell or subject, wherein the cell or subject comprises
(i) at least the level of the protein after providing the second composition of the circular polyribonucleotide, or
(ii) a level of the protein that varies by no more than 20% of the level after providing the second composition of the circular polyribonucleotide;
thereby maintaining expression of the level of the protein in the cell or subject after providing the first composition and the second composition of the circular polyribonucleotide compared to the level of the protein in the cell or subject after providing the first composition and the second composition of a linear counterpart of the circular polyribonucleotide; and optionally wherein providing the first composition is to a first cell in the subject and providing the second composition is to a second cell in the subject and wherein the first cell and second cell are the same cell or different cells.

14. The method of claims 11 or 12, wherein providing the second composition occurs after providing the first composition and
(i) before the first level of protein expressed by the first composition is substantially undetectable in the cell or subject; and/or
(ii) before the first level of protein expressed by the first composition decreases by more than 50% in the cell or subject; and/or
(iii) before the first level of protein expressed by the first composition decreases by 25%-75% in the cell or subject.

15. The method of any one of the claims 12-14, further comprising providing a third composition of the circular polyribonucleotide to the cell or subject after the second composition, thereby maintaining expression of the protein in the cell or subject at least at the first level of protein, and optionally, wherein providing the third composition occurs after providing the second composition and (i) before the second level of the protein expressed by the first and second composition is substantially undetectable in the cell or subject, or (ii) before the second level of the protein expressed by the first and second composition in the cell or subject decreases by more than 50%.

16. The method of any one of claims 12-15, further comprising providing a fourth, fifth, sixth, seventh, eighth, ninth, or tenth composition of the circular polyribonucleotide.

17. The method of any one of claim 13, 15 or 16, wherein the second composition is provided to the cell or subject:
(i) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of protein in the cell or subject expressed by the first composition is substantially undetectable, or
(ii) at least 14 days after the first composition and no more than 90 days after the first composition.

18. The method of any one of claims 12-16, wherein a first level of the protein is
(i) a highest level of the protein one day after providing the first composition, and/or
(ii) 40%, 50%, 60%, 70%, 80%, or 90% of a highest level of the protein one day after providing the first composition.

19. The method of claim 18, wherein:
(i) a second level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, or 30 days after providing the second composition; and/or
(ii) a third level of the protein is at least 30%, 40%, 50%, 60%, 70%, 80%, 90%, 100%, 110%, 120%, or 130% of the highest level of the protein one day after providing the first composition for at least 1, 2, 3, 4, 5, 6, 7, 8, 9, 10, 12, 15, 20, 25, or 30 days after providing the third composition; and/or
(iii) for each subsequent composition provided after the first composition, a subsequent level of the protein expressed after each subsequent composition is at least 30%, 40%, 50%, 60%, 70%, 80%, 90%, 100%, 110%, 120%, or 130% of a highest level of the protein one day after providing the first composition for at least 1, 2, 3, 4, 5, 6, 7, 8, 9, 10, 12, 15, 20, 25, or 30 days after providing each subsequent composition; and/or
(iv) an average level of the protein after providing the second composition is at least 40%, 50%, 60%, 70%, 80%, 90%, 100%, or 110% of the first level, wherein the average level of the protein is measured from one day after providing the second composition to the day when the protein is substantially undetectable; and/or
(v) an average level of the protein after providing each subsequent composition after the first composition is at least 40%, 50%, 60%, 70%, 80%, 90%, 100%, or 110% of the first level, wherein the average level of the protein is measured from one day after providing each subsequent composition to the day when the protein is substantially undetectable; and/or
(vi) the first level of the protein is maintained after providing the first composition and the second composition of the circular polyribonucleotide for at least 6 hours, 1 day, 2 days, 3 days, 5 days, 7 days, 14 days, 21 days, 28 days, or 30 days after providing the first composition; and/or
(vii) the first level of the protein is maintained after providing the first composition, the second composition, and the third composition of the circular polyribonucleotide for at least 6 hours, 1 day, 2 days, 3 days, 5 days, 7 days, 14 days, 21 days, 28 days, or 30 days after providing the first composition; and/or
(viii) the second level of protein in the cell or subject after providing the second composition is at least 1%, 5%, 10%, 20%, 30%, 40%, 50%, or 60% higher than the first level of protein in the cell or subject after providing the first composition; and/or
(ix) a third level of protein produced in the cell or subject after providing the third composition is at least 5%, 10%, 20%, 30%, 40%, 50%, or 60% higher than the first level of protein in the plurality after providing the first composition; and/or
(x) the second level of protein 1 hour, 12 hours, 18 hours, 1 day, 2 days, 3 days, 4 days, 5 days, 6 days, 7 days, 8 days, 9 days, 10 days, 15 days, 20 days, 25 days, 30 days, 40 days, or 45 days after providing the second composition of the circular polyribonucleotide is at least 1%, 5%, 10%, 20%, 30%, 40%, 50%, or 60% higher than the first level of the protein after providing the first composition; and/or
(xi) the third level of protein 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of the protein after providing the first composition.

20. The method of any one of claims 13 or 15-17, wherein:
(i) the level of the protein in the cell or subject after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days; and/or
(ii) the level of the protein in the cell or subject after providing the first composition and the second composition of the circular polyribonucleotide is at least 5%, 10%, 20%, 30%, 40%, 50%, or 60% higher than the level of the protein in the cell or subject after providing the first composition and the second composition of the linear counterpart of the circular polyribonucleotide; and/or
(iii) the level of the protein in the cell or subject after providing the first composition and the second composition of the circular polyribonucleotide is at least 5%, 10%, 20%, 30%, 40%, 50%, or 60% higher than the level of the protein in the cell or subject after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, or 30 days after providing the second composition of the circular polyribonucleotide.

21. The method of any one of claims 11-20, wherein the protein is a therapeutic protein, e.g., erythropoietin, and/or wherein expression of the protein (e.g., erthyropoietin) induces a response (e.g., reticulocyte production) in the cell or subject.

22. A method of producing a circular polyribonucleotide in a cell or subject comprising:
(a) providing a first composition comprising the circular polyribonucleotide to the cell or subject, wherein the cell or subject comprises a first level of circular polyribonucleotide after providing the first composition; and
(b) providing a second composition of a circular polyribonucleotide to the cell or subject, wherein the cell or subject comprises a second level of circular polyribonucleotide and
(i) the second level of circular polyribonucleotide is at least as much as the first level, or
(ii) the second level of circular polyribonucleotide varies by no more than 20% of the first level after providing the second composition;
thereby maintaining circular polyribonucleotide in the cell or subject at least at the first level; optionally, wherein the first composition comprises a first circular polyribonucleotide and the second compositions comprises a second circular polyribonucleotide, wherein:
(i) the first circular polyribonucleotide and the second circular polyribonucleotide are the same; or
(ii) the first circular polyribonucleotide and the second circular polyribonucleotide are different;
optionally, wherein the first circular polyribonucleotide comprises a first binding site and/or encodes a first protein and the second circular polyribonucleotide comprise a second binding site and/or encodes a second protein, wherein the first binding site and the second binding site are the same or are different binding sites and/or the first protein and the second protein encode the same protein or different proteins.

23. A method of producing a level of a circular polyribonucleotide in a cell or subject after providing a first composition and a second composition of the circular polyribonucleotide to the cell or subject compared to a level of a linear counterpart of the circular polyribonucleotide in the cell or subject after providing a first composition and second composition of the linear counterpart of the circular polyribonucleotide, comprising:
(a) providing a first composition of the circular polyribonucleotide to the cell or subject, wherein the cell or subject comprises the level of the circular polyribonucleotide after providing the first composition; and
(b) providing the second composition of the circular polyribonucleotide to the cell or subject, wherein the cell or subject comprises (i) at least the level of the circular polyribonucleotide after providing the second composition, or (ii) a level of the protein after providing the second composition that varies by no more than 20% of the level of the circular polyribonucleotide;
thereby maintaining the level of the circular polyribonucleotide in the cell or subject after providing the first composition and the second composition of the circular polyribonucleotide compared to the level of the linear counterpart in the cell or subject after providing the first composition and the second composition of the linear counterpart of the circular polyribonucleotide.

24. The method of claim 22, wherein providing the second composition occurs after providing the first composition and before the level of circular polyribonucleotide produced by providing the first composition is substantially undetectable in the cell or subject.

25. The method of claim 24, wherein providing the second composition of the circular polyribonucleotide occurs after the first composition and after the level of circular polyribonucleotide in the cell or subject produced by the first composition is substantially undetectable.

26. The method of claim 23 or 25, wherein the second composition is provided to the cell or subject
(i) at least 1 minute, 1 hour, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of circular polyribonucleotide produced by the first composition is substantially undetectable, or
(ii) at least 14 days after the first composition and no more than 90 days after the first composition.

27. The method of claim 22 or 24, further comprising providing a third composition of circular polyribonucleotide to the cell or subject after the second composition, thereby maintaining the level of circular polyribonucleotide after providing the third composition at least at the first level, and, optionally, wherein providing the third composition occurs after providing the second composition and
(i) before the level of circular polyribonucleotide produced by the first and second composition in the cell or subject is substantially undetectable in the cell or subject, or
(ii) before the level of circular polyribonucleotide produced by the first and second composition in the cell or subject decreases by more than 50%; or
(iii) before the level of circular polyribonucleotide produced by the first and second composition in the cell or subject decreases by 25%-75% in the cell or subject.

28. The method of any one of claims 22-27, further comprising providing a fourth, fifth, sixth, seventh, eighth, ninth, or tenth composition of the circular polyribonucleotide to the cell or subject.

29. The method of any one of claims 22, 24, 27, or 28, wherein:
(i) the first level of the circular polyribonucleotide is a highest level of circular polyribonucleotide one day after providing the first composition; and/or
(ii) for each subsequent composition provided after the first composition, a subsequent level of circular polyribonucleotide expressed after each subsequent composition is at least 30%, 40%, 50%, 60%, 70%, 80%, 90%, 100%, 110%, 120%, or 130% of a highest level of circular polyribonucleotide one day after providing the first composition for at least 1, 2, 3, 4, 5, 6, 7, 8, 9, 10, 12, 15, 20, 25, or 30 days after providing each subsequent composition; and/or
(ii) an average level of the circular polyribonucleotide after providing the second composition is at least 40%, 50%, 60%, 70%, 80%, or 90% of the first level, wherein the average level of the circular polyribonucleotide is measured from one day after providing the second composition to the day when the circular polyribonucleotide is substantially undetectable; and/or
(iii) an average level of the circular polyribonucleotide after providing each subsequent composition after the first composition is at least 40%, 50%, 60%, 70%, 80%, or 90% of the first level, wherein the average level of the circular polyribonucleotide is measured from one day after providing each subsequent composition to the day when the circular polyribonucleotide is substantially undetectable; and/or
(iv) the first level of the circular polyribonucleotide is maintained after providing the second composition of the circular polyribonucleotide for at least 6 hours, 1 day, 2 days, 3 days, 5 days, 7 days, 14 days, 21 days, 28 days, or 30 days; and/or
(v) the second level of circular polyribonucleotide in the cell or subject after providing the second composition is at least 1%, 5%, 10%, 20%, 30%, 40%, 50%, or 60% higher than the first level of circular polyribonucleotide in the cell or subject after providing the first composition; and/or
(vi) the second level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the second composition of the circular polyribonucleotide is at least 1%, 5%, 10%, 20%, 30%, 40%, 50%, or 60% higher than the first level of circular polyribonucleotide after providing the first composition.

30. The method of any one of claims 27-29, wherein a third level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of circular polyribonucleotide after providing the first composition.

31. The method of any one of claims 23, 25, 26, or 28, wherein:
(i) the level of circular polyribonucleotide in the cell or subject after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days; and/or
(ii) the level of circular polyribonucleotide produced by the first composition is 40%, 50%, 60%, 70%, 80%, or 90% of a highest level of the circular polyribonucleotide one day after providing the first composition; and/or
(iii) the level of circular polyribonucleotide produced by the second composition is at least 30%, 40%, 50%, 60%, 70%, 80%, 90%, 100%, 110%, 120%, or 130% of a highest level of circular polyribonucleotide one day after providing the first composition, for at least 1, 2, 3, 4, 5, 6, 7, 8, 9, 10, 12, 15, 20, 25, or 30 days after providing the second composition; and/or
(iv) the level of circular polyribonucleotide in the cell or subject after providing the first composition and the second composition of the circular polyribonucleotide is at least 5%, 10%, 20%, 30%, 40%, 50%, or 60% higher than the level of linear counterpart of the circular polyribonucleotide in the cell or subject after providing the first composition and the second composition of the linear counterpart of circular polyribonucleotide; and/or
(v) the level of circular polyribonucleotide after providing the first composition and the second composition of circular polyribonucleotide is at least 5%, 10%, 20%, 30%, 40%, 50%, or 60% higher than the level of linear counterpart of circular polyribonucleotide after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, or 30 days after providing the second composition of the circular polyribonucleotide.

32. The method of any one of claims 27-29, wherein a third level of the circular polyribonucleotide is at least 30%, 40%, 50%, 60%, 70%, 80%, 90%, 100%, 110%, 120%, or 130% of the highest level of circular polyribonucleotide one day after providing the first composition for at least 1, 2, 3, 4, 5, 6, 7, 8, 9, 10, 12, 15, 20, 25, or 30 days after providing the third composition.

33. The method of any one of claims 27-29 or 32, wherein:
(i) the first level of circular polyribonucleotide is maintained after providing the third composition of circular polyribonucleotide for at least 6 hours, 1 day, 2 days, 3 days, 5 days, 7 days, 14 days, 21 days, 28 days or 30 days; and/or
(ii) the third level of circular polyribonucleotide in the cell or subject after providing the third composition is at least 5%, 10%, 20%, 30%, 40%, 50%, or 60% higher than the first level of circular polyribonucleotide in the plurality after providing the first composition; and/or
(iii) the third level of circular polyribonucleotide 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of circular polyribonucleotide after providing the first composition.

34. The method of any one of claims 22-33, wherein the protein (e.g., erthypoietin) induces a response (e.g., production of reticulocytes) in the subject.

35. A method of binding a target in a cell or subject comprising:
(a) providing a first composition comprising a circular polyribonucleotide that comprises a binding site for a target, to the cell or subject, wherein the target binds to the binding site at a first level; and
(b) providing a second composition comprising the circular polyribonucleotide that comprises a binding site for a target to the cell or subject, wherein the target binds to the binding site at a second level and (i) the second level is at least as much as the first level, or (ii) the second level varies by no more than 20% of the first level;
thereby maintaining binding of the target in the cell or subject at least at the first level of binding.

36. A method of binding a target in a cell or subject after providing a first composition and a second composition of a circular polyribonucleotide to the cell or subject compared to a level of binding to the target in the cell or subject after providing a first composition and second composition of a linear counterpart of the circular polyribonucleotide, comprising:
(a) providing a first composition of the circular polyribonucleotide comprising binding site to the cell or subject, wherein the cell or subject comprises the level of the binding to the target after providing the first composition of the circular polyribonucleotide; and
(b) providing the second composition of the circular polyribonucleotide after the first composition to the cell or subject, wherein the cell or subject comprises (i) at least the level of the binding to the target after providing the second composition of the circular polyribonucleotide, or (ii) a level of the binding to a target that varies by no more than 20% of the level after providing the second composition of the circular polyribonucleotide;
thereby maintaining the level of the binding to the target in the cell or subject after providing the first composition and the second composition of the circular polyribonucleotide compared to the level of the binding to the target in the cell or subject after providing the first composition and the second composition of the linear counterpart of the circular polyribonucleotide.

37. The method of claim 35, wherein providing the second composition occurs after providing the first composition and (i) before the first level of binding by the first composition is substantially undetectable in the cell or subject, or (ii) before the first level of binding by the first composition decreases by more than 50% in the cell or subject, or (iii) before the first level of binding by the first composition decreases by 25%-75% in the cell or subject.

38. The method of claim 35 or 37, further comprising providing a third composition of the circular polyribonucleotide to the cell or subject after the second composition, thereby maintaining binding of the target in the cell or subject at least at the first level of binding, and optionally, wherein providing the third composition occurs after providing the second composition and before the second level of the binding of the target in the cell or subject by the first and second composition is substantially undetectable in the cell or subject.

39. The method of claim 38, wherein providing the third composition occurs after providing the second composition and before the second level of the binding by the first and second composition in the cell or subject decreases by more than 50%.

40. The method of any one of claims 35-39, further comprising providing a fourth, fifth, sixth, seventh, eighth, ninth, or tenth composition of the circular polyribonucleotide.

41. The method of claim 36 or 38, wherein:
(i) providing the second composition of circular polyribonucleotide occurs after the first composition and after the level of binding by the first composition is substantially undetectable; and/or
(ii) the second composition is provided to the cell or subject at least 6 hours, 1 day, 2 days, 3 days, 4 days, 5 days, 7 days, 2 weeks, 3 weeks, 4 weeks, 5 weeks, 6 weeks, 2 months, 3 months, 4 months, 5 months, 6 months, 8 months, 9 months, 10 months, 11 months, 12 month, 13 months, 14 months, 15 months, 16 months, 17 months, 18 months, 19 months, 20 months, 21 months, or 22 months after the level of binding by the first composition is substantially undetectable; and/or
(iii) the second composition is provided to the cell or subject at 14 days after the first composition and no more than 90 days after the first composition.

42. The method of any one of claims 35 or 37-40, wherein:
(i) the first level of binding is the highest level of binding one day after providing the first composition; and/or
(ii) the first level of the binding is 40%, 50%, 60%, 70%, 80%, or 90% of the highest level of binding one day after providing the first composition; and/or
(iii) the second level of binding is at least 30%, 40%, 50%, 60%, 70%, 80%, 90%, 100%, 110%, 120%, or 130% of a highest level of binding one day after providing the first composition for at least 1, 2, 3, 4, 5, 6, 7, 8, 9, 10, 12, 15, 20, 25, 30, 35, 40, or 45 days after providing the second composition; and/or
(iv) the third level of binding is at least 30%, 40%, 50%, 60%, 70%, 80%, 90%, 100%, 110%, 120%, or 130% of a highest level of binding one day after providing the first composition for at least 1, 2, 3, 4, 5, 6, 7, 8, 9, 10, 12, 15, 20, 25, or 30 days after providing the third composition; and/or
(v) for each subsequent composition provided after the first composition, a subsequent level of binding after each subsequent composition is at least 30%, 40%, 50%, 60%, 70%, 80%, 90%, 100%, 110%, 120%, or 130% of the highest level of binding one day after providing the first composition for at least 1, 2, 3, 4, 5, 6, 7, 8, 9, 10, 12, 15, 20, 25, or 30 days after providing each subsequent composition; and/or
(vi) an average level of binding after providing the second composition is at least 40%, 50%, 60%, 70%, 80%, 90%, 100%, or 110% of the first level, wherein the average level of binding is measured from one day after providing the second composition to the day when the binding is substantially undetectable; and/or
(vii) an average level of binding after providing each subsequent composition after the first composition is at least 40%, 50%, 60%, 70%, 80%, 90%, 100%, or 110% of the first level, wherein the average level of binding is measured from one day after providing each subsequent composition to the day when the binding is substantially undetectable; and/or
(viii) the first level of the binding is maintained after providing the first composition and the second composition of the circular polyribonucleotide for at least 6 hours, 1 day, 2 days, 3 days, 5 days, 7 days, 14 days, 21 days, 28 days, or 30 days after providing the first composition; and/or
(ix) the second level of binding in the cell or subject after providing the second composition is at least 1%, 5%, 10%, 20%, 30%, 40%, 50%, or 60% higher than the first level of binding in the cell or subject after providing the first composition.
(x) the second level of binding 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the second composition of the circular polyribonucleotide is at least 1%, 5%, 10%, 20%, 30%, 40%, 50%, or 60% higher than the first level of the binding after providing the first composition.

43. The method of claim 36 or 41, wherein:
(i) the level of binding in the cell or subject after providing the first composition and the second composition of the circular polyribonucleotide is maintained for at least 1 hour, 12 hours, 18 hours, 1 day, 2 days, 3 days, 4 days, 5 days, 6 days, 7 days, 8 days, 9 days, 10 days, 15 days, 20 days, 25 days, or 30 days; and/or
(ii) the level of binding in the cell or subject after providing the first composition and the second composition of the circular polyribonucleotide is at least 5%, 10%, 20%, 30%, 40%, 50%, or 60% higher than the level of binding in the cell or subject after providing the first composition and the second composition of the linear counterpart of the circular polyribonucleotide; and/or
(iii) the level of binding in the cell or subject after providing the first composition and the second composition of the circular polyribonucleotide is at least 5%, 10%, 20%, 30%, 40%, 50%, or 60% higher than the level of binding in the cell or subject after providing the first composition and the second composition of the linear counterpart of the circular for at least 1 day, 2 days, 3 days, 4 days, 5 days, 6 days, 7 days, 8 days, 9 days, 10 days, 15 days, 20 days, 25 days, or 30 days after providing the second composition of the circular polyribonucleotide.

44. The method of any one of claims 38-40, or 42, wherein:
(i) the first level of the binding is maintained after providing the first composition, second composition, and third composition of the circular polyribonucleotide for at least 6 hours, 1 day, 2 days, 3 days, 5 days, 7 days, 14 days, 21 days, 28 days, or 30 days after providing the first composition; and/or
(ii) a third level of binding in the cell or subject after providing the third composition is at least 5%, 10%, 20%, 30%, 40%, 50%, or 60% higher than the first level of binding after providing the first composition.
(iii) a third level of binding 1 hour, 12 hours, 18 hours, 1 day, 2 days, 3 days, 4 days, 5 days, 6 days, 7 days, 8 days, 9 days, 10 days, 15 days, 20 days, 25 days, or 30 days after providing the third composition of the circular polyribonucleotide is at least 1%, 5%, 10%, 20%, 30%, 40%, 50%, or 60% higher than the first level of the binding after providing the first composition.

45. The method of any one of claims 1-44, wherein:
(i) the circular polyribonucleotide of the first composition and the circular polyribonucleotide of the second composition are the same; or
(ii) the circular polyribonucleotide of the first composition and the circular polyribonucleotide of the second composition are different.

46. The method of any one of claims 1-45, wherein:
(i) the first composition and the second composition comprise about the same amount of the circular polyribonucleotide; or
(ii) the first composition comprises a higher amount of the circular polyribonucleotides than the second composition; and/or
(iii) the first composition comprises a higher amount of the circular polyribonucleotides than a third, fourth, fifth, sixth, seventh, eighth, ninth, or tenth composition; and/or
(iv) an amount of circular polyribonucleotide of the second composition varies by no more than 1%, 5%, 10%, 15%, 20%, or 25% of an amount of circular polyribonucleotide of the first composition.
(v) an amount of circular polyribonucleotide of the second composition is no more than 1%, 5%, 10%, 15%, 20%, or 25% less than an amount of circular polyribonucleotide of the first composition; and/or
(vi) the first composition further comprises a pharmaceutically acceptable carrier or excipient; and/or
(vii) the second composition further comprises a pharmaceutically acceptable carrier or excipient; and/or
(viii) the third composition further comprises a pharmaceutically acceptable carrier or excipient; and/or
(x) the cell is an animal cell (e.g., a mammalian cell, e.g., a human cell); and/or
(xi) the cell is a plurality of cells in a subject (xii) the first composition and/or the second composition comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules;
(xiii) the first composition and/or the second composition comprises at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to the total ribonucleotide molecules in the first composition and/or the second composition; and/or
(xiv) at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) of total ribonucleotide molecules in first composition and/or the second composition are circular polyribonucleotide molecules.

47. The method of any one of claims 11-46, wherein the subject is an animal (e.g., a mammal).

48. The method of any one of claims 11-47, wherein the subject is a human.

49. The method of any one of claims 1, 11, 12 and 22, wherein the protein is an antigen (e.g., tumor antigen, bacterial antigen, viral antigen).


SEQUENCE LISTING (54 sequences)

SEQ ID NO: 1 - RNA, 3 nt, Artificial Sequence
  source: /note=“Description of Artificial Sequence Synthetic oligonucleotide”
aug

SEQ ID NO: 2 - DNA, 717 bp, Artificial Sequence
  source: /note=“Description of Artificial Sequence Synthetic polynucleotide”
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 3 - DNA, 57 bp, Artificial Sequence
  source: /note=“Description of Artificial Sequence Synthetic oligonucleotide”
gctactaacttcagcctgctgaagcaggctggcgacgtggaggagaaccctggacct

SEQ ID NO: 4 - DNA, 221 bp, Artificial Sequence
  source: /note=“Description of Artificial Sequence Synthetic polynucleotide”
gtaaaaagaggtgaaacctattatgtgtgagcagggcacagacgttgaaactggagccaggagaagtattggcaggctttaggttattaggtggttactctgtcttaaaaatgttctggctttcttcctgcatccactggcatactcatggtctgtttttaaatattttaattcccatttacaaagtgatttacccacaagcccaacctgtctgtcttcag

SEQ ID NO: 5 - DNA, 552 bp, Artificial Sequence
  source: /note=“Description of Artificial Sequence Synthetic polynucleotide”
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataata

SEQ ID NO: 6 - DNA, 6926 bp, Artificial Sequence
  source: /note=“Description of Artificial Sequence Synthetic polynucleotide”
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattccattgagaaatgactgagttccggtgctctcaagtcattgatctttgtcgacttttatttggtctctgtaataacgacttcaaaaacattaaattctgttgcgaagccagtaagctacaaaaagaaaaaacaagagagaatgctatagtcgtatagtatagtttcccgactatctgatacccattacttatctagggggaatgcgaacccaaaattttatcagttttctcggatatcgatagatattggggaataaatttaaataaataaattttgggcgggtttagggcgtggcaaaaagttttttggcaaatcgctagaaatttacaagacttataaaattatgaaaaaatacaacaaaattttaaacacgtgggcgtgacagttttgggcggttttagggcgttagagtaggcgaggacagggttacatcgactaggctttgatcctgatcaagaatatatatactttataccgcttccttctacatgttacctatttttcaacgaatctagtatacctttttactgtacgatttatgggtataataataagctaaatcgagactaagttttattgttatatatattttttttattttatgcagaaattaattaaaccggtcctgcaggtgatcaggcgcgccggttaccggccggccccgcggagcgtaagtattcaaaattccaaaattttttactagaaatattcgattttttaataggcagtttctatactattgtatactattgtagattcgttgaaaagtatgtaacaggaagaataaagcatttccgaccatgtaaagtatatatattcttaataaggatcaatagccgagtcgatctcgccatgtccgtctgtcttattgttttattaccgccgagacatcaggaactataaaagctagaaggatgagttttagcatacagattctagagacaaggacgcagagcaagtttgttgatccatgctgccacgctttaactttctcaaattgcccaaaactgccatgcccacatttttgaactattttcgaaattttttcataattgtattactcgtgtaaatttccatcaatttgccaaaaaactttttgtcacgcgttaacgccctaaagccgccaatttggtcacgcccacactattgagcaattatcaaattttttctcattttattccccaatatctatcgatatccccgattatgaaattattaaatttcgcgttcgcattcacactagctgagtaacgagtatctgatagttggggaaatcgacttattttttatatacaatgaaaatgaatttaatcatatgaatatcgattatagctttttatttaatatgaatatttatttgggcttaaggtgtaacctcctcgacataagactcacatggcgcaggcacattgaagacaaaaatactcattgtcgggtctcgcaccctccagcagcacctaaaattatgtcttcaattattgccaacattggagacacaattagtctgtggcacctcaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 708 bp, Artificial Sequence
  source: /note=“Description of Artificial Sequence Synthetic polynucleotide”
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 8 - DNA, 516 bp, Artificial Sequence
  source: /note=“Description of Artificial Sequence Synthetic polynucleotide”
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 9 - DNA, 330 bp, Artificial Sequence
  source: /note=“Description of Artificial Sequence Synthetic polynucleotide”
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggaagcggagctactaacttcagcctgctgaagcaggctggagacgtggaggagaaccctggacctct

SEQ ID NO: 10 - DNA, 264 bp, Artificial Sequence
  source: /note=“Description of Artificial Sequence Synthetic polynucleotide”
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcct

SEQ ID NO: 11 - DNA, 273 bp, Artificial Sequence
  source: /note=“Description of Artificial Sequence Synthetic polynucleotide”
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgctgatagtaact

SEQ ID NO: 12 - DNA, 873 bp, Artificial Sequence
  source: /note=“Description of Artificial Sequence Synthetic polynucleotide”
gggagccaccatggactacaaggacgacgacgacaagatcatcaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggctccggcgagggcaggggaagtcttctaacatgcggggacgtggaggaaaatcccggcccagactataaggacgacgacgacaaaatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctct

SEQ ID NO: 13 - DNA, 762 bp, Artificial Sequence
  source: /note=“Description of Artificial Sequence Synthetic polynucleotide”
gggagccaccatggactacaaggacgacgacgacaagatcatcaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgctgatagtaagactataaggacgacgacgacaaaatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgatagtaact

SEQ ID NO: 14 - DNA, 54 bp, Artificial Sequence
  source: /note=“Description of Artificial Sequence Synthetic oligonucleotide”
gagggcaggggaagtctactaacatgcggggacgtggaggaaaatcccggccca

SEQ ID NO: 15 - DNA, 60 bp, Artificial Sequence
  source: /note=“Description of Artificial Sequence Synthetic oligonucleotide”
cagtgtactaattatgctctcttgaaattggctggagatgttgagagcaacccaggtccc

SEQ ID NO: 16 - DNA, 796 bp, Artificial Sequence
  source: /note=“Description of Artificial Sequence Synthetic polynucleotide”
gtaagaagcaaggtttcatttaggggaagggaaatgattcaggacgagagtctttgtgctgctgagtgcctgtgatgaagaagcatgttagtcctgggcaacgtagcgagaccccatctctacaaaaaatagaaaaattagccaggtatagtggcgcacacctgtgattccagctacgcaggaggctgaggtgggaggattgcttgagcccaggaggttgaggctgcagtgagctgtaatcatgccactactccaacctgggcaacacagcaaggaccctgtctcaaaagctacttacagaaaagaattaggctcggcacggtagctcacacctgtaatcccagcactttgggaggctgaggcgggcagatcacttgaggtcaggagtttgagaccagcctggccaacatggtgaaaccttgtctctactaaaaatatgaaaattagccaggcatggtggcacattcctgtaatcccagctactcgggaggctgaggcaggagaatcacttgaacccaggaggtggaggttgcagtaagccgagatcgtaccactgtgctctagccttggtgacagagcgagactgtcttaaaaaaaaaaaaaaaaaaaaaagaattaattaaaaatttaaaaaaaaatgaaaaaaagctgcatgcttgttttttgtttttagttattctacattgttgtcattattaccaaatattggggaaaatacaacttacagaccaatctcaggagttaaatgttactacgaaggcaaatgaactatgcgtaatgaacctggtaggcatta

SEQ ID NO: 17 - DNA, 582 bp, Artificial Sequence
  source: /note=“Description of Artificial Sequence Synthetic polynucleotide”
atgggagtgcacgagtgtcccgcgtggttgtggttgctgctgtcgctcttgagcctcccactgggactgcctgtgctgggggcaccacccagattgatctgcgactcacgggtacttgagaggtaccttcttgaagccaaagaagccgaaaacatcacaaccggatgcgccgagcactgctccctcaatgagaacattactgtaccggatacaaaggtcaatttctatgcatggaagagaatggaagtaggacagcaggccgtcgaagtgtggcaggggctcgcgcttttgtcggaggcggtgttgcggggtcaggccctcctcgtcaactcatcacagccgtgggagcccctccaacttcatgtcgataaagcggtgtcggggctccgcagcttgacgacgttgcttcgggctctgggcgcacaaaaggaggctatttcgccgcctgacgcggcctccgcggcacccctccgaacgatcaccgcggacacgtttaggaagctttttagagtgtacagcaatttcctccgcggaaagctgaaattgtatactggtgaagcgtgtaggacaggggatcgctaa

SEQ ID NO: 18 - protein, 8 aa, Artificial Sequence
  source: /note=“Description of Artificial Sequence Synthetic peptide”
  VARIANT (2)..(2): /replace=“Ile”
  MOD_RES (4)..(4): Any amino acid
  SITE (1)..(8): /note=“Variant residues given in the sequence have no preference with respect to those in the annotations for variant positions”
DVEXNPGP

SEQ ID NO: 19 - protein, 15 aa, Artificial Sequence
  source: /note=“Description of Artificial Sequence Synthetic peptide”
GLNDIFEAQKIEWHE

SEQ ID NO: 20 - protein, 26 aa, Artificial Sequence
  source: /note=“Description of Artificial Sequence Synthetic peptide”
KRRWKKNFIAVSAANRFKKISSSGAL

SEQ ID NO: 21 - protein, 6 aa, Artificial Sequence
  source: /note=“Description of Artificial Sequence Synthetic peptide”
EEEEEE

SEQ ID NO: 22 - protein, 13 aa, Artificial Sequence
  source: /note=“Description of Artificial Sequence Synthetic peptide”
GAPVPYPDPLEPR

SEQ ID NO: 23 - protein, 8 aa, Artificial Sequence
  source: /note=“Description of Artificial Sequence Synthetic peptide”
DYKDDDDK

SEQ ID NO: 24 - protein, 9 aa, Artificial Sequence
  source: /note=“Description of Artificial Sequence Synthetic peptide”
YPYDVPDYA

SEQ ID NO: 25 - protein, 6 aa, Artificial Sequence
  source: /note=“Description of Artificial Sequence Synthetic 6xHis tag”
HHHHHH

SEQ ID NO: 26 - protein, 10 aa, Artificial Sequence
  source: /note=“Description of Artificial Sequence Synthetic peptide”
EQKLISEEDL

SEQ ID NO: 27 - protein, 18 aa, Artificial Sequence
  source: /note=“Description of Artificial Sequence Synthetic peptide”
TKENPRSNQEESYDDNES

SEQ ID NO: 28 - protein, 15 aa, Artificial Sequence
  source: /note=“Description of Artificial Sequence Synthetic peptide”
KETAAAKFERQHMDS

SEQ ID NO: 29 - protein, 38 aa, Artificial Sequence
  source: /note=“Description of Artificial Sequence Synthetic polypeptide”
MDEKTTGWRGGHVVEGLAGELEQLRARLEHHPQGQREP

SEQ ID NO: 30 - protein, 13 aa, Artificial Sequence
  source: /note=“Description of Artificial Sequence Synthetic peptide”
SLAELLNAGLGGS

SEQ ID NO: 31 - protein, 8 aa, Artificial Sequence
  source: /note=“Description of Artificial Sequence Synthetic peptide”
TQDPSRVG

SEQ ID NO: 32 - protein, 12 aa, Artificial Sequence
  source: /note=“Description of Artificial Sequence Synthetic peptide”
PDRVRAVSHWSS

SEQ ID NO: 33 - protein, 8 aa, Artificial Sequence
  source: /note=“Description of Artificial Sequence Synthetic peptide”
WSHPQFEK

SEQ ID NO: 34 - protein, 6 aa, Artificial Sequence
  source: /note=“Description of Artificial Sequence Synthetic peptide”
CCPGCC

SEQ ID NO: 35 - protein, 10 aa, Artificial Sequence
  source: /note=“Description of Artificial Sequence Synthetic peptide”
EVHTNQDPLD

SEQ ID NO: 36 - protein, 14 aa, Artificial Sequence
  source: /note=“Description of Artificial Sequence Synthetic peptide”
GKPIPNPLLGLDST

SEQ ID NO: 37 - protein, 11 aa, Artificial Sequence
  source: /note=“Description of Artificial Sequence Synthetic peptide”
YTDIEMNRLGK

SEQ ID NO: 38 - protein, 8 aa, Artificial Sequence
  source: /note=“Description of Artificial Sequence Synthetic peptide”
DLYDDDDK

SEQ ID NO: 39 - DNA, 20 bp, Artificial Sequence
  source: /note=“Description of Artificial Sequence Synthetic primer”
agatttcgttggggactggc

SEQ ID NO: 40 - DNA, 20 bp, Artificial Sequence
  source: /note=“Description of Artificial Sequence Synthetic primer”
caccgctcaggacaatcctt

SEQ ID NO: 41 - DNA, 20 bp, Artificial Sequence
  source: /note=“Description of Artificial Sequence Synthetic primer”
ctggagacgtggaggagaac

SEQ ID NO: 42 - DNA, 20 bp, Artificial Sequence
  source: /note=“Description of Artificial Sequence Synthetic primer”
ccaaaagacggcaatatggt

SEQ ID NO: 43 - DNA, 32 bp, Artificial Sequence
  source: /note=“Description of Artificial Sequence Synthetic oligonucleotide”
gtcaacggattttcccaagtccgtagcgtctc

SEQ ID NO: 44 - DNA, 30 bp, Artificial Sequence
  source: /note=“Description of Artificial Sequence Synthetic oligonucleotide”
gtttttcggctattcccaatagccgttttg

SEQ ID NO: 45 - DNA, 21 bp, Artificial Sequence
  source: /note=“Description of Artificial Sequence Synthetic primer”
agggctgcttttaactctggt

SEQ ID NO: 46 - DNA, 21 bp, Artificial Sequence
  source: /note=“Description of Artificial Sequence Synthetic primer”
ccccacttgattttggaggga

SEQ ID NO: 47 - DNA, 20 bp, Artificial Sequence
  source: /note=“Description of Artificial Sequence Synthetic primer”
tgtgggcaatgtcatcaaaa

SEQ ID NO: 48 - DNA, 21 bp, Artificial Sequence
  source: /note=“Description of Artificial Sequence Synthetic primer”
gaagcacttgctacctcttgc

SEQ ID NO: 49 - DNA, 19 bp, Artificial Sequence
  source: /note=“Description of Artificial Sequence Synthetic primer”
ggcaccatgggaagtgatt

SEQ ID NO: 50 - DNA, 20 bp, Artificial Sequence
  source: /note=“Description of Artificial Sequence Synthetic primer”
atttggtaaggcctgagctg

SEQ ID NO: 51 - DNA, 20 bp, Artificial Sequence
  source: /note=“Description of Artificial Sequence Synthetic primer”
tcgctggtatcactcgtctg

SEQ ID NO: 52 - DNA, 20 bp, Artificial Sequence
  source: /note=“Description of Artificial Sequence Synthetic primer”
gattctgaagaccgccagag

SEQ ID NO: 53 - DNA, 20 bp, Artificial Sequence
  source: /note=“Description of Artificial Sequence Synthetic primer”
ctctcctgttgtgcttctcc

SEQ ID NO: 54 - DNA, 22 bp, Artificial Sequence
  source: /note=“Description of Artificial Sequence Synthetic primer”
gtcaaagttcatcctgtccttg